(19) United States
(12) Patent Application Publication
(10) Pub. No.: US 2024/0035029 A1
(43) Pub. Date: Feb. 1, 2024

(54) RNA COMPOSITIONS AND METHODS FOR INHIBITING LIPOPROTEIN(A)

(71) Applicant: SANOFI, Paris (FR)

(72) Inventors: Bodo BRUNNER, Frankfurt (DE); Bertrand FROTTIER, Paris (FR); Etienne GUILLOT, Paris (FR); Mike HELMS, Frankfurt am Main (DE); Armin HOFMEISTER, Frankfurt am Main (DE); Kerstin JAHN-HOFMANN, Frankfurt am Main (DE); Sabine SCHEIDLER, Frankfurt am Main (DE)

(21) Appl. No.: 18/248,974

(22) PCT Filed: Oct. 15, 2021
(86) PCT No.: PCT/EP2021/078569

§ 371 Date: Apr. 13, 2023

(30) Foreign Application Priority Data
Oct. 16, 2020 (EP) 20306222.9

(51) Int. Cl. C12N 15/113

(52) U.S. Cl. CPC C12N 15/113; C12N 2310/14; C12N 2310/351; C12N 2310/323; C12N 2310/317; C12N 2310/314

(57) ABSTRACT

The present disclosure relates to dsRNAs targeting LPA mRNA and modulating Lp(a) plasma levels, and methods of treating one or more conditions associated with LPA gene expression.

### SEQUENCE LISTING

Nucleic acid sequences are disclosed in the present specification that serve as references. The same sequences are also presented in a sequence listing formatted according to standard requirements for the purpose of patent matters. In case of any sequence discrepancy with the standard sequence listing, the sequences described in the present specification shall be the reference.

### FIELD OF THE INVENTION

The present invention relates to dsRNAs targeting LPA mRNA and modulating Lp(a) plasma levels, and methods of treating one or more conditions associated with LPA gene expression

### BACKGROUND OF THE INVENTION

Lipoproteins are lipid protein particles that play a key role in transporting lipids in plasma. These particles have a single-layer phospholipid and cholesterol membrane with embedded apolipoproteins (proteins that bind lipids) such as apoA, apoB, apoC, and apoE. The membrane encapsulates lipids being transported. Because lipids are not soluble in water, lipoproteins effectively serve as emulsifiers.

Lipoprotein(a) or Lp(a), found only in humans and in old-world monkeys, comprises a low density lipoprotein (LDL) particle. Lp(a) differs from other lipoproteins by the presence of a unique apolipoprotein, apolipoprotein(a) [apo(a)], which is linked to apoB₁₀₀ on the LDL particle outer surface through a disulfide bond (see, e.g., Kronenberg and Utermann, J Intern Med. (2013) 273(1):6-30); Guerra et al., Circulation. (2005) 111:1471-9). Apo(a) is expressed primarily in the liver and contains an inactive peptidase domain. Apo(a) is encoded by the highly polymorphic LPA gene. A variable number of kringle (K) IV type 2 repeats in the gene leads to a wide range of apo(a) isoform sizes. The LPA gene evolved from the plasminogen gene (PLG) and the two genes have highly homologous sequences (Kronenberg, supra).

Plasma Lp(a) levels vary by almost 1000-fold among individuals, with approximately of the population having highly elevated Lp(a) levels (approximately ≥50 mg/dL). See, e.g., Hopewell et al., J Intern Med. (2013) 273(1):260-8; Wilson et al., Clinical Lipidology (2019) 13(3):374-92. High plasma Lp(a) levels and small apo(a) isoform sizes are associated with an increased risk of cardiovascular diseases, including coronary heart disease, myocardial infarction, stroke, peripheral arterial disease, calcific aortic valve disease, and atherosclerosis.

WO 2019/092283 and WO 2020/099476 both disclose nucleic acids for inhibiting expression of LPA in a cell. Also, WO 2014/179625 discloses compositions and methods for modulating apolipoprotein(a) expression.

Double-stranded RNA molecules (dsRNAs) have been shown to block gene expression in a highly conserved regulatory mechanism known as RNA interference (RNAi). This appears to be a different mechanism of action from that of single-stranded oligonucleotides such as antisense oligonucleotides, antimiRs, and antagomiRs. In RNA interference technology, double-stranded RNAs, such as small interfering RNAs (siRNAs), bind to the RNA-induced silencing complex (“RISC”), where one strand (the “passenger strand” or “sense strand”) is displaced and the remaining strand (the “guide strand” or “antisense strand”) cooperates with RISC to bind a complementary RNA (the target RNA). Once bound, the target RNA is cleaved by RNA endonuclease Argonaute (AGO) in RISC and then further degraded by RNA exonucleases. RNAi has now been used to develop a new class of therapeutic agents for treating disorders caused by the aberrant or unwanted expression of a gene.

Due to the importance of Lp(a) in transporting cholesterol and oxidized phospholipids, and in providing lysophosphatidic acid, as well as the prevalence of diseases associated with elevated Lp(a) and atherosclerosis-promoting lipids, there is an urgent need to identify inhibitors of LPA expression and to test such inhibitors for efficacy and unwanted side effects such as cytotoxicity.

### SUMMARY OF THE INVENTION

The present disclosure provides a double-stranded ribonucleic acid (dsRNA) that inhibits expression of a human LPA gene by targeting a target sequence on an RNA transcript of the LPA gene, wherein the dsRNA comprises a sense strand comprising a sense sequence, and an antisense strand comprising an antisense sequence, the target sequence is nucleotides 220-238, 223-241, 302-320, 1236-1254, 2946-2964, 2953-2971, 2954-2972, 2958-2976, 2959-2977, 4635-4653, 4636-4654, 4639-4657, 4842-4860, 4980-4998, 4982-5000, 6385-6403, or 6470-6488 of SEQ ID NO: 1632, and wherein the sense sequence is at least 90% identical to the target sequence. In some embodiments, the sense strand and antisense strand are complementary to each other over a region of 15-25 contiguous nucleotides. In some embodiments, the sense strand and the antisense strand are no more than 30 nucleotides in length. In particular embodiments, the target sequence is nucleotides 2958-2976, 4639-4657, or 4982-5000 of SEQ ID NO: 1632.

Most preferred target sequences are nucleotides 2958-2976, 4639-4657 and 4982-5000.

In some embodiments, one or both strands of the dsRNA comprise one or more compounds having the structure of

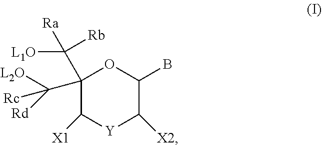

wherein:
B is a heterocyclic nucleobase,
one of L1 and L2 is an internucleoside linking group linking the compound of formula (I) to said strand(s) and the other of L1 and L2 is H, a protecting group, a phosphorus moiety or an internucleoside linking group linking the compound of formula (I) to said strand(s),
Y is O, NH, NR1 or N—C(═O)—R1, wherein R1 is:
   a (C1-C20) alkyl group, optionally substituted by one or more groups selected from an halogen atom, a (C1-C6) alkyl group, a (C3-C8) cycloalkyl group, a (C3-C14) heterocycle, a (C6-C14) aryl group, a (C5-C14) heteroaryl group, —O—Z1, —N(Z1)(Z2), —S—Z1, —CN, —C(=J)-O—Z1, —O—C(=J)-Z1, —C(=J)-N(Z1)(Z2), and —N(Z1)-C(=J)-Z2, wherein J is O or S,
      each of Z1 and Z2 is, independently, H, a (C1-C6) alkyl group, optionally substituted by one or more groups selected from a halogen atom and a (C1-C6) alkyl group,
a (C3-C8) cycloalkyl group, optionally substituted by one or more groups selected from a halogen atom and a (C1-C6) alkyl group,
a group —[C(═O)]m-R2-(O—CH₂—CH₂)p-R3, wherein
   m is an integer meaning 0 or 1,
   p is an integer ranging from 0 to 10,
   R2 is a (C1-C20) alkylene group optionally substituted by a (C1-C6) alkyl group, —O—Z3, —N(Z3)(Z4), —S—Z3, —CN, —C(═K)—O—Z3, —O—C(═K)—Z3, —C(═K)—N(Z3)(Z4), or —N(Z3)-C(═K)—Z4, wherein

### K is O or S,

each of Z3 and Z4 is, independently, H, a (C1-C6) alkyl group, optionally substituted by one or more groups selected from a halogen atom and a (C1-C6) alkyl group, and
R3 is selected from the group consisting of a hydrogen atom, a (C1-C6) alkyl group, a (C1-C6) alkoxy group, a (C3-C8) cycloalkyl group, a (C3-C14) heterocycle, a (C6-C14) aryl group or a (C5-C14) heteroaryl group,
or R3 is a cell targeting moiety,
X1 and X2 are each, independently, a hydrogen atom, a (C1-C6) alkyl group, and
each of Ra, Rb, Rc and Rd is, independently, H or a (C1-C6) alkyl group, or a pharmaceutically acceptable salt thereof.

In another aspect, the present disclosure provides a pharmaceutical composition comprising the present dsRNA and a pharmaceutically acceptable excipient, and the dsRNA and pharmaceutical composition for use in inhibiting LPA expression, reducing Lp(a) levels, or treating an Lp(a)-associated condition in a human in need thereof. In some embodiments, the human has, or is at risk of having, a lipid metabolism disorder or a cardiovascular disease (CVD). In further embodiments, the human has, or is at risk of having, hypercholesterolemia, dyslipidemia, myocardial infarction, atherosclerotic cardiovascular disease, atherosclerosis, peripheral artery disease, calcific aortic valve disease, thrombosis, or stroke.

### BRIEF DESCRIPTION OF THE DRAWINGS

FIGS. 1A and 1B are graphs showing correlation analyses of LPA siRNA screening results. A screening library comprising 299 LPA siRNAs was tested at 1 nM (FIG. 1A) or 10 nM (FIG. 1B) in two independent experiments in Hep3B cells transiently transfected with a pmirGLO-LPA dual luciferase reporter plasmid.

FIGS. 2A-C are graphs showing RT-qPCR analysis of LPA mRNA expression in human HepG2-LPA cells (which stably overexpressed a human LPA cDNA construct) (FIG. 2A), primary transgenic apo(a) mouse hepatocytes (FIG. 2B), or primary cynomolgus hepatocytes (FIG. 2C), following treatment with 34 selected test siRNAs at 1 or 10 nM. Expression of mRNA is represented relative to cells treated with a LV2 non-targeting siRNA control. Error bars indicate standard deviation. LV2 and LV3: negative control siRNA sequences that do not target any human, cynomolgus monkey, or rodent mRNA transcript. s8263 and s8264: positive controls, which are human LPA tool siRNAs (Ambion, now Thermo Fisher).

FIGS. 3A-C are graphs showing RT-qPCR analysis of plasminogen (PLG) mRNA expression in human HuH-7 cells (FIG. 3A), primary human hepatocytes (FIG. 3B), or primary cynomolgus hepatocytes (FIG. 3C) following treatment with 34 selected test siRNAs as indicated at 1 or 10 nM. Expression of mRNA is represented relative to cells treated with a LV2 non-targeting siRNA control. Error bars indicate standard deviation.

FIG. 4 is a graph depicting cytotoxic effects of 34 selected test siRNAs in human HepG2-LPA cells. Cells were treated with siRNAs as indicated at 5 or 50 nM before being analyzed for viability (CellTiter-Glo® assay) and toxicity (ToxiLight™ assay). Ratios of the resulting readings are shown relative to results for a LV2 non-targeting siRNA control. Error bars indicate standard deviation. “AllStars Cell Death”: AllStars Hs Cell Death Control siRNA (Qiagen).

FIG. 5 is a graph depicting relative amount of PLG protein secreted into the supernatant of human hepatocytes treated with indicated concentrations (0.1, 1, or 10 μM) of 17 selected LPA GalNAc-siRNAs under free uptake conditions as determined by ELISA assay. Protein expression is represented relative to cells treated with a LV2 non-targeting siRNA control at 1 μM (dashed line). Error bars indicate standard deviation.

FIG. 6 is a graph depicting analysis of cytotoxic siRNA effects in human HepG2-LPA cells. Cells were treated with 17 selected LPA GalNAc-siRNAs as indicated at 5 and 50 nM before being analyzed for viability (CellTiter-Glo assay) and toxicity (ToxiLight assay). Ratios of the resulting readings are shown relative to results of a LV2 non-targeting siRNA control (dashed line). Error bars indicate standard deviation.

FIG. 7 is a graph depicting the amount of interferon α2a (IFNα2a) protein released into the supernatant of human peripheral blood mononuclear cells (PBMCs) isolated from three different donors and transfected with 100 nM concentration of 17 selected LPA GalNAc-siRNAs or controls. Protein concentration was determined by ELISA. Error bars indicate standard deviation.

FIG. 8 is a graph depicting relative amounts of serum apo(a) protein levels in apo(a) transgenic mice treated subcutaneously with a single dose of 17 selected LPA GalNAc-siRNAs at mg/kg at day 0. Protein expression is represented relative to animals treated with a PBS vehicle control. Human apo(a) levels were quantified by ELISA, error bars indicate standard error of the mean (SEM).

FIG. 9 is a panel of graphs showing RNA-Seq whole transcriptome analysis of primary human hepatocytes from two different donors treated with 5 μM of three selected GalNAc-siRNAs. The number of differentially up- and downregulated genes as compared to a LV2 GalNAc-siRNA non-silencing control are shown applying the filter criteria—absolute foldchange>1.5 and FDR (false discovery rate)<0.05. LPA being the most downregulated transcript in each comparison is indicated by a dashed circle.

FIG. 10 is a graph depicting residual LPA mRNA expression levels normalized to a LV2 non-silencing control in primary hepatocytes isolated from apo(a) transgenic mice treated with 1 nM and 5 nM siRNAs from optimization libraries based on selected sequences siLPA #0307, siLPA #0311, and siLPA #0314.

FIGS. 11A-C are graphs showing relative amounts of serum apo(a) levels in apo(a) transgenic mice treated subcutaneously with a single dose of 41 optimized LPA GalNAc-siRNAs and respective parent molecules at 3 mg/kg at day 0. FIGS. 11A-C represent data for optimized LPA GalNAc-siRNAs based on parent sequences siLPA #0307; siLPA #0311, and siLPA #0314, respectively. Protein expression is represented relative to animals treated with a PBS vehicle control. Human apo(a) levels were quantified by ELISA, error bars indicate SEM.

FIG. 12 is a graph showing the amount of interferon α2a (IFNα2a) protein released into the supernatant of human peripheral blood mononuclear cells (PBMCs) isolated from three different donors and transfected with 100 nM concentration of 41 optimized LPA GalNAc-siRNAs or controls. Protein concentration was determined by ELISA. Error bars indicate standard deviation.

FIG. 13 is a graph showing RT-qPCR analysis of LPA mRNA expression in primary cynomolgus hepatocytes treated under free uptake conditions with 41 optimized LPA GalNAc-siRNAs and respective parent lead molecules as indicated at 100 nM and 1 μM concentration, respectively. mRNA expression is represented relative to cells treated with a LV2 non-targeting GalNAc-siRNA control (dashed line). Error bars indicate standard deviation.

FIG. 14 is a graph showing RT-qPCR analysis of PLG mRNA expression in primary human hepatocytes treated under free uptake conditions with 41 optimized LPA siRNA-GalNAc reagents and respective parent lead molecules as indicated at 10 nM, 100 nM and 1 μM concentration, respectively. mRNA expression is represented relative to cells treated with a LV2 non-targeting siRNA-GalNAc control (dashed line). Error bars indicate standard deviation.

### DETAILED DESCRIPTION OF THE INVENTION

The present disclosure provides novel double-stranded RNAs (dsRNAs) that inhibit expression of an LPA gene. In some embodiments, the dsRNAs are small interfering RNAs (siRNAs). Besides nucleic acids, the present dsRNAs may comprise additional moieties such as targeting moieties that facilitate the delivery of the dsRNAs to a targeted tissue. The dsRNAs can be used to treat conditions such as cardiovascular diseases. Unless otherwise stated, “apo(a)” refers to a human LPA gene product. An mRNA sequence of 6489 nucleotides in length of a human apo(a) protein is available under NCBI Reference Sequence No. NM_005577.2 (SEQ ID NO: 1632). An mRNA sequence of 6414 nucleotides in length, lacking the 75 first nucleotides located at the 5′ end of SEQ ID NO. 1632, of a human apo(a) protein is also available under NCBI Reference Sequence No. NM_005577.3 (SEQ ID NO: 1627) and its polypeptide sequence is available under NCBI Reference Sequence No. NP_005568.2 (SEQ ID NO: 1628). In certain embodiments, the present disclosure refers to cynomolgus apo(a). An mRNA sequence of a cynomolgus apo(a) protein is available under NCBI Reference Sequence No. XM_015448517 (SEQ ID NO: 1629) and its polypeptide sequence is available under NCBI Reference Sequence No. XP_015304003.1 (SEQ ID NO: 1630).

A dsRNA of the present disclosure, such as one comprising a conjugated GalNAc moiety, may have one or more of the following properties: (i) has a half-life of at least 24, 28, 32, 48, 52, 56, 60, 72, 96, or 168 hours in 50% mouse serum; (ii) does not increase production of interferon α secreted from human primary PMBCs; (iii) has an IC₅₀ value of from, e.g., 1 pM to 100 nM, for inhibition of human LPA mRNA expression in transgenic mouse hepatocytes or primary human or cynomolgus liver cells; and (iv) reduces protein levels of apo(a) by at least 75%, 80%, 85%, 90%, 95%, 96%, 97%, 98%, or 99% in vivo in FVB/N mice expressing human LPA.

In some embodiments, a dsRNA of the present disclosure comprising a conjugated GalNAc moiety has at least one of the following properties: (i) has a half-life of at least 24 hours in 50% mouse serum; (ii) does not increase production of interferon α secreted from human primary PMBCs, (iii) has an IC₅₀ value of from, e.g., 1 pM to 50 nM, for inhibition of human LPA mRNA expression in transgenic mouse hepatocytes or primary human or cynomolgus liver cells; and (iv) reduces protein levels of human apo(a) by at least 80% in vivo in FVB/N mice expressing human LPA. In certain embodiments, the dsRNA has all of said properties.

It will be understood by the person skilled in the art that the dsRNAs described herein do not occur in nature (“isolated” dsRNAs).

### I. Double-Stranded RNAs

Certain aspects of the present disclosure relate to double-stranded ribonucleic acid (dsRNA) molecules targeting LPA mRNA. As used herein, the term “double-stranded RNA” or “dsRNA” refers to an oligoribonucleotide molecule comprising a duplex structure having two anti-parallel and substantially complementary nucleic acid strands. The two strands forming the duplex structure may be different portions of one larger RNA molecule, or they may be on separate RNA molecules. When the two strands are on separate RNA molecules, the dsRNA structure may function as short interfering RNA (siRNA). Where the two strands are part of one larger molecule and are connected by an uninterrupted chain of nucleotides between the 3′-end of a first strand and the 5′-end of a second strand, the connecting RNA chain is referred to as a “hairpin loop” and the RNA molecule may be termed “short hairpin RNA,” or “shRNA.” The RNA strands may have the same or a different number of nucleotides. In addition to the duplex structure, a dsRNA may comprise overhangs of one or more (e.g., 1, 2 or 3) nucleotides.

As used herein, the term “polynucleotide” refers to a polymeric form of nucleotides of at least 10 bases in length, either ribonucleotides or deoxyribonucleotides or a modified form of either type of nucleotide. The term includes single and double stranded forms.

A “dsRNA” may include naturally occurring ribonucleotides, and/or chemically modified analogs thereof. As used herein, “dsRNAs” are not limited to those with ribose-containing nucleotides. A dsRNA herein encompasses a double-stranded polynucleotide molecule where the ribose moiety in some or all of its nucleotides has been replaced by another moiety, so long as the resultant double-stranded molecule can inhibit the expression of a target gene by RNA interference. The dsRNA may also include one or more, but not more than 60% (e.g., not more than 50%, 40%, 30%, 20%, or 10%) deoxyribonucleotides or chemically modified analogs thereof.

A dsRNA of the present disclosure comprises a sense strand comprising a sense sequence, and an antisense strand comprising an antisense sequence, wherein the sense strand and the antisense strand are sufficiently complementary to hybridize to form a duplex structure. The term “antisense sequence” refers to a sequence that is substantially or fully complementary, and binds under physiological conditions, to a target RNA sequence in a cell. A “target sequence” refers to a nucleotide sequence on an RNA molecule (e.g., a primary RNA transcript or a messenger RNA transcript) transcribed from a target gene, e.g., an LPA gene. The term “sense sequence” refers to a sequence that is substantially or fully complementary to the antisense sequence.

The LPA mRNA-targeting dsRNA of the present disclosure comprises a sense strand comprising a sense sequence and an antisense strand comprising an antisense sequence, wherein the sense and antisense sequences are substantially or fully complementary to each other. Unless otherwise indicated, the term “complementary” refers herein to the ability of a polynucleotide comprising a first contiguous nucleotide sequence, under certain conditions, e.g., physiological conditions, to hybridize to and form a duplex structure with another polynucleotide comprising a second contiguous nucleotide sequence. This may include base-pairing of the two polynucleotides over the entire length of the first or second contiguous nucleotide sequence; in this case, the two nucleotide sequences are considered “fully complementary” to each other. For example, in a case where a dsRNA comprises a first oligonucleotide 21 nucleotides in length and a second oligonucleotide 23 nucleotides in length, and where the two oligonucleotides form 21 contiguous base-pairs, the two oligonucleotides may be referred to as “fully complementary” to each other. Where a first polynucleotide sequence is referred to as “substantially complementary” to a second polynucleotide sequence, the two sequences may base-pair with each other over 80% or more (e.g., 90% or more) of their length of hybridization, with no more than 20% (e.g., no more than 10%) of mismatching base-pairs (e.g., for a duplex of 20 nucleotides, no more than 4 or no more than 2 mismatched base-pairs). Where two oligonucleotides are designed to form a duplex with one or more single-stranded overhangs, such overhangs shall not be regarded as mismatches for the determination of complementarity. Complementarity of two sequences may be based on Watson-Crick base-pairs and/or non-Watson-Crick base-pairs. As used herein, a polynucleotide which is “substantially complementary to at least part of” an mRNA refers to a polynucleotide which is substantially complementary to a contiguous portion of an mRNA of interest (e.g., an mRNA encoding LPA).

In some embodiments, the LPA-targeting dsRNA is an siRNA where the sense and antisense strands are not covalently linked to each other. In some embodiments, the sense and antisense strands of the LPA-targeting dsRNA are covalently linked to each other, e.g., through a hairpin loop (such as in the case of shRNA), or by means other than a hairpin loop (such as by a connecting structure referred to as a “covalent linker”).

I.1 Lengths

In some embodiments, each of the sense sequence (in the sense strand) and the antisense sequence (in the antisense strand) is 9-30 nucleotides in length. For example, each sequence can be any of a range of nucleotide lengths having an upper limit of 21, 22, 23, 24, 25, 26, 27, 28, 29, or 30 and an independently selected lower limit of 9, 10, 11, 12, 13, 14, 15, 16, 17, 18, 19, or 20. In some embodiments, the number of nucleotides in each sequence may be 15-25 (i.e., 15 to 25 nucleotides in each sequence), 15-30, 16-29, 17-28, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, or 19-21.

In some embodiments, each sequence is greater than 8, 9, 10, 11, 12, 13, 14, 15, 16, 17, 18, 19, 20, 21, 22, 23, 24, 25, 26, 27, 28, 29, or 30 nucleotides in length. In some embodiments, each sequence is less than 21, 22, 23, 24, 25, 26, 27, 28, 29, 30, or 31 nucleotides in length. In some embodiments, each sequence is 9, 10, 11, 12, 13, 14, 15, 16, 17, 18, 19, 20, 21, 22, 23, 24, 25, 26, 27, 28, 29, or 30 nucleotides in length.

In some embodiments, the sense and antisense sequences are each at least 15 and no greater than 25 nucleotides in length. In some embodiments, the sense and antisense sequences are each at least 19 and no greater than 23 nucleotides in length. For example, the sequences are 15, 16, 17, 18, 19, 20, 21, 22, 23, 24, or 25 nucleotides in length.

In some embodiments, the LPA mRNA-targeting dsRNA has sense and antisense strands of the same length or different lengths. For example, the sense strand may be 1, 2, 3, 4, 5, 6, or 7 nucleotides longer than the antisense strand. Alternatively, the sense strand may be 1, 2, 3, 4, 5, 6, or 7 nucleotides shorter than the antisense strand.

In some embodiments, each of the sense strand and the antisense strand is 9-36 nucleotides in length. For example, each strand can be any of a range of nucleotide lengths having an upper limit of 21, 22, 23, 24, 25, 26, 27, 28, 29, 30, 31, 32, 33, 34, 35, or 36 and an independently selected lower limit of 9, 10, 11, 12, 13, 14, 15, 16, 17, 18, 19, or 20. In some embodiments, the number of nucleotides in each strand may be 15-25, 15-30, 16-29, 17-28, 18-28, 18-27, 18-26, 18-18-24, 18-23, 18-22, 18-21, 18-20, 19-30, 19-29, 19-28, 19-27, 19-26, 19-25, 19-24, 19-23, 19-22, or 19-21.

In some embodiments, each strand is greater than 8, 9, 10, 11, 12, 13, 14, 15, 16, 17, 18, 19, 20, 21, 22, 23, 24, 25, 26, 27, 28, 29, or 30 nucleotides in length. In some embodiments, each strand is less than 20, 21, 22, 23, 24, 25, 26, 27, 28, 29, 30, 31, 32, 33, 34, 35, 36, or 37 nucleotides in length. In some embodiments, each strand is 9, 10, 11, 12, 13, 14, 15, 16, 17, 18, 19, 20, 21, 22, 23, 24, 25, 26, 27, 28, 29, 30, 31, 32, 33, 34, 35, or 36 nucleotides in length.

In some embodiments, the sense and antisense strands are each at least 15 and no greater than 25 nucleotides in length. In some embodiments, the sense and antisense strands are each at least 19 and no greater than 23 nucleotides in length. For example, the strands are 19, 20, 21, 22, or 23 nucleotides in length.

In some embodiments, the sense strand may have 21, 22, 23, or 24 nucleotides, including any modified nucleotides, while the antisense strand may have 21 nucleotides, including any modified nucleotides; in certain embodiments, the sense strand may have a sense sequence having 17, 18, or 19 nucleotides, while the antisense strand may have an antisense sequence having 19 nucleotides.

I.2 Overhangs

In some embodiments, a dsRNA of the present disclosure comprises one or more overhangs at the 3′-end, 5′-end, or both ends of one or both of the sense and antisense strands. In some embodiments, the one or more overhangs improve the stability and/or inhibitory activity of the dsRNA.

“Overhang” refers herein to the unpaired nucleotide(s) that protrude from the duplex structure of a dsRNA when a 3′ end of a first strand of the dsRNA extends beyond the 5′ end of a second strand, or vice versa. “Blunt end” means that there are no unpaired nucleotides at that end of the dsRNA, i.e., no nucleotide overhang. A “blunt-ended” dsRNA is a dsRNA that is double-stranded over its entire length, i.e., no nucleotide overhang at either end of the duplex molecule. Chemical caps or non-nucleotide chemical moieties conjugated to the 3′ end and/or the 5′ end of a dsRNA are not considered herein in determining whether a dsRNA has an overhang or not.

In some embodiments, an overhang comprises one or more, two or more, three or more, or four or more nucleotides. For example, the overhang may comprise 1, 2, 3, or 4 nucleotides.

In some embodiments, an overhang of the present disclosure comprises one or more nucleotides (e.g., ribonucleotides or deoxyribonucleotides, naturally occurring or chemically modified analogs thereof). In some embodiments, the overhang comprises one or more thymines or chemically modified analogs thereof. In certain embodiments, the overhang comprises one or more thymines.

In some embodiments, the dsRNA comprises an overhang located at the 3′-end of the antisense strand. In some embodiments, the dsRNA comprises a blunt end at the 5′-end of the antisense strand. In some embodiments, the dsRNA comprises an overhang located at the 3′-end of the antisense strand and a blunt end at the 5′-end of the antisense strand. In some embodiments, the dsRNA comprises an overhang located at the 3′-end of the sense strand. In some embodiments, the dsRNA comprises a blunt end at the 5′-end of the sense strand. In some embodiments, the dsRNA comprises an overhang located at the 3′-end of the sense strand and a blunt end at the 5′-end of the sense strand. In some embodiments, the dsRNA comprises overhangs located at the 3′-end of both the sense and antisense strands of the dsRNA.

In some embodiments, the dsRNA comprises an overhang located at the 5′-end of the antisense strand. In some embodiments, the dsRNA comprises a blunt end at the 3′-end of the antisense strand. In some embodiments, the dsRNA comprises an overhang located at the 5′-end of the antisense strand and a blunt end at the 3′-end of the antisense strand. In some embodiments, the dsRNA comprises an overhang located at the 5′-end of the sense strand. In some embodiments, the dsRNA comprises a blunt end at the 3′-end of the sense strand. In some embodiments, the dsRNA comprises an overhang located at the 5′-end of the sense strand and a blunt end at the 3′-end of the sense strand. In some embodiments, the dsRNA comprises overhangs located at both the 5′-end of the sense and antisense strands of the dsRNA.

In some embodiments, the dsRNA comprises an overhang located at the 3′-end of the antisense strand and an overhang at the 5′-end of the antisense strand. In some embodiments, the dsRNA comprises an overhang located at the 3′-end of the sense strand and an overhang at the 5′-end of the sense strand.

In some embodiments, the dsRNA has two blunt ends.

In some embodiments, the overhang is the result of the sense strand being longer than the antisense strand. In some embodiments, the overhang is the result of the antisense strand being longer than the sense strand. In some embodiments, the overhang is the result of sense and antisense strands of the same length being staggered. In some embodiments, the overhang forms a mismatch with the target mRNA. In some embodiments, the overhang is complementary to the target mRNA.

In some embodiments, one or both of the sense strand and the antisense strand of the dsRNA further comprise:
a) a 5′ overhang comprising one or more nucleotides; and/or
b) a 3′ overhang comprising one or more nucleotides.

In some embodiments, an overhang in the dsRNA comprises two or three nucleotides.

In certain embodiments, a dsRNA of the present disclosure contains a sense strand having the sequence of 5′-CCA-[sense sequence]-invdT, and the antisense strand having the sequence of 5′-[antisense sequence]-dTdT-3′, where the trinucleotide CCA may be modified (e.g., 2′-O-Methyl-C and 2′-O-Methyl-A).

I.3 Target and dsRNA Sequences

The antisense strand of a dsRNA of the present disclosure comprises an antisense sequence that may be substantially or fully complementary to a target sequence of 12-30 nucleotides in length in an LPA RNA (e.g., an mRNA). For example, the target sequence can be any of a range of nucleotide lengths having an upper limit of 19, 20, 21, 22, 23, 24, 25, 26, 27, 28, 29, or 30 and an independently selected lower limit of 12, 13, 14, 15, 16, 17, 18, or 19. In some embodiments, the number of nucleotides in the target sequence may be 15-25, 15-30, 16-29, 17-28, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, or 19-21.

In some embodiments, the target sequence is greater than 12, 13, 14, 15, 16, 17, 18, 19, or 20 nucleotides in length. In some embodiments, the target sequence is less than 21, 22, 23, 24, 26, 27, 28, 29, or 30 nucleotides in length. In some embodiments, the target sequence is 12, 13, 14, 15, 16, 17, 18, 19, 20, 21, 22, 23, 24, 25, 26, 27, 28, 29, or 30 nucleotides in length. In certain embodiments, the target sequence is at least 15 and no greater than 25 nucleotides in length; for example, at least 19 and no greater than 23 nucleotides in length, or 15, 16, 17, 18, 19, 20, 21, 22, 23, 24, or 25 nucleotides in length.

The target sequence may be in the 5′ noncoding region, the coding region, or the 3′ noncoding region of the LPA mRNA transcript. The target sequence may also be located at the junction of the coding and noncoding regions.

In some embodiments, the dsRNA antisense strand comprises an antisense sequence having one or more mismatch (e.g., one, two, three, or four mismatches) to the target sequence. In certain embodiments, the antisense sequence is fully complementary to the corresponding portion in the human LPA mRNA sequence and is fully complementary or substantially complementary (e.g., comprises at least one or two mismatches) to the corresponding portion in a cynomolgus LPA mRNA sequence. One advantage of such dsRNAs is to allow pre-clinical in vivo studies of the dsRNAs in non-human primates such as cynomolgus monkeys. In certain embodiments, the dsRNA sense strand comprises a sense sequence that is at least 70%, 75%, 80%, 85%, 90%, 95%, 96%, 97%, 98%, or 99% identical to the target sequence (e.g., in human or cynomolgus LPA mRNA).

In some embodiments, the target sequence in a human LPA mRNA sequence (SEQ ID NO: 1632) has the start and end nucleotide positions at or around (e.g., within 3 nucleotides of) the following nucleotides: 220 and 238, 223 and 241, 302 and 320, 1236 and 1254, 2946 and 2964, 2953 and 2971, 2954 and 2972, 2958 and 2976, 2959 and 2977, 4635 and 4653, 4636 and 4654, 4639 and 4657, 4842 and 4860, 4980 and 4998, 4982 and 5000, 6385 and 6403, or 6470 and 6488, respectively. In certain embodiments, the target sequence corresponds to nucleotide positions 2958-2976, 4639-4657, or 4982-5000 of the human LPA mRNA sequence, where the start and end positions may vary within 3 nucleotides of the numbered positions. In some embodiments, the target sequence is a sequence listed in Table 1 as a sense sequence, or a sequence that includes at least 80% nucleotides (e.g., at least 90%) of the listed sequence.

In some embodiments, a dsRNA of the present disclosure comprises a sense strand comprising a sense sequence shown in Table 1. For example, the sense strand comprises a sequence selected from SEQ ID NOs: 4, 7, 19, 90, 104, 107, 108, 110, 111, 168, 169, 172, 200, 221, 223, 279, and 298 or a sequence having at least 15, 16, 17, or 18 contiguous nucleotides derived from said selected sequence.

In some embodiments, a dsRNA of the present disclosure comprises an antisense strand comprising an antisense sequence shown in Table 1. In some embodiments, the antisense strand comprises a sequence selected from SEQ ID NOs: 303, 306, 318, 389, 403, 406, 407, 409, 410, 467, 468, 471, 499, 520, 522, 578, and 597 or a sequence having at least 15, 16, 17, or 18 contiguous nucleotides derived from said selected sequence. In a particular embodiment, the dsRNA comprises an antisense sequence that is at least 90% identical to a nucleotide sequence selected from the group consisting of SEQ ID NOs: 303, 306, 318, 389, 403, 406, 407, 409, 410, 467, 468, 471, 499, 520, 522, 578, and 597.

In a particular embodiment, the sense sequence and the antisense sequence are complementary, wherein:
a) the sense sequence comprises a nucleotide sequence selected from the group consisting of SEQ ID NOs: 4, 7, 19, 90, 104, 107, 108, 110, 111, 168, 169, 172, 200, 221, 223, 279, and 298; or
b) the antisense sequence comprises a nucleotide sequence selected from the group consisting of SEQ ID NOs: 303, 306, 318, 389, 403, 406, 407, 409, 410, 467, 468, 471, 499, 520, 522, 578, and 597.

In some embodiments, a dsRNA of the present disclosure comprises a sense strand comprising a sense sequence shown in Table 1 and an antisense strand comprising an antisense sequence shown in Table 1. In some embodiments, the sense and antisense strands respectively comprise the sequences of:
SEQ ID NOs: 4 and 303;
SEQ ID NOs: 7 and 306;
SEQ ID NOs: 19 and 318;
SEQ ID NOs: 90 and 389;
SEQ ID NOs: 104 and 403;
SEQ ID NOs: 107 and 406;
SEQ ID NOs: 108 and 407;
SEQ ID NOs: 110 and 409;
SEQ ID NOs: 111 and 410;
SEQ ID NOs: 168 and 467;
SEQ ID NOs: 169 and 468;
SEQ ID NOs: 172 and 471;
SEQ ID NOs: 200 and 499;
SEQ ID NOs: 221 and 520;
SEQ ID NOs: 223 and 522;
SEQ ID NOs: 279 and 578; or
SEQ ID NOs: 298 and 597.

In certain embodiments, the sense and antisense strands respectively comprise the sequences of:
SEQ ID NOs: 110 and 409;
SEQ ID NOs: 172 and 471; or
SEQ ID NOs: 223 and 522.

In some embodiments, the antisense sequence is fully complementary to a sequence selected from SEQ ID NOs: 110, 172, and 223. In some embodiments, the antisense sequence is substantially complementary to a sequence selected from SEQ ID NOs: 110, 172, and 223, wherein the antisense sequence comprises at least one mismatch (e.g., one, two, three, or four mismatches) to the selected sequence.

In some embodiments, the antisense sequence of the LPA mRNA-targeting dsRNA comprises one or more mismatches to the target sequence (for example, due to allelic differences among individuals in a general population). For example, the antisense sequence comprises one or more mismatches (e.g., one, two, three, or four mismatches) to the target sequence. In some embodiments, the one or more mismatches are not located in the center of the region of complementarity. In some embodiments, the one or more mismatches are located within five, four, three, two, or one nucleotide of the 5′ and/or 3′ ends of the region of complementarity. For example, for a dsRNA containing a 19 nucleotide antisense sequence, in some embodiments the antisense sequence may not contain any mismatch within the central 9 nucleotides of the region of complementarity between it and its target sequence in the LPA mRNA.

Table 1 below lists the sense and antisense sequences of exemplary siRNA constructs (CNST). The start (ST) and end (ED) nucleotide positions in NM_005577.2 (SEQ ID NO: 1632) are indicated. “SEQ” denotes SEQ ID NOs.

TABLE 1
Sequences of LPA siRNA Constructs

| CNS | | | Sense Sequence | | Antisense Sequence | |
|---|---|---|---|---|---|---|
| T# | ST | ED | (5′-3′) | SEQ | (5′-3′) | SEQ |
| 0001 | 5 | 23 | ACCUUUGGGGCUGGCUUUC | 1 | GAAAGCCAGCCCCAAAGGU | 300 |
| 0002 | 185 | 203 | GCCAUGUGGUCCAGGAUUG | 2 | CAAUCCUGGACCACAUGGC | 301 |
| 0003 | 219 | 237 | ACAGAGUUAUCGAGGCACG | 3 | CGUGCCUCGAUAACUCUGU | 302 |
| 0004 | 220 | 238 | CAGAGUUAUCGAGGCACGU | 4 | ACGUGCCUCGAUAACUCUG | 303 |
| 0005 | 221 | 239 | AGAGUUAUCGAGGCACGUA | 5 | UACGUGCCUCGAUAACUCU | 304 |
| 0006 | 222 | 240 | GAGUUAUCGAGGCACGUAC | 6 | GUACGUGCCUCGAUAACUC | 305 |
| 0007 | 223 | 241 | AGUUAUCGAGGCACGUACU | 7 | AGUACGUGCCUCGAUAACU | 306 |
| 0008 | 238 | 256 | UACUCCACCACUGUCACAG | 8 | CUGUGACAGUGGUGGAGUA | 307 |
| 0009 | 240 | 258 | CUCCACCACUGUCACAGGA | 9 | UCCUGUGACAGUGGUGGAG | 308 |
| 0010 | 259 | 277 | AGGACCUGCCAAGCUUGGU | 10 | ACCAAGCUUGGCAGGUCCU | 309 |
| 0011 | 261 | 279 | GACCUGCCAAGCUUGGUCA | 11 | UGACCAAGCUUGGCAGGUC | 310 |
| 0012 | 262 | 280 | ACCUGCCAAGCUUGGUCAU | 12 | AUGACCAAGCUUGGCAGGU | 311 |
| 0013 | 263 | 281 | CCUGCCAAGCUUGGUCAUC | 13 | GAUGACCAAGCUUGGCAGG | 312 |
| 0014 | 266 | 284 | GCCAAGCUUGGUCAUCUAU | 14 | AUAGAUGACCAAGCUUGGC | 313 |
| 0015 | 267 | 285 | CCAAGCUUGGUCAUCUAUG | 15 | CAUAGAUGACCAAGCUUGG | 314 |
| 0016 | 298 | 316 | CAUAAUAGGACCACAGAAA | 16 | UUUCUGUGGUCCUAUUAUG | 315 |
| 0017 | 300 | 318 | UAAUAGGACCACAGAAAAC | 17 | GUUUUCUGUGGUCCUAUUA | 316 |
| 0018 | 301 | 319 | AAUAGGACCACAGAAAACU | 18 | AGUUUUCUGUGGUCCUAUU | 317 |
| 0019 | 302 | 320 | AUAGGACCACAGAAAACUA | 19 | UAGUUUUCUGUGGUCCUAU | 318 |
| 0020 | 303 | 321 | UAGGACCACAGAAAACUAC | 20 | GUAGUUUUCUGUGGUCCUA | 319 |
| 0021 | 324 | 342 | AAAUGCUGGCUUGAUCAUG | 21 | CAUGAUCAAGCCAGCAUUU | 320 |
| 0022 | 330 | 348 | UGGCUUGAUCAUGAACUAC | 22 | GUAGUUCAUGAUCAAGCCA | 321 |
| 0023 | 331 | 349 | GGCUUGAUCAUGAACUACU | 23 | AGUAGUUCAUGAUCAAGCC | 322 |
| 0024 | 372 | 390 | AGCUCCUUAUUGUUAUACG | 24 | CGUAUAACAAUAAGGAGCU | 323 |
| 0025 | 373 | 391 | GCUCCUUAUUGUUAUACGA | 25 | UCGUAUAACAAUAAGGAGC | 324 |
| 0026 | 413 | 431 | AGUACUGCAACCUGACGCA | 26 | UGCGUCAGGUUGCAGUACU | 325 |
| 0027 | 414 | 432 | GUACUGCAACCUGACGCAA | 27 | UUGCGUCAGGUUGCAGUAC | 326 |
| 0028 | 415 | 433 | UACUGCAACCUGACGCAAU | 28 | AUUGCGUCAGGUUGCAGUA | 327 |
| 0029 | 418 | 436 | UGCAACCUGACGCAAUGCU | 29 | AGCAUUGCGUCAGGUUGCA | 328 |
| 0030 | 419 | 437 | GCAACCUGACGCAAUGCUC | 30 | GAGCAUUGCGUCAGGUUGC | 329 |
| 0031 | 420 | 438 | CAACCUGACGCAAUGCUCA | 31 | UGAGCAUUGCGUCAGGUUG | 330 |
| 0032 | 421 | 439 | AACCUGACGCAAUGCUCAG | 32 | CUGAGCAUUGCGUCAGGUU | 331 |
| 0033 | 422 | 440 | ACCUGACGCAAUGCUCAGA | 33 | UCUGAGCAUUGCGUCAGGU | 332 |
| 0034 | 423 | 441 | CCUGACGCAAUGCUCAGAC | 34 | GUCUGAGCAUUGCGUCAGG | 333 |
| 0035 | 424 | 442 | CUGACGCAAUGCUCAGACG | 35 | CGUCUGAGCAUUGCGUCAG | 334 |
| 0036 | 425 | 443 | UGACGCAAUGCUCAGACGC | 36 | GCGUCUGAGCAUUGCGUCA | 335 |
| 0037 | 465 | 483 | UCCGACUGUUACCCCGGUU | 37 | AACCGGGGUAACAGUCGGA | 336 |
| 0038 | 469 | 487 | ACUGUUACCCCGGUUCCAA | 38 | UUGGAACCGGGGUAACAGU | 337 |
| 0039 | 470 | 488 | CUGUUACCCCGGUUCCAAG | 39 | CUUGGAACCGGGGUAACAG | 338 |
| 0040 | 471 | 489 | UGUUACCCCGGUUCCAAGC | 40 | GCUUGGAACCGGGGUAACA | 339 |
| 0041 | 473 | 491 | UUACCCCGGUUCCAAGCCU | 41 | AGGCUUGGAACCGGGGUAA | 340 |
| 0042 | 474 | 492 | UACCCCGGUUCCAAGCCUA | 42 | UAGGCUUGGAACCGGGGUA | 341 |
| 0043 | 480 | 498 | GGUUCCAAGCCUAGAGGCU | 43 | AGCCUCUAGGCUUGGAACC | 342 |
| 0044 | 481 | 499 | GUUCCAAGCCUAGAGGCUC | 44 | GAGCCUCUAGGCUUGGAAC | 343 |
| 0045 | 489 | 507 | CCUAGAGGCUCCUUCCGAA | 45 | UUCGGAAGGAGCCUCUAGG | 344 |
| 0046 | 490 | 508 | CUAGAGGCUCCUUCCGAAC | 46 | GUUCGGAAGGAGCCUCUAG | 345 |
| 0047 | 495 | 513 | GGCUCCUUCCGAACAAGCA | 47 | UGCUUGUUCGGAAGGAGCC | 346 |
| 0048 | 499 | 517 | CCUUCCGAACAAGCACCGA | 48 | UCGGUGCUUGUUCGGAAGG | 347 |
| 0049 | 500 | 518 | CUUCCGAACAAGCACCGAC | 49 | GUCGGUGCUUGUUCGGAAG | 348 |
| 0050 | 501 | 519 | UUCCGAACAAGCACCGACU | 50 | AGUCGGUGCUUGUUCGGAA | 349 |
| 0051 | 502 | 520 | UCCGAACAAGCACCGACUG | 51 | CAGUCGGUGCUUGUUCGGA | 350 |
| 0052 | 503 | 521 | CCGAACAAGCACCGACUGA | 52 | UCAGUCGGUGCUUGUUCGG | 351 |
| 0053 | 505 | 523 | GAACAAGCACCGACUGAGC | 53 | GCUCAGUCGGUGCUUGUUC | 352 |
| 0054 | 506 | 524 | AACAAGCACCGACUGAGCA | 54 | UGCUCAGUCGGUGCUUGUU | 353 |
| 0055 | 507 | 525 | ACAAGCACCGACUGAGCAA | 55 | UUGCUCAGUCGGUGCUUGU | 354 |
| 0056 | 508 | 526 | CAAGCACCGACUGAGCAAA | 56 | UUUGCUCAGUCGGUGCUUG | 355 |
| 0057 | 509 | 527 | AAGCACCGACUGAGCAAAG | 57 | CUUUGCUCAGUCGGUGCUU | 356 |
| 0058 | 510 | 528 | AGCACCGACUGAGCAAAGG | 58 | CCUUUGCUCAGUCGGUGCU | 357 |
| 0059 | 552 | 570 | UGGUAAUGGACAGAGUUAU | 59 | AUAACUCUGUCCAUUACCA | 358 |
| 0060 | 554 | 572 | GUAAUGGACAGAGUUAUCG | 60 | CGAUAACUCUGUCCAUUAC | 359 |
| 0061 | 555 | 573 | UAAUGGACAGAGUUAUCGA | 61 | UCGAUAACUCUGUCCAUUA | 360 |
| 0062 | 563 | 581 | AGAGUUAUCGAGGCACAUA | 62 | UAUGUGCCUCGAUAACUCU | 361 |
| 0063 | 564 | 582 | GAGUUAUCGAGGCACAUAC | 63 | GUAUGUGCCUCGAUAACUC | 362 |
| 0064 | 565 | 583 | AGUUAUCGAGGCACAUACU | 64 | AGUAUGUGCCUCGAUAACU | 363 |
| 0065 | 566 | 584 | GUUAUCGAGGCACAUACUC | 65 | GAGUAUGUGCCUCGAUAAC | 364 |
| 0066 | 567 | 585 | UUAUCGAGGCACAUACUCC | 66 | GGAGUAUGUGCCUCGAUAA | 365 |
| 0067 | 574 | 592 | GGCACAUACUCCACCACUG | 67 | CAGUGGUGGAGUAUGUGCC | 366 |
| 0068 | 578 | 596 | CAUACUCCACCACUGUCAC | 68 | GUGACAGUGGUGGAGUAUG | 367 |
| 0069 | 598 | 616 | GGAAGAACCUGCCAAGCUU | 69 | AAGCUUGGCAGGUUCUUCC | 368 |
| 0070 | 624 | 642 | UAUGACACCACACUCGCAU | 70 | AUGCGAGUGUGGUGUCAUA | 369 |
| 0071 | 625 | 643 | AUGACACCACACUCGCAUA | 71 | UAUGCGAGUGUGGUGUCAU | 370 |
| 0072 | 626 | 644 | UGACACCACACUCGCAUAG | 72 | CUAUGCGAGUGUGGUGUCA | 371 |
| 0073 | 627 | 645 | GACACCACACUCGCAUAGU | 73 | ACUAUGCGAGUGUGGUGUC | 372 |
| 0074 | 628 | 646 | ACACCACACUCGCAUAGUC | 74 | GACUAUGCGAGUGUGGUGU | 373 |
| 0075 | 630 | 648 | ACCACACUCGCAUAGUCGG | 75 | CCGACUAUGCGAGUGUGGU | 374 |
| 0076 | 631 | 649 | CCACACUCGCAUAGUCGGA | 76 | UCCGACUAUGCGAGUGUGG | 375 |
| 0077 | 632 | 650 | CACACUCGCAUAGUCGGAC | 77 | GUCCGACUAUGCGAGUGUG | 376 |
| 0078 | 633 | 651 | ACACUCGCAUAGUCGGACC | 78 | GGUCCGACUAUGCGAGUGU | 377 |
| 0079 | 640 | 658 | CAUAGUCGGACCCCAGAAU | 79 | AUUCUGGGGUCCGACUAUG | 378 |
| 0080 | 641 | 659 | AUAGUCGGACCCCAGAAUA | 80 | UAUUCUGGGGUCCGACUAU | 379 |
| 0081 | 642 | 660 | UAGUCGGACCCCAGAAUAC | 81 | GUAUUCUGGGGUCCGACUA | 380 |
| 0082 | 643 | 661 | AGUCGGACCCCAGAAUACU | 82 | AGUAUUCUGGGGUCCGACU | 381 |
| 0083 | 644 | 662 | GUCGGACCCCAGAAUACUA | 83 | UAGUAUUCUGGGGUCCGAC | 382 |
| 0084 | 645 | 663 | UCGGACCCCAGAAUACUAC | 84 | GUAGUAUUCUGGGGUCCGA | 383 |
| 0085 | 646 | 664 | CGGACCCCAGAAUACUACC | 85 | GGUAGUAUUCUGGGGUCCG | 384 |
| 0086 | 1191 | 1209 | ACAAGCACCGACUGAGCAG | 86 | CUGCUCAGUCGGUGCUUGU | 385 |
| 0087 | 1193 | 1211 | AAGCACCGACUGAGCAGAG | 87 | CUCUGCUCAGUCGGUGCUU | 386 |
| 0088 | 1234 | 1252 | CACGGUAAUGGACAGAGUU | 88 | AACUCUGUCCAUUACCGUG | 387 |
| 0089 | 1235 | 1253 | ACGGUAAUGGACAGAGUUA | 89 | UAACUCUGUCCAUUACCGU | 388 |
| 0090 | 1236 | 1254 | CGGUAAUGGACAGAGUUAU | 90 | AUAACUCUGUCCAUUACCG | 389 |
| 0091 | 2867 | 2885 | UUACCCCGAUUCCAAGCCU | 91 | AGGCUUGGAAUCGGGGUAA | 390 |
| 0092 | 2868 | 2886 | UACCCCGAUUCCAAGCCUA | 92 | UAGGCUUGGAAUCGGGGUA | 391 |
| 0093 | 2869 | 2887 | ACCCCGAUUCCAAGCCUAG | 93 | CUAGGCUUGGAAUCGGGGU | 392 |
| 0094 | 2870 | 2888 | CCCCGAUUCCAAGCCUAGA | 94 | UCUAGGCUUGGAAUCGGGG | 393 |
| 0095 | 2871 | 2889 | CCCGAUUCCAAGCCUAGAG | 95 | CUCUAGGCUUGGAAUCGGG | 394 |
| 0096 | 2872 | 2890 | CCGAUUCCAAGCCUAGAGG | 96 | CCUCUAGGCUUGGAAUCGG | 395 |
| 0097 | 2873 | 2891 | CGAUUCCAAGCCUAGAGGC | 97 | GCCUCUAGGCUUGGAAUCG | 396 |
| 0098 | 2874 | 2892 | GAUUCCAAGCCUAGAGGCU | 98 | AGCCUCUAGGCUUGGAAUC | 397 |
| 0099 | 2907 | 2925 | ACCAACUGAGCAAAGGCCU | 99 | AGGCCUUUGCUCAGUUGGU | 398 |
| 0100 | 2941 | 2959 | UACCACGGAAAUGGACAGA | 100 | UCUGUCCAUUUCCGUGGUA | 399 |
| 0101 | 2942 | 2960 | ACCACGGAAAUGGACAGAG | 101 | CUCUGUCCAUUUCCGUGGU | 400 |
| 0102 | 2944 | 2962 | CACGGAAAUGGACAGAGUU | 102 | AACUCUGUCCAUUUCCGUG | 401 |
| 0103 | 2945 | 2963 | ACGGAAAUGGACAGAGUUA | 103 | UAACUCUGUCCAUUUCCGU | 402 |
| 0104 | 2946 | 2964 | CGGAAAUGGACAGAGUUAU | 104 | AUAACUCUGUCCAUUUCCG | 403 |
| 0105 | 2950 | 2968 | AAUGGACAGAGUUAUCAAG | 105 | CUUGAUAACUCUGUCCAUU | 404 |
| 0106 | 2951 | 2969 | AUGGACAGAGUUAUCAAGG | 106 | CCUUGAUAACUCUGUCCAU | 405 |
| 0107 | 2953 | 2971 | GGACAGAGUUAUCAAGGCA | 107 | UGCCUUGAUAACUCUGUCC | 406 |
| 0108 | 2954 | 2972 | GACAGAGUUAUCAAGGCAC | 108 | GUGCCUUGAUAACUCUGUC | 407 |
| 0109 | 2955 | 2973 | ACAGAGUUAUCAAGGCACA | 109 | UGUGCCUUGAUAACUCUGU | 408 |
| 0110 | 2958 | 2976 | GAGUUAUCAAGGCACAUAC | 110 | GUAUGUGCCUUGAUAACUC | 409 |
| 0111 | 2959 | 2977 | AGUUAUCAAGGCACAUACU | 111 | AGUAUGUGCCUUGAUAACU | 410 |
| 0112 | 2960 | 2978 | GUUAUCAAGGCACAUACUU | 112 | AAGUAUGUGCCUUGAUAAC | 411 |
| 0113 | 3060 | 3078 | AAAUGCUGGCUUGAUCAAG | 113 | CUUGAUCAAGCCAGCAUUU | 412 |
| 0114 | 3068 | 3086 | GCUUGAUCAAGAACUACUG | 114 | CAGUAGUUCUUGAUCAAGC | 413 |
| 0115 | 3109 | 3127 | GCCCCUUGGUGUUAUACAA | 115 | UUGUAUAACACCAAGGGGC | 414 |
| 0116 | 3111 | 3129 | CCCUUGGUGUUAUACAACA | 116 | UGUUGUAUAACACCAAGGG | 415 |
| 0117 | 3114 | 3132 | UUGGUGUUAUACAACAGAU | 117 | AUCUGUUGUAUAACACCAA | 416 |
| 0118 | 3117 | 3135 | GUGUUAUACAACAGAUCCC | 118 | GGGAUCUGUUGUAUAACAC | 417 |
| 0119 | 3121 | 3139 | UAUACAACAGAUCCCAGUG | 119 | CACUGGGAUCUGUUGUAUA | 418 |
| 0120 | 3496 | 3514 | UGCAACCUGACACAAUGCC | 120 | GGCAUUGUGUCAGGUUGCA | 419 |
| 0121 | 3497 | 3515 | GCAACCUGACACAAUGCCU | 121 | AGGCAUUGUGUCAGGUUGC | 420 |
| 0122 | 3540 | 3558 | AACUCUCACGGUGGUCCCA | 122 | UGGGACCACCGUGAGAGUU | 421 |
| 0123 | 3543 | 3561 | UCUCACGGUGGUCCCAGAU | 123 | AUCUGGGACCACCGUGAGA | 422 |
| 0124 | 3547 | 3565 | ACGGUGGUCCCAGAUCCAA | 124 | UUGGAUCUGGGACCACCGU | 423 |
| 0125 | 3551 | 3569 | UGGUCCCAGAUCCAAGCAC | 125 | GUGCUUGGAUCUGGGACCA | 424 |
| 0126 | 3554 | 3572 | UCCCAGAUCCAAGCACAGA | 126 | UCUGUGCUUGGAUCUGGGA | 425 |
| 0127 | 3558 | 3576 | AGAUCCAAGCACAGAGGCU | 127 | AGCCUCUGUGCUUGGAUCU | 426 |
| 0128 | 3559 | 3577 | GAUCCAAGCACAGAGGCUU | 128 | AAGCCUCUGUGCUUGGAUC | 427 |
| 0129 | 3560 | 3578 | AUCCAAGCACAGAGGCUUC | 129 | GAAGCCUCUGUGCUUGGAU | 428 |
| 0130 | 3662 | 3680 | CUACCACUGUCACAGGAAG | 130 | CUUCCUGUGACAGUGGUAG | 429 |
| 0131 | 4155 | 4173 | CUGCAACCUGACGCAAUGU | 131 | ACAUUGCGUCAGGUUGCAG | 430 |
| 0132 | 4156 | 4174 | UGCAACCUGACGCAAUGUC | 132 | GACAUUGCGUCAGGUUGCA | 431 |
| 0133 | 4157 | 4175 | GCAACCUGACGCAAUGUCC | 133 | GGACAUUGCGUCAGGUUGC | 432 |
| 0134 | 4256 | 4274 | CUGAAAACAGCACUGGGGU | 134 | ACCCCAGUGCUGUUUUCAG | 433 |
| 0135 | 4300 | 4318 | CAGAGUUAUCGAGGCACAC | 135 | GUGUGCCUCGAUAACUCUG | 434 |
| 0136 | 4301 | 4319 | AGAGUUAUCGAGGCACACU | 136 | AGUGUGCCUCGAUAACUCU | 435 |
| 0137 | 4302 | 4320 | GAGUUAUCGAGGCACACUC | 137 | GAGUGUGCCUCGAUAACUC | 436 |
| 0138 | 4303 | 4321 | AGUUAUCGAGGCACACUCU | 138 | AGAGUGUGCCUCGAUAACU | 437 |
| 0139 | 4304 | 4322 | GUUAUCGAGGCACACUCUC | 139 | GAGAGUGUGCCUCGAUAAC | 438 |
| 0140 | 4305 | 4323 | UUAUCGAGGCACACUCUCC | 140 | GGAGAGUGUGCCUCGAUAA | 439 |
| 0141 | 4306 | 4324 | UAUCGAGGCACACUCUCCA | 141 | UGGAGAGUGUGCCUCGAUA | 440 |
| 0142 | 4307 | 4325 | AUCGAGGCACACUCUCCAC | 142 | GUGGAGAGUGUGCCUCGAU | 441 |
| 0143 | 4312 | 4330 | GGCACACUCUCCACCACUA | 143 | UAGUGGUGGAGAGUGUGCC | 442 |
| 0144 | 4319 | 4337 | UCUCCACCACUAUCACAGG | 144 | CCUGUGAUAGUGGUGGAGA | 443 |
| 0145 | 4359 | 4377 | GUCUAUGACACCACAUUGG | 145 | CCAAUGUGGUGUCAUAGAC | 444 |
| 0146 | 4362 | 4380 | UAUGACACCACAUUGGCAU | 146 | AUGCCAAUGUGGUGUCAUA | 445 |
| 0147 | 4366 | 4384 | ACACCACAUUGGCAUCGGA | 147 | UCCGAUGCCAAUGUGGUGU | 446 |
| 0148 | 4367 | 4385 | CACCACAUUGGCAUCGGAG | 148 | CUCCGAUGCCAAUGUGGUG | 447 |
| 0149 | 4368 | 4386 | ACCACAUUGGCAUCGGAGG | 149 | CCUCCGAUGCCAAUGUGGU | 448 |
| 0150 | 4369 | 4387 | CCACAUUGGCAUCGGAGGA | 150 | UCCUCCGAUGCCAAUGUGG | 449 |
| 0151 | 4370 | 4388 | CACAUUGGCAUCGGAGGAU | 151 | AUCCUCCGAUGCCAAUGUG | 450 |
| 0152 | 4371 | 4389 | ACAUUGGCAUCGGAGGAUC | 152 | GAUCCUCCGAUGCCAAUGU | 451 |
| 0153 | 4372 | 4390 | CAUUGGCAUCGGAGGAUCC | 153 | GGAUCCUCCGAUGCCAAUG | 452 |
| 0154 | 4373 | 4391 | AUUGGCAUCGGAGGAUCCC | 154 | GGGAUCCUCCGAUGCCAAU | 453 |
| 0155 | 4376 | 4394 | GGCAUCGGAGGAUCCCAUU | 155 | AAUGGGAUCCUCCGAUGCC | 454 |
| 0156 | 4497 | 4515 | CUGCAACCUGACACGAUGU | 156 | ACAUCGUGUCAGGUUGCAG | 455 |
| 0157 | 4498 | 4516 | UGCAACCUGACACGAUGUC | 157 | GACAUCGUGUCAGGUUGCA | 456 |
| 0158 | 4499 | 4517 | GCAACCUGACACGAUGUCC | 158 | GGACAUCGUGUCAGGUUGC | 457 |
| 0159 | 4500 | 4518 | CAACCUGACACGAUGUCCA | 159 | UGGACAUCGUGUCAGGUUG | 458 |
| 0160 | 4501 | 4519 | AACCUGACACGAUGUCCAG | 160 | CUGGACAUCGUGUCAGGUU | 459 |
| 0161 | 4503 | 4521 | CCUGACACGAUGUCCAGUG | 161 | CACUGGACAUCGUGUCAGG | 460 |
| 0162 | 4504 | 4522 | CUGACACGAUGUCCAGUGA | 162 | UCACUGGACAUCGUGUCAG | 461 |
| 0163 | 4505 | 4523 | UGACACGAUGUCCAGUGAC | 163 | GUCACUGGACAUCGUGUCA | 462 |
| 0164 | 4506 | 4524 | GACACGAUGUCCAGUGACA | 164 | UGUCACUGGACAUCGUGUC | 463 |
| 0165 | 4507 | 4525 | ACACGAUGUCCAGUGACAG | 165 | CUGUCACUGGACAUCGUGU | 464 |
| 0166 | 4510 | 4528 | CGAUGUCCAGUGACAGAAU | 166 | AUUCUGUCACUGGACAUCG | 465 |
| 0167 | 4634 | 4652 | GUGAUGGACGGAGUUAUCG | 167 | CGAUAACUCCGUCCAUCAC | 466 |
| 0168 | 4635 | 4653 | UGAUGGACGGAGUUAUCGA | 168 | UCGAUAACUCCGUCCAUCA | 467 |
| 0169 | 4636 | 4654 | GAUGGACGGAGUUAUCGAG | 169 | CUCGAUAACUCCGUCCAUC | 468 |
| 0170 | 4637 | 4655 | AUGGACGGAGUUAUCGAGG | 170 | CCUCGAUAACUCCGUCCAU | 469 |
| 0171 | 4638 | 4656 | UGGACGGAGUUAUCGAGGC | 171 | GCCUCGAUAACUCCGUCCA | 470 |
| 0172 | 4639 | 4657 | GGACGGAGUUAUCGAGGCA | 172 | UGCCUCGAUAACUCCGUCC | 471 |
| 0173 | 4644 | 4662 | GAGUUAUCGAGGCAUAUCC | 173 | GGAUAUGCCUCGAUAACUC | 472 |
| 0174 | 4645 | 4663 | AGUUAUCGAGGCAUAUCCU | 174 | AGGAUAUGCCUCGAUAACU | 473 |
| 0175 | 4646 | 4664 | GUUAUCGAGGCAUAUCCUC | 175 | GAGGAUAUGCCUCGAUAAC | 474 |
| 0176 | 4647 | 4665 | UUAUCGAGGCAUAUCCUCC | 176 | GGAGGAUAUGCCUCGAUAA | 475 |
| 0177 | 4678 | 4696 | GGAAGGACCUGUCAAUCUU | 177 | AAGAUUGACAGGUCCUUCC | 476 |
| 0178 | 4680 | 4698 | AAGGACCUGUCAAUCUUGG | 178 | CCAAGAUUGACAGGUCCUU | 477 |
| 0179 | 4681 | 4699 | AGGACCUGUCAAUCUUGGU | 179 | ACCAAGAUUGACAGGUCCU | 478 |
| 0180 | 4682 | 4700 | GGACCUGUCAAUCUUGGUC | 180 | GACCAAGAUUGACAGGUCC | 479 |
| 0181 | 4753 | 4771 | GGCCUGACCGAGAACUACU | 181 | AGUAGUUCUCGGUCAGGCC | 480 |
| 0182 | 4755 | 4773 | CCUGACCGAGAACUACUGC | 182 | GCAGUAGUUCUCGGUCAGG | 481 |
| 0183 | 4756 | 4774 | CUGACCGAGAACUACUGCA | 183 | UGCAGUAGUUCUCGGUCAG | 482 |
| 0184 | 4757 | 4775 | UGACCGAGAACUACUGCAG | 184 | CUGCAGUAGUUCUCGGUCA | 483 |
| 0185 | 4775 | 4793 | GGAAUCCAGAUUCUGGGAA | 185 | UUCCCAGAAUCUGGAUUCC | 484 |
| 0186 | 4777 | 4795 | AAUCCAGAUUCUGGGAAAC | 186 | GUUUCCCAGAAUCUGGAUU | 485 |
| 0187 | 4786 | 4804 | UCUGGGAAACAACCCUGGU | 187 | ACCAGGGUUGUUUCCCAGA | 486 |
| 0188 | 4787 | 4805 | CUGGGAAACAACCCUGGUG | 188 | CACCAGGGUUGUUUCCCAG | 487 |
| 0189 | 4789 | 4807 | GGGAAACAACCCUGGUGUU | 189 | AACACCAGGGUUGUUUCCC | 488 |
| 0190 | 4791 | 4809 | GAAACAACCCUGGUGUUAC | 190 | GUAACACCAGGGUUGUUUC | 489 |
| 0191 | 4792 | 4810 | AAACAACCCUGGUGUUACA | 191 | UGUAACACCAGGGUUGUUU | 490 |
| 0192 | 4793 | 4811 | AACAACCCUGGUGUUACAC | 192 | GUGUAACACCAGGGUUGUU | 491 |
| 0193 | 4794 | 4812 | ACAACCCUGGUGUUACACA | 193 | UGUGUAACACCAGGGUUGU | 492 |
| 0194 | 4795 | 4813 | CAACCCUGGUGUUACACAA | 194 | UUGUGUAACACCAGGGUUG | 493 |
| 0195 | 4796 | 4814 | AACCCUGGUGUUACACAAC | 195 | GUUGUGUAACACCAGGGUU | 494 |
| 0196 | 4820 | 4838 | CGUGUGUGAGGUGGGAGUA | 196 | UACUCCCACCUCACACACG | 495 |
| 0197 | 4834 | 4852 | GAGUACUGCAAUCUGACAC | 197 | GUGUCAGAUUGCAGUACUC | 496 |
| 0198 | 4840 | 4858 | UGCAAUCUGACACAAUGCU | 198 | AGCAUUGUGUCAGAUUGCA | 497 |
| 0199 | 4841 | 4859 | GCAAUCUGACACAAUGCUC | 199 | GAGCAUUGUGUCAGAUUGC | 498 |
| 0200 | 4842 | 4860 | CAAUCUGACACAAUGCUCA | 200 | UGAGCAUUGUGUCAGAUUG | 499 |
| 0201 | 4886 | 4904 | CUCCCACUGUUGUUCCAGU | 201 | ACUGGAACAACAGUGGGAG | 500 |
| 0202 | 4887 | 4905 | UCCCACUGUUGUUCCAGUU | 202 | AACUGGAACAACAGUGGGA | 501 |
| 0203 | 4889 | 4907 | CCACUGUUGUUCCAGUUCC | 203 | GGAACUGGAACAACAGUGG | 502 |
| 0204 | 4890 | 4908 | CACUGUUGUUCCAGUUCCA | 204 | UGGAACUGGAACAACAGUG | 503 |
| 0205 | 4894 | 4912 | GUUGUUCCAGUUCCAAGCA | 205 | UGCUUGGAACUGGAACAAC | 504 |
| 0206 | 4896 | 4914 | UGUUCCAGUUCCAAGCAUG | 206 | CAUGCUUGGAACUGGAACA | 505 |
| 0207 | 4897 | 4915 | GUUCCAGUUCCAAGCAUGG | 207 | CCAUGCUUGGAACUGGAAC | 506 |
| 0208 | 4911 | 4929 | CAUGGAGGCUCAUUCUGAA | 208 | UUCAGAAUGAGCCUCCAUG | 507 |
| 0209 | 4912 | 4930 | AUGGAGGCUCAUUCUGAAG | 209 | CUUCAGAAUGAGCCUCCAU | 508 |
| 0210 | 4914 | 4932 | GGAGGCUCAUUCUGAAGCA | 210 | UGCUUCAGAAUGAGCCUCC | 509 |
| 0211 | 4921 | 4939 | CAUUCUGAAGCAGCACCAA | 211 | UUGGUGCUGCUUCAGAAUG | 510 |
| 0212 | 4927 | 4945 | GAAGCAGCACCAACUGAGC | 212 | GCUCAGUUGGUGCUGCUUC | 511 |
| 0213 | 4930 | 4948 | GCAGCACCAACUGAGCAAA | 213 | UUUGCUCAGUUGGUGCUGC | 512 |
| 0214 | 4960 | 4978 | CGGCAGUGCUACCAUGGUA | 214 | UACCAUGGUAGCACUGCCG | 513 |
| 0215 | 4963 | 4981 | CAGUGCUACCAUGGUAAUG | 215 | CAUUACCAUGGUAGCACUG | 514 |
| 0216 | 4965 | 4983 | GUGCUACCAUGGUAAUGGC | 216 | GCCAUUACCAUGGUAGCAC | 515 |
| 0217 | 4972 | 4990 | CAUGGUAAUGGCCAGAGUU | 217 | AACUCUGGCCAUUACCAUG | 516 |
| 0218 | 4975 | 4993 | GGUAAUGGCCAGAGUUAUC | 218 | GAUAACUCUGGCCAUUACC | 517 |
| 0219 | 4976 | 4994 | GUAAUGGCCAGAGUUAUCG | 219 | CGAUAACUCUGGCCAUUAC | 518 |
| 0220 | 4977 | 4995 | UAAUGGCCAGAGUUAUCGA | 220 | UCGAUAACUCUGGCCAUUA | 519 |
| 0221 | 4980 | 4998 | UGGCCAGAGUUAUCGAGGC | 221 | GCCUCGAUAACUCUGGCCA | 520 |
| 0222 | 4981 | 4999 | GGCCAGAGUUAUCGAGGCA | 222 | UGCCUCGAUAACUCUGGCC | 521 |
| 0223 | 4982 | 5000 | GCCAGAGUUAUCGAGGCAC | 223 | GUGCCUCGAUAACUCUGGC | 522 |
| 0224 | 4983 | 5001 | CCAGAGUUAUCGAGGCACA | 224 | UGUGCCUCGAUAACUCUGG | 523 |
| 0225 | 4985 | 5003 | AGAGUUAUCGAGGCACAUU | 225 | AAUGUGCCUCGAUAACUCU | 524 |
| 0226 | 4986 | 5004 | GAGUUAUCGAGGCACAUUC | 226 | GAAUGUGCCUCGAUAACUC | 525 |
| 0227 | 4987 | 5005 | AGUUAUCGAGGCACAUUCU | 227 | AGAAUGUGCCUCGAUAACU | 526 |
| 0228 | 4997 | 5015 | GCACAUUCUCCACCACUGU | 228 | ACAGUGGUGGAGAAUGUGC | 527 |
| 0229 | 5001 | 5019 | AUUCUCCACCACUGUCACA | 229 | UGUGACAGUGGUGGAGAAU | 528 |
| 0230 | 5016 | 5034 | CACAGGAAGGACAUGUCAA | 230 | UUGACAUGUCCUUCCUGUG | 529 |
| 0231 | 5021 | 5039 | GAAGGACAUGUCAAUCUUG | 231 | CAAGAUUGACAUGUCCUUC | 530 |
| 0232 | 5149 | 5167 | UUUACCAUGGACCCCAGCA | 232 | UGCUGGGGUCCAUGGUAAA | 531 |
| 0233 | 5150 | 5168 | UUACCAUGGACCCCAGCAU | 233 | AUGCUGGGGUCCAUGGUAA | 532 |
| 0234 | 5180 | 5198 | ACUGCAACCUGACGCGAUG | 234 | CAUCGCGUCAGGUUGCAGU | 533 |
| 0235 | 5186 | 5204 | ACCUGACGCGAUGCUCAGA | 235 | UCUGAGCAUCGCGUCAGGU | 534 |
| 0236 | 5189 | 5207 | UGACGCGAUGCUCAGACAC | 236 | GUGUCUGAGCAUCGCGUCA | 535 |
| 0237 | 5190 | 5208 | GACGCGAUGCUCAGACACA | 237 | UGUGUCUGAGCAUCGCGUC | 536 |
| 0238 | 5191 | 5209 | ACGCGAUGCUCAGACACAG | 238 | CUGUGUCUGAGCAUCGCGU | 537 |
| 0239 | 5192 | 5210 | CGCGAUGCUCAGACACAGA | 239 | UCUGUGUCUGAGCAUCGCG | 538 |
| 0240 | 5761 | 5779 | GAAGUGAACCUCGAAUCUC | 240 | GAGAUUCGAGGUUCACUUC | 539 |
| 0241 | 5922 | 5940 | CAGGACUGAAUGUUACAUC | 241 | GAUGUAACAUUCAGUCCUG | 540 |
| 0242 | 5956 | 5974 | ACCCAAGGUACCUUUGGGA | 242 | UCCCAAAGGUACCUUGGGU | 541 |
| 0243 | 5957 | 5975 | CCCAAGGUACCUUUGGGAC | 243 | GUCCCAAAGGUACCUUGGG | 542 |
| 0244 | 5964 | 5982 | UACCUUUGGGACUGGCCUU | 244 | AAGGCCAGUCCCAAAGGUA | 543 |
| 0245 | 5965 | 5983 | ACCUUUGGGACUGGCCUUC | 245 | GAAGGCCAGUCCCAAAGGU | 544 |
| 0246 | 6323 | 6341 | GACAGCAAUCAAACGAAGA | 246 | UCUUCGUUUGAUUGCUGUC | 545 |
| 0247 | 6324 | 6342 | ACAGCAAUCAAACGAAGAC | 247 | GUCUUCGUUUGAUUGCUGU | 546 |
| 0248 | 6325 | 6343 | CAGCAAUCAAACGAAGACA | 248 | UGUCUUCGUUUGAUUGCUG | 547 |
| 0249 | 6326 | 6344 | AGCAAUCAAACGAAGACAC | 249 | GUGUCUUCGUUUGAUUGCU | 548 |
| 0250 | 6327 | 6345 | GCAAUCAAACGAAGACACU | 250 | AGUGUCUUCGUUUGAUUGC | 549 |
| 0251 | 6328 | 6346 | CAAUCAAACGAAGACACUG | 251 | CAGUGUCUUCGUUUGAUUG | 550 |
| 0252 | 6330 | 6348 | AUCAAACGAAGACACUGUU | 252 | AACAGUGUCUUCGUUUGAU | 551 |
| 0253 | 6331 | 6349 | UCAAACGAAGACACUGUUC | 253 | GAACAGUGUCUUCGUUUGA | 552 |
| 0254 | 6332 | 6350 | CAAACGAAGACACUGUUCC | 254 | GGAACAGUGUCUUCGUUUG | 553 |
| 0255 | 6333 | 6351 | AAACGAAGACACUGUUCCC | 255 | GGGAACAGUGUCUUCGUUU | 554 |
| 0256 | 6334 | 6352 | AACGAAGACACUGUUCCCA | 256 | UGGGAACAGUGUCUUCGUU | 555 |
| 0257 | 6335 | 6353 | ACGAAGACACUGUUCCCAG | 257 | CUGGGAACAGUGUCUUCGU | 556 |
| 0258 | 6336 | 6354 | CGAAGACACUGUUCCCAGC | 258 | GCUGGGAACAGUGUCUUCG | 557 |
| 0259 | 6337 | 6355 | GAAGACACUGUUCCCAGCU | 259 | AGCUGGGAACAGUGUCUUC | 558 |
| 0260 | 6338 | 6356 | AAGACACUGUUCCCAGCUA | 260 | UAGCUGGGAACAGUGUCUU | 559 |
| 0261 | 6339 | 6357 | AGACACUGUUCCCAGCUAC | 261 | GUAGCUGGGAACAGUGUCU | 560 |
| 0262 | 6340 | 6358 | GACACUGUUCCCAGCUACC | 262 | GGUAGCUGGGAACAGUGUC | 561 |
| 0263 | 6341 | 6359 | ACACUGUUCCCAGCUACCA | 263 | UGGUAGCUGGGAACAGUGU | 562 |
| 0264 | 6350 | 6368 | CCAGCUACCAGCUAUGCCA | 264 | UGGCAUAGCUGGUAGCUGG | 563 |
| 0265 | 6351 | 6369 | CAGCUACCAGCUAUGCCAA | 265 | UUGGCAUAGCUGGUAGCUG | 564 |
| 0266 | 6352 | 6370 | AGCUACCAGCUAUGCCAAA | 266 | UUUGGCAUAGCUGGUAGCU | 565 |
| 0267 | 6353 | 6371 | GCUACCAGCUAUGCCAAAC | 267 | GUUUGGCAUAGCUGGUAGC | 566 |
| 0268 | 6354 | 6372 | CUACCAGCUAUGCCAAACC | 268 | GGUUUGGCAUAGCUGGUAG | 567 |
| 0269 | 6355 | 6373 | UACCAGCUAUGCCAAACCU | 269 | AGGUUUGGCAUAGCUGGUA | 568 |
| 0270 | 6376 | 6394 | GCAUUUUUGGUAUUUUUGU | 270 | ACAAAAAUACCAAAAAUGC | 569 |
| 0271 | 6377 | 6395 | CAUUUUUGGUAUUUUUGUG | 271 | CACAAAAAUACCAAAAAUG | 570 |
| 0272 | 6378 | 6396 | AUUUUUGGUAUUUUUGUGU | 272 | ACACAAAAAUACCAAAAAU | 571 |
| 0273 | 6379 | 6397 | UUUUUGGUAUUUUUGUGUA | 273 | UACACAAAAAUACCAAAAA | 572 |
| 0274 | 6380 | 6398 | UUUUGGUAUUUUUGUGUAU | 274 | AUACACAAAAAUACCAAAA | 573 |
| 0275 | 6381 | 6399 | UUUGGUAUUUUUGUGUAUA | 275 | UAUACACAAAAAUACCAAA | 574 |
| 0276 | 6382 | 6400 | UUGGUAUUUUUGUGUAUAA | 276 | UUAUACACAAAAAUACCAA | 575 |
| 0277 | 6383 | 6401 | UGGUAUUUUUGUGUAUAAG | 277 | CUUAUACACAAAAAUACCA | 576 |
| 0278 | 6384 | 6402 | GGUAUUUUUGUGUAUAAGC | 278 | GCUUAUACACAAAAAUACC | 577 |
| 0279 | 6385 | 6403 | GUAUUUUUGUGUAUAAGCU | 279 | AGCUUAUACACAAAAAUAC | 578 |
| 0280 | 6386 | 6404 | UAUUUUUGUGUAUAAGCUU | 280 | AAGCUUAUACACAAAAAUA | 579 |
| 0281 | 6387 | 6405 | AUUUUUGUGUAUAAGCUUU | 281 | AAAGCUUAUACACAAAAAU | 580 |
| 0282 | 6388 | 6406 | UUUUUGUGUAUAAGCUUUU | 282 | AAAAGCUUAUACACAAAAA | 581 |
| 0283 | 6455 | 6473 | UGUUAAAAAUAAACUCUGC | 283 | GCAGAGUUUAUUUUUAACA | 582 |
| 0284 | 6456 | 6474 | GUUAAAAAUAAACUCUGCA | 284 | UGCAGAGUUUAUUUUUAAC | 583 |
| 0285 | 6457 | 6475 | UUAAAAAUAAACUCUGCAC | 285 | GUGCAGAGUUUAUUUUUAA | 584 |
| 0286 | 6458 | 6476 | UAAAAAUAAACUCUGCACU | 286 | AGUGCAGAGUUUAUUUUUA | 585 |
| 0287 | 6459 | 6477 | AAAAAUAAACUCUGCACUU | 287 | AAGUGCAGAGUUUAUUUUU | 586 |
| 0288 | 6460 | 6478 | AAAAUAAACUCUGCACUUA | 288 | UAAGUGCAGAGUUUAUUUU | 587 |
| 0289 | 6461 | 6479 | AAAUAAACUCUGCACUUAU | 289 | AUAAGUGCAGAGUUUAUUU | 588 |
| 0290 | 6462 | 6480 | AAUAAACUCUGCACUUAUU | 290 | AAUAAGUGCAGAGUUUAUU | 589 |
| 0291 | 6463 | 6481 | AUAAACUCUGCACUUAUUU | 291 | AAAUAAGUGCAGAGUUUAU | 590 |
| 0292 | 6464 | 6482 | UAAACUCUGCACUUAUUUU | 292 | AAAAUAAGUGCAGAGUUUA | 591 |
| 0293 | 6465 | 6483 | AAACUCUGCACUUAUUUUG | 293 | CAAAAUAAGUGCAGAGUUU | 592 |
| 0294 | 6466 | 6484 | AACUCUGCACUUAUUUUGA | 294 | UCAAAAUAAGUGCAGAGUU | 593 |
| 0295 | 6467 | 6485 | ACUCUGCACUUAUUUUGAU | 295 | AUCAAAAUAAGUGCAGAGU | 594 |
| 0296 | 6468 | 6486 | CUCUGCACUUAUUUUGAUU | 296 | AAUCAAAAUAAGUGCAGAG | 595 |
| 0297 | 6469 | 6487 | UCUGCACUUAUUUUGAUUU | 297 | AAAUCAAAAUAAGUGCAGA | 596 |
| 0298 | 6470 | 6488 | CUGCACUUAUUUUGAUUUG | 298 | CAAAUCAAAAUAAGUGCAG | 597 |
| 0299 | 6471 | 6489 | UGCACUUAUUUUGAUUUGA | 299 | UCAAAUCAAAAUAAGUGCA | 598 |

I.4 Nucleotide Modifications

A dsRNA of the present disclosure may comprise one or more modifications, e.g., to enhance cellular uptake, affinity for the target sequence, inhibitory activity, and/or stability. Modifications may include any modification known in the art, including, for example, end modifications, base modifications, sugar modifications/replacements, and backbone modifications. End modifications may include, for example, 5′ end modifications (e.g., phosphorylation, conjugation, and inverted linkages) and 3′ end modifications (e.g., conjugation, DNA nucleotides, and inverted linkages). Base modifications may include, e.g., replacement with stabilizing bases, destabilizing bases or bases that base-pair with an expanded repertoire of partners, removal of bases (abasic modifications of nucleotides), or conjugated bases. Sugar modifications or replacements may include, e.g., modifications at the 2′ or 4′ position of the sugar moiety, or replacement of the sugar moiety. Backbone modifications may include, for example, modification or replacement of the phosphodiester linkages, e.g., with one or more phosphorothioates, phosphorodithioates, phosphotriesters, methyl and other alkyl phosphonates, phosphinates, and phosphoramidates.

As used herein, the term “nucleotide” includes naturally occurring or modified nucleotide, or a surrogate replacement moiety. A modified nucleotide is a non-naturally occurring nucleotide and is also referred to herein as a “nucleotide analog.” One of ordinary skill in the art would understand that guanine, cytosine, adenine, uracil, or thymine in a nucleotide may be replaced by other moieties without substantially altering the base-pairing properties of the modified nucleotide. For example, a nucleotide comprising inosine as its base may base-pair with nucleotides containing adenine, cytosine, or uracil. Hence, nucleotides containing uracil, guanine, or adenine may be replaced in the nucleotide sequences of the present disclosure by a nucleotide containing, for example, inosine. Sequences comprising such replacement moieties are included as embodiments of the present disclosure. A modified nucleotide may also be a nucleotide whose ribose moiety is replaced with a non-ribose moiety.

The dsRNAs of the present disclosure may include one or more modified nucleotides known in the art, including, without limitation, 2′-O-methyl modified nucleotides, 2′-fluoro modified nucleotides, 2′-deoxy modified nucleotides, 2′-O-methoxyethyl modified nucleotides, modified nucleotides comprising alternate internucleotide linkages such as thiophosphates and phosphorothioates, phosphotriester modified nucleotides, modified nucleotides terminally linked to a cholesterol derivative or lipophilic moiety, peptide nucleic acids (PNAs; see, e.g., Nielsen et al., Science (1991) 254:1497-500), constrained ethyl (cEt) modified nucleotides, inverted deoxy modified nucleotides, inverted dideoxy modified nucleotides, locked nucleic acid modified nucleotides, abasic modifications of nucleotides, 2′-amino modified nucleotides, 2′-alkyl modified nucleotides, morpholino-modified nucleotides, phosphoramidate modified nucleotides, modified nucleotides comprising modifications at other sites of the sugar or base of an oligonucleotide, and non-natural base-containing modified nucleotides. In some embodiments, at least one of the one or more modified nucleotides is a 2′-O-methyl nucleotide, 5′-phosphorothioate nucleotide, or a terminal nucleotide linked to a cholesterol derivative, lipophilic or other targeting moiety. The incorporation of 2′-O-methyl, 2′-O-ethyl, 2′-O-propryl, 2′-O-alkyl, 2′-O-aminoalkyl, or 2′-deoxy-2′-fluoro (i.e., 2′-fluoro) groups in nucleosides of an oligonucleotide may confer enhanced hybridization properties and/or enhanced nuclease stability to the oligonucleotide. Further, oligonucleotides containing phosphorothioate backbones (e.g., phosphorothioate linkage between two neighboring nucleotides at one or more positions of the dsRNA) may have enhanced nuclease stability. In some embodiments, the dsRNA may contain nucleotides with a modified ribose, such as locked nucleic acid (LNA) units.

In some embodiments, the dsRNA comprises one or more modified nucleotides, wherein at least one of the one or more modified nucleotides is 2′-deoxy-2′-fluoro-ribonucleotide, 2′-deoxyribonucleotide, or 2′-O-methyl-ribonucleotide.

In some embodiments, the dsRNA comprises an inverted 2′-deoxyribonucleotide at the 3′-end of its sense or antisense strand.

In some embodiments, a dsRNA of the present disclosure comprises one or more 2′-O-methyl nucleotides and one or more 2′-fluoro nucleotides. In some embodiments, the dsRNA comprises two or more 2′-O-methyl nucleotides and two or more 2′-fluoro nucleotides. In some embodiments, the dsRNA comprises two or more 2′-O-methyl nucleotides (OMe) and two or more 2′-fluoro nucleotides (F) in an alternating pattern, e.g., the pattern OMe-F-OMe-F or the pattern F-OMe-F-OMe. In some embodiments, the sense sequence and the antisense sequence of the dsRNA comprise alternating 2′-O-methyl ribonucleotides and 2′-deoxy-2′-fluoro ribonucleotides. In some embodiments, the dsRNA comprises up to 10 contiguous nucleotides that are each a 2′-O-methyl nucleotide. In some embodiments, the dsRNA comprises up to 10 contiguous nucleotides that are each a 2′-fluoro nucleotide. In some embodiments, the dsRNA comprises two or more 2′-fluoro nucleotides at the 5′- or 3′-end of the antisense strand.

In some embodiments, a dsRNA of the present disclosure comprises one or more phosphorothioate groups. In some embodiments, a dsRNA of the present disclosure comprises two or more, three or more, four or more, five or more, six or more, seven or more, eight or more, nine or more, or 10 or more phosphorothioate groups. In some embodiments, the dsRNA does not comprise any phosphorothioate group.

In some embodiments, the dsRNA comprises one or more phosphotriester groups. In some embodiments, the dsRNA comprises two or more, three or more, four or more, five or more, six or more, seven or more, eight or more, nine or more, or 10 or more phosphotriester groups. In some embodiments, the dsRNA does not comprise any phosphotriester group.

In some embodiments, the dsRNA comprises a modified ribonucleoside such as a deoxyribonucleoside, including, for example, deoxyribonucleoside overhang(s), and one or more deoxyribonucleosides within the double-stranded portion of a dsRNA. However, it is self-evident that under no circumstances is a double-stranded DNA molecule encompassed by the term “dsRNA.” In some embodiments, the dsRNA comprises two or more, three or more, four or more,

five or more, six or more, seven or more, eight or more, nine or more, or 10 or more different modified nucleotides described herein. In some embodiments, the dsRNA comprises up to two contiguous modified nucleotides, up to three contiguous modified nucleotides, up to four contiguous modified nucleotides, up to five contiguous modified nucleotides, up to six contiguous modified nucleotides, up to seven contiguous modified nucleotides, up to eight contiguous modified nucleotides, up to nine contiguous modified nucleotides, or up to 10 contiguous modified nucleotides. In some embodiments, the contiguous modified nucleotides are the same modified nucleotide. In some embodiments, the contiguous modified nucleotides are two or more, three or more, four or more, five or more, six or more, seven or more, eight or more, nine or more, or ten or more different modified nucleotides.

In some embodiments, the dsRNA is such that:
a) the sense strand comprises a nucleotide sequence selected from the group consisting of SEQ ID NOs: 602, 605, 617, 688, 702, 705, 706, 708, 709, 766, 767, 770, 798, 819, 821, 877, and 896; or
b) the antisense strand comprises a nucleotide sequence selected from the group consisting of SEQ ID NOs: 901, 904, 916, 987, 1001, 1004, 1005, 1007, 1008, 1065, 1066, 1069, 1097, 1118, 1120, 1176, and 1195.

In some embodiments, the dsRNA is such that:
a) the sense strand comprises a nucleotide sequence selected from the group consisting of SEQ ID NOs:708, 770, and 821; or
b) the antisense strand comprises a nucleotide sequence selected from the group consisting of SEQ ID NOs: 1007, 1069, and 1120.

In some embodiments, the sense strand and antisense strand of the dsRNA respectively comprise the nucleotide sequences of:
a) SEQ ID NOs: 602 and 901;
b) SEQ ID NOs: 605 and 904;
c) SEQ ID NOs: 617 and 916;
d) SEQ ID NOs: 688 and 987;
e) SEQ ID NOs: 702 and 1001;
f) SEQ ID NOs: 705 and 1004;
g) SEQ ID NOs: 706 and 1005;
h) SEQ ID NOs: 708 and 1007;
i) SEQ ID NOs: 709 and 1008;
j) SEQ ID NOs: 766 and 1065;
k) SEQ ID NOs: 767 and 1066;
l) SEQ ID NOs: 770 and 1069;
m) SEQ ID NOs: 798 and 1097;
n) SEQ ID NOs: 819 and 1118;
o) SEQ ID NOs: 821 and 1120;
p) SEQ ID NOs: 877 and 1176; or
q) SEQ ID NOs: 896 and 1195.

In some embodiments, the sense strand and antisense strand of the dsRNA respectively comprise the nucleotide sequences of:
a) SEQ ID NOs: 708 and 1007;
b) SEQ ID NOs: 770 and 1069; or
c) SEQ ID NOs: 821 and 1120.

Table 2 below lists the sequences of exemplary siRNA constructs (CNST) with modified nucleotides. The start (ST) and end (ED) nucleotide positions in NM_005577.2 (SEQ ID NO: 1632) are indicated. Abbreviations are as follows: SEQ=SEQ ID NO; x (nucleotide in lower case)=2′-O-Me nucleotide (also denoted as mX elsewhere herein); Xf=2′-F nucleotide (also denoted as fX elsewhere herein); dX=DNA nucleotide; and invdX=inverted dX. In these constructs, the sequences of their sense strands and antisense strands correspond to the sense and antisense sequences of the constructs in Table 1 with the same construct numbers, but for the inclusion of (1) the modified 2′-O-Me nucleotides and 2′-F nucleotides, (2) c-c-a at the 5′ end of the sense strand nucleotide sequence, (3) invdT at the 3′ end of the sense strand nucleotide sequence, and/or (4) dT-dT at the 3′ end of the antisense strand nucleotide sequence. In these constructs, a base-pair of nucleotides may be modified differently in some embodiments, e.g., one nucleotide in the base-pair is a 2′-O-Me ribonucleotide and the other is a 2′-F nucleotide. In some embodiments, the antisense strand comprises two 2′-F nucleotides at its 5′ end.

TABLE 2
Sequences of Modified LPA siRNA Constructs

| siLPA | | | Sense Sequence | | Antisense Sequence | |
|---|---|---|---|---|---|---|
| # | ST | ED | (5′-3′) | SEQ | (5′-3′) | SEQ |
| 0001 | 5 | 23 | ccaAfcCfuUfuGfgGfgCf | 599 | GfAfaAfgCfcAfgCfcCf | 898 |
| | | | uGfgCfuUfuCf(invdT) | | cAfaAfgGfudTdT | |
| 0002 | 185 | 203 | ccaGfcCfaUfgUfgGfuCf | 600 | CfAfaUfcCfuGfgAfcCf | 899 |
| | | | cAfgGfaUfuGf(invdT) | | aCfaUfgGfcdTdT | |
| 0003 | 219 | 237 | ccaAfcAfgAfgUfuAfuCf | 601 | CfGfuGfcCfuCfgAfuAf | 900 |
| | | | gAfgGfcAfcGf(invdT) | | aCfuCfuGfudTdT | |
| 0004 | 220 | 238 | ccaCfaGfaGfuUfaUfcGf | 602 | AfCfgUfgCfcUfcGfaUf | 901 |
| | | | aGfgCfaCfgUf(invdT) | | aAfcUfcUfgdTdT | |
| 0005 | 221 | 239 | ccaAfgAfgUfuAfuCfgAf | 603 | UfAfcGfuGfcCfuCfgAf | 902 |
| | | | gGfcAfcGfuAf(invdT) | | uAfaCfuCfudTdT | |
| 0006 | 222 | 240 | ccaGfaGfuUfaUfcGfaGf | 604 | GfUfaCfgUfgCfcUfcGf | 903 |
| | | | gCfaCfgUfaCf(invdT) | | aUfaAfcUfcdTdT | |
| 0007 | 223 | 241 | ccaAfgUfuAfuCfgAfgGf | 605 | AfGfuAfcGfuGfcCfuCf | 904 |
| | | | cAfcGfuAfcUf(invdT) | | gAfuAfaCfudTdT | |
| 0008 | 238 | 256 | ccaUfaCfuCfcAfcCfaCf | 606 | CfUfgUfgAfcAfgUfgGf | 905 |
| | | | uGfuCfaCfaGf(invdT) | | uGfgAfgUfadTdT | |
| 0009 | 240 | 258 | ccaCfuCfcAfcCfaCfuGf | 607 | UfCfcUfgUfgAfcAfgUf | 906 |
| | | | uCfaCfaGfgAf(invdT) | | gGfuGfgAfgdTdT | |
| 0010 | 259 | 277 | ccaAfgGfaCfcUfgCfcAf | 608 | AfCfcAfaGfcUfuGfgCf | 907 |
| | | | aGfcUfuGfgUf(invdT) | | aGfgUfcCfudTdT | |
| 0011 | 261 | 279 | ccaGfaCfcUfgCfcAfaGf | 609 | UfGfaCfcAfaGfcUfuGf | 908 |
| | | | cUfuGfgUfcAf(invdT) | | gCfaGfgUfcdTdT | |
| 0012 | 262 | 280 | ccaAfcCfuGfcCfaAfgCf | 610 | AfUfgAfcCfaAfgCfuUf | 909 |
| | | | uUfgGfuCfaUf(invdT) | | gGfcAfgGfudTdT | |
| 0013 | 263 | 281 | ccaCfcUfgCfcAfaGfcUf | 611 | GfAfuGfaCfcAfaGfcUf | 910 |
| | | | uGfgUfcAfuCf(invdT) | | uGfgCfaGfgdTdT | |
| 0014 | 266 | 284 | ccaGfcCfaAfgCfuUfgGf | 612 | AfUfaGfaUfgAfcCfaAf | 911 |
| | | | uCfaUfcUfaUf(invdT) | | gCfuUfgGfcdTdT | |
| 0015 | 267 | 285 | ccaCfcAfaGfcUfuGfgUf | 613 | CfAfuAfgAfuGfaCfcAf | 912 |
| | | | cAfuCfuAfuGf(invdT) | | aGfcUfuGfgdTdT | |
| 0016 | 298 | 316 | ccaCfaUfaAfuAfgGfaCf | 614 | UfUfuCfuGfuGfgUfcCf | 913 |
| | | | cAfcAfgAfaAf(invdT) | | uAfuUfaUfgdTdT | |
| 0017 | 300 | 318 | ccaUfaAfuAfgGfaCfcAf | 615 | GfUfuUfuCfuGfuGfgUf | 914 |
| | | | cAfgAfaAfaCf(invdT) | | cCfuAfuUfadTdT | |
| 0018 | 301 | 319 | ccaAfaUfaGfgAfcCfaCf | 616 | AfGfuUfuUfcUfgUfgGf | 915 |
| | | | aGfaAfaAfcUf(invdT) | | uCfcUfaUfudTdT | |
| 0019 | 302 | 320 | ccaAfuAfgGfaCfcAfcAf | 617 | UfAfgUfuUfuCfuGfuGf | 916 |
| | | | gAfaAfaCfuAf(invdT) | | gUfcCfuAfudTdT | |
| 0020 | 303 | 321 | ccaUfaGfgAfcCfaCfaGf | 618 | GfUfaGfuUfuUfcUfgUf | 917 |
| | | | aAfaAfcUfaCf(invdT) | | gGfuCfcUfadTdT | |
| 0021 | 324 | 342 | ccaAfaAfuGfcUfgGfcUf | 619 | CfAfuGfaUfcAfaGfcCf | 918 |
| | | | uGfaUfcAfuGf(invdT) | | aGfcAfuUfudTdT | |
| 0022 | 330 | 348 | ccaUfgGfcUfuGfaUfcAf | 620 | GfUfaGfuUfcAfuGfaUf | 919 |
| | | | uGfaAfcUfaCf(invdT) | | cAfaGfcCfadTdT | |
| 0023 | 331 | 349 | ccaGfgCfuUfgAfuCfaUf | 621 | AfGfuAfgUfuCfaUfgAf | 920 |
| | | | gAfaCfuAfcUf(invdT) | | uCfaAfgCfcdTdT | |
| 0024 | 372 | 390 | ccaAfgCfuCfcUfuAfuUf | 622 | CfGfuAfuAfaCfaAfuAf | 921 |
| | | | gUfuAfuAfcGf(invdT) | | aGfgAfgCfudTdT | |
| 0025 | 373 | 391 | ccaGfcUfcCfuUfaUfuGf | 623 | UfCfgUfaUfaAfcAfaUf | 922 |
| | | | uUfaUfaCfgAf(invdT) | | aAfgGfaGfcdTdT | |
| 0026 | 413 | 431 | ccaAfgUfaCfuGfcAfaCf | 624 | UfGfcGfuCfaGfgUfuGf | 923 |
| | | | cUfgAfcGfcAf(invdT) | | cAfgUfaCfudTdT | |
| 0027 | 414 | 432 | ccaGfuAfcUfgCfaAfcCf | 625 | UfUfgCfgUfcAfgGfuUf | 924 |
| | | | uGfaCfgCfaAf(invdT) | | gCfaGfuAfcdTdT | |
| 0028 | 415 | 433 | ccaUfaCfuGfcAfaCfcUf | 626 | AfUfuGfcGfuCfaGfgUf | 925 |
| | | | gAfcGfcAfaUf(invdT) | | uGfcAfgUfadTdT | |
| 0029 | 418 | 436 | ccaUfgCfaAfcCfuGfaCf | 627 | AfGfcAfuUfgCfgUfcAf | 926 |
| | | | gCfaAfuGfcUf(invdT) | | gGfuUfgCfadTdT | |
| 0030 | 419 | 437 | ccaGfcAfaCfcUfgAfcGf | 628 | GfAfgCfaUfuGfcGfuCf | 927 |
| | | | cAfaUfgCfuCf(invdT) | | aGfgUfuGfcdTdT | |
| 0031 | 420 | 438 | ccaCfaAfcCfuGfaCfgCf | 629 | UfGfaGfcAfuUfgCfgUf | 928 |
| | | | aAfuGfcUfcAf(invdT) | | cAfgGfuUfgdTdT | |
| 0032 | 421 | 439 | ccaAfaCfcUfgAfcGfcAf | 630 | CfUfgAfgCfaUfuGfcGf | 929 |
| | | | aUfgCfuCfaGf(invdT) | | uCfaGfgUfudTdT | |
| 0033 | 422 | 440 | ccaAfcCfuGfaCfgCfaAf | 631 | UfCfuGfaGfcAfuUfgCf | 930 |
| | | | uGfcUfcAfgAf(invdT) | | gUfcAfgGfudTdT | |
| 0034 | 423 | 441 | ccaCfcUfgAfcGfcAfaUf | 632 | GfUfcUfgAfgCfaUfuGf | 931 |
| | | | gCfuCfaGfaCf(invdT) | | cGfuCfaGfgdTdT | |
| 0035 | 424 | 442 | ccaCfuGfaCfgCfaAfuGf | 633 | CfGfuCfuGfaGfcAfuUf | 932 |
| | | | cUfcAfgAfcGf(invdT) | | gCfgUfcAfgdTdT | |
| 0036 | 425 | 443 | ccaUfgAfcGfcAfaUfgCf | 634 | GfCfgUfcUfgAfgCfaUf | 933 |
| | | | uCfaGfaCfgCf(invdT) | | uGfcGfuCfadTdT | |
| 0037 | 465 | 483 | ccaUfcCfgAfcUfgUfuAf | 635 | AfAfcCfgGfgGfuAfaCf | 934 |
| | | | cCfcCfgGfuUf(invdT) | | aGfuCfgGfadTdT | |
| 0038 | 469 | 487 | ccaAfcUfgUfuAfcCfcCf | 636 | UfUfgGfaAfcCfgGfgGf | 935 |
| | | | gGfuUfcCfaAf(invdT) | | uAfaCfaGfudTdT | |
| 0039 | 470 | 488 | ccaCfuGfuUfaCfcCfcGf | 637 | CfUfuGfgAfaCfcGfgGf | 936 |
| | | | gUfuCfcAfaGf(invdT) | | gUfaAfcAfgdTdT | |
| 0040 | 471 | 489 | ccaUfgUfuAfcCfcCfgGf | 638 | GfCfuUfgGfaAfcCfgGf | 937 |
| | | | uUfcCfaAfgCf(invdT) | | gGfuAfaCfadTdT | |
| 0041 | 473 | 491 | ccaUfuAfcCfcCfgGfuUf | 639 | AfGfgCfuUfgGfaAfcCf | 938 |
| | | | cCfaAfgCfcUf(invdT) | | gGfgGfuAfadTdT | |
| 0042 | 474 | 492 | ccaUfaCfcCfcGfgUfuCf | 640 | UfAfgGfcUfuGfgAfaCf | 939 |
| | | | cAfaGfcCfuAf(invdT) | | cGfgGfgUfadTdT | |
| 0043 | 480 | 498 | ccaGfgUfuCfcAfaGfcCf | 641 | AfGfcCfuCfuAfgGfcUf | 940 |
| | | | uAfgAfgGfcUf(invdT) | | uGfgAfaCfcdTdT | |
| 0044 | 48 | 499 | ccaGfuUfcCfaAfgCfcUf | 642 | GfAfgCfcUfcUfaGfgCf | 941 |
| | | | aGfaGfgCfuCf(invdT) | | uUfgGfaAfcdTdT | |
| 0045 | 489 | 507 | ccaCfcUfaGfaGfgCfuCf | 643 | UfUfcGfgAfaGfgAfgCf | 942 |
| | | | cUfuCfcGfaAf(invdT) | | cUfcUfaGfgdTdT | |
| 0046 | 490 | 508 | ccaCfuAfgAfgGfcUfcCf | 644 | GfUfuCfgGfaAfgGfaGf | 943 |
| | | | uUfcCfgAfaCf(invdT) | | cCfuCfuAfgdTdT | |
| 0047 | 495 | 513 | ccaGfgCfuCfcUfuCfcGf | 645 | UfGfcUfuGfuUfcGfgAf | 944 |
| | | | aAfcAfaGfcAf(invdT) | | aGfgAfgCfcdTdT | |
| 0048 | 499 | 517 | ccaCfcUfuCfcGfaAfcAf | 646 | UfCfgGfuGfcUfuGfuUf | 945 |
| | | | aGfcAfcCfgAf(invdT) | | cGfgAfaGfgdTdT | |
| 0049 | 500 | 518 | ccaCfuUfcCfgAfaCfaAf | 647 | GfUfcGfgUfgCfuUfgUf | 946 |
| | | | gCfaCfcGfaCf(invdT) | | uCfgGfaAfgdTdT | |
| 0050 | 501 | 519 | ccaUfuCfcGfaAfcAfaGf | 648 | AfGfuCfgGfuGfcUfuGf | 947 |
| | | | cAfcCfgAfcUf(invdT) | | uUfcGfgAfadTdT | |
| 0051 | 502 | 520 | ccaUfcCfgAfaCfaAfgCf | 649 | CfAfgUfcGfgUfgCfuUf | 948 |
| | | | aCfcGfaCfuGf(invdT) | | gUfuCfgGfadTdT | |
| 0052 | 503 | 521 | ccaCfcGfaAfcAfaGfcAf | 650 | UfCfaGfuCfgGfuGfcUf | 949 |
| | | | cCfgAfcUfgAf(invdT) | | uGfuUfcGfgdTdT | |
| 0053 | 505 | 523 | ccaGfaAfcAfaGfcAfcCf | 651 | GfCfuCfaGfuCfgGfuGf | 950 |
| | | | gAfcUfgAfgCf(invdT) | | cUfuGfuUfcdTdT | |
| 0054 | 506 | 524 | ccaAfaCfaAfgCfaCfcGf | 652 | UfGfcUfcAfgUfcGfgUf | 951 |
| | | | aCfuGfaGfcAf(invdT) | | gCfuUfgUfudTdT | |
| 0055 | 507 | 525 | ccaAfcAfaGfcAfcCfgAf | 653 | UfUfgCfuCfaGfuCfgGf | 952 |
| | | | cUfgAfgCfaAf(invdT) | | uGfcUfuGfudTdT | |
| 0056 | 508 | 526 | ccaCfaAfgCfaCfcGfaCf | 654 | UfUfuGfcUfcAfgUfcGf | 953 |
| | | | uGfaGfcAfaAf(invdT) | | gUfgCfuUfgdTdT | |
| 0057 | 509 | 527 | ccaAfaGfcAfcCfgAfcUf | 655 | CfUfuUfgCfuCfaGfuCf | 954 |
| | | | gAfgCfaAfaGf(invdT) | | gGfuGfcUfudTdT | |
| 0058 | 510 | 528 | ccaAfgCfaCfcGfaCfuGf | 656 | CfCfuUfuGfcUfcAfgUf | 955 |
| | | | aGfcAfaAfgGf(invdT) | | cGfgUfgCfudTdT | |
| 0059 | 552 | 570 | ccaUfgGfuAfaUfgGfaCf | 657 | AfUfaAfcUfcUfgUfcCf | 956 |
| | | | aGfaGfuUfaUf(invdT) | | aUfuAfcCfadTdT | |
| 0060 | 554 | 572 | ccaGfuAfaUfgGfaCfaGf | 658 | CfGfaUfaAfcUfcUfgUf | 957 |
| | | | aGfuUfaUfcGf(invdT) | | cCfaUfuAfcdTdT | |
| 0061 | 555 | 573 | ccaUfaAfuGfgAfcAfgAf | 659 | UfCfgAfuAfaCfuCfuGf | 958 |
| | | | gUfuAfuCfgAf(invdT) | | uCfcAfuUfadTdT | |
| 0062 | 563 | 581 | ccaAfgAfgUfuAfuCfgAf | 660 | UfAfuGfuGfcCfuCfgAf | 959 |
| | | | gGfcAfcAfuAf(invdT) | | uAfaCfuCfudTdT | |
| 0063 | 564 | 582 | ccaGfaGfuUfaUfcGfaGf | 661 | GfUfaUfgUfgCfcUfcGf | 960 |
| | | | gCfaCfaUfaCf(invdT) | | aUfaAfcUfcdTdT | |
| 0064 | 565 | 583 | ccaAfgUfuAfuCfgAfgGf | 662 | AfGfuAfuGfuGfcCfuCf | 961 |
| | | | cAfcAfuAfcUf(invdT) | | gAfuAfaCfudTdT | |
| 0065 | 566 | 584 | ccaGfuUfaUfcGfaGfgCf | 663 | GfAfgUfaUfgUfgCfcUf | 962 |
| | | | aCfaUfaCfuCf(invdT) | | CGfaUfaAfcdTdT | |
| 0066 | 567 | 585 | ccaUfuAfuCfgAfgGfcAf | 664 | GfGfaGfuAfuGfuGfcCf | 963 |
| | | | cAfuAfcUfcCf(invdT) | | uCfgAfuAfadTdT | |
| 0067 | 574 | 592 | ccaGfgCfaCfaUfaCfuCf | 665 | CfAfgUfgGfuGfgAfgUf | 964 |
| | | | cAfcCfaCfuGf(invdT) | | aUfgUfgCfcdTdT | |
| 0068 | 578 | 596 | ccaCfaUfaCfuCfcAfcCf | 666 | GfUfgAfcAfgUfgGfuGf | 965 |
| | | | aCfuGfuCfaCf(invdT) | | gAfgUfaUfgdTdT | |
| 0069 | 598 | 616 | ccaGfgAfaGfaAfcCfuGf | 667 | AfAfgCfuUfgGfcAfgGf | 966 |
| | | | cCfaAfgCfuUf(invdT) | | uUfcUfuCfcdTdT | |
| 0070 | 624 | 642 | ccaUfaUfgAfcAfcCfaCf | 668 | AfUfgCfgAfgUfgUfgGf | 967 |
| | | | aCfuCfgCfaUf(invdT) | | uGfuCfaUfadTdT | |
| 0071 | 625 | 643 | ccaAfuGfaCfaCfcAfcAf | 669 | UfAfuGfcGfaGfuGfuGf | 968 |
| | | | cUfcGfcAfuAf(invdT) | | gUfgUfcAfudTdT | |
| 0072 | 626 | 644 | ccaUfgAfcAfcCfaCfaCf | 670 | CfUfaUfgCfgAfgUfgUf | 969 |
| | | | uCfgCfaUfaGf(invdT) | | gGfuGfuCfadTdT | |
| 0073 | 627 | 645 | ccaGfaCfaCfcAfcAfcUf | 671 | AfCfuAfuGfcGfaGfuGf | 970 |
| | | | cGfcAfuAfgUf(invdT) | | uGfgUfgUfcdTdT | |
| 0074 | 628 | 646 | ccaAfcAfcCfaCfaCfuCf | 672 | GfAfcUfaUfgCfgAfgUf | 971 |
| | | | gCfaUfaGfuCf(invdT) | | gUfgGfuGfudTdT | |
| 0075 | 630 | 648 | ccaAfcCfaCfaCfuCfgCf | 673 | CfCfgAfcUfaUfgCfgAf | 972 |
| | | | aUfaGfuCfgGf(invdT) | | gUfgUfgGfudTdT | |
| 0076 | 63 | 649 | ccaCfcAfcAfcUfcGfcAf | 674 | UfCfcGfaCfuAfuGfcGf | 973 |
| | | | uAfgUfcGfgAf(invdT) | | aGfuGfuGfgdTdT | |
| 0077 | 632 | 650 | ccaCfaCfaCfuCfgCfaUf | 675 | GfUfcCfgAfcUfaUfgCf | 974 |
| | | | aGfuCfgGfaCf(invdT) | | gAfgUfgUfgdTdT | |
| 0078 | 633 | 651 | ccaAfcAfcUfcGfcAfuAf | 676 | GfGfuCfcGfaCfuAfuGf | 975 |
| | | | gUfcGfgAfcCf(invdT) | | CGfaGfuGfudTdT | |
| 0079 | 640 | 658 | ccaCfaUfaGfuCfgGfaCf | 677 | AfUfuCfuGfgGfgUfcCf | 976 |
| | | | cCfcAfgAfaUf(invdT) | | gAfcUfaUfgdTdT | |
| 0080 | 641 | 659 | ccaAfuAfgUfcGfgAfcCf | 678 | UfAfuUfcUfgGfgGfuCf | 977 |
| | | | cCfaGfaAfuAf(invdT) | | cGfaCfuAfudTdT | |
| 0081 | 642 | 660 | ccaUfaGfuCfgGfaCfcCf | 679 | GfUfaUfuCfuGfgGfgUf | 978 |
| | | | cAfgAfaUfaCf(invdT) | | cCfgAfcUfadTdT | |
| 0082 | 643 | 661 | ccaAfgUfcGfgAfcCfcCf | 680 | AfGfuAfuUfcUfgGfgGf | 979 |
| | | | aGfaAfuAfcUf(invdT) | | uCfcGfaCfudTdT | |
| 0083 | 644 | 662 | ccaGfuCfgGfaCfcCfcAf | 681 | UfAfgUfaUfuCfuGfgGf | 980 |
| | | | gAfaUfaCfuAf(invdT) | | gUfcCfgAfcdTdT | |
| 0084 | 645 | 663 | ccaUfcGfgAfcCfcCfaGf | 682 | GfUfaGfuAfuUfcUfgGf | 981 |
| | | | aAfuAfcUfaCf(invdT) | | gGfuCfcGfadTdT | |
| 0085 | 646 | 664 | ccaCfgGfaCfcCfcAfgAf | 683 | GfGfuAfgUfaUfuCfuGf | 982 |
| | | | aUfaCfuAfcCf(invdT) | | gGfgUfcCfgdTdT | |
| 0086 | 1191 | 1209 | ccaAfcAfaGfcAfcCfgAf | 684 | CfUfgCfuCfaGfuCfgGf | 983 |
| | | | cUfgAfgCfaGf(invdT) | | uGfcUfuGfudTdT | |
| 0087 | 1193 | 1211 | ccaAfaGfcAfcCfgAfcUf | 685 | CfUfcUfgCfuCfaGfuCf | 984 |
| | | | gAfgCfaGfaGf(invdT) | | gGfuGfcUfudTdT | |
| 0088 | 1234 | 1252 | ccaCfaCfgGfuAfaUfgGf | 686 | AfAfcUfcUfgUfcCfaUf | 985 |
| | | | aCfaGfaGfuUf(invdT) | | uAfcCfgUfgdTdT | |
| 0089 | 1235 | 1253 | ccaAfcGfgUfaAfuGfgAf | 687 | UfAfaCfuCfuGfuCfcAf | 986 |
| | | | cAfgAfgUfuAf(invdT) | | uUfaCfcGfudTdT | |
| 0090 | 1236 | 1254 | ccaCfgGfuAfaUfgGfaCf | 688 | AfUfaAfcUfcUfgUfcCf | 987 |
| | | | aGfaGfuUfaUf(invdT) | | aUfuAfcCfgdTdT | |
| 0091 | 2867 | 2885 | ccaUfuAfcCfcCfgAfuUf | 689 | AfGfgCfuUfgGfaAfuCf | 988 |
| | | | cCfaAfgCfcUf(invdT) | | gGfgGfuAfadTdT | |
| 0092 | 2868 | 2886 | ccaUfaCfcCfcGfaUfuCf | 690 | UfAfgGfcUfuGfgAfaUf | 989 |
| | | | cAfaGfcCfuAf(invdT) | | cGfgGfgUfadTdT | |
| 0093 | 2869 | 2887 | ccaAfcCfcCfgAfuUfcCf | 691 | CfUfaGfgCfuUfgGfaAf | 990 |
| | | | aAfgCfcUfaGf(invdT) | | uCfgGfgGfudTdT | |
| 0094 | 2870 | 2888 | ccaCfcCfcGfaUfuCfcAf | 692 | UfCfuAfgGfcUfuGfgAf | 991 |
| | | | aGfcCfuAfgAf(invdT) | | aUfcGfgGfgdTdT | |
| 0095 | 2871 | 2889 | ccaCfcCfgAfuUfcCfaAf | 693 | CfUfcUfaGfgCfuUfgGf | 992 |
| | | | gCfcUfaGfaGf(invdT) | | aAfuCfgGfgdTdT | |
| 0096 | 2872 | 2890 | ccaCfcGfaUfuCfcAfaGf | 694 | CfCfuCfuAfgGfcUfuGf | 993 |
| | | | cCfuAfgAfgGf(invdT) | | gAfaUfcGfgdTdT | |
| 0097 | 2873 | 2891 | ccaCfgAfuUfcCfaAfgCf | 695 | GfCfcUfcUfaGfgCfuUf | 994 |
| | | | cUfaGfaGfgCf(invdT) | | gGfaAfuCfgdTdT | |
| 0098 | 2874 | 2892 | ccaGfaUfuCfcAfaGfcCf | 696 | AfGfcCfuCfuAfgGfcUf | 995 |
| | | | uAfgAfgGfcUf(invdT) | | uGfgAfaUfcdTdT | |
| 0099 | 2907 | 2925 | ccaAfcCfaAfcUfgAfgCf | 697 | AfGfgCfcUfuUfgCfuCf | 996 |
| | | | aAfaGfgCfcUf(invdT) | | aGfuUfgGfudTdT | |
| 0100 | 2941 | 2959 | ccaUfaCfcAfcGfgAfaAf | 698 | UfCfuGfuCfcAfuUfuCf | 997 |
| | | | uGfgAfcAfgAf(invdT) | | cGfuGfgUfadTdT | |
| 0101 | 2942 | 2960 | ccaAfcCfaCfgGfaAfaUf | 699 | CfUfcUfgUfcCfaUfuUf | 998 |
| | | | gGfaCfaGfaGf(invdT) | | cCfgUfgGfudTdT | |
| 0102 | 2944 | 2962 | ccaCfaCfgGfaAfaUfgGf | 700 | AfAfcUfcUfgUfcCfaUf | 999 |
| | | | aCfaGfaGfuUf(invdT) | | uUfcCfgUfgdTdT | |
| 0103 | 2945 | 2963 | ccaAfcGfgAfaAfuGfgAf | 701 | UfAfaCfuCfuGfuCfcAf | 1000 |
| | | | cAfgAfgUfuAf(invdT) | | uUfuCfcGfudTdT | |
| 0104 | 2946 | 2964 | ccaCfgGfaAfaUfgGfaCf | 702 | AfUfaAfcUfcUfgUfcCf | 1001 |
| | | | aGfaGfuUfaUf(invdT) | | aUfuUfcCfgdTdT | |
| 0105 | 2950 | 2968 | ccaAfaUfgGfaCfaGfaGf | 703 | CfUfuGfaUfaAfcUfcUf | 1002 |
| | | | uUfaUfcAfaGf(invdT) | | gUfcCfaUfudTdT | |
| 0106 | 2951 | 2969 | ccaAfuGfgAfcAfgAfgUf | 704 | CfCfuUfgAfuAfaCfuCf | 1003 |
| | | | uAfuCfaAfgGf(invdT) | | uGfuCfcAfudTdT | |
| 0107 | 2953 | 2971 | ccaGfgAfcAfgAfgUfuAf | 705 | UfGfcCfuUfgAfuAfaCf | 1004 |
| | | | uCfaAfgGfcAf(invdT) | | uCfuGfuCfcdTdT | |
| 0108 | 2954 | 2972 | ccaGfaCfaGfaGfuUfaUf | 706 | GfUfgCfcUfuGfaUfaAf | 1005 |
| | | | cAfaGfgCfaCf(invdT) | | cUfcUfgUfcdTdT | |
| 0109 | 2955 | 2973 | ccaAfcAfgAfgUfuAfuCf | 707 | UfGfuGfcCfuUfgAfuAf | 1006 |
| | | | aAfgGfcAfcAf(invdT) | | aCfuCfuGfudTdT | |
| 0110 | 2958 | 2976 | ccaGfaGfuUfaUfcAfaGf | 708 | GfUfaUfgUfgCfcUfuGf | 1007 |
| | | | gCfaCfaUfaCf(invdT) | | aUfaAfcUfcdTdT | |
| 0111 | 2959 | 2977 | ccaAfgUfuAfuCfaAfgGf | 709 | AfGfuAfuGfuGfcCfuUf | 1008 |
| | | | cAfcAfuAfcUf(invdT) | | gAfuAfaCfudTdT | |
| 0112 | 2960 | 2978 | ccaGfuUfaUfcAfaGfgCf | 710 | AfAfgUfaUfgUfgCfcUf | 1009 |
| | | | aCfaUfaCfuUf(invdT) | | uGfaUfaAfcdTdT | |
| 0113 | 3060 | 3078 | ccaAfaAfuGfcUfgGfcUf | 711 | CfUfuGfaUfcAfaGfcCf | 1010 |
| | | | uGfaUfcAfaGf(invdT) | | aGfcAfuUfudTdT | |
| 0114 | 3068 | 3086 | ccaGfcUfuGfaUfcAfaGf | 712 | CfAfgUfaGfuUfcUfuGf | 1011 |
| | | | aAfcUfaCfuGf(invdT) | | aUfcAfaGfcdTdT | |
| 0115 | 3109 | 3127 | ccaGfcCfcCfuUfgGfuGf | 713 | UfUfgUfaUfaAfcAfcCf | 1012 |
| | | | uUfaUfaCfaAf(invdT) | | aAfgGfgGfcdTdT | |
| 0116 | 3111 | 3129 | ccaCfcCfuUfgGfuGfuUf | 714 | UfGfuUfgUfaUfaAfcAf | 1013 |
| | | | aUfaCfaAfcAf(invdT) | | cCfaAfgGfgdTdT | |
| 0117 | 3114 | 3132 | ccaUfuGfgUfgUfuAfuAf | 715 | AfUfcUfgUfuGfuAfuAf | 1014 |
| | | | cAfaCfaGfaUf(invdT) | | aCfaCfcAfadTdT | |
| 0118 | 3117 | 3135 | ccaGfuGfuUfaUfaCfaAf | 716 | GfGfgAfuCfuGfuUfgUf | 1015 |
| | | | cAfgAfuCfcCf(invdT) | | aUfaAfcAfcdTdT | |
| 0119 | 3121 | 3139 | ccaUfaUfaCfaAfcAfgAf | 717 | CfAfcUfgGfgAfuCfuGf | 1016 |
| | | | uCfcCfaGfuGf(invdT) | | uUfgUfaUfadTdT | |
| 0120 | 3496 | 3514 | ccaUfgCfaAfcCfuGfaCf | 718 | GfGfcAfuUfgUfgUfcAf | 1017 |
| | | | aCfaAfuGfcCf(invdT) | | gGfuUfgCfadTdT | |
| 0121 | 3497 | 3515 | ccaGfcAfaCfcUfgAfcAf | 719 | AfGfgCfaUfuGfuGfuCf | 1018 |
| | | | cAfaUfgCfcUf(invdT) | | aGfgUfuGfcdTdT | |
| 0122 | 3540 | 3558 | ccaAfaCfuCfuCfaCfgGf | 720 | UfGfgGfaCfcAfcCfgUf | 1019 |
| | | | uGfgUfcCfcAf(invdT) | | gAfgAfgUfudTdT | |
| 0123 | 3543 | 3561 | ccaUfcUfcAfcGfgUfgGf | 721 | AfUfcUfgGfgAfcCfaCf | 1020 |
| | | | uCfcCfaGfaUf(invdT) | | cGfuGfaGfadTdT | |
| 0124 | 3547 | 3565 | ccaAfcGfgUfgGfuCfcCf | 722 | UfUfgGfaUfcUfgGfgAf | 1021 |
| | | | aGfaUfcCfaAf(invdT) | | cCfaCfcGfudTdT | |
| 0125 | 3551 | 3569 | ccaUfgGfuCfcCfaGfaUf | 723 | GfUfgCfuUfgGfaUfcUf | 1022 |
| | | | cCfaAfgCfaCf(invdT) | | gGfgAfcCfadTdT | |
| 0126 | 3554 | 3572 | ccaUfcCfcAfgAfuCfcAf | 724 | UfCfuGfuGfcUfuGfgAf | 1023 |
| | | | aGfcAfcAfgAf(invdT) | | uCfuGfgGfadTdT | |
| 0127 | 3558 | 3576 | ccaAfgAfuCfcAfaGfcAf | 725 | AfGfcCfuCfuGfuGfcUf | 1024 |
| | | | cAfgAfgGfcUf(invdT) | | uGfgAfuCfudTdT | |
| 0128 | 3559 | 3577 | ccaGfaUfcCfaAfgCfaCf | 726 | AfAfgCfcUfcUfgUfgCf | 1025 |
| | | | aGfaGfgCfuUf(invdT) | | uUfgGfaUfcdTdT | |
| 0129 | 3560 | 3578 | ccaAfuCfcAfaGfcAfcAf | 727 | GfAfaGfcCfuCfuGfuGf | 1026 |
| | | | gAfgGfcUfuCf(invdT) | | cUfuGfgAfudTdT | |
| 0130 | 3662 | 3680 | ccaCfuAfcCfaCfuGfuCf | 728 | CfUfuCfcUfgUfgAfcAf | 1027 |
| | | | aCfaGfgAfaGf(invdT) | | gUfgGfuAfgdTdT | |
| 0131 | 4155 | 4173 | ccaCfuGfcAfaCfcUfgAf | 729 | AfCfaUfuGfcGfuCfaGf | 1028 |
| | | | cGfcAfaUfgUf(invdT) | | gUfuGfcAfgdTdT | |
| 0132 | 4156 | 4174 | ccaUfgCfaAfcCfuGfaCf | 730 | GfAfcAfuUfgCfgUfcAf | 1029 |
| | | | gCfaAfuGfuCf(invdT) | | gGfuUfgCfadTdT | |
| 0133 | 4157 | 4175 | ccaGfcAfaCfcUfgAfcGf | 731 | GfGfaCfaUfuGfcGfuCf | 1030 |
| | | | cAfaUfgUfcCf(invdT) | | aGfgUfuGfcdTdT | |
| 0134 | 4256 | 4274 | ccaCfuGfaAfaAfcAfgCf | 732 | AfCfcCfcAfgUfgCfuGf | 1031 |
| | | | aCfuGfgGfgUf(invdT) | | uUfuUfcAfgdTdT | |
| 0135 | 4300 | 4318 | ccaCfaGfaGfuUfaUfcGf | 733 | GfUfgUfgCfcUfcGfaUf | 1032 |
| | | | aGfgCfaCfaCf(invdT) | | aAfcUfcUfgdTdT | |
| 0136 | 4301 | 4319 | ccaAfgAfgUfuAfuCfgAf | 734 | AfGfuGfuGfcCfuCfgAf | 1033 |
| | | | gGfcAfcAfcUf(invdT) | | uAfaCfuCfudTdT | |
| 0137 | 4302 | 4320 | ccaGfaGfuUfaUfcGfaGf | 735 | GfAfgUfgUfgCfcUfcGf | 1034 |
| | | | gCfaCfaCfuCf(invdT) | | aUfaAfcUfcdTdT | |
| 0138 | 4303 | 4321 | ccaAfgUfuAfuCfgAfgGf | 736 | AfGfaGfuGfuGfcCfuCf | 1035 |
| | | | cAfcAfcUfcUf(invdT) | | gAfuAfaCfudTdT | |
| 0139 | 4304 | 4322 | ccaGfuUfaUfcGfaGfgCf | 737 | GfAfgAfgUfgUfgCfcUf | 1036 |
| | | | aCfaCfuCfuCf(invdT) | | CGfaUfaAfcdTdT | |
| 0140 | 4305 | 4323 | ccaUfuAfuCfgAfgGfcAf | 738 | GfGfaGfaGfuGfuGfcCf | 1037 |
| | | | cAfcUfcUfcCf(invdT) | | uCfgAfuAfadTdT | |
| 0141 | 4306 | 4324 | ccaUfaUfcGfaGfgCfaCf | 739 | UfGfgAfgAfgUfgUfgCf | 1038 |
| | | | aCfuCfuCfcAf(invdT) | | cUfcGfaUfadTdT | |
| 0142 | 4307 | 4325 | ccaAfuCfgAfgGfcAfcAf | 740 | GfUfgGfaGfaGfuGfuGf | 1039 |
| | | | cUfcUfcCfaCf(invdT) | | cCfuCfgAfudTdT | |
| 0143 | 4312 | 4330 | ccaGfgCfaCfaCfuCfuCf | 741 | UfAfgUfgGfuGfgAfgAf | 1040 |
| | | | cAfcCfaCfuAf(invdT) | | gUfgUfgCfcdTdT | |
| 0144 | 4319 | 4337 | ccaUfcUfcCfaCfcAfcUf | 742 | CfCfuGfuGfaUfaGfuGf | 1041 |
| | | | aUfcAfcAfgGf(invdT) | | gUfgGfaGfadTdT | |
| 0145 | 4359 | 4377 | ccaGfuCfuAfuGfaCfaCf | 743 | CfCfaAfuGfuGfgUfgUf | 1042 |
| | | | cAfcAfuUfgGf(invdT) | | cAfuAfgAfcdTdT | |
| 0146 | 4362 | 4380 | ccaUfaUfgAfcAfcCfaCf | 744 | AfUfgCfcAfaUfgUfgGf | 1043 |
| | | | aUfuGfgCfaUf(invdT) | | uGfuCfaUfadTdT | |
| 0147 | 4366 | 4384 | ccaAfcAfcCfaCfaUfuGf | 745 | UfCfcGfaUfgCfcAfaUf | 1044 |
| | | | gCfaUfcGfgAf(invdT) | | gUfgGfuGfudTdT | |
| 0148 | 4367 | 4385 | ccaCfaCfcAfcAfuUfgGf | 746 | CfUfcCfgAfuGfcCfaAf | 1045 |
| | | | cAfuCfgGfaGf(invdT) | | uGfuGfgUfgdTdT | |
| 0149 | 4368 | 4386 | ccaAfcCfaCfaUfuGfgCf | 747 | CfCfuCfcGfaUfgCfcAf | 1046 |
| | | | aUfcGfgAfgGf(invdT) | | aUfgUfgGfudTdT | |
| 0150 | 4369 | 4387 | ccaCfcAfcAfuUfgGfcAf | 748 | UfCfcUfcCfgAfuGfcCf | 1047 |
| | | | uCfgGfaGfgAf(invdT) | | aAfuGfuGfgdTdT | |
| 0151 | 4370 | 4388 | ccaCfaCfaUfuGfgCfaUf | 749 | AfUfcCfuCfcGfaUfgCf | 1048 |
| | | | cGfgAfgGfaUf(invdT) | | cAfaUfgUfgdTdT | |
| 0152 | 4371 | 4389 | ccaAfcAfuUfgGfcAfuCf | 750 | GfAfuCfcUfcCfgAfuGf | 1049 |
| | | | gGfaGfgAfuCf(invdT) | | cCfaAfuGfudTdT | |
| 0153 | 4372 | 4390 | ccaCfaUfuGfgCfaUfcGf | 751 | GfGfaUfcCfuCfcGfaUf | 1050 |
| | | | gAfgGfaUfcCf(invdT) | | gCfcAfaUfgdTdT | |
| 0154 | 4373 | 4391 | ccaAfuUfgGfcAfuCfgGf | 752 | GfGfgAfuCfcUfcCfgAf | 1051 |
| | | | aGfgAfuCfcCf(invdT) | | uGfcCfaAfudTdT | |
| 0155 | 4376 | 4394 | ccaGfgCfaUfcGfgAfgGf | 753 | AfAfuGfgGfaUfcCfuCf | 1052 |
| | | | aUfcCfcAfuUf(invdT) | | cGfaUfgCfcdTdT | |
| 0156 | 4497 | 4515 | ccaCfuGfcAfaCfcUfgAf | 754 | AfCfaUfcGfuGfuCfaGf | 1053 |
| | | | cAfcGfaUfgUf(invdT) | | gUfuGfcAfgdTdT | |
| 0157 | 4498 | 4516 | ccaUfgCfaAfcCfuGfaCf | 755 | GfAfcAfuCfgUfgUfcAf | 1054 |
| | | | aCfgAfuGfuCf(invdT) | | gGfuUfgCfadTdT | |
| 0158 | 4499 | 4517 | ccaGfcAfaCfcUfgAfcAf | 756 | GfGfaCfaUfcGfuGfuCf | 1055 |
| | | | cGfaUfgUfcCf(invdT) | | aGfgUfuGfcdTdT | |
| 0159 | 4500 | 4518 | ccaCfaAfcCfuGfaCfaCf | 757 | UfGfgAfcAfuCfgUfgUf | 1056 |
| | | | gAfuGfuCfcAf(invdT) | | cAfgGfuUfgdTdT | |
| 0160 | 4501 | 4519 | ccaAfaCfcUfgAfcAfcGf | 758 | CfUfgGfaCfaUfcGfuGf | 1057 |
| | | | aUfgUfcCfaGf(invdT) | | uCfaGfgUfudTdT | |
| 0161 | 4503 | 4521 | ccaCfcUfgAfcAfcGfaUf | 759 | CfAfcUfgGfaCfaUfcGf | 1058 |
| | | | gUfcCfaGfuGf(invdT) | | uGfuCfaGfgdTdT | |
| 0162 | 4504 | 4522 | ccaCfuGfaCfaCfgAfuGf | 760 | UfCfaCfuGfgAfcAfuCf | 1059 |
| | | | uCfcAfgUfgAf(invdT) | | gUfgUfcAfgdTdT | |
| 0163 | 4505 | 4523 | ccaUfgAfcAfcGfaUfgUf | 761 | GfUfcAfcUfgGfaCfaUf | 1060 |
| | | | cCfaGfuGfaCf(invdT) | | cGfuGfuCfadTdT | |
| 0164 | 4506 | 4524 | ccaGfaCfaCfgAfuGfuCf | 762 | UfGfuCfaCfuGfgAfcAf | 1061 |
| | | | cAfgUfgAfcAf(invdT) | | uCfgUfgUfcdTdT | |
| 0165 | 4507 | 4525 | ccaAfcAfcGfaUfgUfcCf | 763 | CfUfgUfcAfcUfgGfaCf | 1062 |
| | | | aGfuGfaCfaGf(invdT) | | aUfcGfuGfudTdT | |
| 0166 | 4510 | 4528 | ccaCfgAfuGfuCfcAfgUf | 764 | AfUfuCfuGfuCfaCfuGf | 1063 |
| | | | gAfcAfgAfaUf(invdT) | | gAfcAfuCfgdTdT | |
| 0167 | 4634 | 4652 | ccaGfuGfaUfgGfaCfgGf | 765 | CfGfaUfaAfcUfcCfgUf | 1064 |
| | | | aGfuUfaUfcGf(invdT) | | cCfaUfcAfcdTdT | |
| 0168 | 4635 | 4653 | ccaUfgAfuGfgAfcGfgAf | 766 | UfCfgAfuAfaCfuCfcGf | 1065 |
| | | | gUfuAfuCfgAf(invdT) | | uCfcAfuCfadTdT | |
| 0169 | 4636 | 4654 | ccaGfaUfgGfaCfgGfaGf | 767 | CfUfcGfaUfaAfcUfcCf | 1066 |
| | | | uUfaUfcGfaGf(invdT) | | gUfcCfaUfcdTdT | |
| 0170 | 4637 | 4655 | ccaAfuGfgAfcGfgAfgUf | 768 | CfCfuCfgAfuAfaCfuCf | 1067 |
| | | | uAfuCfgAfgGf(invdT) | | cGfuCfcAfudTdT | |
| 0171 | 4638 | 4656 | ccaUfgGfaCfgGfaGfuUf | 769 | GfCfcUfcGfaUfaAfcUf | 1068 |
| | | | aUfcGfaGfgCf(invdT) | | cCfgUfcCfadTdT | |
| 0172 | 4639 | 4657 | ccaGfgAfcGfgAfgUfuAf | 770 | UfGfcCfuCfgAfuAfaCf | 1069 |
| | | | uCfgAfgGfcAf(invdT) | | uCfcGfuCfcdTdT | |
| 0173 | 4644 | 4662 | ccaGfaGfuUfaUfcGfaGf | 771 | GfGfaUfaUfgCfcUfcGf | 1070 |
| | | | gCfaUfaUfcCf(invdT) | | aUfaAfcUfcdTdT | |
| 0174 | 4645 | 4663 | ccaAfgUfuAfuCfgAfgGf | 772 | AfGfgAfuAfuGfcCfuCf | 1071 |
| | | | cAfuAfuCfcUf(invdT) | | gAfuAfaCfudTdT | |
| 0175 | 4646 | 4664 | ccaGfuUfaUfcGfaGfgCf | 773 | GfAfgGfaUfaUfgCfcUf | 1072 |
| | | | aUfaUfcCfuCf(invdT) | | CGfaUfaAfcdTdT | |
| 0176 | 4647 | 4665 | ccaUfuAfuCfgAfgGfcAf | 774 | GfGfaGfgAfuAfuGfcCf | 1073 |
| | | | uAfuCfcUfcCf(invdT) | | uCfgAfuAfadTdT | |
| 0177 | 4678 | 4696 | ccaGfgAfaGfgAfcCfuGf | 775 | AfAfgAfuUfgAfcAfgGf | 1074 |
| | | | uCfaAfuCfuUf(invdT) | | uCfcUfuCfcdTdT | |
| 0178 | 4680 | 4698 | ccaAfaGfgAfcCfuGfuCf | 776 | CfCfaAfgAfuUfgAfcAf | 1075 |
| | | | aAfuCfuUfgGf(invdT) | | gGfuCfcUfudTdT | |
| 0179 | 4681 | 4699 | ccaAfgGfaCfcUfgUfcAf | 777 | AfCfcAfaGfaUfuGfaCf | 1076 |
| | | | aUfcUfuGfgUf(invdT) | | aGfgUfcCfudTdT | |
| 0180 | 4682 | 4700 | ccaGfgAfcCfuGfuCfaAf | 778 | GfAfcCfaAfgAfuUfgAf | 1077 |
| | | | uCfuUfgGfuCf(invdT) | | cAfgGfuCfcdTdT | |
| 0181 | 4753 | 4771 | ccaGfgCfcUfgAfcCfgAf | 779 | AfGfuAfgUfuCfuCfgGf | 1078 |
| | | | gAfaCfuAfcUf(invdT) | | uCfaGfgCfcdTdT | |
| 0182 | 4755 | 4773 | ccaCfcUfgAfcCfgAfgAf | 780 | GfCfaGfuAfgUfuCfuCf | 1079 |
| | | | aCfuAfcUfgCf(invdT) | | gGfuCfaGfgdTdT | |
| 0183 | 4756 | 4774 | ccaCfuGfaCfcGfaGfaAf | 781 | UfGfcAfgUfaGfuUfcUf | 1080 |
| | | | cUfaCfuGfcAf(invdT) | | cGfgUfcAfgdTdT | |
| 0184 | 4757 | 4775 | ccaUfgAfcCfgAfgAfaCf | 782 | CfUfgCfaGfuAfgUfuCf | 1081 |
| | | | uAfcUfgCfaGf(invdT) | | uCfgGfuCfadTdT | |
| 0185 | 4775 | 4793 | ccaGfgAfaUfcCfaGfaUf | 783 | UfUfcCfcAfgAfaUfcUf | 1082 |
| | | | uCfuGfgGfaAf(invdT) | | gGfaUfuCfcdTdT | |
| 0186 | 4777 | 4795 | ccaAfaUfcCfaGfaUfuCf | 784 | GfUfuUfcCfcAfgAfaUf | 1083 |
| | | | uGfgGfaAfaCf(invdT) | | cUfgGfaUfudTdT | |
| 0187 | 4786 | 4804 | ccaUfcUfgGfgAfaAfcAf | 785 | AfCfcAfgGfgUfuGfuUf | 1084 |
| | | | aCfcCfuGfgUf(invdT) | | uCfcCfaGfadTdT | |
| 0188 | 4787 | 4805 | ccaCfuGfgGfaAfaCfaAf | 786 | CfAfcCfaGfgGfuUfgUf | 1085 |
| | | | cCfcUfgGfuGf(invdT) | | uUfcCfcAfgdTdT | |
| 0189 | 4789 | 4807 | ccaGfgGfaAfaCfaAfcCf | 787 | AfAfcAfcCfaGfgGfuUf | 1086 |
| | | | cUfgGfuGfuUf(invdT) | | gUfuUfcCfcdTdT | |
| 0190 | 4791 | 4809 | ccaGfaAfaCfaAfcCfcUf | 788 | GfUfaAfcAfcCfaGfgGf | 1087 |
| | | | gGfuGfuUfaCf(invdT) | | uUfgUfuUfcdTdT | |
| 0191 | 4792 | 4810 | ccaAfaAfcAfaCfcCfuGf | 789 | UfGfuAfaCfaCfcAfgGf | 1088 |
| | | | gUfgUfuAfcAf(invdT) | | gUfuGfuUfudTdT | |
| 0192 | 4793 | 4811 | ccaAfaCfaAfcCfcUfgGf | 790 | GfUfgUfaAfcAfcCfaGf | 1089 |
| | | | uGfuUfaCfaCf(invdT) | | gGfuUfgUfudTdT | |
| 0193 | 4794 | 4812 | ccaAfcAfaCfcCfuGfgUf | 791 | UfGfuGfuAfaCfaCfcAf | 1090 |
| | | | gUfuAfcAfcAf(invdT) | | gGfgUfuGfudTdT | |
| 0194 | 4795 | 4813 | ccaCfaAfcCfcUfgGfuGf | 792 | UfUfgUfgUfaAfcAfcCf | 1091 |
| | | | uUfaCfaCfaAf(invdT) | | aGfgGfuUfgdTdT | |
| 0195 | 4796 | 4814 | ccaAfaCfcCfuGfgUfgUf | 793 | GfUfuGfuGfuAfaCfaCf | 1092 |
| | | | uAfcAfcAfaCf(invdT) | | cAfgGfgUfudTdT | |
| 0196 | 4820 | 4838 | ccaCfgUfgUfgUfgAfgGf | 794 | UfAfcUfcCfcAfcCfuCf | 1093 |
| | | | uGfgGfaGfuAf(invdT) | | aCfaCfaCfgdTdT | |
| 0197 | 4834 | 4852 | ccaGfaGfuAfcUfgCfaAf | 795 | GfUfgUfcAfgAfuUfgCf | 1094 |
| | | | uCfuGfaCfaCf(invdT) | | aGfuAfcUfcdTdT | |
| 0198 | 4840 | 4858 | ccaUfgCfaAfuCfuGfaCf | 796 | AfGfcAfuUfgUfgUfcAf | 1095 |
| | | | aCfaAfuGfcUf(invdT) | | gAfuUfgCfadTdT | |
| 0199 | 4841 | 4859 | ccaGfcAfaUfcUfgAfcAf | 797 | GfAfgCfaUfuGfuGfuCf | 1096 |
| | | | cAfaUfgCfuCf(invdT) | | aGfaUfuGfcdTdT | |
| 0200 | 4842 | 4860 | ccaCfaAfuCfuGfaCfaCf | 798 | UfGfaGfcAfuUfgUfgUf | 1097 |
| | | | aAfuGfcUfcAf(invdT) | | cAfgAfuUfgdTdT | |
| 0201 | 4886 | 4904 | ccaCfuCfcCfaCfuGfuUf | 799 | AfCfuGfgAfaCfaAfcAf | 1098 |
| | | | gUfuCfcAfgUf(invdT) | | gUfgGfgAfgdTdT | |
| 0202 | 4887 | 4905 | ccaUfcCfcAfcUfgUfuGf | 800 | AfAfcUfgGfaAfcAfaCf | 1099 |
| | | | uUfcCfaGfuUf(invdT) | | aGfuGfgGfadTdT | |
| 0203 | 4889 | 4907 | ccaCfcAfcUfgUfuGfuUf | 801 | GfGfaAfcUfgGfaAfcAf | 1100 |
| | | | cCfaGfuUfcCf(invdT) | | aCfaGfuGfgdTdT | |
| 0204 | 4890 | 4908 | ccaCfaCfuGfuUfgUfuCf | 802 | UfGfgAfaCfuGfgAfaCf | 1101 |
| | | | cAfgUfuCfcAf(invdT) | | aAfcAfgUfgdTdT | |
| 0205 | 4894 | 4912 | ccaGfuUfgUfuCfcAfgUf | 803 | UfGfcUfuGfgAfaCfuGf | 1102 |
| | | | uCfcAfaGfcAf(invdT) | | gAfaCfaAfcdTdT | |
| 0206 | 4896 | 4914 | ccaUfgUfuCfcAfgUfuCf | 804 | CfAfuGfcUfuGfgAfaCf | 1103 |
| | | | cAfaGfcAfuGf(invdT) | | uGfgAfaCfadTdT | |
| 0207 | 4897 | 4915 | ccaGfuUfcCfaGfuUfcCf | 805 | CfCfaUfgCfuUfgGfaAf | 1104 |
| | | | aAfgCfaUfgGf(invdT) | | cUfgGfaAfcdTdT | |
| 0208 | 4911 | 4929 | ccaCfaUfgGfaGfgCfuCf | 806 | UfUfcAfgAfaUfgAfgCf | 1105 |
| | | | aUfuCfuGfaAf(invdT) | | cUfcCfaUfgdTdT | |
| 0209 | 4912 | 4930 | ccaAfuGfgAfgGfcUfcAf | 807 | CfUfuCfaGfaAfuGfaGf | 1106 |
| | | | uUfcUfgAfaGf(invdT) | | cCfuCfcAfudTdT | |
| 0210 | 4914 | 4932 | ccaGfgAfgGfcUfcAfuUf | 808 | UfGfcUfuCfaGfaAfuGf | 1107 |
| | | | cUfgAfaGfcAf(invdT) | | aGfcCfuCfcdTdT | |
| 0211 | 4921 | 4939 | ccaCfaUfuCfuGfaAfgCf | 809 | UfUfgGfuGfcUfgCfuUf | 1108 |
| | | | aGfcAfcCfaAf(invdT) | | cAfgAfaUfgdTdT | |
| 0212 | 4927 | 4945 | ccaGfaAfgCfaGfcAfcCf | 810 | GfCfuCfaGfuUfgGfuGf | 1109 |
| | | | aAfcUfgAfgCf(invdT) | | cUfgCfuUfcdTdT | |
| 0213 | 4930 | 4948 | ccaGfcAfgCfaCfcAfaCf | 811 | UfUfuGfcUfcAfgUfuGf | 1110 |
| | | | uGfaGfcAfaAf(invdT) | | gUfgCfuGfcdTdT | |
| 0214 | 4960 | 4978 | ccaCfgGfcAfgUfgCfuAf | 812 | UfAfcCfaUfgGfuAfgCf | 1111 |
| | | | cCfaUfgGfuAf(invdT) | | aCfuGfcCfgdTdT | |
| 0215 | 4963 | 4981 | ccaCfaGfuGfcUfaCfcAf | 813 | CfAfuUfaCfcAfuGfgUf | 1112 |
| | | | uGfgUfaAfuGf(invdT) | | aGfcAfcUfgdTdT | |
| 0216 | 4965 | 4983 | ccaGfuGfcUfaCfcAfuGf | 814 | GfCfcAfuUfaCfcAfuGf | 1113 |
| | | | gUfaAfuGfgCf(invdT) | | gUfaGfcAfcdTdT | |
| 0217 | 4972 | 4990 | ccaCfaUfgGfuAfaUfgGf | 815 | AfAfcUfcUfgGfcCfaUf | 1114 |
| | | | cCfaGfaGfuUf(invdT) | | uAfcCfaUfgdTdT | |
| 0218 | 4975 | 4993 | ccaGfgUfaAfuGfgCfcAf | 816 | GfAfuAfaCfuCfuGfgCf | 1115 |
| | | | gAfgUfuAfuCf(invdT) | | cAfuUfaCfcdTdT | |
| 0219 | 4976 | 4994 | ccaGfuAfaUfgGfcCfaGf | 817 | CfGfaUfaAfcUfcUfgGf | 1116 |
| | | | aGfuUfaUfcGf(invdT) | | cCfaUfuAfcdTdT | |
| 0220 | 4977 | 4995 | ccaUfaAfuGfgCfcAfgAf | 818 | UfCfgAfuAfaCfuCfuGf | 1117 |
| | | | gUfuAfuCfgAf(invdT) | | gCfcAfuUfadTdT | |
| 0221 | 4980 | 4998 | ccaUfgGfcCfaGfaGfuUf | 819 | GfCfcUfcGfaUfaAfcUf | 1118 |
| | | | aUfcGfaGfgCf(invdT) | | cUfgGfcCfadTdT | |
| 0222 | 4981 | 4999 | ccaGfgCfcAfgAfgUfuAf | 820 | UfGfcCfuCfgAfuAfaCf | 1119 |
| | | | uCfgAfgGfcAf(invdT) | | uCfuGfgCfcdTdT | |
| 0223 | 4982 | 5000 | ccaGfcCfaGfaGfuUfaUf | 821 | GfUfgCfcUfcGfaUfaAf | 1120 |
| | | | cGfaGfgCfaCf(invdT) | | cUfcUfgGfcdTdT | |
| 0224 | 4983 | 5001 | ccaCfcAfgAfgUfuAfuCf | 822 | UfGfuGfcCfuCfgAfuAf | 1121 |
| | | | gAfgGfcAfcAf(invdT) | | aCfuCfuGfgdTdT | |
| 0225 | 4985 | 5003 | ccaAfgAfgUfuAfuCfgAf | 823 | AfAfuGfuGfcCfuCfgAf | 1122 |
| | | | gGfcAfcAfuUf(invdT) | | uAfaCfuCfudTdT | |
| 0226 | 4986 | 5004 | ccaGfaGfuUfaUfcGfaGf | 824 | GfAfaUfgUfgCfcUfcGf | 1123 |
| | | | gCfaCfaUfuCf(invdT) | | aUfaAfcUfcdTdT | |
| 0227 | 4987 | 5005 | ccaAfgUfuAfuCfgAfgGf | 825 | AfGfaAfuGfuGfcCfuCf | 1124 |
| | | | cAfcAfuUfcUf(invdT) | | gAfuAfaCfudTdT | |
| 0228 | 4997 | 5015 | ccaGfcAfcAfuUfcUfcCf | 826 | AfCfaGfuGfgUfgGfaGf | 1125 |
| | | | aCfcAfcUfgUf(invdT) | | aAfuGfuGfcdTdT | |
| 0229 | 5001 | 5019 | ccaAfuUfcUfcCfaCfcAf | 827 | UfGfuGfaCfaGfuGfgUf | 1126 |
| | | | cUfgUfcAfcAf(invdT) | | gGfaGfaAfudTdT | |
| 0230 | 5016 | 5034 | ccaCfaCfaGfgAfaGfgAf | 828 | UfUfgAfcAfuGfuCfcUf | 1127 |
| | | | CAfuGfuCfaAf(invdT) | | uCfcUfgUfgdTdT | |
| 0231 | 5021 | 5039 | ccaGfaAfgGfaCfaUfgUf | 829 | CfAfaGfaUfuGfaCfaUf | 1128 |
| | | | cAfaUfcUfuGf(invdT) | | gUfcCfuUfcdTdT | |
| 0232 | 5149 | 5167 | ccaUfuUfaCfcAfuGfgAf | 830 | UfGfcUfgGfgGfuCfcAf | 1129 |
| | | | cCfcCfaGfcAf(invdT) | | uGfgUfaAfadTdT | |
| 0233 | 5150 | 5168 | ccaUfuAfcCfaUfgGfaCf | 831 | AfUfgCfuGfgGfgUfcCf | 1130 |
| | | | cCfcAfgCfaUf(invdT) | | aUfgGfuAfadTdT | |
| 0234 | 5180 | 5198 | ccaAfcUfgCfaAfcCfuGf | 832 | CfAfuCfgCfgUfcAfgGf | 1131 |
| | | | aCfgCfgAfuGf(invdT) | | uUfgCfaGfudTdT | |
| 0235 | 5186 | 5204 | ccaAfcCfuGfaCfgCfgAf | 833 | UfCfuGfaGfcAfuCfgCf | 1132 |
| | | | uGfcUfcAfgAf(invdT) | | gUfcAfgGfudTdT | |
| 0236 | 5189 | 5207 | ccaUfgAfcGfcGfaUfgCf | 834 | GfUfgUfcUfgAfgCfaUf | 1133 |
| | | | uCfaGfaCfaCf(invdT) | | cGfcGfuCfadTdT | |
| 0237 | 5190 | 5208 | ccaGfaCfgCfgAfuGfcUf | 835 | UfGfuGfuCfuGfaGfcAf | 1134 |
| | | | cAfgAfcAfcAf(invdT) | | uCfgCfgUfcdTdT | |
| 0238 | 5191 | 5209 | ccaAfcGfcGfaUfgCfuCf | 836 | CfUfgUfgUfcUfgAfgCf | 1135 |
| | | | aGfaCfaCfaGf(invdT) | | aUfcGfcGfudTdT | |
| 0239 | 5192 | 5210 | ccaCfgCfgAfuGfcUfcAf | 837 | UfCfuGfuGfuCfuGfaGf | 1136 |
| | | | gAfcAfcAfgAf(invdT) | | cAfuCfgCfgdTdT | |
| 0240 | 5761 | 5779 | ccaGfaAfgUfgAfaCfcUf | 838 | GfAfgAfuUfcGfaGfgUf | 1137 |
| | | | cGfaAfuCfuCf(invdT) | | uCfaCfuUfcdTdT | |
| 0241 | 5922 | 5940 | ccaCfaGfgAfcUfgAfaUf | 839 | GfAfuGfuAfaCfaUfuCf | 1138 |
| | | | gUfuAfcAfuCf(invdT) | | aGfuCfcUfgdTdT | |
| 0242 | 5956 | 5974 | ccaAfcCfcAfaGfgUfaCf | 840 | UfCfcCfaAfaGfgUfaCf | 1139 |
| | | | cUfuUfgGfgAf(invdT) | | cUfuGfgGfudTdT | |
| 0243 | 5957 | 5975 | ccaCfcCfaAfgGfuAfcCf | 841 | GfUfcCfcAfaAfgGfuAf | 1140 |
| | | | uUfuGfgGfaCf(invdT) | | cCfuUfgGfgdTdT | |
| 0244 | 5964 | 5982 | ccaUfaCfcUfuUfgGfgAf | 842 | AfAfgGfcCfaGfuCfcCf | 1141 |
| | | | cUfgGfcCfuUf(invdT) | | aAfaGfgUfadTdT | |
| 0245 | 5965 | 5983 | ccaAfcCfuUfuGfgGfaCf | 843 | GfAfaGfgCfcAfgUfcCf | 1142 |
| | | | uGfgCfcUfuCf(invdT) | | cAfaAfgGfudTdT | |
| 0246 | 6323 | 6341 | ccaGfaCfaGfcAfaUfcAf | 844 | UfCfuUfcGfuUfuGfaUf | 1143 |
| | | | aAfcGfaAfgAf(invdT) | | uGfcUfgUfcdTdT | |
| 0247 | 6324 | 6342 | ccaAfcAfgCfaAfuCfaAf | 845 | GfUfcUfuCfgUfuUfgAf | 1144 |
| | | | aCfgAfaGfaCf(invdT) | | uUfgCfuGfudTdT | |
| 0248 | 6325 | 6343 | ccaCfaGfcAfaUfcAfaAf | 846 | UfGfuCfuUfcGfuUfuGf | 1145 |
| | | | cGfaAfgAfcAf(invdT) | | aUfuGfcUfgdTdT | |
| 0249 | 6326 | 6344 | ccaAfgCfaAfuCfaAfaCf | 847 | GfUfgUfcUfuCfgUfuUf | 1146 |
| | | | gAfaGfaCfaCf(invdT) | | gAfuUfgCfudTdT | |
| 0250 | 6327 | 6345 | ccaGfcAfaUfcAfaAfcGf | 848 | AfGfuGfuCfuUfcGfuUf | 1147 |
| | | | aAfgAfcAfcUf(invdT) | | uGfaUfuGfcdTdT | |
| 0251 | 6328 | 6346 | ccaCfaAfuCfaAfaCfgAf | 849 | CfAfgUfgUfcUfuCfgUf | 1148 |
| | | | aGfaCfaCfuGf(invdT) | | uUfgAfuUfgdTdT | |
| 0252 | 6330 | 6348 | ccaAfuCfaAfaCfgAfaGf | 850 | AfAfcAfgUfgUfcUfuCf | 1149 |
| | | | aCfaCfuGfuUf(invdT) | | gUfuUfgAfudTdT | |
| 0253 | 6331 | 6349 | ccaUfcAfaAfcGfaAfgAf | 851 | GfAfaCfaGfuGfuCfuUf | 1150 |
| | | | cAfcUfgUfuCf(invdT) | | cGfuUfuGfadTdT | |
| 0254 | 6332 | 6350 | ccaCfaAfaCfgAfaGfaCf | 852 | GfGfaAfcAfgUfgUfcUf | 1151 |
| | | | aCfuGfuUfcCf(invdT) | | uCfgUfuUfgdTdT | |
| 0255 | 6333 | 6351 | ccaAfaAfcGfaAfgAfcAf | 853 | GfGfgAfaCfaGfuGfuCf | 1152 |
| | | | cUfgUfuCfcCf(invdT) | | uUfcGfuUfudTdT | |
| 0256 | 6334 | 6352 | ccaAfaCfgAfaGfaCfaCf | 854 | UfGfgGfaAfcAfgUfgUf | 1153 |
| | | | uGfuUfcCfcAf(invdT) | | cUfuCfgUfudTdT | |
| 0257 | 6335 | 6353 | ccaAfcGfaAfgAfcAfcUf | 855 | CfUfgGfgAfaCfaGfuGf | 1154 |
| | | | gUfuCfcCfaGf(invdT) | | uCfuUfcGfudTdT | |
| 0258 | 6336 | 6354 | ccaCfgAfaGfaCfaCfuGf | 856 | GfCfuGfgGfaAfcAfgUf | 1155 |
| | | | uUfcCfcAfgCf(invdT) | | gUfcUfuCfgdTdT | |
| 0259 | 6337 | 6355 | ccaGfaAfgAfcAfcUfgUf | 857 | AfGfcUfgGfgAfaCfaGf | 1156 |
| | | | uCfcCfaGfcUf(invdT) | | uGfuCfuUfcdTdT | |
| 0260 | 6338 | 6356 | ccaAfaGfaCfaCfuGfuUf | 858 | UfAfgCfuGfgGfaAfcAf | 1157 |
| | | | cCfcAfgCfuAf(invdT) | | gUfgUfcUfudTdT | |
| 0261 | 6339 | 6357 | ccaAfgAfcAfcUfgUfuCf | 859 | GfUfaGfcUfgGfgAfaCf | 1158 |
| | | | cCfaGfcUfaCf(invdT) | | aGfuGfuCfudTdT | |
| 0262 | 6340 | 6358 | ccaGfaCfaCfuGfuUfcCf | 860 | GfGfuAfgCfuGfgGfaAf | 1159 |
| | | | cAfgCfuAfcCf(invdT) | | cAfgUfgUfcdTdT | |
| 0263 | 6341 | 6359 | ccaAfcAfcUfgUfuCfcCf | 861 | UfGfgUfaGfcUfgGfgAf | 1160 |
| | | | aGfcUfaCfcAf(invdT) | | aCfaGfuGfudTdT | |
| 0264 | 6350 | 6368 | ccaCfcAfgCfuAfcCfaGf | 862 | UfGfgCfaUfaGfcUfgGf | 1161 |
| | | | cUfaUfgCfcAf(invdT) | | uAfgCfuGfgdTdT | |
| 0265 | 6351 | 6369 | ccaCfaGfcUfaCfcAfgCf | 863 | UfUfgGfcAfuAfgCfuGf | 1162 |
| | | | uAfuGfcCfaAf(invdT) | | gUfaGfcUfgdTdT | |
| 0266 | 6352 | 6370 | ccaAfgCfuAfcCfaGfcUf | 864 | UfUfuGfgCfaUfaGfcUf | 1163 |
| | | | aUfgCfcAfaAf(invdT) | | gGfuAfgCfudTdT | |
| 0267 | 6353 | 6371 | ccaGfcUfaCfcAfgCfuAf | 865 | GfUfuUfgGfcAfuAfgCf | 1164 |
| | | | uGfcCfaAfaCf(invdT) | | uGfgUfaGfcdTdT | |
| 0268 | 6354 | 6372 | ccaCfuAfcCfaGfcUfaUf | 866 | GfGfuUfuGfgCfaUfaGf | 1165 |
| | | | gCfcAfaAfcCf(invdT) | | cUfgGfuAfgdTdT | |
| 0269 | 6355 | 6373 | ccaUfaCfcAfgCfuAfuGf | 867 | AfGfgUfuUfgGfcAfuAf | 1166 |
| | | | cCfaAfaCfcUf(invdT) | | gCfuGfgUfadTdT | |
| 0270 | 6376 | 6394 | ccaGfcAfuUfuUfuGfgUf | 868 | AfCfaAfaAfaUfaCfcAf | 1167 |
| | | | aUfuUfuUfgUf(invdT) | | aAfaAfuGfcdTdT | |
| 0271 | 6377 | 6395 | ccaCfaUfuUfuUfgGfuAf | 869 | CfAfcAfaAfaAfuAfcCf | 1168 |
| | | | uUfuUfuGfuGf(invdT) | | aAfaAfaUfgdTdT | |
| 0272 | 6378 | 6396 | ccaAfuUfuUfuGfgUfaUf | 870 | AfCfaCfaAfaAfaUfaCf | 1169 |
| | | | uUfuUfgUfgUf(invdT) | | cAfaAfaAfudTdT | |
| 0273 | 6379 | 6397 | ccaUfuUfuUfgGfuAfuUf | 871 | UfAfcAfcAfaAfaAfuAf | 1170 |
| | | | uUfuGfuGfuAf(invdT) | | cCfaAfaAfadTdT | |
| 0274 | 6380 | 6398 | ccaUfuUfuGfgUfaUfuUf | 872 | AfUfaCfaCfaAfaAfaUf | 1171 |
| | | | uUfgUfgUfaUf(invdT) | | aCfcAfaAfadTdT | |
| 0275 | 6381 | 6399 | ccaUfuUfgGfuAfuUfuUf | 873 | UfAfuAfcAfcAfaAfaAf | 1172 |
| | | | uGfuGfuAfuAf(invdT) | | uAfcCfaAfadTdT | |
| 0276 | 6382 | 6400 | ccaUfuGfgUfaUfuUfuUf | 874 | UfUfaUfaCfaCfaAfaAf | 1173 |
| | | | gUfgUfaUfaAf(invdT) | | aUfaCfcAfadTdT | |
| 0277 | 6383 | 6401 | ccaUfgGfuAfuUfuUfuGf | 875 | CfUfuAfuAfcAfcAfaAf | 1174 |
| | | | uGfuAfuAfaGf(invdT) | | aAfuAfcCfadTdT | |
| 0278 | 6384 | 6402 | ccaGfgUfaUfuUfuUfgUf | 876 | GfCfuUfaUfaCfaCfaAf | 1175 |
| | | | gUfaUfaAfgCf(invdT) | | aAfaUfaCfcdTdT | |
| 0279 | 6385 | 6403 | ccaGfuAfuUfuUfuGfuGf | 877 | AfGfcUfuAfuAfcAfcAf | 1176 |
| | | | uAfuAfaGfcUf(invdT) | | aAfaAfuAfcdTdT | |
| 0280 | 6386 | 6404 | ccaUfaUfuUfuUfgUfgUf | 878 | AfAfgCfuUfaUfaCfaCf | 1177 |
| | | | aUfaAfgCfuUf(invdT) | | aAfaAfaUfadTdT | |
| 0281 | 6387 | 6405 | ccaAfuUfuUfuGfuGfuAf | 879 | AfAfaGfcUfuAfuAfcAf | 1178 |
| | | | uAfaGfcUfuUf(invdT) | | cAfaAfaAfudTdT | |
| 0282 | 6388 | 6406 | ccaUfuUfuUfgUfgUfaUf | 880 | AfAfaAfgCfuUfaUfaCf | 1179 |
| | | | aAfgCfuUfuUf(invdT) | | aCfaAfaAfadTdT | |
| 0283 | 6455 | 6473 | ccaUfgUfuAfaAfaAfuAf | 881 | GfCfaGfaGfuUfuAfuUf | 1180 |
| | | | aAfcUfcUfgCf(invdT) | | uUfuAfaCfadTdT | |
| 0284 | 6456 | 6474 | ccaGfuUfaAfaAfaUfaAf | 882 | UfGfcAfgAfgUfuUfaUf | 1181 |
| | | | aCfuCfuGfcAf(invdT) | | uUfuUfaAfcdTdT | |
| 0285 | 6457 | 6475 | ccaUfuAfaAfaAfuAfaAf | 883 | GfUfgCfaGfaGfuUfuAf | 1182 |
| | | | cUfcUfgCfaCf(invdT) | | uUfuUfuAfadTdT | |
| 0286 | 6458 | 6476 | ccaUfaAfaAfaUfaAfaCf | 884 | AfGfuGfcAfgAfgUfuUf | 1183 |
| | | | uCfuGfcAfcUf(invdT) | | aUfuUfuUfadTdT | |
| 0287 | 6459 | 6477 | ccaAfaAfaAfuAfaAfcUf | 885 | AfAfgUfgCfaGfaGfuUf | 1184 |
| | | | cUfgCfaCfuUf(invdT) | | uAfuUfuUfudTdT | |
| 0288 | 6460 | 6478 | ccaAfaAfaUfaAfaCfuCf | 886 | UfAfaGfuGfcAfgAfgUf | 1185 |
| | | | uGfcAfcUfuAf(invdT) | | uUfaUfuUfudTdT | |
| 0289 | 6461 | 6479 | ccaAfaAfuAfaAfcUfcUf | 887 | AfUfaAfgUfgCfaGfaGf | 1186 |
| | | | gCfaCfuUfaUf(invdT) | | uUfuAfuUfudTdT | |
| 0290 | 6462 | 6480 | ccaAfaUfaAfaCfuCfuGf | 888 | AfAfuAfaGfuGfcAfgAf | 1187 |
| | | | cAfcUfuAfuUf(invdT) | | gUfuUfaUfudTdT | |
| 0291 | 6463 | 6481 | ccaAfuAfaAfcUfcUfgCf | 889 | AfAfaUfaAfgUfgCfaGf | 1188 |
| | | | aCfuUfaUfuUf(invdT) | | aGfuUfuAfudTdT | |
| 0292 | 6464 | 6482 | ccaUfaAfaCfuCfuGfcAf | 890 | AfAfaAfuAfaGfuGfcAf | 1189 |
| | | | cUfuAfuUfuUf(invdT) | | gAfgUfuUfadTdT | |
| 0293 | 6465 | 6483 | ccaAfaAfcUfcUfgCfaCf | 891 | CfAfaAfaUfaAfgUfgCf | 1190 |
| | | | uUfaUfuUfuGf(invdT) | | aGfaGfuUfudTdT | |
| 0294 | 6466 | 6484 | ccaAfaCfuCfuGfcAfcUf | 892 | UfCfaAfaAfuAfaGfuGf | 1191 |
| | | | uAfuUfuUfgAf(invdT) | | cAfgAfgUfudTdT | |
| 0295 | 6467 | 6485 | ccaAfcUfcUfgCfaCfuUf | 893 | AfUfcAfaAfaUfaAfgUf | 1192 |
| | | | aUfuUfuGfaUf(invdT) | | gCfaGfaGfudTdT | |
| 0296 | 6468 | 6486 | ccaCfuCfuGfcAfcUfuAf | 894 | AfAfuCfaAfaAfuAfaGf | 1193 |
| | | | uUfuUfgAfuUf(invdT) | | uGfcAfgAfgdTdT | |
| 0297 | 6469 | 6487 | ccaUfcUfgCfaCfuUfaUf | 895 | AfAfaUfcAfaAfaUfaAf | 1194 |
| | | | uUfuGfaUfuUf(invdT) | | gUfgCfaGfadTdT | |
| 0298 | 6470 | 6488 | ccaCfuGfcAfcUfuAfuUf | 896 | CfAfaAfuCfaAfaAfuAf | 1195 |
| | | | uUfgAfuUfuGf(invdT) | | aGfuGfcAfgdTdT | |
| 0299 | 6471 | 6489 | ccaUfgCfaCfuUfaUfuUf | 897 | UfCfaAfaUfcAfaAfaUf | 1196 |
| | | | uGfaUfuUfgAf(invdT) | | aAfgUfgCfadTdT | |

In some embodiments, the dsRNA comprises one or more modified nucleotides described in PCT Publication WO 2019/170731, the disclosure of which is incorporated herein in its entirety. In such modified nucleotides, the ribose ring has been replaced by a six-membered heterocyclic ring. Such a modified nucleotide has the structure of formula (I):

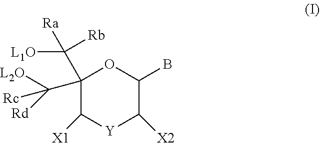

wherein:
B is a heterocyclic nucleobase;
one of L1 and L2 is an internucleoside linking group linking the compound of formula (I) to a polynucleotide and the other of L1 and L2 is H, a protecting group, a phosphorus moiety or an internucleoside linking group linking the compound of formula (I) to a polynucleotide,
Y is O, NH, NR1 or N—C(═O)—R1, wherein R1 is:
a (C1-C20) alkyl group, optionally substituted by one or more groups selected from an halogen atom, a (C1-C6) alkyl group, a (C3-C8) cycloalkyl group, a (C3-C14) heterocycle, a (C6-C14) aryl group, a (C5-C14) heteroaryl group, —O—Z1, —N(Z1)(Z2), —S—Z1, —CN, —C(=J)-O—Z1, —O—C(=J)-Z1, —C(=J)-N(Z1)(Z2), and —N(Z1)-C(=J)-Z2, wherein

### J is O or S,

each of Z1 and Z2 is, independently, H, a (C1-C6) alkyl group, optionally substituted by one or more groups selected from a halogen atom and a (C1-C6) alkyl group,
a (C3-C8) cycloalkyl group, optionally substituted by one or more groups selected from a halogen atom and a (C1-C6) alkyl group,
a group —[C(═O)]m-R2-(O—CH₂—CH₂)p-R3, wherein
m is an integer meaning 0 or 1,
p is an integer ranging from 0 to 10,
R2 is a (C1-C20) alkylene group optionally substituted by a (C1-C6) alkyl group, —O—Z3, —N(Z3)(Z4), —S—Z3, —CN, —C(═K)—O—Z3, —O—C(═K)—Z3, —C(═K)—N(Z3)(Z4), or —N(Z3)-C(═K)—Z4, wherein

### K is O or S,

each of Z3 and Z4 is, independently, H, a (C1-C6) alkyl group, optionally substituted by one or more groups selected from a halogen atom and a (C1-C6) alkyl group, and
R3 is selected from the group consisting of a hydrogen atom, a (C1-C6) alkyl group, a (C1-C6) alkoxy group, a (C3-C8) cycloalkyl group, a (C3-C14) heterocycle, a (C6-C14) aryl group or a (C5-C14) heteroaryl group, or R3 is a cell targeting moiety,
X1 and X2 are each, independently, a hydrogen atom, a (C1-C6) alkyl group, and
each of Ra, Rb, Rc and Rd is, independently, H or a (C1-C6) alkyl group,
   or is a pharmaceutically acceptable salt thereof.

In some embodiments, Y is NR1, R1 is a non-substituted (C1-C20) alkyl group, and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a non-substituted (C1-C16) alkyl group, which includes an alkyl group selected from a group comprising methyl, isopropyl, butyl, octyl, hexadecyl, and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a (C3-C8) cycloalkyl group, optionally substituted by one or more groups selected from a halogen atom and a (C1-C6) alkyl group, and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a cyclohexyl group, and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a (C1-C20) alkyl group substituted by a (C6-C14) aryl group and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a methyl group substituted by a phenyl group, and L1, L2, Ra, Rb, Rc, Rd, X1, X2, R2, R3 and B have the same meaning as defined for the general formula (I), or a pharmaceutically acceptable salt thereof.

In some embodiments, Y is N—C(═O)—R1, R1 is an optionally substituted (C1-C20) alkyl group, and L1, L2, Ra, Rb, Rc, Rd, X1, X2, R2, R3 and B have the same meaning as defined for the general formula (I), or a pharmaceutically acceptable salt thereof.

In some embodiments, Y is N—C(═O)—R1, R1 is selected from a group comprising methyl and pentadecyl and L1, L2, Ra, Rb, Rc, Rd, X1, X2, R2, R3 and B have the same meaning as defined for the general formula (I), or a pharmaceutically acceptable salt thereof.

In some embodiments, the dsRNA comprises one or more compounds of formula (I) wherein Y is
a) NR1, wherein R1 is a non-substituted (C1-C20) alkyl group;
b) NR1, wherein R1 is a non-substituted (C1-C16) alkyl group, which includes an alkyl group selected from a group comprising methyl, isopropyl, butyl, octyl, and hexadecyl;
c) NR1, wherein R1 is a (C3-C8) cycloalkyl group, optionally substituted by one or more groups selected from a halogen atom and a (C1-C6) alkyl group;
d) NR1, wherein R1 is a cyclohexyl group;
e) NR1, wherein R1 is a (C1-C20) alkyl group substituted by a (C6-C14) aryl group;
f) NR1, wherein R1 is a methyl group substituted by a phenyl group;
g) N—C(═O)—R1, wherein R1 is an optionally substituted (C1-C20) alkyl group; or
h) N—C(═O)—R1, wherein R1 is methyl or pentadecyl.

In some embodiments, B is selected from a group comprising a pyrimidine, a substituted pyrimidine, a purine and a substituted purine, or a pharmaceutically acceptable salt thereof.

In some embodiments, the internucleoside linking group in the dsRNA is independently selected from the group consisting of phosphodiester, phosphotriester, phosphorothioate, phosphorodithioate, alkyl-phosphonate and phosphoramidate backbone linking groups, or a pharmaceutically acceptable salt thereof. In some embodiments, the dsRNA comprises one or more internucleoside linking groups independently selected from the group consisting of phosphodiester, phosphotriester, phosphorothioate, phosphorodithioate, alkyl-phosphonate and phosphoramidate backbone linking groups, or a pharmaceutically acceptable salt thereof.

In some embodiments, the dsRNA comprises from 2 to 10 compounds of formula (I), or a pharmaceutically acceptable salt thereof. In a particular embodiment, the 2 to 10 compounds of formula (I) are on the sense strand.

In further embodiments, the dsRNA comprises one or more targeted nucleotides or a pharmaceutically acceptable salt thereof.

In some embodiments, R3 is of the formula (II):

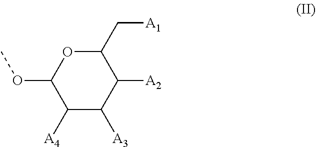

wherein A1, A2 and A3 are OH,
A4 is OH or NHC(═O)—R5, wherein R5 is a (C1-C6) alkyl group, optionally substituted by a halogen atom. or a pharmaceutically acceptable salt thereof

In some embodiments, R3 is N-acetyl-galactosamine, or a pharmaceutically acceptable salt thereof The precursors that can be used to make modified siRNAs having nucleotides of

formula (I) are exemplified in Table A below. Table A shows examples of phosphoramidite nucleotide analogs for oligonucleotide synthesis. In the (2S,6R) diastereomeric series, the phosphoramidites as nucleotide precursors are abbreviated with a “pre-1”, the nucleotide analogs are abbreviated with an “l”, followed by the nucleobase and a number, which specifies the group Y in formula (I). To distinguish both stereochemistries, the analogues (2R,6R)-diastereoisomers are indicated with an additional “b.” Targeted nucleotide precursors, targeted nucleotide analogs and solid supports are abbreviated as described above, but with an “lg” instead of the “l.”

TABLE A

| | | | Name in | |
|---|---|---|---|---|
| | | Precursor | oligo- | |
| No | Structure | name | sequence | Stereochemistry |
| 1 | 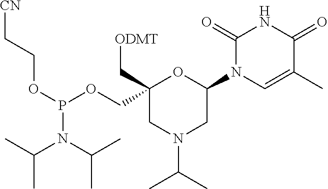 | pre-lT3 | lT3 | (2S,6R) |
| 2 | 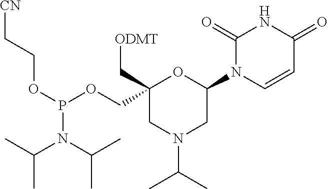 | pre-lU3 | lU3 | (2S,6R) |
| 3 | 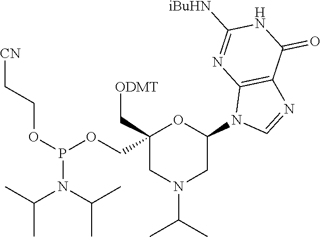 | pre-lG3 | lG3 | (2S,6R) |
| 4 | 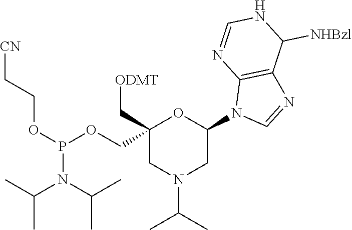 | pre-lA3 | lA3 | (2S,6R) |
| 5 | 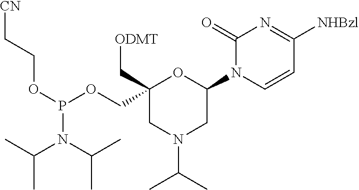 | pre-lC3 | lC3 | (2S,6R) |
| 6 | 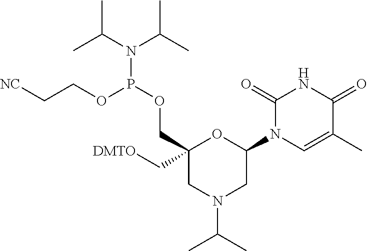 | pre-lT3b | lT3b | (2R,6R) |
| 7 | 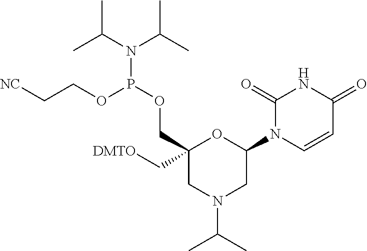 | pre-lU3b | lU3b | (2R,6R) |
| 8 | 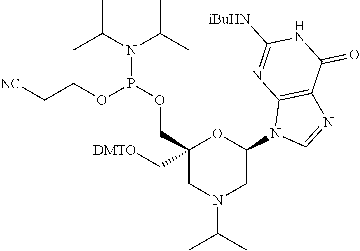 | pre-lG3b | lG3b | (2R,6R) |
| 9 | 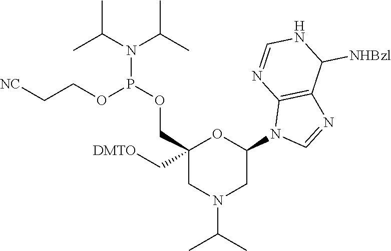 | pre-lA3b | lA3b | (2R,6R) |
| 10 | 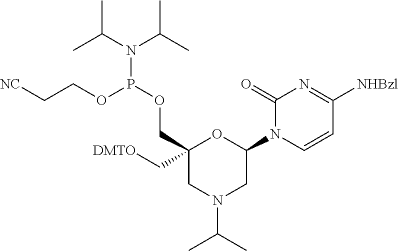 | pre-lC3b | lC3b | (2R,6R) |
| 11 | 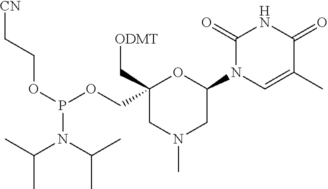 | pre-lT2 | lT2 | (2S,6R) |
| 12 | 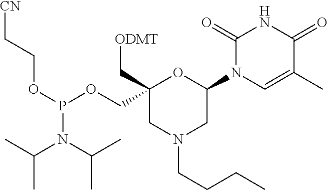 | pre-lT6 | lT6 | (2S,6R) |
| 13 | 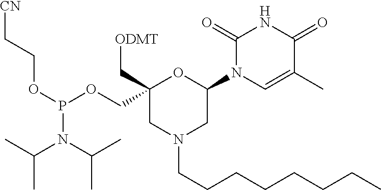 | pre-lT7 | lT7 | (2S,6R) |
| 14 | 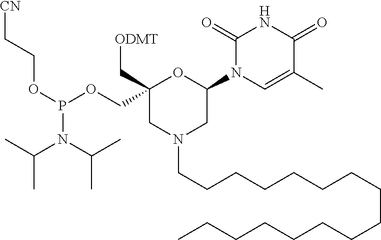 | pre-lT8 | lT8 | (2S,6R) |
| 15 | 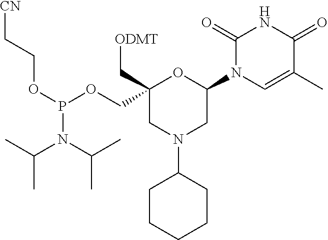 | pre-lT4 | lT4 | (2S,6R) |
| 16 | 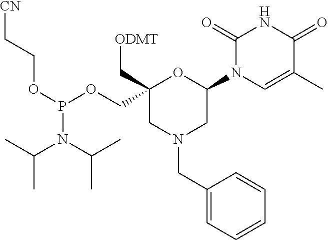 | pre-lT5 | lT5 | (2S,6R) |
| 17 | 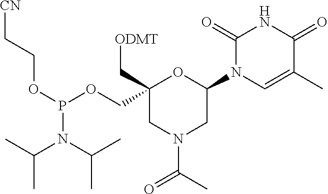 | pre-lT9 | lT9 | (2S,6R) |
| 18 | 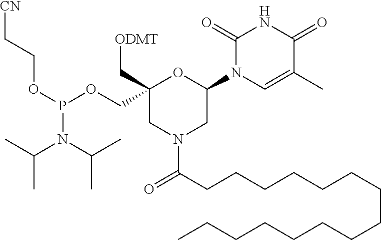 | pre-lT10 | lT10 | (2S,6R) |
| 19 | 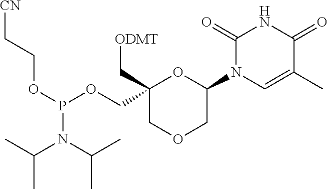 | pre-lT1 | lT1 | (2S,6R) |
| 20 | 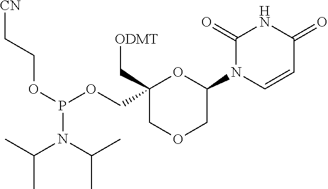 | pre-lU1 | lU1 | (2S,6R) |
| 21 | 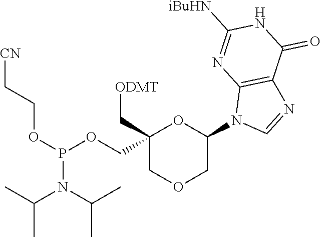 | pre-lG1 | lG1 | (2S,6R) |
| 22 | 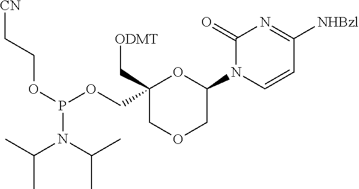 | pre-lC1 | lC1 | (2S,6R) |
| 23 | 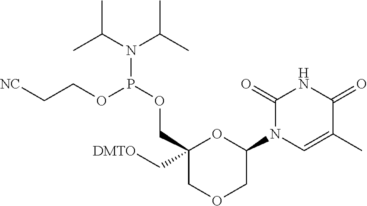 | pre-lT1b | lT1b | (2R,6R) |
| 24 | 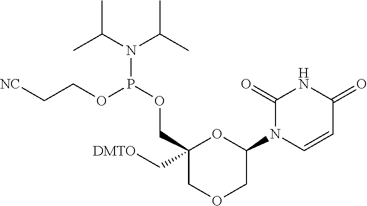 | pre-lU1b | lU1b | (2R,6R) |
| 25 | 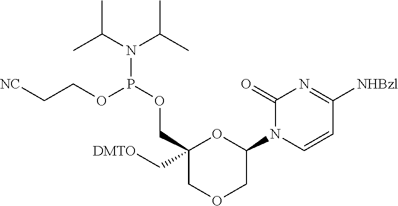 | pre-lC1b | lC1b | (2R,6R) |
| 26 | 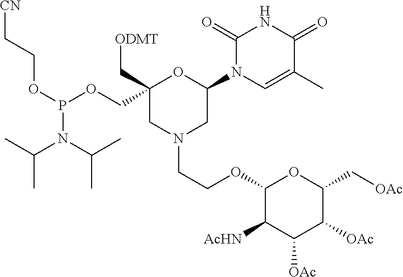 | pre-lgT9 | lgT9 | (2S,6R) |
| 27 | 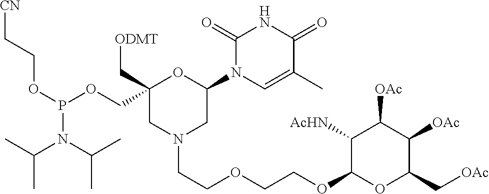 | pre-lgT8 | lgT8 | (2S,6R) |
| 28 | 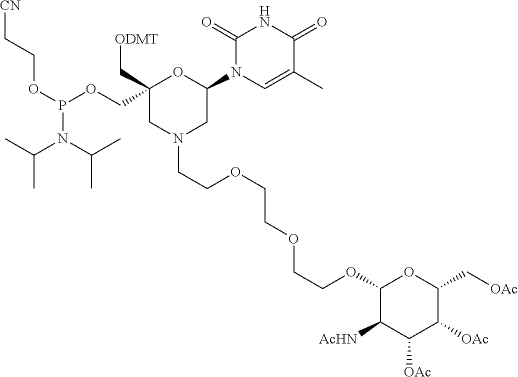 | pre-lgT7 | lgT7 | (2S,6R) |
| 29 | 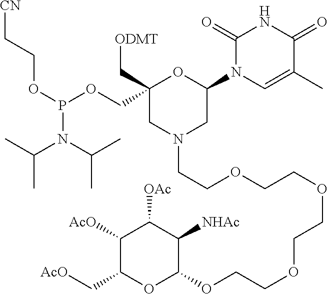 | pre-lgT6 | lgT6 | (2S,6R) |
| 30 | 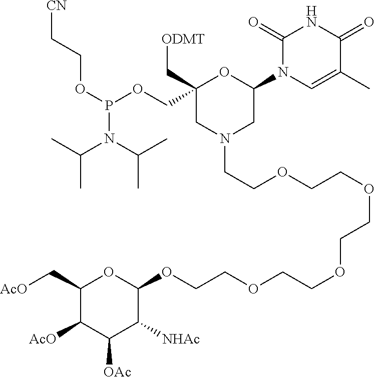 | pre-lgT5 | lgT5 | (2S,6R) |
| 31 | 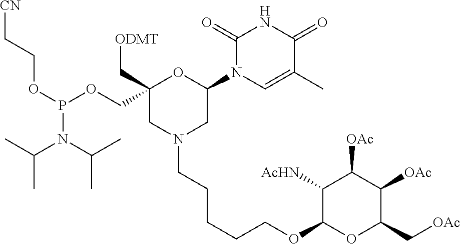 | pre-lgT3 | lgT3 | (2S,6R) |
| 32 | 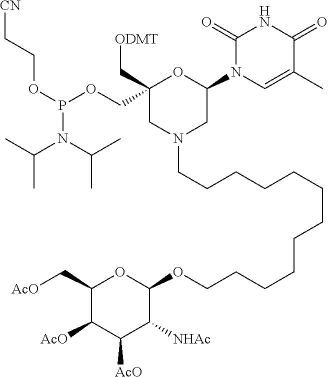 | pre-lgT4 | lgT4 | (2S,6R) |
| 33 | 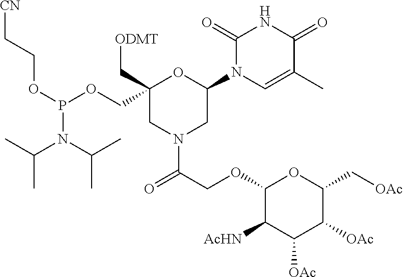 | pre-lgT12 | lgT12 | (2S,6R) |
| 34 | 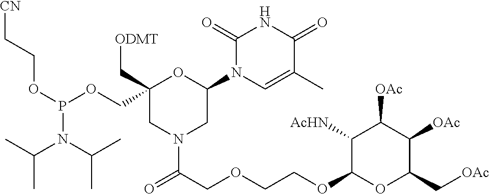 | pre-lgT11 | lgT11 | (2S,6R) |
| 35 | 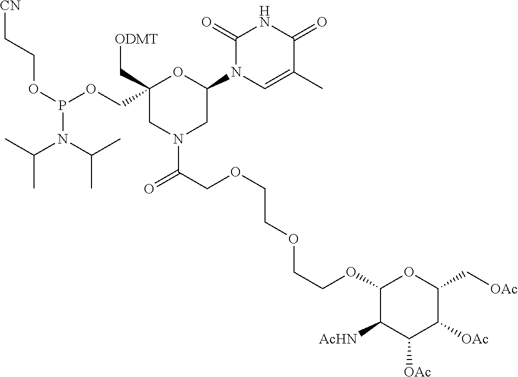 | pre-lgT10 | lgT10 | (2S,6R) |
| 36 | 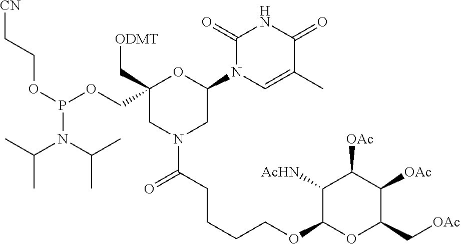 | pre-lgT1 | lgT1 | (2S,6R) |
| 37 | 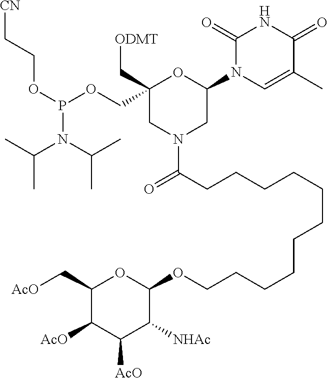 | pre-lgT2 | lgT2 | (2S,6R) |
| 38 | 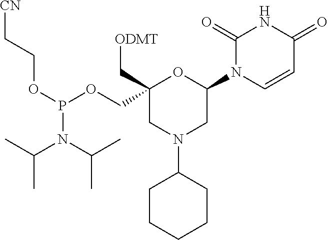 | pre-lU4 | lU4 | (2S,6R) |
| 39 | 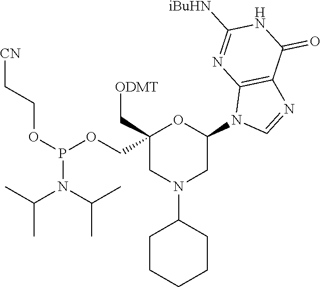 | pre-lG4 | lG4 | (2S,6R) |
| 40 | 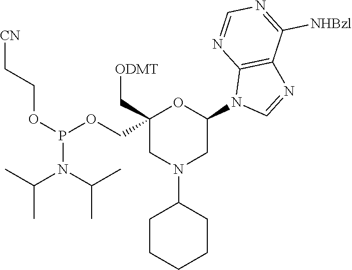 | pre-lA4 | lA4 | (2S,6R) |
| 41 | 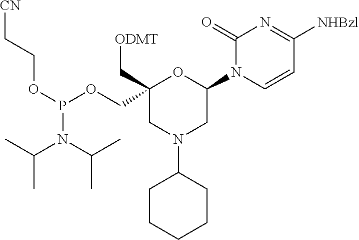 | pre-lC4 | lC4 | (2S,6R) |
| 42 | 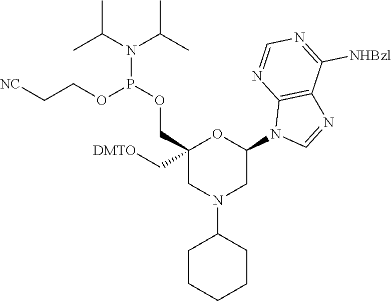 | pre-lA4b | lA4b | (2R,6R) |
| 43 | 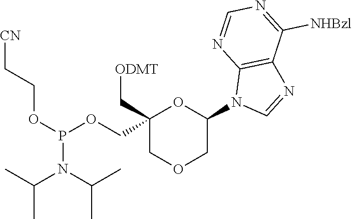 | pre-lA1 | lA1 | (2S,6R) |
| 44 | 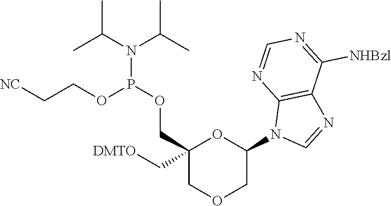 | pre-lA1b | lA1b | (2R,6R) |
| 45 | 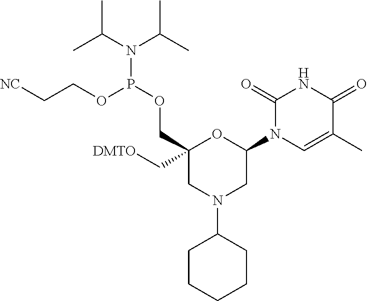 | pre-lT4b | lT4b | (2R,6R) |
| 46 | 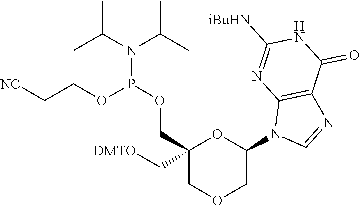 | pre-lG1b | lG1b | (2R,6R) |

The modified nucleotides of formula (I) may be incorporated at the 5′, 3′, or both ends of the sense strand and/or antisense strand of the dsRNA. By way of example, one or more (e.g., 1, 2, 3, 4, or 5 or more) modified nucleotides may be incorporated at the 5′ end of the sense strand of the dsRNA. In some embodiments, one or more (e.g., 1, 2, 3, or more) modified nucleotides are positioned in the 5′ end of the sense strand, where the modified nucleotides do not complement the antisense sequence but may be optionally paired with an equal or smaller number of complementary nucleotides at the corresponding 3′ end of the antisense strand. In a particular embodiment, the sense strand comprises two to five compounds of formula (I) at the 5′ end, and/or comprises one to three compounds of formula (I) at the 3′ end.

In some embodiments,
a) the two to five compounds of formula (I) at the 5′ end of the sense strand comprise lgT3, optionally comprising three consecutive lgT3 nucleotides; and/or
b) the one to three compounds of formula (I) at the 3′ end of the sense strand comprise lT4; optionally comprising two consecutive lT4.

In some embodiments, the dsRNA may comprise a sense strand having a sense sequence of 17, 18, or 19 nucleotides in length, where three to five nucleotides of formula (I) (e.g., three consecutive lgT3 or lgT7 with or without additional nucleotides of formula (I)) are placed in the 5′ end of the sense sequence, making the sense strand 20, 21, or 22 nucleotides in length. In such embodiments, the sense strand may additionally comprise two consecutive nucleotides of formula (I) (e.g., 1T4 or lT3) at the 3′ of the sense sequence, making the sense strand 22, 23, or 24 nucleotides in length. The dsRNA may comprise an antisense sequence of 19 nucleotides in length, where the antisense sequence may additionally be linked to 2 modified nucleotides or deoxyribonucleotides (e.g., dT) at its 3′ end, making the antisense strand 21 nucleotides in length. In further embodiments, the sense strand of the dsRNA contains only naturally occurring internucleotide bonds (phosphodiester bond), where the antisense strand may optionally contain non-naturally occurring internucleotide bonds. For example, the antisense strand may contain phosphorothioate bonds in the backbone near or at its 5′ and/or 3′ ends.

In some embodiments, the use of modified nucleotides of formula (I) circumvents the need for other RNA modifications such as the use of non-naturally occurring internucleotide bonds, thereby simplifying the chemical synthesis of dsRNAs. Moreover, the modified nucleotides of formula (I) can be readily made to contain cell targeted moieties such as GalNAc derivatives (which include GalNAc itself), enhancing the delivery efficiency of dsRNAs incorporating such nucleotides. Further, it has been shown that dsRNAs incorporating modified nucleotides of formula (I), e.g., at the sense strand, significantly improve the stability and therapeutic potency of the dsRNAs.

Table 3 below lists the sequences of exemplary modified GalNAc-siRNA constructs derived from selected siRNA constructs listed in Table 2. In the table, mX=2′-O-Me nucleotide; fX=2′-F nucleotide; dX=DNA nucleotide; PO=phosphodiester linkage; PS=phosphorothioate bond. In these constructs, the sequences of their sense strands and antisense strands correspond to the sense and antisense sequences of the constructs in Table 1 with the same construct numbers, but for the inclusion of (1) the modified 2′-O-Me nucleotides and 2′-F nucleotides, (2) 3 lgT3 nucleotides at the 5′ end of the sense strand sequence, and (3) phosphorothioate bonds.

TABLE 3
Exemplary LPA GalNAc-siRNA Constructs

| siLPA | Parent | Sense strand sequence | | Antisense strand sequence | |
|---|---|---|---|---|---|
| # | CNST# | (5′-3′) | SEQ | (5′-3′) | SEQ |
| 300 | 4 | lgT3-PO-lgT3-PO-lgT3-PO- | 1197 | fA-PS-fC-PS-mG-PO- | 1214 |
| | | fC-PO-mA-PO-fG-PO-mA-PO- | | fU-PO-mG-PO-fC-PO- | |
| | | fG-PO-mU-PO-fU-PO-mA-PO- | | mC-PO-fU-PO-mC-PO- | |
| | | fU-PO-mC-PO-fG-PO-mA-PO- | | fG-PO-mA-PO-fU-PO- | |
| | | fG-PO-mG-PO-fC-PO-mA-PO- | | mA-PO-fA-PO-mC-PO- | |
| | | fC-PS-mG-PS-fU | | fU-PO-mC-PO-fU-PO- | |
| | | | | mG-PS-dT-PS-dT | |
| 301 | 7 | lgT3-PO-lgT3-PO-lgT3-PO- | 1198 | fA-PS-fG-PS-mU-PO- | 1215 |
| | | fA-PO-mG-PO-fU-PO-mU-PO- | | fA-PO-mC-PO-fG-PO- | |
| | | fA-PO-mU-PO-fC-PO-mG-PO- | | mU-PO-fG-PO-mC-PO- | |
| | | fA-PO-mG-PO-fG-PO-mC-PO- | | fC-PO-mU-PO-fC-PO- | |
| | | fA-PO-mC-PO-fG-PO-mU-PO- | | mG-PO-fA-PO-mU-PO- | |
| | | fA-PS-mC-PS-fU | | fA-PO-mA-PO-fC-PO- | |
| | | | | mU-PS-dT-PS-dT | |
| 302 | 19 | lgT3-PO-lgT3-PO-lgT3-PO- | 1199 | fU-PS-fA-PS-mG-PO- | 1216 |
| | | fA-PO-mU-PO-fA-PO-mG-PO- | | fU-PO-mU-PO-fU-PO- | |
| | | fG-PO-mA-PO-fC-PO-mC-PO- | | mU-PO-fC-PO-mU-PO- | |
| | | fA-PO-mC-PO-fA-PO-mG-PO- | | fG-PO-mU-PO-fG-PO- | |
| | | fA-PO-mA-PO-fA-PO-mA-PO- | | mG-PO-fU-PO-mC-PO- | |
| | | fC-PS-mU-PS-fA | | fC-PO-mU-PO-fA-PO- | |
| | | | | mU-PS-dT-PS-dT | |
| 303 | 90 | lgT3-PO-lgT3-PO-lgT3-PO- | 1200 | fA-PS-fU-PS-mA-PO- | 1217 |
| | | fC-PO-mG-PO-fG-PO-mU-PO- | | fA-PO-mC-PO-fU-PO- | |
| | | fA-PO-mA-PO-fU-PO-mG-PO- | | mC-PO-fU-PO-mG-PO- | |
| | | fG-PO-mA-PO-fC-PO-mA-PO- | | fU-PO-mC-PO-fC-PO- | |
| | | fG-PO-mA-PO-fG-PO-mU-PO- | | mA-PO-fU-PO-mU-PO- | |
| | | fU-PS-mA-PS-fU | | fA-PO-mC-PO-fC-PO- | |
| | | | | mG-PS-dT-PS-dT | |
| 304 | 104 | lgT3-PO-lgT3-PO-lgT3-PO- | 1201 | fA-PS-fU-PS-mA-PO- | 1218 |
| | | fC-PO-mG-PO-fG-PO-mA-PO- | | fA-PO-mC-PO-fU-PO- | |
| | | fA-PO-mA-PO-fU-PO-mG-PO- | | mC-PO-fU-PO-mG-PO- | |
| | | fG-PO-mA-PO-fC-PO-mA-PO- | | fU-PO-mC-PO-fC-PO- | |
| | | fG-PO-mA-PO-fG-PO-mU-PO- | | mA-PO-fU-PO-mU-PO- | |
| | | fU-PS-mA-PS-fU | | fU-PO-mC-PO-fC-PO- | |
| | | | | mG-PS-dT-PS-dT | |
| 305 | 107 | lgT3-PO-lgT3-PO-lgT3-PO- | 1202 | fU-PS-fG-PS-mC-PO- | 1219 |
| | | fG-PO-mG-PO-fA-PO-mC-PO- | | fC-PO-mU-PO-fU-PO- | |
| | | fA-PO-mG-PO-fA-PO-mG-PO- | | mG-PO-LA-PO-mU-PO- | |
| | | fU-PO-mU-PO-fA-PO-mU-PO- | | fA-PO-mA-PO-fC-PO- | |
| | | fC-PO-mA-PO-fA-PO-mG-PO- | | mU-PO-fC-PO-mU-PO- | |
| | | fG-PS-mC-PS-fA | | fG-PO-mU-PO-fC-PO- | |
| | | | | mC-PS-dT-PS-dT | |
| 306 | 108 | lgT3-PO-lgT3-PO-lgT3-PO- | 1203 | fG-PS-fU-PS-mG-PO- | 1220 |
| | | fG-PO-mA-PO-fC-PO-mA-PO- | | fC-PO-mC-PO-fU-PO- | |
| | | fG-PO-mA-PO-fG-PO-mU-PO- | | mU-PO-fG-PO-mA-PO- | |
| | | fU-PO-mA-PO-fU-PO-mC-PO- | | fU-PO-mA-PO-fA-PO- | |
| | | fA-PO-mA-PO-fG-PO-mG-PO- | | mC-PO-fU-PO-mC-PO- | |
| | | fC-PS-mA-PS-fC | | fU-PO-mG-PO-fU-PO- | |
| | | | | mC-PS-dT-PS-dT | |
| 307 | 110 | lgT3-PO-lgT3-PO-lgT3-PO- | 1204 | fG-PS-fU-PS-mA-PO- | 1221 |
| | | fG-PO-mA-PO-fG-PO-mU-PO- | | fU-PO-mG-PO-fU-PO- | |
| | | fU-PO-mA-PO-fU-PO-mC-PO- | | mG-PO-fC-PO-mC-PO- | |
| | | fA-PO-mA-PO-fG-PO-mG-PO- | | fU-PO-mU-PO-fG-PO- | |
| | | fC-PO-mA-PO-fC-PO-mA-PO- | | mA-PO-fU-PO-mA-PO- | |
| | | fU-PS-mA-PS-fC | | fA-PO-mC-PO-fU-PO- | |
| | | | | mC-PS-dT-PS-dT | |
| 308 | 111 | lgT3-PO-lgT3-PO-lgT3-PO- | 1205 | fA-PS-fG-PS-mU-PO- | 1222 |
| | | fA-PO-mG-PO-fU-PO-mU-PO- | | fA-PO-mU-PO-fG-PO- | |
| | | fA-PO-mU-PO-fC-PO-mA-PO- | | mU-PO-fG-PO-mC-PO- | |
| | | fA-PO-mG-PO-fG-PO-mC-PO- | | fC-PO-mU-PO-fU-PO- | |
| | | fA-PO-mC-PO-LA-PO-mU-PO- | | mG-PO-fA-PO-mU-PO- | |
| | | fA-PS-mC-PS-fU | | fA-PO-mA-PO-fC-PO- | |
| | | | | mU-PS-dT-PS-dT | |
| 309 | 168 | lgT3-PO-lgT3-PO-lgT3-PO- | 1206 | fU-PS-fC-PS-mG-PO- | 1223 |
| | | fU-PO-mG-PO-fA-PO-mU-PO- | | fA-PO-mU-PO-fA-PO- | |
| | | fG-PO-mG-PO-LA-PO-mC-PO- | | mA-PO-fC-PO-mU-PO- | |
| | | fG-PO-mG-PO-LA-PO-mG-PO- | | fC-PO-mC-PO-fG-PO- | |
| | | fU-PO-mU-PO-fA-PO-mU-PO- | | mU-PO-fC-PO-mC-PO- | |
| | | fC-PS-mG-PS-fA | | fA-PO-mU-PO-fC-PO- | |
| | | | | mA-PS-dT-PS-dT | |
| 310 | 169 | lgT3-PO-lgT3-PO-lgT3-PO- | 1207 | fC-PS-fU-PS-mC-PO- | 1224 |
| | | fG-PO-mA-PO-fU-PO-mG-PO- | | fG-PO-mA-PO-fU-PO- | |
| | | fG-PO-mA-PO-fC-PO-mG-PO- | | mA-PO-fA-PO-mC-PO- | |
| | | fG-PO-mA-PO-fG-PO-mU-PO- | | fU-PO-mC-PO-fC-PO- | |
| | | fU-PO-mA-PO-fU-PO-mC-PO- | | mG-PO-fU-PO-mC-PO- | |
| | | fG-PS-mA-PS-fG | | fC-PO-mA-PO-fU-PO- | |
| | | | | mC-PS-dT-PS-dT | |
| 311 | 172 | lgT3-PO-lgT3-PO-lgT3-PO- | 1208 | fU-PS-fG-PS-mC-PO- | 1225 |
| | | fG-PO-mG-PO-fA-PO-mC-PO- | | fC-PO-mU-PO-fC-PO- | |
| | | fG-PO-mG-PO-fA-PO-mG-PO- | | mG-PO-fA-PO-mU-PO- | |
| | | fU-PO-mU-PO-fA-PO-mU-PO- | | fA-PO-mA-PO-fC-PO- | |
| | | fC-PO-mG-PO-fA-PO-mG-PO- | | mU-PO-fC-PO-mC-PO- | |
| | | fG-PS-mC-PS-fA | | fG-PO-mU-PO-fC-PO- | |
| | | | | mC-PS-dT-PS-dT | |
| 312 | 200 | lgT3-PO-lgT3-PO-lgT3-PO- | 1209 | fU-PS-fG-PS-mA-PO- | 1226 |
| | | fC-PO-mA-PO-fA-PO-mU-PO- | | fG-PO-mC-PO-fA-PO- | |
| | | fC-PO-mU-PO-fG-PO-mA-PO- | | mU-PO-fU-PO-mG-PO- | |
| | | fC-PO-mA-PO-fC-PO-mA-PO- | | fU-PO-mG-PO-fU-PO- | |
| | | fA-PO-mU-PO-fG-PO-mC-PO- | | mC-PO-fA-PO-mG-PO- | |
| | | fU-PS-mC-PS-fA | | fA-PO-mU-PO-fU-PO- | |
| | | | | mG-PS-dT-PS-dT | |
| 313 | 221 | lgT3-PO-lgT3-PO-lgT3-PO- | 1210 | fG-PS-fC-PS-mC-PO- | 1227 |
| | | fU-PO-mG-PO-fG-PO-mC-PO- | | fU-PO-mC-PO-fG-PO- | |
| | | fC-PO-mA-PO-fG-PO-mA-PO- | | mA-PO-fU-PO-mA-PO- | |
| | | fG-PO-mU-PO-fU-PO-mA-PO- | | fA-PO-mC-PO-fU-PO- | |
| | | fU-PO-mC-PO-fG-PO-mA-PO- | | mC-PO-fU-PO-mG-PO- | |
| | | fG-PS-mG-PS-fC | | fG-PO-mC-PO-fC-PO- | |
| | | | | mA-PS-dT-PS-dT | |
| 314 | 223 | lgT3-PO-lgT3-PO-lgT3-PO- | 1211 | fG-PS-fU-PS-mG-PO- | 1228 |
| | | fG-PO-mC-PO-fC-PO-mA-PO- | | fC-PO-mC-PO-fU-PO- | |
| | | fG-PO-mA-PO-fG-PO-mU-PO- | | mC-PO-fG-PO-mA-PO- | |
| | | fU-PO-mA-PO-fU-PO-mC-PO- | | fU-PO-mA-PO-fA-PO- | |
| | | fG-PO-mA-PO-fG-PO-mG-PO- | | mC-PO-fU-PO-mC-PO- | |
| | | fC-PS-mA-PS-fC | | fU-PO-mG-PO-fG-PO- | |
| | | | | mC-PS-dT-PS-dT | |
| 315 | 279 | lgT3-PO-lgT3-PO-lgT3-PO- | 1212 | fA-PS-fG-PS-mC-PO- | 1229 |
| | | fG-PO-mU-PO-fA-PO-mU-PO- | | fU-PO-mU-PO-LA-PO- | |
| | | fU-PO-mU-PO-fU-PO-mU-PO- | | mU-PO-LA-PO-mC-PO- | |
| | | fG-PO-mU-PO-fG-PO-mU-PO- | | fA-PO-mC-PO-fA-PO- | |
| | | fA-PO-mU-PO-fA-PO-mA-PO- | | mA-PO-LA-PO-mA-PO- | |
| | | fG-PS-mC-PS-fU | | fA-PO-mU-PO-fA-PO- | |
| | | | | mC-PS-dT-PS-dT | |
| 316 | 298 | lgT3-PO-lgT3-PO-lgT3-PO- | 1213 | fC-PS-fA-PS-mA-PO- | 1230 |
| | | fC-PO-mU-PO-fG-PO-mC-PO- | | fA-PO-mU-PO-fC-PO- | |
| | | fA-PO-mC-PO-fU-PO-mU-PO- | | mA-PO-fA-PO-mA-PO- | |
| | | fA-PO-mU-PO-fU-PO-mU-PO- | | fA-PO-mU-PO-LA-PO- | |
| | | fU-PO-mG-PO-fA-PO-mU-PO- | | mA-PO-fG-PO-mU-PO- | |
| | | fU-PS-mU-PS-fG | | fG-PO-mC-PO-fA-PO- | |
| | | | | mG-PS-dT-PS-dT | |

The sense strand and antisense strand of the dsRNA respectively comprise the nucleotide sequences of:
a) SEQ ID NOs: 1231 and 1429;
b) SEQ ID NOs: 1307 and 1505;
c) SEQ ID NOs: 1308 and 1506;
d) SEQ ID NOs: 1325 and 1523;
e) SEQ ID NOs: 1328 and 1526; or
f) SEQ ID NOs: 1369 and 1567.

Table 4 below lists the sequences of optimized GalNAc-siRNA constructs derived from selected LPA GalNAc-siRNA constructs listed in Table 3. In Table 4, mX=2′-O-Me nucleotide; fX=2′-F nucleotide; dX=DNA nucleotide; lx=locked nucleic acid (LNA) nucleotide; PO=phosphodiester linkage; and PS=phosphorothioate bond. In these constructs, the sequences of their sense strands and antisense strands correspond to the sense and antisense sequences of the corresponding constructs in Table 1, but for the inclusion of (1) the modified 2′-O-Me nucleotides and 2′-F nucleotides, (2) 3 lgT3 nucleotides at the 5′ end of the sense strands, (3) 2 lT4 nucleotides at the 3′ end of the sense strands, (4) one or more LNA nucleotides in the sense and/or antisense strands, and/or (5) phosphorothioate bonds.

TABLE 4
Exemplary Optimized LPA GalNAc-siRNA Constructs

| SiLPA | Parent | Sense strand sequence | | Antisense strand sequence | |
|---|---|---|---|---|---|
| # | siLPA# | (5′-3′) | SEQ | (5′-3′) | SEQ |
| 317 | 307 | lgT3-PO-lgT3-PO-lgT3- | 1231 | mG-PS-fU-PS-mA-PO- | 1429 |
| | | PO-mG-PO-mA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | mA-PS-mC | | mC-PS-mA-PS-mA | |
| 318 | 307 | lgT3-PO-lgT3-PO-lgT3- | 1232 | mG-PS-fU-PS-mA-PO- | 1430 |
| | | PO-mG-PO-mA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-LA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | mA-PS-mC | | mC-PS-mA-PS-mA | |
| 319 | 307 | lgT3-PO-lgT3-PO-lgT3- | 1233 | mG-PS-fU-PS-mA-PO- | 1431 |
| | | PO-mG-PO-mA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | mA-PS-mC | | mC-PS-mA-PS-mA | |
| 320 | 307 | lgT3-PO-lgT3-PO-lgT3- | 1234 | mG-PS-fU-PS-mA-PO- | 1432 |
| | | PO-mG-PO-mA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | mA-PS-mC | | mC-PS-mA-PS-mA | |
| 321 | 307 | lgT3-PO-lgT3-PO-lgT3- | 1235 | fG-PS-fU-PS-mA-PO- | 1433 |
| | | PO-mG-PO-mA-PO-mG-PO- | | fU-PO-mG-PO-fU-PO- | |
| | | mU-PO-fU-PO-mA-PO-fU- | | mG-PO-fC-PO-mC-PO- | |
| | | PO-fC-PO-fA-PO-mA-PO- | | fU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-fU-PO- | |
| | | mA-PS-mC | | mC-PS-dT-PS-dT | |
| 322 | 307 | lgT3-PO-lgT3-PO-lgT3- | 1236 | mG-PS-fU-PS-mA-PO- | 1434 |
| | | PO-1G-PO-LA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | mA-PS-mC | | mC-PS-mA-PS-mA | |
| 323 | 307 | lgT3-PO-lgT3-PO-lgT3- | 1237 | mG-PS-fU-PS-mA-PO- | 1435 |
| | | PO-1G-PO-LA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | mA-PS-mC | | mC-PS-mA-PS-mA | |
| 324 | 307 | lgT3-PO-lgT3-PO-lgT3- | 1238 | mG-PS-fU-PS-mA-PO- | 1436 |
| | | PO-1G-PO-lA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-LA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | mA-PS-mC | | mC-PS-mA-PS-mA | |
| 325 | 307 | lgT3-PO-lgT3-PO-lgT3- | 1239 | mG-PS-fU-PS-mA-PO- | 1437 |
| | | PO-1G-PO-LA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | mA-PS-mC | | mC-PS-mA-PS-mA | |
| 326 | 307 | lgT3-PO-lgT3-PO-lgT3- | 1240 | fG-PS-fU-PS-mA-PO- | 1438 |
| | | PO-1G-PO-LA-PO-mG-PO- | | fU-PO-mG-PO-fU-PO- | |
| | | mU-PO-fU-PO-mA-PO-fU- | | mG-PO-fC-PO-mC-PO- | |
| | | PO-fC-PO-fA-PO-mA-PO- | | fU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-fU-PO- | |
| | | mA-PS-mC | | mC-PS-dT-PS-dT | |
| 327 | 307 | lgT3-PO-lgT3-PO-lgT3- | 1241 | mG-PS-fU-PS-mA-PO- | 1439 |
| | | PO-mG-PO-mA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | lT4-PO-lT4 | | mC-PS-mA-PS-mA | |
| 328 | 307 | lgT3-PO-lgT3-PO-lgT3- | 1242 | mG-PS-fU-PS-mA-PO- | 1440 |
| | | PO-mG-PO-mA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | lT4-PO-lT4 | | mC-PS-mA-PS-mA | |
| 329 | 307 | lgT3-PO-lgT3-PO-lgT3- | 1243 | mG-PS-fU-PS-mA-PO- | 1441 |
| | | PO-mG-PO-mA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | lT4-PO-lT4 | | mC-PS-mA-PS-mA | |
| 330 | 307 | lgT3-PO-lgT3-PO-lgT3- | 1244 | mG-PS-fU-PS-mA-PO- | 1442 |
| | | PO-mG-PO-mA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | lT4-PO-lT4 | | mC-PS-mA-PS-mA | |
| 331 | 307 | lgT3-PO-lgT3-PO-lgT3- | 1245 | fG-PS-fU-PS-mA-PO- | 1443 |
| | | PO-mG-PO-mA-PO-mG-PO- | | fU-PO-mG-PO-fU-PO- | |
| | | mU-PO-fU-PO-mA-PO-fU- | | mG-PO-fC-PO-mC-PO- | |
| | | PO-fC-PO-fA-PO-mA-PO- | | fU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-fU-PO- | |
| | | lT4-PO-lT4 | | mC-PS-dT-PS-dT | |
| 332 | 307 | lgT3-PO-lgT3-PO-lgT3- | 1246 | mG-PS-fU-PS-mA-PO- | 1444 |
| | | PO-mG-PO-mA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | lT4-PO-lT4 | | mC-PS-LA-PS-lA | |
| 333 | 307 | lgT3-PO-lgT3-PO-lgT3- | 1247 | mG-PS-fU-PS-mA-PO- | 1445 |
| | | PO-mG-PO-mA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | lT4-PO-lT4 | | mC-PS-LA-PS-lA | |
| 334 | 307 | lgT3-PO-lgT3-PO-lgT3- | 1248 | mG-PS-fU-PS-mA-PO- | 1446 |
| | | PO-mG-PO-mA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | lT4-PO-lT4 | | mC-PS-LA-PS-lA | |
| 335 | 307 | lgT3-PO-lgT3-PO-lgT3- | 1249 | mG-PS-fU-PS-mA-PO- | 1447 |
| | | PO-mG-PO-mA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | lT4-PO-lT4 | | mC-PS-LA-PS-lA | |
| 336 | 307 | lgT3-PS-lgT3-PS-lgT3- | 1250 | mG-PS-fU-PS-mA-PO- | 1448 |
| | | PO-mG-PO-mA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | lT4-PS-lT4 | | mC-PS-mA-PS-mA | |
| 337 | 307 | lgT3-PS-lgT3-PS-lgT3- | 1251 | mG-PS-fU-PS-mA-PO- | 1449 |
| | | PO-mG-PO-mA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | lT4-PS-lT4 | | mC-PS-mA-PS-mA | |
| 338 | 307 | lgT3-PS-lgT3-PS-lgT3- | 1252 | mG-PS-fU-PS-mA-PO- | 1450 |
| | | PO-mG-PO-mA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | lT4-PS-lT4 | | mC-PS-mA-PS-mA | |
| 339 | 307 | lgT3-PS-lgT3-PS-lgT3- | 1253 | mG-PS-fU-PS-mA-PO- | 1451 |
| | | PO-mG-PO-mA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | lT4-PS-lT4 | | mC-PS-mA-PS-mA | |
| 340 | 307 | lgT3-PS-lgT3-PS-lgT3- | 1254 | fG-PS-fU-PS-mA-PO- | 1452 |
| | | PO-mG-PO-mA-PO-mG-PO- | | fU-PO-mG-PO-fU-PO- | |
| | | mU-PO-fU-PO-mA-PO-fU- | | mG-PO-fC-PO-mC-PO- | |
| | | PO-fC-PO-fA-PO-mA-PO- | | fU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-fU-PO- | |
| | | lT4-PS-lT4 | | mC-PS-dT-PS-dT | |
| 341 | 307 | lgT3-PS-lgT3-PS-lgT3- | 1255 | mG-PS-fU-PS-mA-PO- | 1453 |
| | | PO-mG-PO-mA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | lT4-PS-lT4 | | mC-PS-LA-PS-lA | |
| 342 | 307 | lgT3-PS-lgT3-PS-lgT3- | 1256 | mG-PS-fU-PS-mA-PO- | 1454 |
| | | PO-mG-PO-mA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | lT4-PS-lT4 | | mC-PS-LA-PS-lA | |
| 343 | 307 | lgT3-PS-lgT3-PS-lgT3- | 1257 | mG-PS-fU-PS-mA-PO- | 1455 |
| | | PO-mG-PO-mA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | lT4-PS-lT4 | | mC-PS-LA-PS-lA | |
| 344 | 307 | lgT3-PS-lgT3-PS-lgT3- | 1258 | mG-PS-fU-PS-mA-PO- | 1456 |
| | | PO-mG-PO-mA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | lT4-PS-lT4 | | mC-PS-LA-PS-lA | |
| 345 | 307 | lgT3-PO-lgT3-PO-lgT3- | 1259 | mG-PS-fU-PS-mA-PO- | 1457 |
| | | PO-1G-PO-LA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | lT4-PO-lT4 | | mC-PS-mA-PS-mA | |
| 346 | 307 | lgT3-PO-lgT3-PO-lgT3- | 1260 | mG-PS-fU-PS-mA-PO- | 1458 |
| | | PO-1G-PO-LA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | lT4-PO-lT4 | | mC-PS-mA-PS-mA | |
| 347 | 307 | lgT3-PO-lgT3-PO-lgT3- | 1261 | mG-PS-fU-PS-mA-PO- | 1459 |
| | | PO-1G-PO-lA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | lT4-PO-lT4 | | mC-PS-mA-PS-mA | |
| 348 | 307 | lgT3-PO-lgT3-PO-lgT3- | 1262 | mG-PS-fU-PS-mA-PO- | 1460 |
| | | PO-1G-PO-LA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | lT4-PO-lT4 | | mC-PS-mA-PS-mA | |
| 349 | 307 | lgT3-PO-lgT3-PO-lgT3- | 1263 | fG-PS-fU-PS-mA-PO- | 1461 |
| | | PO-1G-PO-LA-PO-mG-PO- | | fU-PO-mG-PO-fU-PO- | |
| | | mU-PO-fU-PO-mA-PO-fU- | | mG-PO-fC-PO-mC-PO- | |
| | | PO-fC-PO-fA-PO-mA-PO- | | fU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-fU-PO- | |
| | | lT4-PO-lT4 | | mC-PS-dT-PS-dT | |
| 350 | 307 | lgT3-PS-lgT3-PS-lgT3- | 1264 | mG-PS-fU-PS-mA-PO- | 1462 |
| | | PO-1G-PO-lA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | lT4-PS-lT4 | | mC-PS-mA-PS-mA | |
| 351 | 307 | lgT3-PS-lgT3-PS-lgT3- | 1265 | mG-PS-fU-PS-mA-PO- | 1463 |
| | | PO-1G-PO-LA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | lT4-PS-lT4 | | mC-PS-mA-PS-mA | |
| 352 | 307 | lgT3-PS-lgT3-PS-lgT3- | 1266 | mG-PS-fU-PS-mA-PO- | 1464 |
| | | PO-1G-PO-LA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | lT4-PS-lT4 | | mC-PS-mA-PS-mA | |
| 353 | 307 | lgT3-PS-lgT3-PS-lgT3- | 1267 | mG-PS-fU-PS-mA-PO- | 1465 |
| | | PO-1G-PO-lA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-mU-PO- | |
| | | lT4-PS-lT4 | | mC-PS-mA-PS-mA | |
| 354 | 307 | lgT3-PS-lgT3-PS-lgT3- | 1268 | fG-PS-fU-PS-mA-PO- | 1466 |
| | | PO-1G-PO-LA-PO-mG-PO- | | fU-PO-mG-PO-fU-PO- | |
| | | MU-PO-fU-PO-mA-PO-fU- | | mG-PO-fC-PO-mC-PO- | |
| | | PO-fC-PO-fA-PO-mA-PO- | | fU-PO-mU-PO-mG-PO- | |
| | | mG-PO-mG-PO-mC-PO-mA- | | mA-PO-fU-PO-mA-PO- | |
| | | PO-mC-PO-mA-PO-mU-PS- | | fA-PO-mC-PO-fU-PO- | |
| | | lT4-PS-lT4 | | mC-PS-dT-PS-dT | |
| 355 | 307 | lgT3-PO-lgT3-PO-lgT3- | 1269 | mG-PS-fU-PS-mA-PO- | 1467 |
| | | PO-mG-PO-mA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PO-lT4-PO-lT4 | | mC-PS-mA-PS-mA | |
| 356 | 307 | lgT3-PO-lgT3-PO-lgT3- | 1270 | mG-PS-fU-PS-mA-PO- | 1468 |
| | | PO-mG-PO-mA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PO-lT4-PO-lT4 | | mC-PS-mA-PS-mA | |
| 357 | 307 | lgT3-PO-lgT3-PO-lgT3- | 1271 | mG-PS-fU-PS-mA-PO- | 1469 |
| | | PO-mG-PO-mA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PO-lT4-PO-lT4 | | mC-PS-mA-PS-mA | |
| 358 | 307 | lgT3-PO-lgT3-PO-lgT3- | 1272 | mG-PS-fU-PS-mA-PO- | 1470 |
| | | PO-mG-PO-mA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PO-lT4-PO-lT4 | | mC-PS-mA-PS-mA | |
| 359 | 307 | lgT3-PO-lgT3-PO-lgT3- | 1273 | fG-PS-fU-PS-mA-PO- | 1471 |
| | | PO-mG-PO-mA-PO-mG-PO- | | fU-PO-mG-PO-fU-PO- | |
| | | mU-PO-fU-PO-mA-PO-fU- | | mG-PO-fC-PO-mC-PO- | |
| | | PO-fC-PO-fA-PO-mA-PO- | | fU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-fU-PO- | |
| | | mA-PO-mC-PO-lT4-PO-lT4 | | mC-PS-dT-PS-dT | |
| 360 | 307 | lgT3-PO-lgT3-PO-lgT3- | 1274 | mG-PS-fU-PS-mA-PO- | 1472 |
| | | PO-mG-PO-mA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PO-lT4-PO-lT4 | | mC-PS-LA-PS-lA | |
| 36 | 307 | lgT3-PO-lgT3-PO-lgT3- | 1275 | mG-PS-fU-PS-mA-PO- | 1473 |
| | | PO-mG-PO-mA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PO-lT4-PO-lT4 | | mC-PS-LA-PS-lA | |
| 362 | 307 | lgT3-PO-lgT3-PO-lgT3- | 1276 | mG-PS-fU-PS-mA-PO- | 1474 |
| | | PO-mG-PO-mA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PO-lT4-PO-lT4 | | mC-PS-LA-PS-lA | |
| 363 | \|307 | lgT3-PO-lgT3-PO-lgT3- | 1277 | mG-PS-fU-PS-mA-PO- | 1475 |
| | | PO-mG-PO-mA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-A-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PO-lT4-PO-lT4 | | mC-PS-LA-PS-lA | |
| 364 | 307 | lgT3-PS-lgT3-PS-lgT3- | 1278 | mG-PS-fU-PS-mA-PO- | 1476 |
| | | PO-mG-PO-mA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PS-lT4-PS-lT4 | | mC-PS-mA-PS-mA | |
| 365 | 307 | lgT3-PS-lgT3-PS-lgT3- | 1279 | mG-PS-fU-PS-mA-PO- | 1477 |
| | | PO-mG-PO-mA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PS-lT4-PS-lT4 | | mC-PS-mA-PS-mA | |
| 366 | 307 | lgT3-PS-lgT3-PS-lgT3- | 1280 | mG-PS-fU-PS-mA-PO- | 1478 |
| | | PO-mG-PO-mA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PS-lT4-PS-lT4 | | mC-PS-mA-PS-mA | |
| 367 | 307 | lgT3-PS-lgT3-PS-lgT3- | 1281 | mG-PS-fU-PS-mA-PO- | 1479 |
| | | PO-mG-PO-mA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PS-lT4-PS-lT4 | | mC-PS-mA-PS-mA | |
| 368 | 307 | lgT3-PS-lgT3-PS-lgT3- | 1282 | fG-PS-fU-PS-mA-PO- | 1480 |
| | | PO-mG-PO-mA-PO-mG-PO- | | fU-PO-mG-PO-fU-PO- | |
| | | mU-PO-fU-PO-mA-PO-fU- | | mG-PO-fC-PO-mC-PO- | |
| | | PO-fC-PO-fA-PO-mA-PO- | | fU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-fU-PO- | |
| | | mA-PO-mC-PS-lT4-PS-lT4 | | mC-PS-dT-PS-dT | |
| 369 | 307 | lgT3-PS-lgT3-PS-lgT3- | 1283 | mG-PS-fU-PS-mA-PO- | 1481 |
| | | PO-mG-PO-mA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PS-lT4-PS-lT4 | | mC-PS-LA-PS-lA | |
| 370 | 307 | lgT3-PS-lgT3-PS-lgT3- | 1284 | mG-PS-fU-PS-mA-PO- | 1482 |
| | | PO-mG-PO-mA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PS-lT4-PS-lT4 | | mC-PS-LA-PS-lA | |
| 371 | 307 | lgT3-PS-lgT3-PS-lgT3- | 1285 | mG-PS-fU-PS-mA-PO- | 1483 |
| | | PO-mG-PO-mA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PS-lT4-PS-lT4 | | mC-PS-LA-PS-lA | |
| 372 | 307 | lgT3-PS-lgT3-PS-lgT3- | 1286 | mG-PS-fU-PS-mA-PO- | 1484 |
| | | PO-mG-PO-mA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PS-lT4-PS-lT4 | | mC-PS-LA-PS-lA | |
| 373 | 307 | lgT3-PO-lgT3-PO-lgT3- | 1287 | mG-PS-fU-PS-mA-PO- | 1485 |
| | | PO-1G-PO-lA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PO-lT4-PO-lT4 | | mC-PS-mA-PS-mA | |
| 374 | 307 | lgT3-PO-lgT3-PO-lgT3- | 1288 | mG-PS-fU-PS-mA-PO- | 1486 |
| | | PO-1G-PO-LA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PO-lT4-PO-lT4 | | mC-PS-mA-PS-mA | |
| 375 | 307 | lgT3-PO-lgT3-PO-lgT3- | 1289 | mG-PS-fU-PS-mA-PO- | 1487 |
| | | PO-1G-PO-lA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PO-lT4-PO-lT4 | | mC-PS-mA-PS-mA | |
| 376 | 307 | lgT3-PO-lgT3-PO-lgT3- | 1290 | mG-PS-fU-PS-mA-PO- | 1488 |
| | | PO-1G-PO-LA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PO-lT4-PO-lT4 | | mC-PS-mA-PS-mA | |
| 377 | 307 | lgT3-PO-lgT3-PO-lgT3- | 1291 | fG-PS-fU-PS-mA-PO- | 1489 |
| | | PO-1G-PO-LA-PO-mG-PO- | | fU-PO-mG-PO-fU-PO- | |
| | | mU-PO-fU-PO-mA-PO-fU- | | mG-PO-fC-PO-mC-PO- | |
| | | PO-fC-PO-fA-PO-mA-PO- | | fU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-fU-PO- | |
| | | mA-PO-mC-PO-lT4-PO-lT4 | | mC-PS-dT-PS-dT | |
| 378 | 307 | lgT3-PS-lgT3-PS-lgT3- | 1292 | mG-PS-fU-PS-mA-PO- | 1490 |
| | | PO-1G-PO-LA-PO-mG-PO- | | fU-PO-mG-PO-mU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PS-lT4-PS-lT4 | | mC-PS-mA-PS-mA | |
| 379 | 307 | lgT3-PS-lgT3-PS-lgT3- | 1293 | mG-PS-fU-PS-mA-PO- | 1491 |
| | | PO-1G-PO-lA-PO-mG-PO- | | mU-PO-mG-PO-fU-PO- | |
| | | mU-PO-fU-PO-mA-PO-fU- | | mG-PO-mC-PO-mC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PS-lT4-PS-lT4 | | mC-PS-mA-PS-mA | |
| 380 | 307 | lgT3-PS-lgT3-PS-lgT3- | 1294 | mG-PS-fU-PS-mA-PO- | 1492 |
| | | PO-1G-PO-LA-PO-mG-PO- | | fU-PO-mG-PO-mU-PO- | |
| | | mU-PO-fU-PO-mA-PO-fU- | | mG-PO-fC-PO-fC-PO- | |
| | | PO-fC-PO-LA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PS-lT4-PS-lT4 | | mC-PS-mA-PS-mA | |
| 381 | \|307 | lgT3-PS-lgT3-PS-lgT3- | 1295 | mG-PS-fU-PS-mA-PO- | 1493 |
| | | PO-1G-PO-LA-PO-mG-PO- | | fU-PO-mG-PO-mU-PO- | |
| | | mU-PO-fU-PO-mA-PO-fU- | | mG-PO-mC-PO-fC-PO- | |
| | | PO-fC-PO-fA-PO-mA-PO- | | mU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-mU-PO- | |
| | | mA-PO-mC-PS-lT4-PS-lT4 | | mC-PS-mA-PS-mA | |
| 382 | 307 | lgT3-PS-lgT3-PS-lgT3- | 1296 | fG-PS-fU-PS-mA-PO- | 1494 |
| | | PO-1G-PO-lA-PO-mG-PO- | | fU-PO-mG-PO-fU-PO- | |
| | | mU-PO-fU-PO-mA-PO-fU- | | mG-PO-fC-PO-mC-PO- | |
| | | PO-fC-PO-fA-PO-mA-PO- | | fU-PO-mU-PO-mG-PO- | |
| | | mG-PO-mG-PO-mC-PO-mA- | | mA-PO-fU-PO-mA-PO- | |
| | | PO-mC-PO-mA-PO-mU-PO- | | fA-PO-mC-PO-fU-PO- | |
| | | mA-PO-mC-PS-lT4-PS-lT4 | | mC-PS-dT-PS-dT | |
| 383 | 311 | lgT3-PO-lgT3-PO-lgT3- | 1297 | mU-PS-fG-PS-mC-PO- | 1495 |
| | | PO-mG-PO-mG-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | mC-PS-mA | | mC-PS-mA-PS-mA | |
| 384 | 311 | lgT3-PO-lgT3-PO-lgT3- | 1298 | mU-PS-fG-PS-mC-PO- | 1496 |
| | | PO-mG-PO-mG-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | mC-PS-mA | | mC-PS-mA-PS-mA | |
| 385 | 311 | lgT3-PO-lgT3-PO-lgT3- | 1299 | Hy-mU-PS-fG-PS-mC- | 1497 |
| | | PO-mG-PO-mG-PO-mA-PO- | | PO-fC-PO-mU-PO-mC- | |
| | | mC-PO-fG-PO-mG-PO-fA- | | PO-mG-PO-fA-PO-fU- | |
| | | PO-fG-PO-fU-PO-mU-PO- | | PO-mA-PO-mA-PO-mC- | |
| | | mA-PO-mU-PO-mC-PO-mG- | | PO-mU-PO-fC-PO-mC- | |
| | | PO-mA-PO-mG-PO-mG-PS- | | PO-fG-PO-mU-PO-mC- | |
| | | mC-PS-mA | | PO-mC-PS-mA-PS-mA | |
| 386 | 311 | lgT3-PO-lgT3-PO-lgT3- | 1300 | mU-PS-fG-PS-mC-PO- | 1498 |
| | | PO-mG-PO-mG-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | mC-PS-mA | | mC-PS-mA-PS-mA | |
| 387 | 311 | lgT3-PO-lgT3-PO-lgT3- | 1301 | fU-PS-fG-PS-mC-PO- | 1499 |
| | | PO-mG-PO-mG-PO-mA-PO- | | fC-PO-mU-PO-fC-PO- | |
| | | mC-PO-fG-PO-mG-PO-fA- | | mG-PO-fA-PO-mU-PO- | |
| | | PO-fG-PO-fU-PO-mU-PO- | | fA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-fC-PO- | |
| | | mC-PS-mA | | mC-PS-dT-PS-dT | |
| 388 | 311 | lgT3-PO-lgT3-PO-lgT3- | 1302 | mU-PS-fG-PS-mC-PO- | 1500 |
| | | PO-1G-PO-1G-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | mC-PS-mA | | mC-PS-mA-PS-mA | |
| 389 | 311 | lgT3-PO-lgT3-PO-lgT3- | 1303 | mU-PS-fG-PS-mC-PO- | 1501 |
| | | PO-1G-PO-1G-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | mC-PS-mA | | mC-PS-mA-PS-mA | |
| 390 | 311 | lgT3-PO-lgT3-PO-lgT3- | 1304 | mU-PS-fG-PS-mC-PO- | 1502 |
| | | PO-1G-PO-1G-PO-mA-PO- | | fC-PO-mU-PO-mC-PO- | |
| | | mC-PO-fG-PO-mG-PO-fA- | | mG-PO-fA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | mC-PS-mA | | mC-PS-mA-PS-mA | |
| 391 | 311 | lgT3-PO-lgT3-PO-lgT3- | 1305 | mU-PS-fG-PS-mC-PO- | 1503 |
| | | PO-1G-PO-1G-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | mC-PS-mA | | mC-PS-mA-PS-mA | |
| 392 | 311 | lgT3-PO-lgT3-PO-lgT3- | 1306 | fU-PS-fG-PS-mC-PO- | 1504 |
| | | PO-1G-PO-1G-PO-mA-PO- | | fC-PO-mU-PO-fC-PO- | |
| | | mC-PO-fG-PO-mG-PO-fA- | | mG-PO-fA-PO-mU-PO- | |
| | | PO-fG-PO-fU-PO-mU-PO- | | fA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-fC-PO- | |
| | | mC-PS-mA | | mC-PS-dT-PS-dT | |
| 393 | 311 | lgT3-PO-lgT3-PO-lgT3- | 1307 | mU-PS-fG-PS-mC-PO- | 1505 |
| | | PO-mG-PO-mG-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | lT4-PO-lT4 | | mC-PS-mA-PS-mA | |
| 394 | 311 | lgT3-PO-lgT3-PO-lgT3- | 1308 | mU-PS-fG-PS-mC-PO- | 1506 |
| | | PO-mG-PO-mG-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | lT4-PO-lT4 | | mC-PS-mA-PS-mA | |
| 395 | 311 | lgT3-PO-lgT3-PO-lgT3- | 1309 | mU-PS-fG-PS-mC-PO- | 1507 |
| | | PO-mG-PO-mG-PO-mA-PO- | | fC-PO-mU-PO-mC-PO- | |
| | | mC-PO-fG-PO-mG-PO-fA- | | mG-PO-fA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | lT4-PO-lT4 | | mC-PS-mA-PS-mA | |
| 396 | 311 | lgT3-PO-lgT3-PO-lgT3- | 1310 | mU-PS-fG-PS-mC-PO- | 1508 |
| | | PO-mG-PO-mG-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | lT4-PO-lT4 | | mC-PS-mA-PS-mA | |
| 397 | 311 | lgT3-PO-lgT3-PO-lgT3- | 1311 | fU-PS-fG-PS-mC-PO- | 1509 |
| | | PO-mG-PO-mG-PO-mA-PO- | | fC-PO-mU-PO-fC-PO- | |
| | | mC-PO-fG-PO-mG-PO-fA- | | mG-PO-fA-PO-mU-PO- | |
| | | PO-fG-PO-fU-PO-mU-PO- | | fA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-fC-PO- | |
| | | lT4-PO-lT4 | | mC-PS-dT-PS-dT | |
| 398 | 311 | lgT3-PO-lgT3-PO-lgT3- | 1312 | mU-PS-fG-PS-mC-PO- | 1510 |
| | | PO-mG-PO-mG-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | lT4-PO-lT4 | | mC-PS-LA-PS-lA | |
| 399 | 311 | lgT3-PO-lgT3-PO-lgT3- | 1313 | mU-PS-fG-PS-mC-PO- | 1511 |
| | | PO-mG-PO-mG-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | lT4-PO-lT4 | | mC-PS-LA-PS-lA | |
| 400 | 311 | lgT3-PO-lgT3-PO-lgT3- | 1314 | mU-PS-fG-PS-mC-PO- | 1512 |
| | | PO-mG-PO-mG-PO-mA-PO- | | fC-PO-mU-PO-mC-PO- | |
| | | mC-PO-fG-PO-mG-PO-fA- | | mG-PO-fA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | lT4-PO-lT4 | | mC-PS-LA-PS-lA | |
| 401 | 311 | lgT3-PO-lgT3-PO-lgT3- | 1315 | mU-PS-fG-PS-mC-PO- | 1513 |
| | | PO-mG-PO-mG-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | lT4-PO-lT4 | | mC-PS-LA-PS-lA | |
| 402 | 311 | lgT3-PS-lgT3-PS-lgT3- | 1316 | mU-PS-fG-PS-mC-PO- | 1514 |
| | | PO-mG-PO-mG-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-fG- | |
| | | PO-mA-PO-mG-PO-mG-PS- | | PO-mU-PO-mC-PO-mC- | |
| | | lT4-PS-lT4 | | PS-mA-PS-mA | |
| 403 | 311 | lgT3-PS-lgT3-PS-lgT3- | 1317 | mU-PS-fG-PS-mC-PO- | 1515 |
| | | PO-mG-PO-mG-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | lT4-PS-lT4 | | mC-PS-mA-PS-mA | |
| 404 | 311 | lgT3-PS-lgT3-PS-lgT3- | 1318 | mU-PS-fG-PS-mC-PO- | 1516 |
| | | PO-mG-PO-mG-PO-mA-PO- | | fC-PO-mU-PO-mC-PO- | |
| | | mC-PO-fG-PO-mG-PO-fA- | | mG-PO-fA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | lT4-PS-lT4 | | mC-PS-mA-PS-mA | |
| 405 | 311 | lgT3-PS-lgT3-PS-lgT3- | 1319 | mU-PS-fG-PS-mC-PO- | 1517 |
| | | PO-mG-PO-mG-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | lT4-PS-lT4 | | mC-PS-mA-PS-mA | |
| 406 | 311 | lgT3-PS-lgT3-PS-lgT3- | 1320 | fU-PS-fG-PS-mC-PO- | 1518 |
| | | PO-mG-PO-mG-PO-mA-PO- | | fC-PO-mU-PO-fC-PO- | |
| | | mC-PO-fG-PO-mG-PO-fA- | | mG-PO-fA-PO-mU-PO- | |
| | | PO-fG-PO-fU-PO-mU-PO- | | fA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-fC-PO- | |
| | | lT4-PS-lT4 | | mC-PS-dT-PS-dT | |
| 407 | 311 | lgT3-PS-lgT3-PS-lgT3- | 1321 | mU-PS-fG-PS-mC-PO- | 1519 |
| | | PO-mG-PO-mG-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | lT4-PS-lT4 | | mC-PS-lA-PS-lA | |
| 408 | 311 | lgT3-PS-lgT3-PS-lgT3- | 1322 | mU-PS-fG-PS-mC-PO- | 1520 |
| | | PO-mG-PO-mG-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | lT4-PS-lT4 | | mC-PS-LA-PS-lA | |
| 409 | 311 | lgT3-PS-lgT3-PS-lgT3- | 1323 | mU-PS-fG-PS-mC-PO- | 1521 |
| | | PO-mG-PO-mG-PO-mA-PO- | | fC-PO-mU-PO-mC-PO- | |
| | | mC-PO-fG-PO-mG-PO-fA- | | mG-PO-fA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | lT4-PS-lT4 | | mC-PS-lA-PS-lA | |
| 410 | 311 | lgT3-PS-lgT3-PS-lgT3- | 1324 | mU-PS-fG-PS-mC-PO- | 1522 |
| | | PO-mG-PO-mG-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | EG-PO-mU-PO-mC-PO- | |
| | | lT4-PS-lT4 | | mC-PS-lA-PS-lA | |
| 411 | 311 | lgT3-PO-lgT3-PO-lgT3- | 1325 | mU-PS-fG-PS-mC-PO- | 1523 |
| | | PO-1G-PO-1G-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | lT4-PO-lT4 | | mC-PS-mA-PS-mA | |
| 412 | 311 | lgT3-PO-lgT3-PO-lgT3- | 1326 | mU-PS-fG-PS-mC-PO- | 1524 |
| | | PO-1G-PO-1G-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | lT4-PO-lT4 | | mC-PS-mA-PS-mA | |
| 413 | 311 | lgT3-PO-lgT3-PO-lgT3- | 1327 | mU-PS-fG-PS-mC-PO- | 1525 |
| | | PO-1G-PO-1G-PO-mA-PO- | | fC-PO-mU-PO-mC-PO- | |
| | | mC-PO-fG-PO-mG-PO-fA- | | mG-PO-fA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | lT4-PO-lT4 | | mC-PS-mA-PS-mA | |
| 414 | 311 | lgT3-PO-lgT3-PO-lgT3- | 1328 | mU-PS-fG-PS-mC-PO- | 1526 |
| | | PO-1G-PO-1G-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | lT4-PO-lT4 | | mC-PS-mA-PS-mA | |
| 415 | 311 | lgT3-PO-lgT3-PO-lgT3- | 1329 | fU-PS-fG-PS-mC-PO- | 1527 |
| | | PO-1G-PO-1G-PO-mA-PO- | | fC-PO-mU-PO-fC-PO- | |
| | | mC-PO-fG-PO-mG-PO-fA- | | mG-PO-LA-PO-mU-PO- | |
| | | PO-fG-PO-fU-PO-mU-PO- | | fA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-fC-PO- | |
| | | lT4-PO-lT4 | | mC-PS-dT-PS-dT | |
| 416 | 311 | lgT3-PS-lgT3-PS-lgT3- | 1330 | mU-PS-fG-PS-mC-PO- | 1528 |
| | | PO-1G-PO-1G-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | lT4-PS-lT4 | | mC-PS-mA-PS-mA | |
| 417 | 311 | lgT3-PS-lgT3-PS-lgT3- | 1331 | mU-PS-fG-PS-mC-PO- | 1529 |
| | | PO-1G-PO-1G-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | lT4-PS-lT4 | | mC-PS-mA-PS-mA | |
| 418 | 311 | lgT3-PS-lgT3-PS-lgT3- | 1332 | mU-PS-fG-PS-mC-PO- | 1530 |
| | | PO-1G-PO-1G-PO-mA-PO- | | fC-PO-mU-PO-mC-PO- | |
| | | mC-PO-fG-PO-mG-PO-fA- | | mG-PO-fA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | lT4-PS-lT4 | | mC-PS-mA-PS-mA | |
| 419 | 311 | lgT3-PS-lgT3-PS-lgT3- | 1333 | mU-PS-fG-PS-mC-PO- | 1531 |
| | | PO-1G-PO-1G-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-mC-PO- | |
| | | lT4-PS-lT4 | | mC-PS-mA-PS-mA | |
| 420 | 311 | lgT3-PS-lgT3-PS-lgT3- | 1334 | fU-PS-fG-PS-mC-PO- | 1532 |
| | | PO-1G-PO-1G-PO-mA-PO- | | fC-PO-mU-PO-fC-PO- | |
| | | mC-PO-fG-PO-mG-PO-fA- | | mG-PO-fA-PO-mU-PO- | |
| | | PO-fG-PO-fU-PO-mU-PO- | | fA-PO-mA-PO-mC-PO- | |
| | | mA-PO-mU-PO-mC-PO-mG- | | mU-PO-fC-PO-mC-PO- | |
| | | PO-mA-PO-mG-PO-mG-PS- | | fG-PO-mU-PO-fC-PO- | |
| | | lT4-PS-lT4 | | mC-PS-dT-PS-dT | |
| 421 | 311 | lgT3-PO-lgT3-PO-lgT3- | 1335 | mU-PS-fG-PS-mC-PO- | 1533 |
| | | PO-mG-PO-mG-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PO-lT4-PO-lT4 | | mC-PS-mA-PS-mA | |
| 422 | 311 | lgT3-PO-lgT3-PO-lgT3- | 1336 | mU-PS-fG-PS-mC-PO- | 1534 |
| | | PO-mG-PO-mG-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PO-lT4-PO-lT4 | | mC-PS-mA-PS-mA | |
| 423 | 311 | lgT3-PO-lgT3-PO-lgT3- | 1337 | mU-PS-fG-PS-mC-PO- | 1535 |
| | | PO-mG-PO-mG-PO-mA-PO- | | fC-PO-mU-PO-mC-PO- | |
| | | mC-PO-fG-PO-mG-PO-fA- | | mG-PO-LA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PO-lT4-PO-lT4 | | mC-PS-mA-PS-mA | |
| 424 | 311 | lgT3-PO-lgT3-PO-lgT3- | 1338 | mU-PS-fG-PS-mC-PO- | 1536 |
| | | PO-mG-PO-mG-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PO-lT4-PO-lT4 | | mC-PS-mA-PS-mA | |
| 425 | 311 | lgT3-PO-lgT3-PO-lgT3- | 1339 | fU-PS-fG-PS-mC-PO- | 1537 |
| | | PO-mG-PO-mG-PO-mA-PO- | | fC-PO-mU-PO-fC-PO- | |
| | | mC-PO-fG-PO-mG-PO-fA- | | mG-PO-fA-PO-mU-PO- | |
| | | PO-fG-PO-fU-PO-mU-PO- | | fA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-fC-PO- | |
| | | mC-PO-mA-PO-lT4-PO-lT4 | | mC-PS-dT-PS-dT | |
| 426 | 311 | lgT3-PO-lgT3-PO-lgT3- | 1340 | mU-PS-fG-PS-mC-PO- | 1538 |
| | | PO-mG-PO-mG-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PO-lT4-PO-lT4 | | mC-PS-lA-PS-lA | |
| 427 | 311 | lgT3-PO-lgT3-PO-lgT3- | 1341 | mU-PS-fG-PS-mC-PO- | 1539 |
| | | PO-mG-PO-mG-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PO-lT4-PO-lT4 | | mC-PS-LA-PS-LA | |
| 428 | 311 | lgT3-PO-lgT3-PO-lgT3- | 1342 | mU-PS-fG-PS-mC-PO- | 1540 |
| | | PO-mG-PO-mG-PO-mA-PO- | | fC-PO-mU-PO-mC-PO- | |
| | | mC-PO-fG-PO-mG-PO-fA- | | mG-PO-fA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PO-lT4-PO-lT4 | | mC-PS-LA-PS-lA | |
| 429 | 311 | lgT3-PO-lgT3-PO-lgT3- | 1343 | mU-PS-fG-PS-mC-PO- | 1541 |
| | | PO-mG-PO-mG-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PO-lT4-PO-lT4 | | mC-PS-LA-PS-lA | |
| 430 | 311 | lgT3-PS-lgT3-PS-lgT3- | 1344 | mU-PS-fG-PS-mC-PO- | 1542 |
| | | PO-mG-PO-mG-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PS-lT4-PS-lT4 | | mC-PS-mA-PS-mA | |
| 431 | 311 | lgT3-PS-lgT3-PS-lgT3- | 1345 | mU-PS-fG-PS-mC-PO- | 1543 |
| | | PO-mG-PO-mG-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PS-lT4-PS-lT4 | | mC-PS-mA-PS-mA | |
| 432 | 311 | lgT3-PS-lgT3-PS-lgT3- | 1346 | mU-PS-fG-PS-mC-PO- | 1544 |
| | | PO-mG-PO-mG-PO-mA-PO- | | fC-PO-mU-PO-mC-PO- | |
| | | mC-PO-fG-PO-mG-PO-fA- | | mG-PO-fA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PS-lT4-PS-lT4 | | mC-PS-mA-PS-mA | |
| 433 | 311 | lgT3-PS-lgT3-PS-lgT3- | 1347 | mU-PS-fG-PS-mC-PO- | 1545 |
| | | PO-mG-PO-mG-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PS-lT4-PS-lT4 | | mC-PS-mA-PS-mA | |
| 434 | 311 | lgT3-PS-lgT3-PS-lgT3- | 1348 | fU-PS-fG-PS-mC-PO- | 1546 |
| | | PO-mG-PO-mG-PO-mA-PO- | | fC-PO-mU-PO-fC-PO- | |
| | | mC-PO-fG-PO-mG-PO-fA- | | mG-PO-fA-PO-mU-PO- | |
| | | PO-fG-PO-fU-PO-mU-PO- | | fA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-fC-PO- | |
| | | mC-PO-mA-PS-lT4-PS-lT4 | | mC-PS-dT-PS-dT | |
| 435 | 311 | lgT3-PS-lgT3-PS-lgT3- | 1349 | mU-PS-fG-PS-mC-PO- | 1547 |
| | | PO-mG-PO-mG-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PS-lT4-PS-lT4 | | mC-PS-LA-PS-lA | |
| 436 | 311 | lgT3-PS-lgT3-PS-lgT3- | \|1350 | mU-PS-fG-PS-mC-PO- | 1548 |
| | | PO-mG-PO-mG-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PS-lT4-PS-lT4 | | mC-PS-LA-PS-lA | |
| 437 | 311 | lgT3-PS-lgT3-PS-lgT3- | 1351 | mU-PS-fG-PS-mC-PO- | 1549 |
| | | PO-mG-PO-mG-PO-mA-PO- | | fC-PO-mU-PO-mC-PO- | |
| | | mC-PO-fG-PO-mG-PO-fA- | | mG-PO-fA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PS-lT4-PS-lT4 | | mC-PS-LA-PS-lA | |
| 438 | 311 | lgT3-PS-lgT3-PS-lgT3- | 1352 | mU-PS-fG-PS-mC-PO- | 1550 |
| | | PO-mG-PO-mG-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PS-lT4-PS-lT4 | | mC-PS-LA-PS-LA | |
| 439 | 311 | lgT3-PO-lgT3-PO-lgT3- | 1353 | mU-PS-fG-PS-mC-PO- | 1551 |
| | | PO-1G-PO-1G-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PO-lT4-PO-lT4 | | mC-PS-mA-PS-mA | |
| 440 | 311 | lgT3-PO-lgT3-PO-lgT3- | 1354 | mU-PS-fG-PS-mC-PO- | 1552 |
| | | PO-1G-PO-1G-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PO-lT4-PO-lT4 | | mC-PS-mA-PS-mA | |
| 441 | 311 | lgT3-PO-lgT3-PO-lgT3- | 1355 | mU-PS-fG-PS-mC-PO- | 1553 |
| | | PO-1G-PO-1G-PO-mA-PO- | | fC-PO-mU-PO-mC-PO- | |
| | | mC-PO-fG-PO-mG-PO-fA- | | mG-PO-fA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PO-lT4-PO-lT4 | | mC-PS-mA-PS-mA | |
| 442 | 311 | lgT3-PO-lgT3-PO-lgT3- | 1356 | mU-PS-fG-PS-mC-PO- | 1554 |
| | | PO-1G-PO-1G-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PO-lT4-PO-lT4 | | mC-PS-mA-PS-mA | |
| 443 | 311 | lgT3-PO-lgT3-PO-lgT3- | 1357 | fU-PS-fG-PS-mC-PO- | 1555 |
| | | PO-1G-PO-1G-PO-mA-PO- | | fC-PO-mU-PO-fC-PO- | |
| | | mC-PO-fG-PO-mG-PO-fA- | | mG-PO-LA-PO-mU-PO- | |
| | | PO-fG-PO-fU-PO-mU-PO- | | fA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-fC-PO- | |
| | | mC-PO-mA-PO-lT4-PO-lT4 | | mC-PS-dT-PS-dT | |
| 444 | 311 | lgT3-PS-lgT3-PS-lgT3- | 1358 | mU-PS-fG-PS-mC-PO- | 1556 |
| | | PO-1G-PO-1G-PO-mA-PO- | | fC-PO-mU-PO-mC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PS-lT4-PS-lT4 | | mC-PS-mA-PS-mA | |
| 445 | 311 | lgT3-PS-lgT3-PS-lgT3- | 1359 | mU-PS-fG-PS-mC-PO- | 1557 |
| | | PO-1G-PO-1G-PO-mA-PO- | | mC-PO-mU-PO-fC-PO- | |
| | | mC-PO-fG-PO-mG-PO-fA- | | mG-PO-mA-PO-mU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PS-lT4-PS-lT4 | | mC-PS-mA-PS-mA | |
| 446 | 311 | lgT3-PS-lgT3-PS-lgT3- | 1360 | mU-PS-fG-PS-mC-PO- | 1558 |
| | | PO-1G-PO-1G-PO-mA-PO- | | fC-PO-mU-PO-mC-PO- | |
| | | mC-PO-fG-PO-mG-PO-fA- | | mG-PO-fA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PS-lT4-PS-lT4 | | mC-PS-mA-PS-mA | |
| 447 | 311 | lgT3-PS-lgT3-PS-lgT3- | 1361 | mU-PS-fG-PS-mC-PO- | 1559 |
| | | PO-1G-PO-1G-PO-mA-PO- | | fC-PO-mU-PO-mC-PO- | |
| | | mC-PO-fG-PO-mG-PO-fA- | | mG-PO-mA-PO-fU-PO- | |
| | | PO-fG-PO-fU-PO-mU-PO- | | mA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-mC-PO- | |
| | | mC-PO-mA-PS-lT4-PS-lT4 | | mC-PS-mA-PS-mA | |
| 448 | 311 | lgT3-PS-lgT3-PS-lgT3- | 1362 | fU-PS-fG-PS-mC-PO- | 1560 |
| | | PO-1G-PO-1G-PO-mA-PO- | | fC-PO-mU-PO-fC-PO- | |
| | | mC-PO-fG-PO-mG-PO-fA- | | mG-PO-fA-PO-mU-PO- | |
| | | PO-fG-PO-fU-PO-mU-PO- | | fA-PO-mA-PO-mC-PO- | |
| | | mA-PO-mU-PO-mC-PO-mG- | | mU-PO-fC-PO-mC-PO- | |
| | | PO-mA-PO-mG-PO-mG-PO- | | fG-PO-mU-PO-fC-PO- | |
| | | mC-PO-mA-PS-lT4-PS-lT4 | | mC-PS-dT-PS-dT | |
| 449 | 314 | lgT3-PO-lgT3-PO-lgT3- | 1363 | mG-PS-fU-PS-mG-PO- | 1561 |
| | | PO-mG-PO-mC-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | mA-PS-mC | | mC-PS-mA-PS-mA | |
| 450 | 314 | lgT3-PO-lgT3-PO-lgT3- | 1364 | mG-PS-fU-PS-mG-PO- | 1562 |
| | | PO-mG-PO-mC-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | mA-PS-mC | | mC-PS-mA-PS-mA | |
| 451 | 314 | lgT3-PO-lgT3-PO-lgT3- | 1365 | mG-PS-fU-PS-mG-PO- | 1563 |
| | | PO-mG-PO-mC-PO-mC-PO- | | fC-PO-mC-PO-mU-PO- | |
| | | mA-PO-fG-PO-mA-PO-fG- | | mC-PO-fG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | mA-PS-mC | | mC-PS-mA-PS-mA | |
| 452 | 314 | lgT3-PO-lgT3-PO-lgT3- | 1366 | mG-PS-fU-PS-mG-PO- | 1564 |
| | | PO-mG-PO-mC-PO-mC-PO- | | fC-PO-mC-PO-mU-PO- | |
| | | mA-PO-fG-PO-mA-PO-fG- | | mC-PO-mG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | mA-PS-mC | | mC-PS-mA-PS-mA | |
| 453 | 314 | lgT3-PO-lgT3-PO-lgT3- | 1367 | fG-PS-fU-PS-mG-PO- | 1565 |
| | | PO-mG-PO-mC-PO-mC-PO- | | fC-PO-mC-PO-fU-PO- | |
| | | mA-PO-fG-PO-mA-PO-fG- | | mC-PO-fG-PO-mA-PO- | |
| | | PO-fU-PO-fU-PO-mA-PO- | | fU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-fG-PO- | |
| | | mA-PS-mC | | mC-PS-dT-PS-dT | |
| 454 | 314 | lgT3-PO-lgT3-PO-lgT3- | 1368 | mG-PS-fU-PS-mG-PO- | 1566 |
| | | PO-1G-PO-1C-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | mA-PS-mC | | mC-PS-mA-PS-mA | |
| 455 | 314 | lgT3-PO-lgT3-PO-lgT3- | 1369 | mG-PS-fU-PS-mG-PO- | 1567 |
| | | PO-1G-PO-1C-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | mA-PS-mC | | mC-PS-mA-PS-mA | |
| 456 | 314 | lgT3-PO-lgT3-PO-lgT3- | 1370 | mG-PS-fU-PS-mG-PO- | 1568 |
| | | PO-1G-PO-1C-PO-mC-PO- | | fC-PO-mC-PO-mU-PO- | |
| | | mA-PO-fG-PO-mA-PO-fG- | | mC-PO-fG-PO-LA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | mA-PS-mC | | mC-PS-mA-PS-mA | |
| 457 | 314 | lgT3-PO-lgT3-PO-lgT3- | 1371 | mG-PS-fU-PS-mG-PO- | 1569 |
| | | PO-1G-PO-1C-PO-mC-PO- | | fC-PO-mC-PO-mU-PO- | |
| | | mA-PO-fG-PO-mA-PO-fG- | | mC-PO-mG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | mA-PS-mC | | mC-PS-mA-PS-mA | |
| 458 | 314 | lgT3-PO-lgT3-PO-lgT3- | 1372 | fG-PS-fU-PS-mG-PO- | 1570 |
| | | PO-1G-PO-1C-PO-mC-PO- | | fC-PO-mC-PO-fU-PO- | |
| | | mA-PO-fG-PO-mA-PO-fG- | | mC-PO-fG-PO-mA-PO- | |
| | | PO-fU-PO-fU-PO-mA-PO- | | fU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-fG-PO- | |
| | | mA-PS-mC | | mC-PS-dT-PS-dT | |
| 459 | 314 | lgT3-PO-lgT3-PO-lgT3- | 1373 | mG-PS-fU-PS-mG-PO- | 1571 |
| | | PO-mG-PO-mC-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | lT4-PO-lT4 | | mC-PS-mA-PS-mA | |
| 460 | 314 | lgT3-PO-lgT3-PO-lgT3- | 1374 | mG-PS-fU-PS-mG-PO- | 1572 |
| | | PO-mG-PO-mC-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | lT4-PO-lT4 | | mC-PS-mA-PS-mA | |
| 461 | 314 | lgT3-PO-lgT3-PO-lgT3- | 1375 | mG-PS-fU-PS-mG-PO- | 1573 |
| | | PO-mG-PO-mC-PO-mC-PO- | | fC-PO-mC-PO-mU-PO- | |
| | | mA-PO-fG-PO-mA-PO-fG- | | mC-PO-fG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | lT4-PO-lT4 | | mC-PS-mA-PS-mA | |
| 462 | 314 | lgT3-PO-lgT3-PO-lgT3- | 1376 | mG-PS-fU-PS-mG-PO- | 1574 |
| | | PO-mG-PO-mC-PO-mC-PO- | | fC-PO-mC-PO-mU-PO- | |
| | | mA-PO-fG-PO-mA-PO-fG- | | mC-PO-mG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | lT4-PO-lT4 | | mC-PS-mA-PS-mA | |
| 463 | 314 | lgT3-PO-lgT3-PO-lgT3- | 1377 | fG-PS-fU-PS-mG-PO- | 1575 |
| | | PO-mG-PO-mC-PO-mC-PO- | | fC-PO-mC-PO-fU-PO- | |
| | | mA-PO-fG-PO-mA-PO-fG- | | mC-PO-fG-PO-mA-PO- | |
| | | PO-fU-PO-fU-PO-mA-PO- | | fU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-fG-PO- | |
| | | lT4-PO-lT4 | | mC-PS-dT-PS-dT | |
| 464 | 314 | lgT3-PO-lgT3-PO-lgT3- | 1378 | mG-PS-fU-PS-mG-PO- | 1576 |
| | | PO-mG-PO-mC-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | lT4-PO-lT4 | | mC-PS-LA-PS-lA | |
| 465 | 314 | lgT3-PO-lgT3-PO-lgT3- | 1379 | mG-PS-fU-PS-mG-PO- | 1577 |
| | | PO-mG-PO-mC-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | lT4-PO-lT4 | | mC-PS-LA-PS-lA | |
| 466 | 314 | lgT3-PO-lgT3-PO-lgT3- | 1380 | mG-PS-fU-PS-mG-PO- | 1578 |
| | | PO-mG-PO-mC-PO-mC-PO- | | fC-PO-mC-PO-mU-PO- | |
| | | mA-PO-fG-PO-mA-PO-fG- | | mC-PO-fG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | lT4-PO-lT4 | | mC-PS-LA-PS-lA | |
| 467 | 314 | lgT3-PO-lgT3-PO-lgT3- | 1381 | mG-PS-fU-PS-mG-PO- | 1579 |
| | | PO-mG-PO-mC-PO-mC-PO- | | fC-PO-mC-PO-mU-PO- | |
| | | mA-PO-fG-PO-mA-PO-fG- | | mC-PO-mG-PO-LA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | lT4-PO-lT4 | | mC-PS-LA-PS-lA | |
| 468 | 314 | lgT3-PS-lgT3-PS-lgT3- | 1382 | mG-PS-fU-PS-mG-PO- | 1580 |
| | | PO-mG-PO-mC-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | lT4-PS-lT4 | | mC-PS-mA-PS-mA | |
| 469 | 314 | lgT3-PS-lgT3-PS-lgT3- | 1383 | mG-PS-fU-PS-mG-PO- | 1581 |
| | | PO-mG-PO-mC-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | lT4-PS-lT4 | | mC-PS-mA-PS-mA | |
| 470 | 314 | lgT3-PS-lgT3-PS-lgT3- | 1384 | mG-PS-fU-PS-mG-PO- | 1582 |
| | | PO-mG-PO-mC-PO-mC-PO- | | fC-PO-mC-PO-mU-PO- | |
| | | mA-PO-fG-PO-mA-PO-fG- | | mC-PO-fG-PO-A-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | lT4-PS-lT4 | | mC-PS-mA-PS-mA | |
| 471 | 314 | lgT3-PS-lgT3-PS-lgT3- | 1385 | mG-PS-fU-PS-mG-PO- | 1583 |
| | | PO-mG-PO-mC-PO-mC-PO- | | fC-PO-mC-PO-mU-PO- | |
| | | mA-PO-fG-PO-mA-PO-fG- | | mC-PO-mG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | lT4-PS-lT4 | | mC-PS-mA-PS-mA | |
| 472 | 314 | lgT3-PS-lgT3-PS-lgT3- | 1386 | £G-PS-fU-PS-mG-PO- | 1584 |
| | | PO-mG-PO-mC-PO-mC-PO- | | fC-PO-mC-PO-fU-PO- | |
| | | mA-PO-fG-PO-mA-PO-fG- | | mC-PO-fG-PO-mA-PO- | |
| | | PO-fU-PO-fU-PO-mA-PO- | | fU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-fG-PO- | |
| | | lT4-PS-lT4 | | mC-PS-dT-PS-dT | |
| 473 | 314 | lgT3-PS-lgT3-PS-lgT3- | 1387 | mG-PS-fU-PS-mG-PO- | 1585 |
| | | PO-mG-PO-mC-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | lT4-PS-lT4 | | mC-PS-LA-PS-lA | |
| 474 | 314 | lgT3-PS-lgT3-PS-lgT3- | 1388 | mG-PS-fU-PS-mG-PO- | 1586 |
| | | PO-mG-PO-mC-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | lT4-PS-lT4 | | mC-PS-LA-PS-LA | |
| 475 | 314 | lgT3-PS-lgT3-PS-lgT3- | 1389 | mG-PS-fU-PS-mG-PO- | 1587 |
| | | PO-mG-PO-mC-PO-mC-PO- | | fC-PO-mC-PO-mU-PO- | |
| | | mA-PO-fG-PO-mA-PO-fG- | | mC-PO-fG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | lT4-PS-lT4 | | mC-PS-LA-PS-lA | |
| 476 | 314 | lgT3-PS-lgT3-PS-lgT3- | 1390 | mG-PS-fU-PS-mG-PO- | 1588 |
| | | PO-mG-PO-mC-PO-mC-PO- | | fC-PO-mC-PO-mU-PO- | |
| | | mA-PO-fG-PO-mA-PO-fG- | | mC-PO-mG-PO-LA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | lT4-PS-lT4 | | mC-PS-LA-PS-lA | |
| 477 | 314 | lgT3-PO-lgT3-PO-lgT3- | 1391 | mG-PS-fU-PS-mG-PO- | 1589 |
| | | PO-1G-PO-1C-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | lT4-PO-lT4 | | mC-PS-mA-PS-mA | |
| 478 | 314 | lgT3-PO-lgT3-PO-lgT3- | 1392 | mG-PS-fU-PS-mG-PO- | 1590 |
| | | PO-1G-PO-1C-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | lT4-PO-lT4 | | mC-PS-mA-PS-mA | |
| 479 | 314 | lgT3-PO-lgT3-PO-lgT3- | 1393 | mG-PS-fU-PS-mG-PO- | 1591 |
| | | PO-1G-PO-1C-PO-mC-PO- | | fC-PO-mC-PO-mU-PO- | |
| | | mA-PO-fG-PO-mA-PO-fG- | | mC-PO-fG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | lT4-PO-lT4 | | mC-PS-mA-PS-mA | |
| 480 | 314 | lgT3-PO-lgT3-PO-lgT3- | 1394 | mG-PS-fU-PS-mG-PO- | 1592 |
| | | PO-1G-PO-1C-PO-mC-PO- | | fC-PO-mC-PO-mU-PO- | |
| | | mA-PO-fG-PO-mA-PO-fG- | | mC-PO-mG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | lT4-PO-lT4 | | mC-PS-mA-PS-mA | |
| 48 | 314 | lgT3-PO-lgT3-PO-lgT3- | 1395 | fG-PS-fU-PS-mG-PO- | 1593 |
| | | PO-1G-PO-1C-PO-mC-PO- | | fC-PO-mC-PO-fU-PO- | |
| | | mA-PO-fG-PO-mA-PO-fG- | | mC-PO-fG-PO-mA-PO- | |
| | | PO-fU-PO-fU-PO-mA-PO- | | fU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-fG-PO- | |
| | | lT4-PO-lT4 | | mC-PS-dT-PS-dT | |
| 482 | 314 | lgT3-PS-lgT3-PS-lgT3- | 1396 | mG-PS-fU-PS-mG-PO- | 1594 |
| | | PO-1G-PO-1C-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | lT4-PS-lT4 | | mC-PS-mA-PS-mA | |
| 483 | 314 | lgT3-PS-lgT3-PS-lgT3- | 1397 | mG-PS-fU-PS-mG-PO- | 1595 |
| | | PO-1G-PO-1C-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | lT4-PS-lT4 | | mC-PS-mA-PS-mA | |
| 484 | 314 | lgT3-PS-lgT3-PS-lgT3- | 1398 | mG-PS-fU-PS-mG-PO- | 1596 |
| | | PO-1G-PO-1C-PO-mC-PO- | | fC-PO-mC-PO-mU-PO- | |
| | | mA-PO-fG-PO-mA-PO-fG- | | mC-PO-fG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | lT4-PS-lT4 | | mC-PS-mA-PS-mA | |
| 485 | 314 | lgT3-PS-lgT3-PS-lgT3- | 1399 | mG-PS-fU-PS-mG-PO- | \|1597 |
| | | PO-1G-PO-1C-PO-mC-PO- | | fC-PO-mC-PO-mU-PO- | |
| | | mA-PO-fG-PO-mA-PO-fG- | | mC-PO-mG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-mG-PO- | |
| | | lT4-PS-lT4 | | mC-PS-mA-PS-mA | |
| 486 | 314 | lgT3-PS-lgT3-PS-lgT3- | 1400 | fG-PS-fU-PS-mG-PO- | 1598 |
| | | PO-1G-PO-1C-PO-mC-PO- | | fC-PO-mC-PO-fU-PO- | |
| | | mA-PO-fG-PO-mA-PO-fG- | | mC-PO-fG-PO-mA-PO- | |
| | | PO-fU-PO-fU-PO-mA-PO- | | fU-PO-mA-PO-mA-PO- | |
| | | mU-PO-mC-PO-mG-PO-mA- | | mC-PO-fU-PO-mC-PO- | |
| | | PO-mG-PO-mG-PO-mC-PS- | | fU-PO-mG-PO-fG-PO- | |
| | | lT4-PS-lT4 | | mC-PS-dT-PS-dT | |
| 487 | 314 | lgT3-PO-lgT3-PO-lgT3- | 1401 | mG-PS-fU-PS-mG-PO- | 1599 |
| | | PO-mG-PO-mC-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PO-lT4-PO-lT4 | | mC-PS-mA-PS-mA | |
| 488 | 314 | lgT3-PO-lgT3-PO-lgT3- | 1402 | mG-PS-fU-PS-mG-PO- | 1600 |
| | | PO-mG-PO-mC-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PO-lT4-PO-lT4 | | mC-PS-mA-PS-mA-Hy | |
| 489 | 314 | lgT3-PO-lgT3-PO-lgT3- | 1403 | mG-PS-fU-PS-mG-PO- | 1601 |
| | | PO-mG-PO-mC-PO-mC-PO- | | fC-PO-mC-PO-mU-PO- | |
| | | mA-PO-fG-PO-mA-PO-fG- | | mC-PO-fG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PO-lT4-PO-lT4 | | mC-PS-mA-PS-mA | |
| 490 | 314 | lgT3-PO-lgT3-PO-lgT3- | 1404 | mG-PS-fU-PS-mG-PO- | \|1602 |
| | | PO-mG-PO-mC-PO-mC-PO- | | fC-PO-mC-PO-mU-PO- | |
| | | mA-PO-fG-PO-mA-PO-fG- | | mC-PO-mG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PO-lT4-PO-lT4 | | mC-PS-mA-PS-mA | |
| 491 | 314 | lgT3-PO-lgT3-PO-lgT3- | 1405 | fG-PS-fU-PS-mG-PO- | 1603 |
| | | PO-mG-PO-mC-PO-mC-PO- | | fC-PO-mC-PO-fU-PO- | |
| | | mA-PO-fG-PO-mA-PO-fG- | | mC-PO-fG-PO-mA-PO- | |
| | | PO-fU-PO-fU-PO-mA-PO- | | fU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-fG-PO- | |
| | | mA-PO-mC-PO-lT4-PO-lT4 | | mC-PS-dT-PS-dT | |
| 492 | 314 | lgT3-PO-lgT3-PO-lgT3- | 1406 | mG-PS-fU-PS-mG-PO- | 1604 |
| | | PO-mG-PO-mC-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PO-lT4-PO-lT4 | | mC-PS-LA-PS-lA | |
| 493 | 314 | lgT3-PO-lgT3-PO-lgT3- | 1407 | mG-PS-fU-PS-mG-PO- | 1605 |
| | | PO-mG-PO-mC-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PO-lT4-PO-lT4 | | mC-PS-LA-PS-lA | |
| 494 | 314 | lgT3-PO-lgT3-PO-lgT3- | 1408 | mG-PS-fU-PS-mG-PO- | 1606 |
| | | PO-mG-PO-mC-PO-mC-PO- | | fC-PO-mC-PO-mU-PO- | |
| | | mA-PO-fG-PO-mA-PO-fG- | | mC-PO-fG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PO-lT4-PO-lT4 | | mC-PS-lA-PS-lA | |
| 495 | 314 | lgT3-PO-lgT3-PO-lgT3- | 1409 | mG-PS-fU-PS-mG-PO- | 1607 |
| | | PO-mG-PO-mC-PO-mC-PO- | | fC-PO-mC-PO-mU-PO- | |
| | | mA-PO-fG-PO-mA-PO-fG- | | mC-PO-mG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PO-lT4-PO-lT4 | | mC-PS-LA-PS-lA | |
| 496 | 314 | lgT3-PS-lgT3-PS-lgT3- | 1410 | mG-PS-fU-PS-mG-PO- | 1608 |
| | | PO-mG-PO-mC-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PS-lT4-PS-lT4 | | mC-PS-mA-PS-mA | |
| 497 | 314 | lgT3-PS-lgT3-PS-lgT3- | 1411 | mG-PS-fU-PS-mG-PO- | 1609 |
| | | PO-mG-PO-mC-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PS-lT4-PS-lT4 | | mC-PS-mA-PS-mA | |
| 498 | 314 | lgT3-PS-lgT3-PS-lgT3- | 1412 | mG-PS-fU-PS-mG-PO- | 1610 |
| | | PO-mG-PO-mC-PO-mC-PO- | | fC-PO-mC-PO-mU-PO- | |
| | | mA-PO-fG-PO-mA-PO-fG- | | mC-PO-fG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PS-lT4-PS-lT4 | | mC-PS-mA-PS-mA | |
| 499 | 314 | lgT3-PS-lgT3-PS-lgT3- | 1413 | mG-PS-fU-PS-mG-PO- | \|1611 |
| | | PO-mG-PO-mC-PO-mC-PO- | | fC-PO-mC-PO-mU-PO- | |
| | | mA-PO-fG-PO-mA-PO-fG- | | mC-PO-mG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PS-lT4-PS-lT4 | | mC-PS-mA-PS-mA | |
| 500 | 314 | lgT3-PS-lgT3-PS-lgT3- | 1414 | fG-PS-fU-PS-mG-PO- | 1612 |
| | | PO-mG-PO-mC-PO-mC-PO- | | fC-PO-mC-PO-fU-PO- | |
| | | mA-PO-fG-PO-mA-PO-fG- | | mC-PO-fG-PO-mA-PO- | |
| | | PO-fU-PO-fU-PO-mA-PO- | | fU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-fG-PO- | |
| | | mA-PO-mC-PS-lT4-PS-lT4 | | mC-PS-dT-PS-dT | |
| 501 | 314 | lgT3-PS-lgT3-PS-lgT3- | 1415 | mG-PS-fU-PS-mG-PO- | 1613 |
| | | PO-mG-PO-mC-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PS-lT4-PS-lT4 | | mC-PS-lA-PS-lA | |
| 502 | 314 | lgT3-PS-lgT3-PS-lgT3- | 1416 | mG-PS-fU-PS-mG-PO- | 1614 |
| | | PO-mG-PO-mC-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PS-lT4-PS-lT4 | | mC-PS-LA-PS-lA | |
| 503 | 314 | lgT3-PS-lgT3-PS-lgT3- | 1417 | mG-PS-fU-PS-mG-PO- | 1615 |
| | | PO-mG-PO-mC-PO-mC-PO- | | fC-PO-mC-PO-mU-PO- | |
| | | mA-PO-fG-PO-mA-PO-fG- | | mC-PO-fG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PS-lT4-PS-lT4 | | mC-PS-LA-PS-lA | |
| 504 | 314 | lgT3-PS-lgT3-PS-lgT3- | 1418 | mG-PS-fU-PS-mG-PO- | 1616 |
| | | PO-mG-PO-mC-PO-mC-PO- | | fC-PO-mC-PO-mU-PO- | |
| | | mA-PO-fG-PO-mA-PO-fG- | | mC-PO-mG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PS-lT4-PS-lT4 | | mC-PS-LA-PS-lA | |
| 505 | 314 | lgT3-PO-lgT3-PO-lgT3- | 1419 | mG-PS-fU-PS-mG-PO- | 1617 |
| | | PO-1G-PO-1C-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PO-lT4-PO-lT4 | | mC-PS-mA-PS-mA | |
| 506 | 314 | lgT3-PO-lgT3-PO-lgT3- | 1420 | mG-PS-fU-PS-mG-PO- | 1618 |
| | | PO-1G-PO-1C-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PO-lT4-PO-lT4 | | mC-PS-mA-PS-mA | |
| 507 | 314 | lgT3-PO-lgT3-PO-lgT3- | 1421 | mG-PS-fU-PS-mG-PO- | 1619 |
| | | PO-1G-PO-1C-PO-mC-PO- | | fC-PO-mC-PO-mU-PO- | |
| | | mA-PO-fG-PO-mA-PO-fG- | | mC-PO-fG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PO-lT4-PO-lT4 | | mC-PS-mA-PS-mA | |
| 508 | 314 | lgT3-PO-lgT3-PO-lgT3- | 1422 | mG-PS-fU-PS-mG-PO- | 1620 |
| | | PO-1G-PO-1C-PO-mC-PO- | | fC-PO-mC-PO-mU-PO- | |
| | | mA-PO-fG-PO-mA-PO-fG- | | mC-PO-mG-PO-LA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PO-lT4-PO-lT4 | | mC-PS-mA-PS-mA | |
| 509 | 314 | lgT3-PO-lgT3-PO-lgT3- | 1423 | fG-PS-fU-PS-mG-PO- | 1621 |
| | | PO-1G-PO-1C-PO-mC-PO- | | fC-PO-mC-PO-fU-PO- | |
| | | mA-PO-fG-PO-mA-PO-fG- | | mC-PO-fG-PO-mA-PO- | |
| | | PO-fU-PO-fU-PO-mA-PO- | | fU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-fG-PO- | |
| | | mA-PO-mC-PO-lT4-PO-lT4 | | mC-PS-dT-PS-dT | |
| 510 | 314 | lgT3-PS-lgT3-PS-lgT3- | 1424 | mG-PS-fU-PS-mG-PO- | 1622 |
| | | PO-1G-PO-1C-PO-mC-PO- | | fC-PO-mC-PO-mU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PS-lT4-PS-lT4 | | mC-PS-mA-PS-mA | |
| 511 | 314 | lgT3-PS-lgT3-PS-lgT3- | 1425 | mG-PS-fU-PS-mG-PO- | 1623 |
| | | PO-1G-PO-1C-PO-mC-PO- | | mC-PO-mC-PO-fU-PO- | |
| | | mA-PO-fG-PO-mA-PO-fG- | | mC-PO-mG-PO-mA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PS-lT4-PS-lT4 | | mC-PS-mA-PS-mA | |
| 512 | 314 | lgT3-PS-lgT3-PS-lgT3- | 1426 | mG-PS-fU-PS-mG-PO- | 1624 |
| | | PO-1G-PO-1C-PO-mC-PO- | | fC-PO-mC-PO-mU-PO- | |
| | | mA-PO-fG-PO-mA-PO-fG- | | mC-PO-fG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PS-lT4-PS-lT4 | | mC-PS-mA-PS-mA | |
| 513 | 314 | lgT3-PS-lgT3-PS-lgT3- | 1427 | mG-PS-fU-PS-mG-PO- | 1625 |
| | | PO-1G-PO-1C-PO-mC-PO- | | fC-PO-mC-PO-mU-PO- | |
| | | mA-PO-fG-PO-mA-PO-fG- | | mC-PO-mG-PO-fA-PO- | |
| | | PO-fU-PO-fU-PO-mA-PO- | | mU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-mG-PO- | |
| | | mA-PO-mC-PS-lT4-PS-lT4 | | mC-PS-mA-PS-mA-Hy | |
| 514 | 314 | lgT3-PS-lgT3-PS-lgT3- | 1428 | fG-PS-fU-PS-mG-PO- | 1626 |
| | | PO-1G-PO-1C-PO-mC-PO- | | fC-PO-mC-PO-fU-PO- | |
| | | mA-PO-fG-PO-mA-PO-fG- | | mC-PO-fG-PO-mA-PO- | |
| | | PO-fU-PO-fU-PO-mA-PO- | | fU-PO-mA-PO-mA-PO- | |
| | | mU-PO-mC-PO-mG-PO-mA- | | mC-PO-fU-PO-mC-PO- | |
| | | PO-mG-PO-mG-PO-mC-PO- | | fU-PO-mG-PO-fG-PO- | |
| | | mA-PO-mC-PS-lT4-PS-lT4 | | mC-PS-dT-PS-dT | |

While the exemplary siRNAs shown in Tables 2, 3, and 4 include nucleotide modifications, siRNAs having the same or substantially the same sequences but different numbers, patterns, and/or types of modifications, are also contemplated.

In some embodiments, a dsRNA comprises a sense strand shown in Table 1 with the addition of nucleotides (or modified versions thereof) at either or both of its termini. For example, the dsRNA comprises a sense strand shown in Table 1 with the addition of a 5′ CCA and/or a 3′ invdT. In some embodiments, a dsRNA comprises an antisense strand shown in Table 1 with the addition of nucleotides (or modified versions thereof) at either or both of its termini. For example, the dsRNA comprises an antisense strand shown in Table 1 with the addition of a 3′ dTdT. In certain embodiments, a dsRNA comprises a pair of sense and antisense strands as shown in Table 1, with the addition of a 5′ CCA and a 3′ invdT to the sense strand and with the addition of a 3′ dTdT to the antisense strand. In certain embodiments, a dsRNA comprises a pair of sense and antisense strands as shown in Table 2, with the addition of a 5′ lgT3-1gT3-1gT3 and a 3′ 1T4-lT4 to the sense strand.

In some embodiments, a dsRNA of the present disclosure comprises a sense sequence that is at least 70%, 75%, 80%, 85%, 90%, 95%, 96%, 97%, 98%, or 99% identical in sequence to a sense sequence shown in Table 1. In some embodiments, a dsRNA of the present disclosure comprises an antisense sequence that is at least 70%, 75%, 80%, 85%, 90%, 95%, 96%, 97%, 98%, or 99% identical in sequence to an antisense sequence shown in Table 1. In some embodiments, a dsRNA of the present disclosure comprises sense and antisense sequences that are at least 70%, 75%, 80%, 85%, 90%, 95%, 96%, 97%, 98%, or 99% identical in sequence to sense and antisense sequences, respectively, shown in Table 1. In certain embodiments, the dsRNA comprises sense and antisense strands having the sequences shown in Table 2. In certain embodiments, the dsRNA comprises sense and antisense strands having the sequences shown in Tables 3 and 4. In certain embodiments, the dsRNA is selected from the dsRNA in Tables 1-4.

The “percentage identity” between two nucleotide sequences is determined by comparing the two optimally-aligned sequences in which the nucleic acid sequence to compare can have additions or deletions compared to the reference sequence for optimal alignment between the two sequences. “Percentage identity” is calculated by determining the number of positions at which the nucleotide residue is identical between the two sequences, preferably between the two complete sequences, dividing the number of identical positions by the total number of positions in the alignment window and multiplying the result by 100 to obtain the percentage identity between the two sequences. For purposes herein, when determining “percentage identity” between two nucleotide sequences, modifications to the nucleotides are not considered. For example, a sequence of 5′-mC-fU-mA-fG-3′ is considered having 100% sequence identity as a sequence of 5′-CUAG-3′.

I.5 dsRNA Conjugates

The present dsRNAs may be covalently or noncovalently linked to one or more ligands or moieties. Examples of such ligands and moieties may be found, e.g., in Jeong et al., Bioconjugate Chem. (2009) 20:5-14 and Sebestyen et al., Methods Mol Biol. (2015) 1218:163-86. In some embodiments, the dsRNA is conjugated/attached to one or more ligands via a linker. Any linker known in the art may be used, including, for example, multivalent (e.g., bivalent, trivalent, or tetravalent) branched linkers. The linker may be cleavable or non-cleavable. Conjugating a ligand to a dsRNA may alter its distribution, enhance its cellular absorption and/or targeting to a particular tissue and/or uptake by one or more specific cell types (e.g., liver cells), and/or enhance the lifetime or half-life of the dsRNA. In some embodiments, a hydrophobic ligand is conjugated to the dsRNA to facilitate direct permeation of the cellular membrane and/or uptake across cells (e.g., liver cells). For LPA mRNA-targeting dsRNAs (e.g., siRNAs), the target tissue may be the liver, including parenchymal cells of the liver (e.g., hepatocytes). In some embodiments, the dsRNA is conjugated to one or more ligands with or without a linker.

In some embodiments, the dsRNA of the present disclosure is conjugated to a cell-targeting ligand. A cell-targeting ligand refers to a molecular moiety that facilitates delivery of the dsRNA to the target cell, which encompasses (i) increased specificity of the dsRNA to bind to cells expressing the selected target receptors (e.g., target proteins); (ii) increased uptake of the dsRNA by the target cells; and (iii) increased ability of the dsRNA to be appropriately processed once it has entered into a target cell, such as increased intracellular release of an siRNA, e.g., by facilitating the translocation of the siRNA from transport vesicles into the cytoplasm. The ligand may be, for example, a protein (e.g., a glycoprotein), a peptide, a lipid, a carbohydrate, an aptamer, or a molecule having a specific affinity for a co-ligand.

Specific examples of ligands include, without limitation, an antibody or antigen-binding fragment thereof that binds to a specific receptor on a liver cell, thyrotropin, melanotropin, surfactant protein A, mucin carbohydrate, multivalent lactose, multivalent galactose, multivalent mannose, multivalent fucose, N-acetylgalactosamine, N-acetylglucosamine, transferrin, bisphosphonate, a steroid, bile acid, lipopolysaccharide, a recombinant or synthetic molecule such as a synthetic polymer, polyamino acids, an alpha helical peptide, polyglutamate, polyaspartate, lectins, and cofactors. In some embodiments, the ligand is one or more dyes, crosslinkers, polycyclic aromatic hydrocarbons, peptide conjugates (e.g., antennapedia peptide, Tat peptide), polyethylene glycol (PEG), enzymes, haptens, transport/absorption facilitators, synthetic ribonucleases (e.g., imidazole, bisimidazole, histamine, or imidazole clusters), human serum albumin (HSA), or LDL.

In some embodiments, the dsRNA is conjugated to one or more cholesterol derivatives or lipophilic moieties such as cholesterol or a cholesterol derivative; cholic acid; a vitamin (such as folate, vitamin A, vitamin E (tocopherol), biotin, or pyridoxal); bile or fatty acid conjugates, including both saturated and non-saturated (such as lauroyl (C12), myristoyl (C14), palmitoyl (C16), stearoyl (C18) and docosanyl (C22), lithocholic acid and/or lithocholic acid oleylamine conjugate (lithocholic-oleyl, C43)); polymeric backbones or scaffolds (such as PEG, triethylene glycol (TEG), hexaethylene glycol (HEG), poly(lactic-co-glycolic acid) (PLGA), poly(lactide-co-glycolide) (PLG), hydrodynamic polymers); steroids (such as dihydrotestosterone); terpene (such as triterpene); cationic lipids or peptides; and/or a lipid or lipid-based molecule. Such a lipid or lipid-based molecule may bind a serum protein, e.g., human serum albumin (HSA). A lipid-based ligand may be used to modulate (e.g., control) the binding of the conjugate to a target tissue. For example, a lipid or lipid-based ligand that binds to HSA more strongly will be less likely to be targeted to the kidney and therefore less likely to be cleared from the body.

In some embodiments, the cell-targeting moiety or ligand is a N-acetylgalactosamine (GalNAc) derivative. In some embodiments, the dsRNA is attached to one or more (e.g., two, three, four, or more) GalNAc derivatives. The attachment may be via one or more linkers (e.g., two, three, four, or more linkers). In some embodiments, a linker described herein is a multivalent (e.g., bivalent, trivalent, or tetravalent) branched linker. In some embodiments, the dsRNA is attached to two or more GalNAc derivatives via a bivalent branched linker. In some embodiments, the dsRNA is attached to three or more GalNAc derivatives via a trivalent branched linker. In some embodiments, the dsRNA is attached to three or more GalNAc derivatives with or without linkers. In some embodiments, the dsRNA is attached to four or more GalNAc derivatives via four separate linkers. In some embodiments, the dsRNA is attached to four or more GalNAc derivatives via a tetravalent branched linker. In some embodiments, the one or more GalNAc derivatives is attached to the 3′-end of the sense strand, the 3′-end of the antisense strand, the 5′-end of the sense strand, and/or the 5′-end of the antisense strand of the dsRNA. Exemplary and non-limiting conjugates and linkers are described, e.g., in Biessen et al., Bioconjugate Chem. (2002) 13(2):295-302; Cedillo et al., Molecules (2017) 22(8):E1356; Grijalvo et al., Genes (2018) 9(2):E74; Huang et al., Molecular Therapy: Nucleic Acids (2017) 6:116-32; Nair et al., J Am Chem Soc. (2014) 136:16958-61; Ostergaard et al., Bioconjugate Chem. (2015) 26:1451-5; Springer et al., Nucleic Acid Therapeutics (2018) 28(3):109-18; and U.S. Pat. Nos. 8,106,022, 9,127,276, and 8,927,705. GalNAc conjugation can be readily performed by methods well known in the art (e.g., as described in the above documents).

In some embodiments, the ligand is N-acetylgalactosamine (GalNAc) and the dsRNA is conjugated to one or more GalNAc.

### II. Methods of Making dsRNAs

A dsRNA of the present disclosure may be synthesized by any method known in the art. For example, a dsRNA may be synthesized by use of an automated synthesizer, by in vitro transcription and purification (e.g., using commercially available in vitro RNA synthesis kits), by transcription and purification from cells (e.g., cells comprising an expression cassette/vector encoding the dsRNA), and the like. In some embodiments, the sense and antisense strands of the dsRNA are synthesized separately and then annealed to form the dsRNA. In some embodiments, the dsRNA comprising modified nucleotides of formula (I) and optionally conjugated to a cell targeting moiety (e.g., GalNAc) may be prepared according to the disclosure of PCT Publication WO 2019/170731.

Ligand-conjugated dsRNAs and ligand molecules bearing sequence-specific linked nucleosides of the present disclosure may be assembled by any method known in the art, including, for example, assembly on a suitable polynucleotide synthesizer utilizing standard nucleotide or nucleoside precursors, or nucleotide or nucleoside conjugate precursors that already bear the linking moiety, ligand-nucleotide, or nucleoside-conjugated precursors that already bear the ligand molecule, or non-nucleoside ligand-bearing building blocks.

Ligand-conjugated dsRNAs of the present disclosure may be synthesized by any method known in the art, including, for example, by the use of a dsRNA bearing a pendant reactive functionality such as that derived from the attachment of a linking molecule onto the dsRNA. In some embodiments, this reactive oligonucleotide may be reacted directly with commercially-available ligands, ligands that are synthesized bearing any of a variety of protecting groups, or ligands that have a linking moiety attached thereto. In some embodiments, the methods facilitate the synthesis of ligand-conjugated dsRNA by the use of nucleoside monomers that have been appropriately conjugated with ligands and that may further be attached to a solid support material. In some embodiments, a dsRNA bearing an aralkyl ligand attached to the 3′-end of the dsRNA is prepared by first covalently attaching a monomer building block to a controlled-pore-glass support via a long-chain aminoalkyl group; then, nucleotides are bonded via standard solid-phase synthesis techniques to the monomer building-block bound to the solid support. The monomer building-block may be a nucleoside or other organic compound that is compatible with solid-phase synthesis.

In some embodiments, functionalized nucleoside sequences of the present disclosure possessing an amino group at the 5′-terminus are prepared using a polynucleotide synthesizer, and then reacted with an active ester derivative of a selected ligand. Active ester derivatives are well known to one of ordinary skill in the art. The reaction of the amino group and the active ester produces an oligonucleotide in which the selected ligand is attached to the 5′-position through a linking group. The amino group at the 5′-terminus can be prepared utilizing a 5′-amino-modifier C6 reagent. In some embodiments, ligand molecules are conjugated to oligonucleotides at the 5′-position by the use of a ligand-nucleoside phosphoramidite wherein the ligand is linked to the 5′-hydroxy group directly or indirectly via a linker. Such ligand-nucleoside phosphoramidites are typically used at the end of an automated synthesis procedure to provide a ligand-conjugated oligonucleotide bearing the ligand at the 5′-terminus.

In some embodiments, click chemistry is used to synthesize siRNA conjugates. See, e.g., Astakhova et al., Mol Pharm. (2018) 15(8):2892-9; Mercier et al., Bioconjugate Chem. (2011) 22(1):108-14.

### III. Compositions and Delivery of dsRNAs

Certain aspects of the present disclosure relate to compositions (e.g., pharmaceutical compositions) comprising a dsRNA as described herein. In some embodiments, the composition further comprises a pharmaceutically acceptable excipient. In some embodiments, the composition is useful for treating a disease or disorder associated with the expression or activity of the LPA gene. In some embodiments, the disease or disorder associated with the expression of the LPA gene is a lipid metabolism disorder such as hypertriglyceridemia and/or any other condition described herein. Compositions of the present disclosure may be formulated based upon the mode of delivery, including, for example, compositions formulated for delivery to the liver via parenteral administration.

The present dsRNAs can be formulated with a pharmaceutically acceptable excipient. Pharmaceutically acceptable excipients can be liquid or solid, and may be selected with the planned manner of administration in mind so as to provide for the desired bulk, consistency, and other pertinent transport and chemical properties. Any known pharmaceutically acceptable excipient may be used, including, for example, water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), calcium salts (e.g., calcium sulfate, calcium chloride, calcium phosphate, and hydroxyapatite), and wetting agents (e.g., sodium lauryl sulfate).

The present dsRNAs can be formulated into compositions (e.g., pharmaceutical compositions) containing the dsRNA admixed, encapsulated, conjugated, or otherwise associated with other molecules, molecular structures, or mixtures of nucleic acids. For example, a composition comprising one or more dsRNAs as described herein can contain other therapeutic agents such as other lipid lowering agents (e.g., statins). In some embodiments, the composition (e.g., pharmaceutical composition) further comprises a delivery vehicle as described herein.

A dsRNA of the present disclosure may be delivered directly or indirectly. In some embodiments, the dsRNA is delivered directly by administering a pharmaceutical composition comprising the dsRNA to a subject. In some embodiments, the dsRNA is delivered indirectly by administering one or more vectors described below.

A dsRNA of the present disclosure may be delivered by any method known in the art, including, for example, by adapting a method of delivering a nucleic acid molecule for use with a dsRNA (see, e.g., Akhtar et al., Trends Cell Biol. (1992) 2(5):139-44; PCT Publication WO 94/02595), or via additional methods known in the art (see, e.g., Kanasty et al., Nature Materials (2013) 12:967-77; Wittrup and Lieberman, Nature Reviews Genetics (2015) 16:543-52; Whitehead et al., Nature Reviews Drug Discovery (2009) 8:129-38; Gary et al., J Control Release (2007) 121(1-2):64-73; Wang et al., AAPS J. (2010) 12(4):492-503; Draz et al., Theranostics (2014) 4(9):872-92; Wan et al., Drug Deliv Transl Res. (2013) 4(1):74-83; Erdmann and Barciszewski (eds.) (2010) “RNA Technologies and Their Applications,” Springer-Verlag Berlin Heidelberg, DOI 10.1007/978-3-642-12168-5; Xu and Wang, Asian Journal of Pharmaceutical Sciences (2015) 10(1):1-12). For in vivo delivery, dsRNA can be injected into a tissue site or administered systemically (e.g., in nanoparticle form via inhalation). In vivo delivery can also be mediated by a beta-glucan delivery system (see, e.g., Tesz et al., Biochem J. (2011) 436(2):351-62). In vitro introduction into a cell includes methods known in the art such as electroporation and lipofection.

In some embodiments, a dsRNA of the present disclosure is delivered by a delivery vehicle comprising the dsRNA. In some embodiments, the delivery vehicle is a liposome, lipoplex, complex, or nanoparticle.

III.1 Liposomal Formulations

Liposomes are unilamellar or multilamellar vesicles which have a membrane formed from a lipophilic material and an aqueous interior. In some embodiments, a liposome is a vesicle composed of amphiphilic lipids arranged in a spherical bilayer or bilayers. The aqueous portion contains the composition to be delivered. Cationic liposomes possess the advantage of being able to fuse to the cell wall. Advantages of liposomes include, e.g., that liposomes obtained from natural phospholipids are biocompatible and biodegradable; that liposomes can incorporate a wide range of water and lipid soluble drugs; and that liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes. For example, engineered cationic liposomes and sterically stabilized liposomes can be used to deliver the dsRNA. See, e.g., Podesta et al., Methods Enzymol. (2009) 464:343-54; U.S. Pat. No. 5,665,710.

III.2 Nucleic Acid-Lipid Particles

In some embodiments, a dsRNA of the present disclosure is fully encapsulated in a lipid formulation, e.g., to form a nucleic acid-lipid particle such as, without limitation, a SPLP, pSPLP, or SNALP. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. Nucleic acid-lipid particles, e.g., SNALPs, typically contain a cationic lipid, a non-cationic lipid, and a lipid that prevents aggregation of the particle (e.g., a PEG-lipid conjugate). SNALPs and SPLPs are useful for systemic applications, as they exhibit extended circulation lifetimes following intravenous (i.v.) injection and accumulate at distal sites (e.g., sites physically separated from the administration site). SPLPs include “pSPLPs,” which include an encapsulated condensing agent-nucleic acid complex as set forth in PCT Publication WO 00/03683.

In some embodiments, dsRNAs when present in nucleic acid-lipid particles are resistant in aqueous solution to degradation with a nuclease. Nucleic acid-lipid particles and their methods of preparation are disclosed in, e.g., U.S. Pat. Nos. 5,976,567; 5,981,501; 6,534,484; 6,586,410; and 6,815,432; and PCT Publication WO 96/40964.

In some embodiments, the nucleic acid-lipid particles comprise a cationic lipid. Any cationic lipid or mixture thereof known in the art may be used. In some embodiments, the nucleic acid-lipid particles comprise a non-cationic lipid. Any non-cationic lipid or mixture thereof known in the art may be used. In some embodiments, the nucleic acid-lipid particle comprises a conjugated lipid (e.g., to prevent aggregation). Any conjugated lipid known in the art may be used.

III.3 Additional Formulations

Factors that are important to consider in order to successfully deliver a dsRNA molecule in vivo include: (1) biological stability of the delivered molecule, (2) preventing nonspecific effects, and (3) accumulation of the delivered molecule in the target tissue. The nonspecific effects of a dsRNA can be minimized by local administration, for example by direct injection or implantation into a tissue or topically administering the preparation. For administering a dsRNA systemically for the treatment of a disease, the dsRNA may be modified or alternatively delivered using a drug delivery system; both methods act to prevent the rapid degradation of the dsRNA by endo- and exonucleases in vivo. Modification of the RNA or the pharmaceutical excipient may also permit targeting of the dsRNA composition to the target tissue and avoid undesirable off-target effects. As described above, dsRNA molecules may be modified by chemical conjugation to lipophilic groups such as cholesterol to enhance cellular uptake and prevent degradation. In some embodiments, the dsRNA is delivered using drug delivery systems such as a nanoparticle (e.g., a calcium phosphate nanoparticle), a dendrimer, a polymer, liposomes, or a cationic delivery system. Positively charged cationic delivery systems facilitate binding of a dsRNA molecule (negatively charged) and also enhance interactions at the negatively charged cell membrane to permit efficient uptake of a dsRNA by the cell. Cationic lipids, dendrimers, or polymers can either be bound to a dsRNA, or induced to form a vesicle or micelle (See, e.g., Kim et al., Journal of Controlled Release (2008) 129(2):107-16) that encases a dsRNA. The formation of vesicles or micelles further prevents degradation of the dsRNA when administered systemically. Methods for making and administering cationic-dsRNA complexes are known in the art. In some embodiments, a dsRNA may form a complex with cyclodextrin for systemic administration.

III.4 Vector-Encoded dsRNAs

A dsRNA of the present disclosure may be delivered to the target cell indirectly by introducing into the target cell a recombinant vector (DNA or RNA vector) encoding the dsRNA. The dsRNA will be expressed from the vector inside the cell, e.g., in the form of shRNA, where the shRNA is subsequently processed into siRNA intracellularly. In some embodiments, the vector is a plasmid, cosmid, or viral vector. In some embodiments, the vector is compatible with expression in prokaryotic cells. In some embodiments, the vector is compatible with expression in E. coli. In some embodiments, the vector is compatible with expression in eukaryotic cells. In some embodiments, the vector is compatible with expression in yeast cells. In some embodiments, the vector is compatible with expression in vertebrate cells. Any expression vector capable of encoding dsRNA known in the art may be used, including, for example, vectors derived from adenovirus (AV), adeno-associated virus (AAV), retroviruses (e.g., lentiviruses (LV), Rhabdoviruses, murine leukemia virus, etc.), herpes virus, SV40 virus, polyoma virus, papilloma virus, picornavirus, pox virus (e.g., orthopox or avipox), and the like. The tropism of viral vectors or viral-derived vectors may be modified by pseudotyping the vectors with envelope proteins or other surface antigens from one or more other viruses, or by substituting different viral capsid proteins, as appropriate. For example, lentiviral vectors may be pseudotypes with surface proteins from vesicular stomatitis virus (VSV), rabies, Ebola, Mokola, and the like. AAV vectors may be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype capsid gene to produce an AAV 2/5 vector. Techniques for constructing AAV vectors which express different capsid protein serotypes have been described previously (see, e.g., Rabinowitz et al., J. Virol. (2002) 76:791-801).

Selection of recombinant vectors, methods for inserting nucleic acid sequences into the vector for expressing a dsRNA, and methods of delivering vectors into one or more cells of interest are known in the art. See, e.g., Domburg, Gene Therap. (1995) 2:301-10; Eglitis et al., Biotechniques (1998) 6:608-14; Miller, Hum Gene Therap. (1990) 1:5-14; Anderson et al., Nature (1998) 392:25-30; Xia et al., Nat. Biotech. (2002) 20:1006-10; Robinson et al., Nat Genet. (2003) 33:401-6; Samulski et al., J. Virol. (1987) 61:3096-101; Fisher et al., J Virol. (1996) 70:520-32; Samulski et al., J Virol. (1989) 63:3822-6; U.S. Pat. Nos. 5,252,479 and 5,139,941; and PCT Publications WO 94/13788 and WO 93/24641.

Vectors useful for the delivery of a dsRNA as described herein may include regulatory elements (e.g., heterologous promoter, enhancer, etc.) sufficient for expression of the dsRNA in the desired target cell or tissue. In some embodiments, the vector comprises one or more sequences encoding the dsRNA linked to one or more heterologous promoters. Any heterologous promoter known in the art capable of expressing a dsRNA may be used, including, for example, the U6 or H1 RNA pol III promoters, the T7 promoter, and the cytomegalovirus promoter. The one or more heterologous promoters may be an inducible promoter, a repressible promoter, a regulatable promoter, and/or a tissue-specific promoter. Selection of additional promoters is within the abilities of one of ordinary skill in the art. In some embodiments, the regulatory elements are selected to provide constitutive expression. In some embodiments, the regulatory elements are selected to provide regulated/inducible/repressible expression. In some embodiments, the regulatory elements are selected to provide tissue-specific expression. In some embodiments, the regulatory elements and sequence encoding the dsRNA form a transcription unit.

A dsRNA of the present disclosure may be expressed from transcription units inserted into DNA or RNA vectors (see, e.g., Couture et al., TIG (1996) 12:5-10; PCT Patent Publications WO 00/22113 and WO 00/22114; and U.S. Pat. No. 6,054,299). Expression may be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann et al., PNAS (1995) 92:1292).

In some embodiments, the sense and antisense strands of a dsRNA are encoded on separate expression vectors. In some embodiments, the sense and antisense strands are expressed on two separate expression vectors that are co-introduced (e.g., by transfection or infection) into the same target cell. In some embodiments, the sense and antisense strands are encoded on the same expression vector. In some embodiments, the sense and antisense strands are transcribed from separate promoters which are located on the same expression vector. In some embodiments, the sense and antisense strands are transcribed from the same promoter on the same expression vector. In some embodiments, the sense and antisense strands are transcribed from the same promoter as an inverted repeat joined by a linker polynucleotide sequence such that the dsRNA has a stem and loop structure.

### IV. dsRNA Therapy

Certain aspects of the present disclosure relate to methods for inhibiting the expression of the LPA gene in a subject (e.g., a primate subject such as a human) comprising administering a therapeutically effective amount of one or more dsRNAs of the present disclosure, one or more vectors of the present disclosure, or one or more pharmaceutical compositions of the present disclosure. Certain aspects of the present disclosure relate to methods of treating and/or preventing one or more conditions described herein (e.g., an Lp(a)-associated condition such as a cardiovascular disease (CVD) including atherosclerosis, peripheral artery disease, aortic valve calcification, thrombosis, or stroke), comprising administering one or more dsRNAs of the present disclosure and/or one or more vectors of the present disclosure and/or one or more pharmaceutical compositions comprising one or more dsRNAs as described herein. In some embodiments, downregulating LPA expression in a subject alleviates one or more symptoms of a condition described herein (e.g., a high Lp(a)-associated condition such as a CVD) in the subject.

The pharmaceutical composition of the present disclosure may be administered in dosages sufficient to inhibit expression of the LPA gene. In some embodiments, a suitable dose of a dsRNA described herein is in the range of 0.001 mg/kg-200 mg/kg body weight of the recipient. In certain embodiments, a suitable dose is in the range of 0.001 mg/kg-50 mg/kg body weight of the recipient, e.g., in the range of 0.001 mg/kg-20 mg/kg body weight of the recipient. Treatment of a subject with a therapeutically effective amount of a pharmaceutical composition can include a single treatment or a series of treatments.

As used herein, the terms “therapeutically effective amount” and “prophylactically effective amount” refer to an amount that provides a therapeutic benefit in the treatment, prevention, or management of pathological processes mediated by LPA expression, or an overt symptom of pathological processes mediated by LPA expression.

As used herein, the term “Lp(a)-associated condition” or “high Lp(a)-associated condition” is intended to include any condition in which decreasing the plasma concentration of Lp(a) is beneficial. Such a condition may be caused, for example, by excessive production of Lp(a), production of certain apo(a) isoforms linked to diseased conditions, LPA gene mutations that increase Lp(a) levels, abnormal apo(a) cleavage that leads to increased levels, or decreased degradation and clearance, and/or abnormal interactions between Lp(a) and other proteins or other endogenous or exogenous substances (e.g., plasminogen receptor) such that Lp(a) level is increased or degradation is decreased. A Lp(a)-associated condition may be, e.g., a cardiovascular disease. A condition associated with high Lp(a) levels may be relatively insensitive to life style changes and common statin drugs, and are therefore hard to treat. An Lp(a) associated condition as defined herein may be selected from lipidemia (e.g., hyperlipidemia), dyslipidemia (e.g., atherogenic dyslipidemia, diabetic dyslipidemia, or mixed dyslipidemia), hyperlipoproteinemia, hyperapobetalipoproteinemia, coronary artery disease, myocardial infarction, peripheral artery disease, metabolic syndrome, acute coronary syndrome, aortic valve stenosis, aortic valve calcification, aortic valve regurgitation, aortic dissection, retinal artery occlusion, cerebrovascular disease, mesenteric ischemia, superior mesenteric artery occlusion, restenosis, renal artery stenosis, angina, cerebrovascular atherosclerosis, cerebrovascular disease, and venous thrombosis.

In some embodiments, a dsRNA described herein is used to treat a subject with a cardiovascular disease (CVD) such as chronic heart disease (CHD) or any symptoms or conditions associated with a CVD. In certain embodiments, a dsRNA described herein is used to treat a patient with hypercholesterolemia (e.g., statin-resistant hypercholesterolemia, and heterozygous or homozygous familial hypercholesterolemia) myocardial infarction (MI), peripheral arterial disease (PAD), calcific aortic valve disease (CAVD), atherosclerotic cardiovascular disease (ASCVD), atherosclerosis, dyslipidemia, thrombosis, or stroke.

In some embodiments, a dsRNA described herein is used to treat a subject having one or more conditions selected from: lipidemia (e.g., hyperlipidemia), dyslipidemia (e.g., atherogenic dyslipidemia, diabetic dyslipidemia, or mixed dyslipidemia), hyperlipoproteinemia, hyperapobetalipoproteinemia, coronary artery disease, metabolic syndrome, acute coronary syndrome, aortic valve stenosis, aortic valve calcification, aortic valve regurgitation, aortic dissection, retinal artery occlusion, cerebrovascular disease, mesenteric ischemia, superior mesenteric artery occlusion, restenosis, renal artery stenosis, angina, cerebrovascular atherosclerosis, cerebrovascular disease, and venous thrombosis.

In some embodiments, a dsRNA described herein may be used to manage body weight or reduce fat mass in a subject.

In some embodiments, a dsRNA as described herein inhibits expression of the human LPA gene, or both human and cynomolgus LPA genes. The expression of the LPA gene in a subject may be inhibited, or Lp(a) levels in the subject may be reduced, by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or about 100% after treatment as compared to pretreatment levels. In some embodiments, expression of the LPA gene is inhibited, or Lp(a) levels in the subject may be reduced, by at least about 2, at least about 5, at least about 10, at least about 15, at least about 20, at least about 25, at least about 50, at least about 75, or at least about 100-fold after treatment as compared to pretreatment levels. In some embodiments, the LPA gene is inhibited, or Lp(a) levels are reduced, in the liver of the subject.

In some embodiments, expression of the LPA gene is decreased by the dsRNA for about 12 or more, 24 or more, or 36 or more hours. In some embodiments, expression of the LPA gene is decreased for an extended duration, e.g., at least about two, three, four, five, or six days or more, e.g., about one week, two weeks, three weeks, or four weeks or longer.

As used herein, the terms “inhibit the expression of” or “inhibiting expression of,” insofar as they refer to the LPA gene, refer to at least partial suppression of expression of the LPA gene, as manifested by a reduction in the amount of mRNA transcribed from the LPA gene in a first cell or group of cells treated such that expression of the LPA gene is inhibited, as compared to a second cell or group of cells substantially identical to the first cell or group of cells but which has or have not been so treated (control cells). Such inhibition can be assessed, e.g., by Northern analysis, in situ hybridization, B-DNA analysis, expression profiling, transcription of reporter constructs, and other techniques known in the art. As used herein, the term “inhibiting” is used interchangeably with “reducing,” “silencing,” “downregulating,” “suppressing,” and other similar terms, and include any level of inhibition. The degree of inhibition is usually expressed in terms of (((mRNA in control cells)−(mRNA in treated cells))/(mRNA in control cells))×100%.

Alternatively, the degree of inhibition may be given in terms of a reduction of a parameter that is functionally linked to LPA gene transcription, e.g., the amount of protein encoded by the LPA gene in a cell (as assessed, e.g., by Western analysis, expression of a reporter protein, ELISA, immunoprecipitation, or other techniques known in the art), or the number of cells displaying a certain phenotype, e.g., apoptosis. In principle, LPA gene silencing may be determined in any cell expressing the target, either constitutively or by genomic engineering, and by any appropriate assay. However, when a reference is needed in order to determine whether a given dsRNA inhibits the expression of the LPA gene by a certain degree and therefore is encompassed by the present disclosure, the assays provided in the Examples below shall serve as such a reference.

A dsRNA or pharmaceutical composition described herein may be administered by any means known in the art, including, without limitation, oral or parenteral routes, including intravenous, intramuscular, subcutaneous, pulmonary, transdermal, and airway (aerosol) administration. Typically, when treating a patient with hypercholesterolemia or another CVD condition, the dsRNA molecules are administered systemically via parenteral means. In some embodiments, the dsRNAs and/or compositions are administered by subcutaneous administration. In some embodiments, the dsRNAs and/or compositions are administered by intravenous administration. In some embodiments, the dsRNAs and/or compositions are administered by pulmonary administration.

As used herein, in the context of LPA expression, the terms “treat,” “treatment” and the like refer to relief from or alleviation of pathological processes mediated by target gene expression. In the context of the present disclosure, insofar as it relates to any of the conditions recited herein, the terms “treat,” “treatment,” and the like refer to relieving or alleviating one or more symptoms associated with said condition. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

As used herein, the terms “prevent” or “delay progression of” (and grammatical variants thereof), with respect to a condition relate to prophylactic treatment of a condition, e.g., in an individual suspected to have or be at risk for developing the condition. Prevention may include, but is not limited to, preventing or delaying onset or progression of the condition and/or maintaining one or more symptoms of the disease at a desired or sub-pathological level.

It is understood that the dsRNAs of the present disclosure may be for use in a treatment as described herein, may be used in a method of treatment as described herein, and/or may be for use in the manufacture of a medicament for a treatment as described herein.

In some embodiments, a dsRNA of the present disclosure is administered in combination with one or more additional therapeutic agents, such as other siRNA therapeutic agents, monoclonal antibodies, and small molecules, to provide a greater improvement to the condition of the patient than administration of the dsRNA alone. In certain embodiments, the additional therapeutic agent provides an anti-inflammatory effect. In certain embodiments, the additional therapeutic agent is an agent that treats hypertriglyceridemia, such as a lipid-lowering agent.

In some embodiments, the additional agent may be one or more of a PCSK9 inhibitor, an HMG-CoA reductase inhibitor (e.g., a statin), an ANGPTL3 or ANGPTL8 inhibitor, a fibrate, a bile acid sequestrant, niacin (nicotinic acid), an antiplatelet agent, an angiotensin converting enzyme inhibitor, an angiotensin II receptor antagonist (e.g., losartan potassium), an acyl-CoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, an omega-3 fatty acid (e.g., fish oil or flaxseed oil), and insulin or an insulin analog. Particular examples include, without limitation, atorvastatin, pravastatin, simvastatin, lovastatin, fluvastatin, cerivastatin, rosuvastatin, pitavastatin, ezetimibe, bezafibrate, clofibrate, fenofibrate, gemfibrozil, ciprofibrate, cholestyramine, colestipol, colesevelam, and niacin.

In certain embodiments, a dsRNA as described herein may be administered in combination with another therapeutic intervention such as lipid lowering, weight loss, dietary modification, and/or moderate exercise.

Genetic predisposition plays a role in the development of target gene associated diseases, e.g., high Lp(a) levels. Therefore, a subject in need of treatment with one or more dsRNAs of the present disclosure may be identified by taking a family history, or, for example, screening for one or more genetic markers or variants, in particular Lp(a) KIV2 polymorphism. In certain embodiments, a subject in need of treatment with one or more dsRNAs of the present disclosure may be identified by screening for variants in any of these genes or any combination thereof.

A healthcare provider, such as a doctor, nurse, or family member, can take a family history before prescribing or administering a dsRNA of the present disclosure. In addition, a test may be performed to determine a genotype or phenotype. For example, a DNA test or an apo(a) isoform separation test may be performed on a sample from the subject, e.g., a blood sample, to identify the LPA genotype and the circulating Lp(a) phenotype before the dsRNA is administered to the subject.

### V. Kits and Articles of Manufacture

Certain aspects of the present disclosure relate to an article of manufacture or a kit comprising one or more of the dsRNAs, vectors, or compositions (e.g., pharmaceutical compositions) as described herein useful for the treatment and/or prevention of a high Lp(a)-associated condition (e.g., a peripheral artery disease, atherosclerosis, or aortic valve calcification). The article of manufacture or kit may further comprise a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating or preventing the disease and may have a sterile access port (for example the container may be an intravenous solution bag or a vial having a stopper pierceable by a hypodermic injection needle). At least one active agent in the composition is a dsRNA as described herein. The label or package insert indicates that the composition is used for treating a high Lp(a)-associated condition. In some embodiments, the condition is a CVD and/or another condition described herein. Moreover, the article of manufacture or kit may comprise (a) a first container with a composition contained therein, wherein the composition comprises a dsRNA as described herein; and (b) a second container with a composition contained therein, wherein the composition comprises a second therapeutic agent (e.g., an additional agent as described herein). The article of manufacture or kit in this aspect of the present disclosure may further comprise a package insert indicating that the compositions can be used to treat a particular disease. Alternatively, or additionally, the article of manufacture or kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and/or user standpoint, including other buffers, diluents, filters, needles, and syringes.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control.

Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to the manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

All publications and other references mentioned herein are incorporated by reference in their entirety. Although a number of documents are cited herein, this citation does not constitute an admission that any of these documents form part of the common general knowledge in the art.

### EXAMPLES

In order for the present disclosure to be better understood, the following examples are set forth. These examples are for illustration only and are not to be construed as limiting the scope of the present disclosure in any manner.

### Example 1: siRNA Synthesis and Purification

siRNAs, including non-targeting control siRNAs (NT control), were produced using solid phase oligonucleotide synthesis.

An LPA siRNA screening library comprising 299 19-mer LPA siRNA sequences with G+C content was designed to fully match the human mRNA transcript (NM_005577.2) with maximum one mismatch allowed to the orthologous cynomolgus mRNA sequence (XM_015448517). These LPA siRNA sequences comprise a fixed pattern of 2′-O-methyl and 2′-fluoro modified nucleotides (Table 1). All sense and antisense strand sequences were in silico profiled against the human RefSeq RNA database version 2016-02-23. Off-target transcripts with RNA-Seq expression (Illumina Body Atlas) FPKM<0.5 in human liver tissue were not considered. The only exception represents the LPAL2 pseudogene where off-target hits were accepted. siRNA sequences with >2 mismatches to any other potential human off-target transcript expressed in human liver were used for the library design.

Unconjugated LPA siRNAs, including non-targeting control siRNAs (“LV2” and “LV3”), were synthesized at a scale of 1 μmol (in vitro) or 10 μmol (in vivo) on a ABI 394 DNA/RNA or BioAutomation MerMade 12 synthesizer using commercially available 5′-O-DMT-3′-O-(2-cyanoethyl-N,N-diisopropyl) phosphoramidite monomers (SAFC) of uridine, 4-N-acetylcytidine (C^{Ac}), 6-N-benzoyladenosine (A B z) and 2-N-isobutyrylguanosine (G′ B ‘) with 2’- or 2′-F modification, and the solid supports 5′-O-DMT-thymidine-CPG and 3′-O-DMT-thymidine-CPG (invdT, Link) following standard protocols for solid phase synthesis and deprotection (Beaucage, Curr Opi Drug Discov Devel. (2008) 11:203-16; Mueller et al., Curr Org Synth. (2004) 1:293-307).

Phosphoramidite building blocks were used as 0.1 M solutions in acetonitrile and activated with 5-(bis-3,5-trifluoromethylphenyl)-1H-tetrazole (activator 42, 0.25 M in acetonitrile, Sigma Aldrich). Reaction times of 300 s were used for the phosphoramidite couplings. As capping reagents, acetic anhydride in THF (CapA for ABI, Sigma Aldrich) and N-methylimidazole in THF (CapB for ABI, Sigma Aldrich) were used. As oxidizing reagent, iodine in THF/pyridine/water (0.02 M; oxidizer for ABI, Sigma Aldrich) was used. Deprotection of the DMT-protecting group was done using dichloroacetic acid in DCM (DCA deblock, Sigma Aldrich). Final cleavage from solid support and deprotection (acyl- and cyanoethyl-protecting groups) was achieved with NH₃ (32% aqueous solution/ethanol, v/v 3:1).

The crude oligonucleotides were analyzed by IEX and LC-MS, and purified by anion-exchange high-performance liquid chromatography (IEX-HPLC) using a linear gradient of 10-65% buffer B in 30 min. ÄKTA purifier (Thermo Fisher Scientific DNAPac PA200 semi prep ion exchange column, 8 μm particles, width 22 mm×length 250 mm).
Buffer A: 1.50 l H₂O, 2.107 g NaClO₄, 438 mg EDTA, 1.818 g TRIS, 540.54 g urea, pH 7.4.
Buffer B: 1.50 l H₂O, 105.34 g NaClO₄, 438 mg EDTA, 1.818 g TRIS, 540.54 g urea, pH 7.4.

Isolation of the oligonucleotides was achieved by precipitation, induced by the addition of 4 volumes of ethanol and storing at −20° C.

To ensure high fidelity of the data, all single strands were HPLC purified to >85% purity. The purity and identity of the oligonucleotides was confirmed by ion exchange chromatography and LC-MS, respectively.

Positive control LPA siRNAs s8263 and s8264 were purchased from Ambion (now Thermo Fisher Scientific).

For the in vitro and in vivo experiments, stock solutions (100 μM and 10 mg/ml, respectively) of siRNAs in PBS were prepared by mixing equimolar amounts of complementary sense and antisense strands in 1×PBS buffer. The solutions were heated to 90° C. for 10 min and allowed to slowly cool to room temperature to complete the annealing process. siRNAs were further characterized by HPLC and were stored frozen until use.

siRNA Sequences

The sequences of each siRNA, and sequences including nucleotide modifications, are shown in Tables 1, 2, 3, and 4, supra.

### Example 2: Identification of siRNAs for Inhibition of Human LPA Expression

Methods

Cells and Tissue Culture

Human Hep3B cells were grown at 37° C., 5% CO₂ and 95% RH, and cultivated in EMEM medium (ATCC®, cat. no. 30-2003™) supplemented with 10% FBS.

Human HuH-7 cells were grown at 37° C., 5% CO₂ and 95% RH, and cultivated in MEM medium (ThermoFisher, cat. no. 41090) supplemented with 1×NEAA (ThermoFisher, cat. no. 11140035), 1% sodium pyruvate (Sigma, cat. no. S8636) and 10% FBS.

HepG2 cells stably overexpressing a pmirGLO-LPA dual luciferase reporter plasmid (see below) were grown at 37° C., 5% CO₂ and 95% RH, and cultivated in MEM medium (ThermoFisher, cat. no. 41090) supplemented with 1×NEAA (ThermoFisher, cat. no. 11140035), 1% sodium pyruvate (Sigma, cat. no. S8636), 10% FBS and 600 μg/ml G418 sulfate (Geneticin™ Selective Antibiotic; ThermoFisher, cat. no. 10131035).

HepG2 cells stably overexpressing a human LPA cDNA construct (Brunner et al., Proc Natl Acad Sci. (1993) 90(24):11643-7) were grown at 37° C., 5% CO₂ and 95% RH, and cultivated in DMEM/F12 medium (Lonza, cat. no. BE12-719F) supplemented with 10% FBS.

Primary human (BioreclamationlVT, cat. no. M00995-P) and cynomolgus (Primacyt, cat. no. CHCP-I-T) hepatocytes were cultured as follows: cryopreserved cells were thawed and plated using a plating and thawing kit (Primacyt, cat. no. PTK-1), and were incubated at 37° C., 5% CO₂ and 95% RH. 6 hours after plating, the medium was changed to maintenance medium (KaLy-Cell, cat. no. KLC-MM) supplemented with 1% FBS.

Primary hepatocytes from female apo(a) transgenic mice (see below) were isolated freshly before the experiments based on a protocol adapted from Seglen, P. O. (1976): Preparation of Isolated Rat Liver Cells; Methods in Cell Biology, 13: 29-83. Plating of isolated hepatocytes was done for 3-5 hours at 37° C., 5% CO₂ and 95% RH in Williams' E medium (Thermo Fisher, cat. no. 22551) supplemented with 2 mM glutamine (Thermo Fisher, cat. no. 25030), 100 U/ml Penicillin-Streptomycin (Thermo Fisher, cat. no. 15140), 1 μg/ml Dexamethason (Sigma, cat. no. D1756), 1×ITS solution (Thermo Fisher, cat. no. 41400), and 5% FBS. After plating, the medium was changed to cultivation medium that was identical to plating medium except for the addition of 1% FBS. No further medium change was done during the incubation period of 48 or 72 hours.

pmirGLO Dual Luciferase Reporter Assay

For siRNA screening purposes, the full-length human LPA cDNA sequence (NM_005577.2) was sub-cloned into the multiple cloning site of a commercially available, dual luciferase reporter-based pmirGLO screening plasmid (Promega, cat. no. E1330) which generated a Firefly luciferase/LPA fusion mRNA. For transient plasmid transfections, 45 μg of the pmirGLO-LPA plasmid was transfected in a fast-forward setup for 18 hours into 18 mio. Hep3B cells in T225 flasks (Falcon®, cat. no. 353138) using FuGene® HD transfection reagent (Promega, cat. no. E2311). 1 nM and 10 nM siRNA transfections of 5000 plasmid pre-transfected Hep3B cells per well in 384 well plates (Greiner-Bio CELLSTAR®, cat. no. 781098) using Lipofectamine™ RNAiMAX (ThermoFisher, cat. no. 13778150) was done next day in a reverse setup and cells were incubated for 48 hours. Gene knockdown was determined by measuring Firefly luciferase levels normalized to the levels of constitutively-expressed Renilla luciferase, also encoded by the pmirGLO plasmid, using the Dual-Glo® Luciferase Assay (Promega, cat. no. E2940).

IC₅₀ Measurements

For IC₅₀ experiments with the pmirGLO-LPA reporter plasmid in a stable HepG2 cell clone, 2 μg of Cla-I linearized pmirGLO-LPA plasmid was transfected per well in Collagen-I coated 6-well plates (BD, cat. no. 356400) using 80-90% confluent HepG2 cells and FuGene HD transfection reagent in a 3.5:1 ratio (μl FuGene HD vs. μg plasmid). Polyclonal cells were expanded in Collagen-I coated T75 flasks (Corning, cat. no. 356485) by adding 600 μg/ml G418 to the culture medium, and single cell cloned in Collagen-I coated 384-well plates (Corning, cat. no. 354664) using an IncuCyte® ZOOM Live-Cell Imaging System (Essen BioScience). Single cell clones were characterized by qPCR analysis for LPA expression levels (see below) as well as relative Firefly and Renilla luciferase abundance.

For IC₅₀ measurements with a transfection reagent, 30,000 primary transgenic apo(a) mouse hepatocytes in Collagen-I coated human Hep3B cells in 96-well plates were transfected with Lipofectamine™ RNAiMAX in a fast-forward setup for 72 hours with the indicated LPA siRNAs at 7 concentrations starting from 25 nM-0.1 pM using 8-fold dilution steps. The half maximal inhibitory concentration (IC₅₀) for each siRNA was determined by nonlinear regression using iterative fitting procedures developed on SAS9.4 software. Results were obtained using the 4-parameter logistic model according to Ratkovsky and Reedy (Biometrics (1986) 42(3):575-82). The adjustment was obtained by non-linear regression using the Levenberg-Marquardt algorithm in SAS software.

IC₅₀ values using the stable HepG2-pmirGLO-LPA cell clone were generated as follows: 5000 cells per well in Collagen-I coated 384 well plates were reverse transfected with Lipofectamine™ RNAiMAX and LPA siRNA reagents for 48 hours at 9 concentrations ranging from 40 nM-0.6 pM using 4-fold dilution steps.

siRNA Transfections

For knockdown experiments in HepG2-LPA and HuH-7 cells, 17,000 and 25,000 cells/well were used in Collagen-I coated (Corning® Biocoat™, cat. no. 356407) and non-coated 96-well plates (Greiner CELLSTAR®, cat. no. 655180), respectively. For knockdown experiments in primary human, cynomolgus, and transgenic apo(a) mouse hepatocytes, 40,000-50,000 cells/well were used in Collagen-I coated 96-well plates. The cells were transfected with LPA siRNAs at 1 or 10 nM using 0.2 μL/well of Lipofectamine™ RNAiMAX transfection reagent (Thermo Fisher) according to the manufacturer's protocol in a reverse (HepG2-LPA) or fast-forward (primary hepatocytes) transfection setup, and incubated for 48-72 h without medium change. Usually, N=4 technical replicates were carried out per test sample.

mRNA Expression Analysis

48 or 72 hours after siRNA transfection or free siRNA uptake, the cellular RNA was harvested by usage of Promega's SV96 total RNA isolation system (cat. no. Z3500) according to the manufacturer's protocol, including a DNase step during the procedure.

For cDNA synthesis, the ThermoFisher TaqMan™ Reverse Transcriptase kit (cat. no. N8080234) was used. cDNA was synthesized from 30 ng RNA using 1.2 μL 10×RT buffer, 2.64 μL MgCl₂ (25 mM), 2.4 μL dNTPs (10 mM), 0.6 μL random hexamers (50 μM), 0.6 μL Oligo(dT)16 (SEQ ID NO: 1631) (50 μM), 0.24 μL RNase inhibitor (20 U/μL) and 0.3 μL Multiscribe™ (50 U/μL) in a total volume of 12 μL. Samples were incubated at 25° C. for 10 minutes and 42° C. for 60 minutes. The reaction was stopped by heating to 95° C. for 5 minutes.

Human and cynomolgus LPA mRNA levels were quantified by qPCR using the ThermoFisher TaqMan™ Universal PCR Master Mix (cat. no. 4305719) and the following TaqMan Gene Expression assays:

| | LPA | PLG |
|---|---|---|
| Human | Hs00916691_m1 | Hs00264877_m1 |
| | Hs00534377_m1 | |
| Cynomolgus | Rh02789265_m1 | Mf02789292_m1 |

PCR was performed in technical duplicates with an ABI Prism 7900 system under the following PCR conditions: 2 minutes at 50° C., 10 minutes at 95° C., 40 cycles with 95° C. for 15 seconds and 1 minute at 60° C. PCR was set up as a simplex PCR detecting the target gene in one reaction and the housekeeping gene (human/cynomolgus RPL37A) for normalization in a parallel reaction. The final volume for the PCR reaction was 12.5 μL in a 1×PCR master mix; RPL37A primers were used at a final concentration of 50 nM and the probe was used at a final concentration of 200 nM. The ΔΔCt method was applied to calculate relative expression levels of the target transcripts. Percentage of target gene expression was calculated by normalization based on the levels of the LV2 or LV3 non-silencing siRNA control sequence.

Cytotoxicity Measurement

Cytotoxicity was measured 72 hours after 5 nM and 50 nM siRNA transfections of a culture of 20,000 HepG2-LPA cells per 96-well by determining the ratio of cellular viability/toxicity in each sample. Cell viability was measured by determination of the intracellular ATP content using the CellTiter-Glo assay (Promega, cat. no. G7570) according to the manufacturer's protocol. Cell toxicity was measured in the supernatant using the ToxiLight assay (Lonza, cat. no. LT07-217) according to the manufacturer's protocol. AllStars Hs Cell Death siRNA (Qiagen, cat. no. SI04381048), 25 μM Ketoconazole (Calbiochem, cat. no. 420600) and 1% Triton X-100 (Sigma, cat. no. T9284) were used as positive controls.

Results

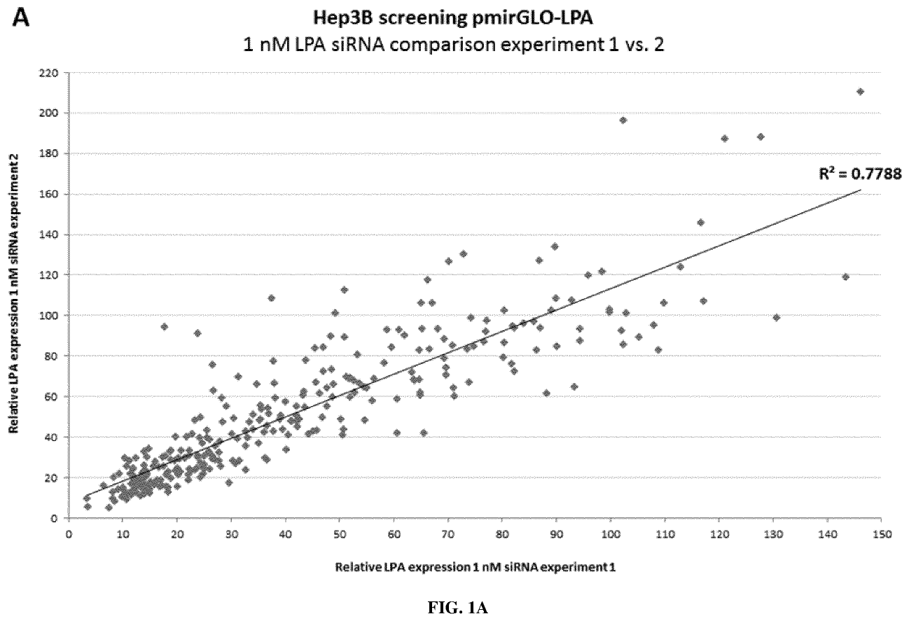

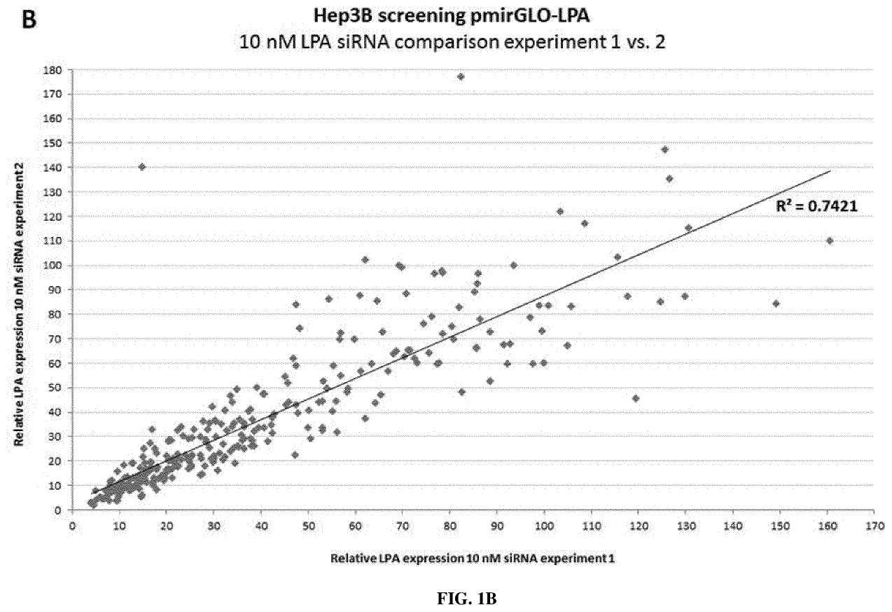

As shown in FIGS. 1A and 1B, transient transfection of Hep3B cells with the pmirGLO-LPA plasmid followed by transfection of the LPA siRNA library with said Hep3B cells at 1 or 10 nM correlated very well with correlation coefficients of R₂=0.78 (1 nM LPA siRNA) and R₂=0.74 (10 nM LPA siRNA). FIGS. 1A and 1B also demonstrate the identification of highly potent LPA siRNA reagents. Only a small fraction of LPA siRNA sequences exhibited knockdown activities>75% (1 nM siRNA concentration) and >85% (10 nM siRNA concentration). 34 active LPA siRNA reagents with only a single 100% matching site within the human LPA mRNA sequence were selected for further characterization using in vitro assays.

IC₅₀ and Iₘₐₓ values of the 34 selected LPA siRNAs from two independent experiments are depicted in Table 5.

TABLE 5
Activity of selected siRNAs in HepG2
cells transfected with pmirGLO-LPA

| | |
|---|---|
| Experiment 1 | Experiment 2 |

| Compound | Iₘₐₓ % | IC₅₀ [nM] | Iₘₐₓ % | IC₅₀ [nM] |
|---|---|---|---|---|
| siLPA#0004 | 92.6 | 0.546 | 93.1 | 0.481 |
| siLPA#0007 | 87.4 | 0.376 | 93.2 | 0.299 |
| siLPA#0019 | 80.8 | 0.943 | 90.4 | 1.09 |
| siLPA#0059 | 93.6 | 0.554 | 92.6 | 0.274 |
| siLPA#0102 | 81.3 | 0.388 | 86.3 | 0.578 |
| siLPA#0103 | 83.1 | 8.96 | 90.4 | 22.5 |
| siLPA#0104 | 96.3 | 0.239 | 94.5 | 0.285 |
| siLPA#0105 | 85.0 | 1.66 | 94.1 | 4.78 |
| siLPA#0107 | 94.4 | 0.384 | 95.3 | 0.733 |
| siLPA#0108 | 85.2 | 0.269 | 90.3 | 0.774 |
| siLPA#0109 | 88.7 | 1.28 | 87.4 | 5.76 |
| siLPA#0110 | 90.8 | 0.475 | 93.0 | 5.92 |
| siLPA#0111 | 91.9 | 0.272 | 91.8 | 2.13 |
| siLPA#0138 | 87.9 | 0.752 | 86.9 | 1.08 |
| siLPA#0141 | 77.5 | 0.595 | 88.9 | 1.75 |
| siLPA#0168 | 78.3 | 0.886 | 90.4 | 1.30 |
| siLPA#0169 | 84.1 | 1.63 | 92.9 | 0.780 |
| siLPA#0172 | 92.0 | 0.115 | 96.3 | 0.684 |
| siLPA#0174 | 84.5 | 1.11 | 86.1 | 1.2 |
| siLPA#0200 | 90.8 | 0.865 | 89.5 | 0.228 |
| siLPA#0204 | 83.6 | 1.19 | 87.2 | 0.740 |
| siLPA#0208 | 93.4 | 1.24 | 84.7 | 1.38 |
| siLPA#0214 | 88.2 | 4.33 | 91.0 | 4.71 |
| siLPA#0217 | 76.0 | 5.06 | 83.0 | 3.13 |
| siLPA#0220 | 78.6 | 1.31 | 88.2 | 0.969 |
| siLPA#0221 | 92.5 | 0.955 | 89.9 | 0.848 |
| siLPA#0223 | 95.2 | 0.534 | 96.5 | 0.502 |
| siLPA#0224 | 81.3 | 1.7 | 87.7 | 0.861 |
| siLPA#0228 | 88.7 | 0.268 | 88.7 | 0.687 |
| siLPA#0277 | 79.9 | 0.823 | 88.8 | 1.39 |
| siLPA#0279 | 92.1 | 0.521 | 91.3 | 0.471 |
| siLPA#0282 | 78.6 | 1.61 | 86.5 | 0.394 |
| siLPA#0296 | 74.3 | 3.46 | 85.3 | 1.96 |
| siLPA#0298 | 90.4 | 0.656 | 91.1 | 0.913 |

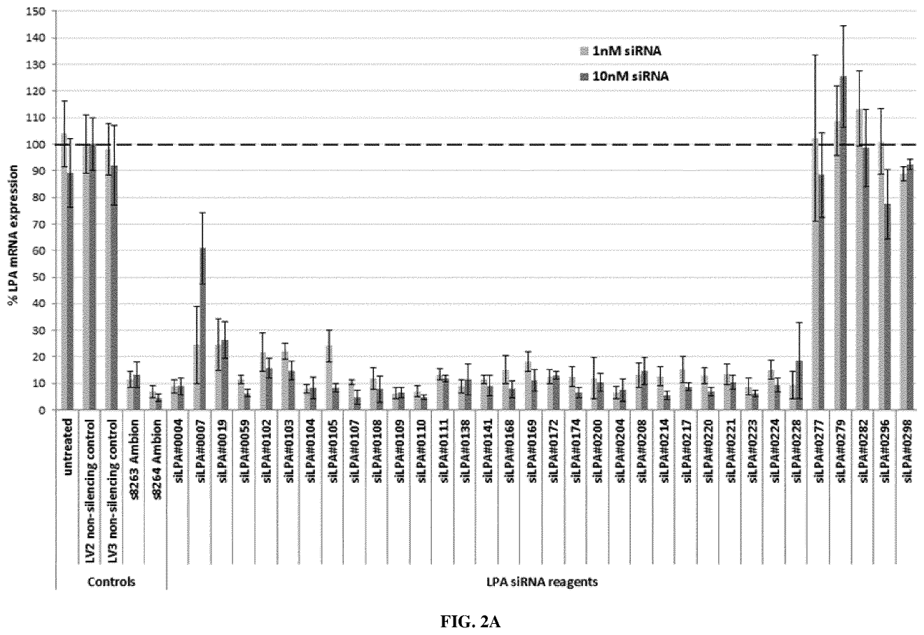

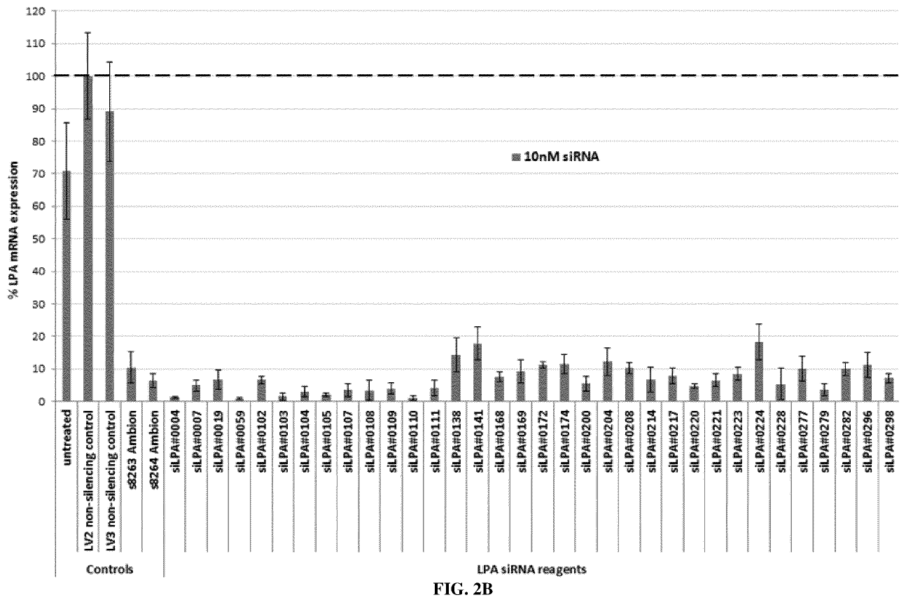

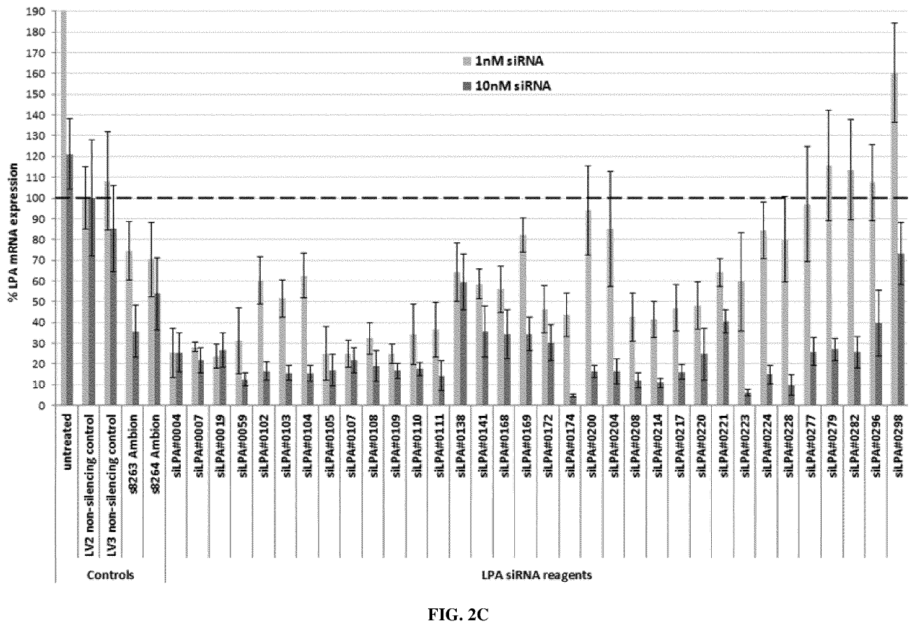

The 34 selected siRNAs were further evaluated for LPA mRNA knockdown activity in HepG2-LPA cells stably overexpressing a human LPA cDNA construct (FIG. 2A). This cell line was identified as being not suitable for the characterization of all LPA siRNAs regarding mRNA knockdown activity because the cDNA clone misses the last 196 nucleotides of the 3′ untranslated region (UTR) of the human LPA mRNA (NM_005577.2) (Brunner et al., Proc Natl Acad Sci. (1993) 90(24):11643-7). Therefore, the 34 LPA siRNA reagents were further investigated for LPA mRNA knockdown activity in primary transgenic apo(a) mouse hepatocytes (FIG. 2B) and in primary cynomolgus hepatocytes (FIG. 2C).

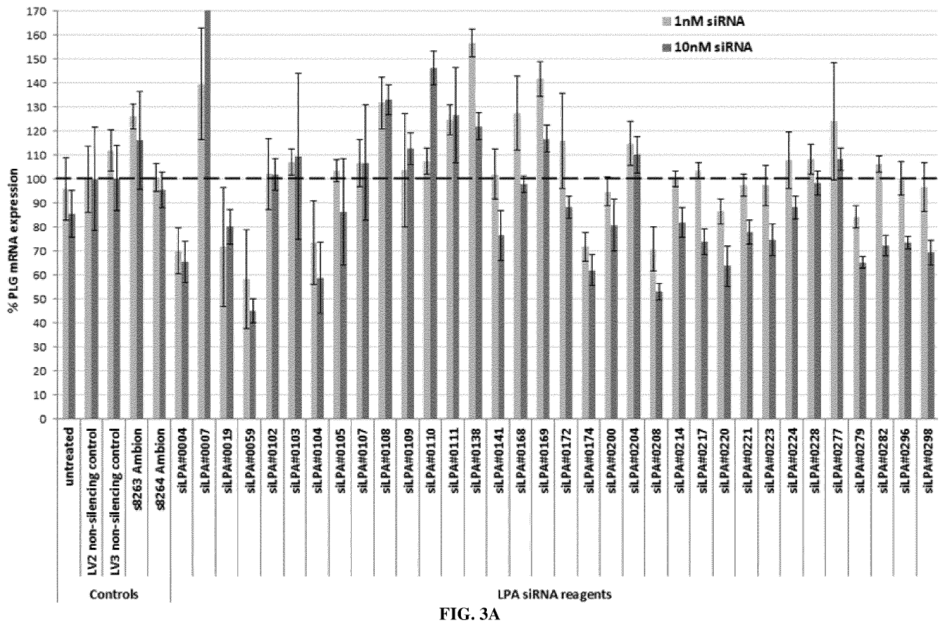

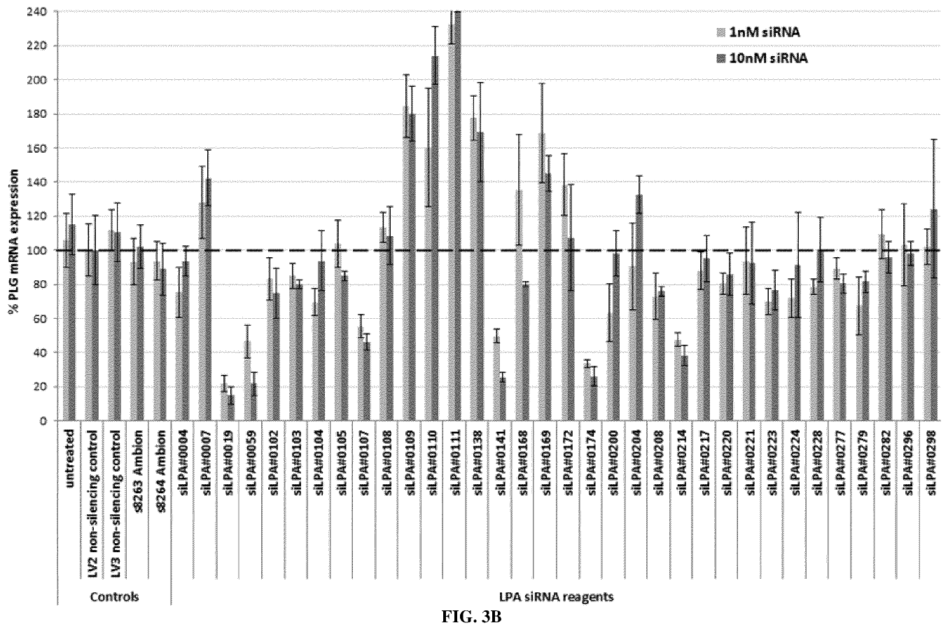

The specificity of the 34 selected LPA siRNAs was evaluated by assessing their ability to repress the mRNA expression levels of human plasminogen, the closest protein-coding orthologue of apo(a). PLG mRNA levels were determined in the human HuH-7 cell line (FIG. 3A) as well as in primary human (FIG. 3B) and cynomolgus (FIG. 3C) hepatocytes transfected with LPA siRNAs.

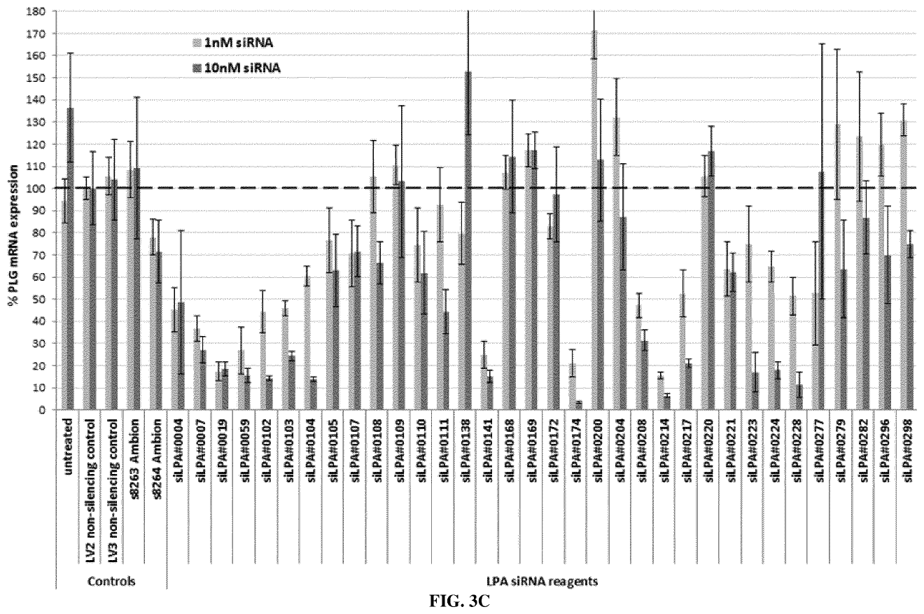

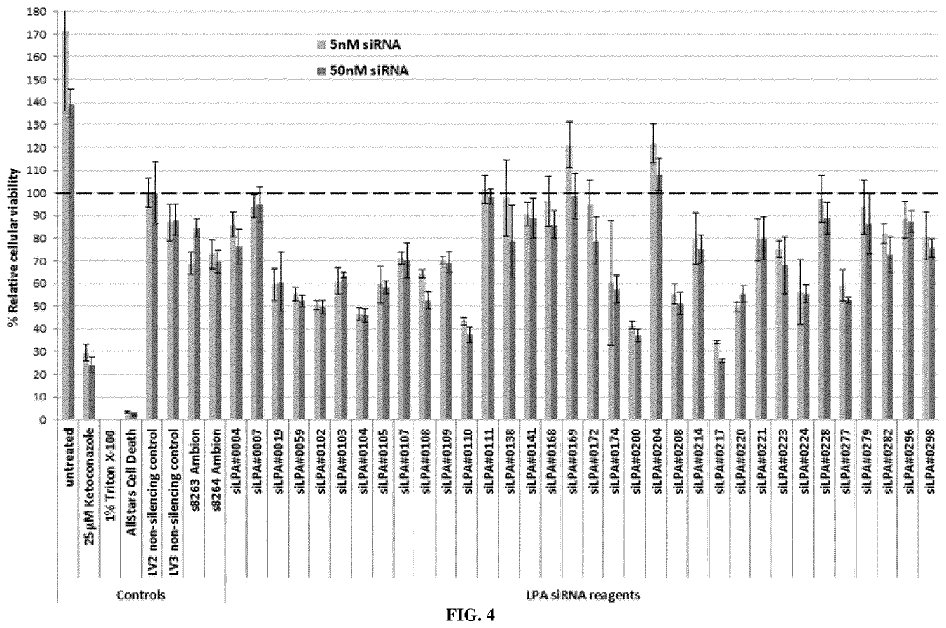

Next, the 34 selected LPA siRNAs were transfected into HepG2-LPA overexpressing cells and assayed for off-target effects by measuring cellular viability (intracellular ATP content) and toxicity (extracellular adenylate kinase levels) from the same cell culture well (FIG. 4).

Subsequently, less potent siRNAs with IC₅₀>1 nM or Iₘₐₓ<90% in both pmirGLO-LPA experiments in the stable HepG2 cell clone (see Table 5) were filtered out. In total, 17 LPA siRNAs were selected for additional IC₅₀ experiments in primary transgenic apo(a) mouse hepatocytes. IC₅₀ and Iₘₐₓ values are listed in Table 6.

Taken together, these results highlight the identification of siRNAs capable of potent and specific inhibition of human and cynomolgus LPA mRNA expression in human cells.

TABLE 6
Activity of selected siRNAs in apo(a) mouse hepatocytes

| Compound | Iₘₐₓ % | IC₅₀ [nM] |
|---|---|---|
| siLPA#0004 | 91.1 | 0.0049 |
| siLPA#0007 | 88.4 | 0.0058 |
| siLPA#0019 | 84.8 | 0.013 |
| siLPA#0090 | 90.8 | 0.0113 |
| siLPA#0104 | 92.1 | 0.0197 |
| siLPA#0107 | 92.9 | 0.003 |
| siLPA#0108 | 93.2 | 0.0076 |
| siLPA#0110 | 95.6 | 0.009 |
| siLPA#0111 | 94.8 | 0.0115 |
| siLPA#0168 | 92.9 | 0.021 |
| siLPA#0169 | 96.2 | 0.0204 |
| siLPA#0172 | 92.9 | 0.0025 |
| siLPA#0200 | 94.5 | 0.003 |
| siLPA#0221 | 91.8 | 0.0139 |
| siLPA#0223 | 91.4 | 0.0041 |
| siLPA#0279 | 95.2 | 0.0393 |
| siLPA#0298 | 93.0 | 0.0343 |

### Example 3: Identification of Active GalNAc-Conjugated siRNAs for Inhibition of Human and Cynomolgus LPA Expression

Methods

GalNAc-siRNAs, including non-targeting control siRNAs (NT control), were generated based on the indicated sequences (see sequence listings above) as described in WO 2019/170731.

Cell and Tissue Culture

Human (BioreclamationlVT, cat. no. M00995-P) and cynomolgus (Primacyt, cat. no. CHCP-I-T) primary hepatocytes were cultured as described above in Example 2.

Human peripheral blood mononuclear cells (PBMCs) were isolated from approximately 16 ml of blood from three healthy donors that were collected in Vacutainer® CPT™ tubes coated with sodium heparin (BD, cat. no. 362780) according to manufacturer's instructions.

Human Apo(a) Transgenic Mouse Model

The female mice used in the following experiments carried a YAC genomic locus comprising the full-length human LPA gene [Nat Genet. 1995 9(4):424-31]. The transgenic model, strain FVB/N-Tg(LPA,LPAL2,PLG)1Hgc/Mmmh, was in-licensed from University of California, Berkeley, USA.

Assays

mRNA expression analysis was performed as described above in Example 2.

For IC₅₀ measurements in primary human, cynomolgus and transgenic apo(a) mouse hepatocytes under free uptake conditions, 70,000 (human and cynomolgus) or 30,000 (transgenic apo(a) mouse) cells in Collagen-I coated 96-well plates were incubated for 72 hours without medium change with the siRNA-GalNAc conjugates at concentrations ranging from 10 μM-0.01 nM (human and cynomolgus) or 1 μM-0.001 μM (transgenic apo(a) mouse) using 10-fold dilution steps.

Cytotoxicity and cell viability were measured as described above in Example 2.

siRNA Stability in Mouse Serum

Modified siRNAs were tested for nuclease stability in 50% mouse serum. 160 μl of 2.5 μM siRNA in 1×DPBS (Life Technologies, cat. no. 14190-094) and 160 μl mouse serum (Sigma, cat. no. M5905) were incubated at 37° C. for up to 168 h. At each time-point (0 h, 8 h, 24 h, 48 h, 72 h, 96 h and 168 h), 20 μl of the reaction was taken out and quenched with a stop solution (Tissue & Cell Lysis Solution (Epicentre, cat. no. MTC096H), Proteinase K (Sigma, cat. no. P2308), water) at 65° C. for 30 min. Prior to HPLC analysis on a Waters 2695 Separation Module and a 2487 Dual Absorbance Detector, RNase-free water was added to each sample. The solution was analyzed by HPLC using a DNAPac PA200 analytical column (Thermo Scientific, cat. no. 063000).

| Time (min) | Flow (mL/min) | % Buffer A* | % Buffer B** |
|---|---|---|---|
| 0 | 1 | 75 | 25 |
| 20 | 1 | 35 | 65 |

Buffer A: 20 mM sodium phosphate (Sigma, Cat. No. 342483), pH 11;
Buffer B: 20 mM sodium phosphate (Sigma, Cat. No. 342483), 1 M sodium bromide (Sigma, Cat. No. 02119), pH 11.

Serum half-lives were estimated for both strands of the siRNA.

apo(a) ELISA Assay

100 μl of 1:4 pre-diluted supernatants from primary transgenic apo(a) mouse hepatocytes treated with the indicated concentrations of LPA GalNAc-siRNA conjugates were used for apo(a) protein determination by CellBiolabs ELISA kit (cat. no. STA-359) according to the supplier's manual. OD450 measurements were done with a TECAN Infinite M1000 Pro instrument and TECAN's Magellan software module. Percentage of apo(a) protein expression was calculated by normalization based on the mean levels of the LV2 non-silencing siRNA control sequence.

For apo(a) determination from transgenic apo(a) mouse serum samples, blood was drawn as follows: for generation of maximum 30 μl serum, blood was taken from the vena saphena using Minivette® and microvettes from Sarstedt (cat. no. 17.2111.050 and 20.1280). For generation of maximum 100 μl serum, retroorbital blood was taken using a micropipette (Sigma, cat. no. BR709109) and a microvette (Sarstedt, cat. no. 20.1291). Prior to centrifugation at 4° C. for 10 minutes at 3500×g, the coagulation of the samples was done for 30 minutes at room temperature. Serum samples were diluted 1:5,000-1:20,000 for apo(a) ELISA measurement.

PLG ELISA Assay

100 μl of 1:4 pre-diluted supernatants from primary human hepatocytes treated with the indicated concentrations of LPA GalNAc-siRNA conjugates were used for plasminogen protein determination by Abnova ELISA kit (cat. no. KA3897) according to the supplier's manual. OD450 measurements were done with a TECAN Infinite M1000 Pro instrument and TECAN's Magellan software module. Percentage of PLG protein expression was calculated by normalization based on the mean levels of the LV2 non-silencing siRNA control sequence.

IFNα Determination

Protein concentration of human IFNα2a and 7 other cytokines was quantified in the supernatant of human PBMCs by using 25 μl of the cell culture supernatant and applying MesoScale Discovery's electrochemiluminescence U-PLEX assay technology (cat. no. K151VHK) according to the supplier's protocol.

RNA-Seq Off-Target Analysis

In order to test for potential off-target activities of LPA GalNAc-siRNA conjugates, RNA-Seq analysis was undertaken by using primary human hepatocytes. For this purpose, 400,000 primary human hepatocytes from two different donors with N=2 technical replicates each were seeded per well of Collagen-I coated 24-well plates (Corning, cat. no. 354408). Incubation with 5 μM of LPA GalNAc-siRNA conjugate without medium change was done for 72 hours. Cell lysis was undertaken with 350 μl RLT buffer (Qiagen, cat. no. 79216) per well and one freeze-thaw cycle at −80° C. Isolation of total RNA including small RNAs<200 nucleotides was done using a miRNeasy Mini kit (Qiagen, cat. no. 217004) including an optional on-column DNase digestion step (Qiagen, cat. no. 79254) according to the manufacturer's protocol. Integrity of the RNA samples was examined by applying Agilent's 2100 Bioanalyzer Total RNA Nano assay (cat. no. 5067-1511). RNA samples with RIN values>8 were included for RNA-Seq profiling. 400 ng of the RNA samples were then converted into RNA-Seq libraries using the TruSeq Stranded Total RNA LT Sample Prep Kit (with Ribo-Zero Gold) from Illumina (cat. no. RS-122-2301 and RS-122-2302). The resulting libraries were sequenced by paired-end sequencing (2×75 bp) on a NextSeq 500 instrument at ˜45 million reads per library using the NextSeq® 500/550 High Output v2 Kit (cat. no. FC-404-2002).

RNA-Seq data analysis pipeline is based on Array Studio (Qiagen). Briefly, raw data QC was performed, then a filtering step was applied to remove reads corresponding to rRNAs as well as reads having low quality score. Mapping and quantification were performed using OSA4 (Hu et al., Bioinformatics (2012) 28(14):1933-4) (Omicsoft Sequence Aligner, version 4). Reference Human Genome B38 was used for mapping and genes or transcripts were quantified based on Ensembl gene model. Differentially expressed transcripts were identified with DESeq2 (http://www.bioconductor.org/packages/3.2/bioc/html/DESeq2.html) and Voom (Law et al., Genome Biology (2014) 15:R29]. The variable multiplicity was taken into account and false discovery rate (FDR) adjusted p-values were calculated with the Benjamini-Hochberg (BH) correction (Benjamini & Hochberg, J Roy Statist Soc. (1995) B57:289-300).

Results

Following identification of potent LPA siRNAs as described in Example 2, the inventors went on to demonstrate whether the selected molecules retain their activity in the context of a GalNAc-conjugate suitable for liver-specific siRNA delivery in vivo. The inventors also assessed whether this activity holds up in additional hepatocytes from M. fascicularis (cynomolgus monkey), a pre-clinical species. For this purpose, the 17 selected LPA siRNAs were conjugated to three consecutive modified GalNAc conjugated nucleotides at the 5′ end of respective siRNA sense strands as shown in Table 3.

The results of the IC₅₀ measurements by free uptake experiments in primary human and transgenic apo(a) mouse hepatocytes (Table 7) demonstrate the identification of potent LPA GalNAc-siRNAs in both cell types in the absence of transfection conditions.

Interestingly, the same IC₅₀ experiment described above but using primary cynomolgus hepatocytes (Table 7) shows that the presence of a mismatch of an LPA GalNAc-siRNA to the cynomolgus LPA mRNA sequence has mixed impact on retained siRNA knockdown activity. The activity of human LPA GalNAc-siRNAs with a mismatch to cynomolgus species could therefore not be predicted per se, but is dependent on the sequence context and needs to be tested experimentally.

TABLE 7
Iₘₐₓ and IC₅₀ of selected LPA GalNAc-siRNAs in primary hepatocytes

| | human | | transgenic apo(a) mouse | | cynomolgus | |
|---|---|---|---|---|---|---|
| Compound | Iₘₐₓ % | IC₅₀ [nM] | Iₘₐₓ % | IC₅₀ [nM] | Iₘₐₓ % | IC₅₀ [nM] |
| siLPA #0300 | 76.3 | 6.0 | 88.4 | 0.27 | 66.7 | 3.1 |
| siLPA #0301 | 55.0 | 306.0 | 90.7 | 0.266 | 64.5 | 5.9 |
| siLPA #0302 | 55.9 | 41.2 | 94.6 | 0.242 | 54.7 | 1.2 |
| siLPA #0303 | 63.7 | 2.0 | 92.2 | 0.0117 | 84.1 | 3.8 |
| siLPA #0304 | 53.6 | 11.7 | 90.0 | 0.0369 | 81.4 | 2.4 |
| siLPA #0305 | 77.7 | 2.5 | 91.1 | 0.107 | 96.2 | 2.6 |
| siLPA #0306 | 77.0 | 4.4 | 90.7 | 0.0911 | 87.8 | 8.8 |
| siLPA #0307 | 79.2 | 4.7 | 90.9 | 0.173 | 84.5 | 15.1 |
| siLPA #0308 | 44.7 | 104.0 | 89.1 | 0.95 | 53.4 | 69.4 |
| siLPA #0309 | 61.3 | 52.4 | 91.1 | 0.6 | 90.3 | 914.0 |
| siLPA #0310 | 50.9 | 210.0 | 93.2 | 0.158 | 74.2 | 21.8 |
| siLPA #0311 | 52.3 | 0.3 | 90.6 | 0.96 | 86.0 | 13.3 |
| siLPA #0312 | 63.0 | 0.8 | 94.8 | 0.577 | 69.7 | 33.2 |
| siLPA #0313 | 73.8 | n.d. | 91.8 | 0.293 | 87.3 | 89.7 |
| siLPA #0314 | 50.7 | 4.4 | 89.8 | 0.101 | 83.7 | 5.3 |
| siLPA #0315 | 82.6 | 68.6 | 92.3 | 0.0877 | 21.3 | 232.0 |
| siLPA #0316 | 65.5 | n.d. | 88.9 | 0.308 | 1.5 | n.a. |

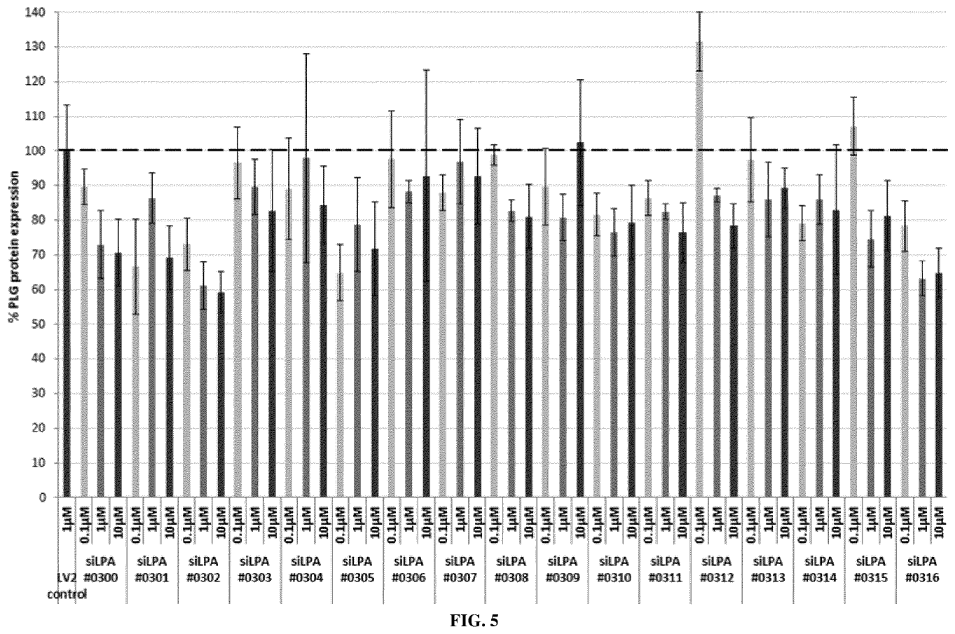

The specificity of the 17 selected LPA GalNAc-siRNAs was evaluated by IC₅₀-based testing of their ability to repress mRNA expression levels of human plasminogen in primary human hepatocytes under free uptake conditions. As shown in Table 8, some sequences with a clear effect on plasminogen mRNA reduction were identified. In order to confirm an effect on the protein level, cell culture supernatants of three siRNA concentrations from the same human hepatocyte experiment were used for a plasminogen ELISA readout (FIG. 5).

TABLE 8
Iₘₐₓ and IC₅₀ of selected GalNAc-siRNAs for PLG
mRNA expression in primary human hepatocytes

| Compound | Iₘₐₓ % | IC₅₀ [nM] |
|---|---|---|
| siLPA#0300 | −0.5 | n.a. |
| siLPA#0301 | 20.7 | >10000 |
| siLPA#0302 | 38.7 | 157.0 |
| siLPA#0303 | 23.3 | >10000 |
| siLPA#0304 | 4.8 | 110.0 |
| siLPA#0305 | 59.7 | 1060.0 |
| siLPA#0306 | −4.3 | n.a. |
| siLPA#0307 | −51.5 | n.a. |
| siLPA#0308 | −7.9 | n.a. |
| siLPA#0309 | 23.8 | >10000 |
| siLPA#0310 | 12.9 | n.a. |
| siLPA#0311 | 13.5 | n.a. |
| siLPA#0312 | −3.1 | n.a. |
| siLPA#0313 | 17.5 | n.a. |
| siLPA#0314 | 13.3 | n.a. |
| siLPA#0315 | 7.6 | n.a. |
| siLPA#0316 | 38.1 | >10000 |

n.a. = not active

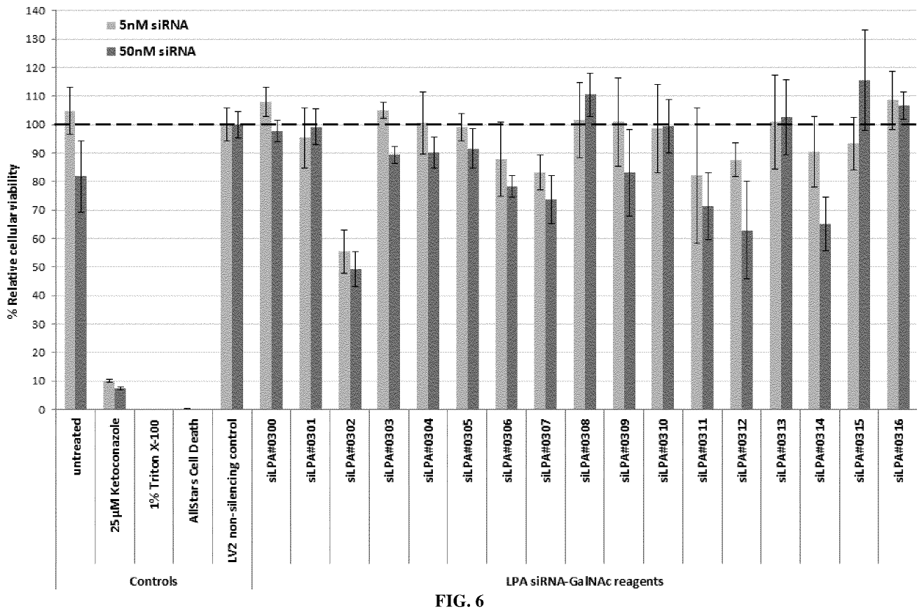

Next, a cytotoxicity assay was performed in HepG2-LPA overexpressing cells to exclude potentially toxic LPA GalNAc-siRNAs (FIG. 6).

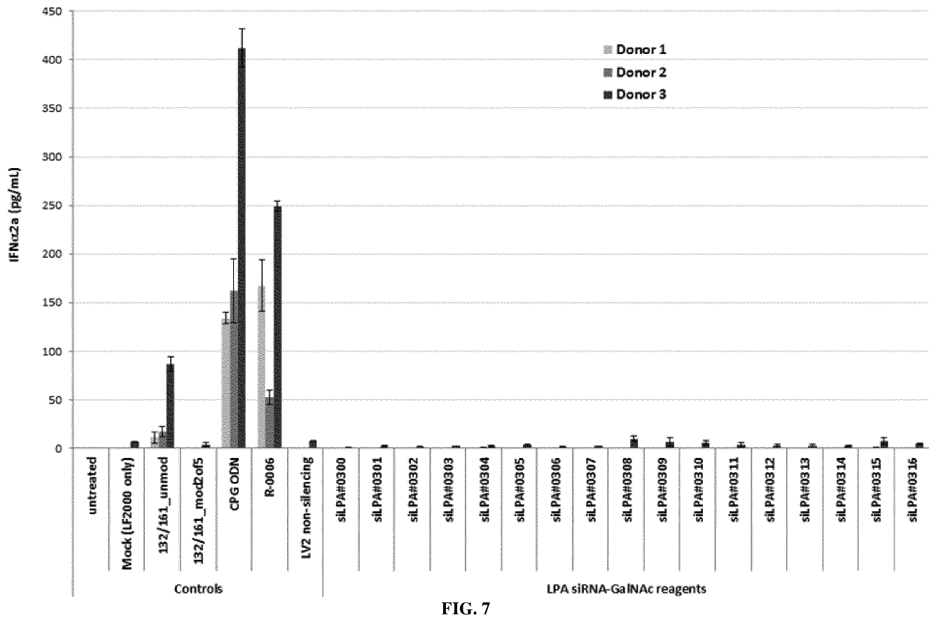

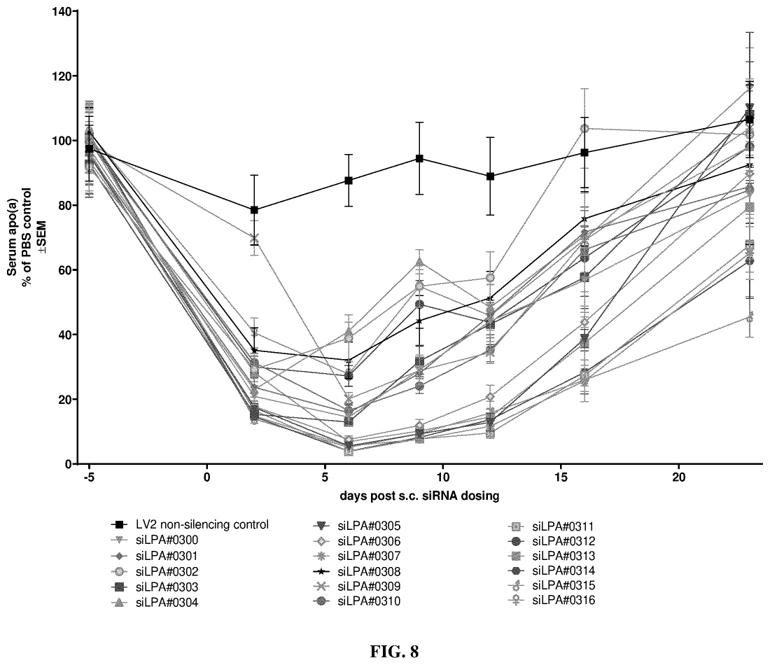

The innate immune response to the 17 selected LPA GalNAc-siRNAs was measured in vitro in human cells by examining the production of interferon α2a secreted from human primary PMBCs isolated from three different healthy donors in response to transfection of the siRNAs. No signs of immune stimulation in human PBMCs were observed for any of the tested LPA GalNAc-siRNAs (FIG. 7).

The LPA GalNAc-siRNAs were also tested for their in vitro nuclease stability in 50% murine serum by determining their relative stability and half-lives (Table 9). Half-lives ranged between ˜24 and ˜96 hours.

TABLE 9
Nuclease stability of selected GalNAc-
siRNAs in 50% mouse serum

| Compound | t_{1/2} | |
|---|---|---|
| siLPA#0300 | >24 | h |
| siLPA#0301 | >48 | h |
| siLPA#0302 | >24 | h |
| siLPA#0303 | >48 | h |
| siLPA#0304 | >48 | h |
| siLPA#0305 | 96 | h |
| siLPA#0306 | >48 | h |
| siLPA#0307 | >48 | h |
| siLPA#0308 | >48 | h |
| siLPA#0309 | >96 | h |
| siLPA#0310 | >48 | h |
| siLPA#0311 | >72 | h |
| siLPA#0312 | >24 | h |
| siLPA#0313 | >24 | h |
| siLPA#0314 | 72 | h |
| siLPA#0315 | >96 | h |
| siLPA#0316 | >48 | h |

Finally, the 17 selected LPA GalNAc-siRNAs were tested in vivo in a transgenic mouse model secreting human apo(a) protein from murine liver tissue (FIG. 8). After subcutaneous administration of the selected compounds at a single 5 mg/kg dose, target protein levels were reduced between 68% and 96% (KD max) compared to animals treated with PBS vehicle control. Depending on the compound, the levels returned to 50% of the maximum knockdown (KD₅₀) between ˜day 7 and ˜day 25 post treatment.

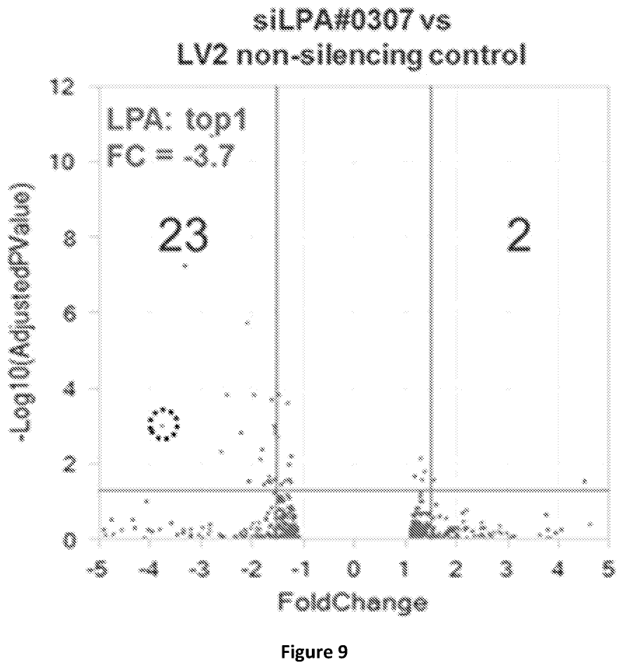

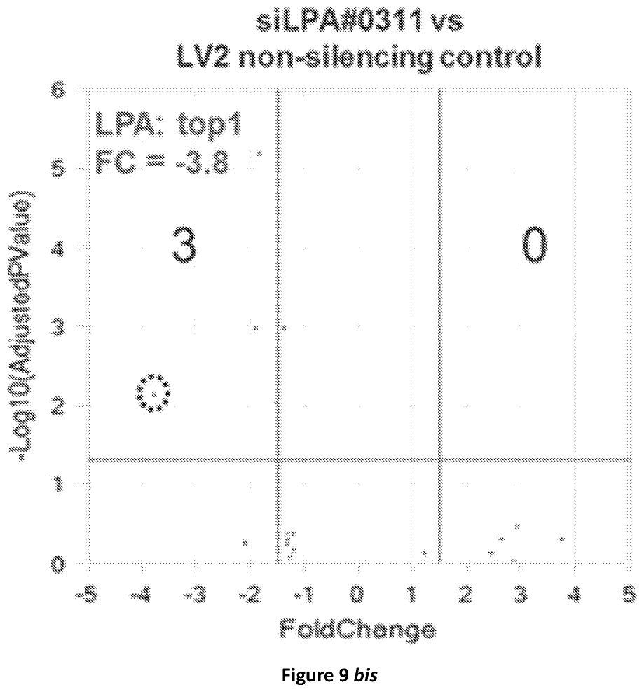

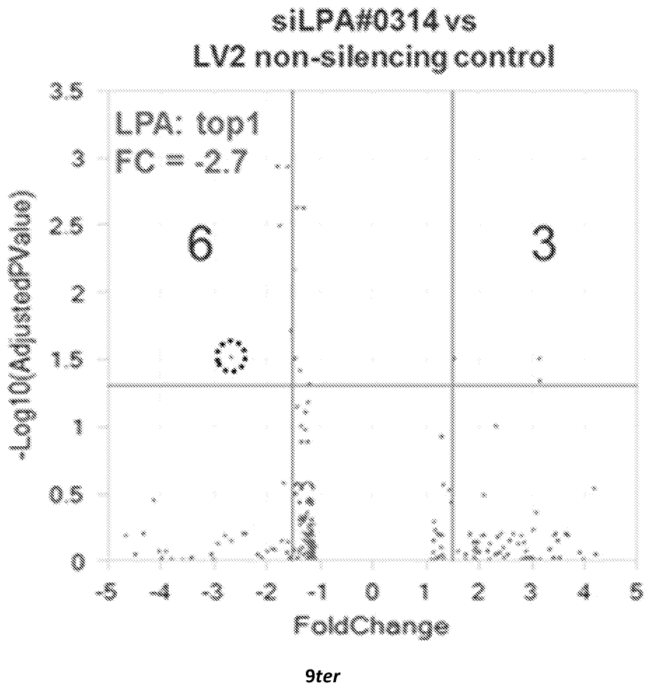

Three LPA GalNAc-siRNAs were selected that comprise a strong in vitro and in vivo on-target activity, retained cross-species activity in cynomolgus hepatocytes, and no off-target activity on plasminogen in human hepatocytes. The overall specificity of siLPA #0307, siLPA #0311 and siLPA #0314 was tested by RNA-Seq whole transcriptome analysis using primary human hepatocytes from two different donors treated with 5 μM LPA GalNAc-siRNAs for 72 hours. As shown in FIG. 9, the specificity of the three selected LPA GalNAc-siRNAs was confirmed, LPA being the most downregulated transcript in all of the three analyses.

In summary, the inventors have demonstrated the successful identification of potent, specific, and non-immunogenic LPA GalNAc-siRNAs that strongly reduce expression of the human LPA mRNA and translated apo(a) protein in relevant in vitro and in vivo models.

### Example 4: Lead Optimization of GalNAc-Conjugated LPA siRNA Sequences

Based on the results from Example 3, the three parent sequences of the selected LPA GalNAc-siRNAs (siLPA #0307, siLPA #0311, and siLPA #0314) were used for an optimization campaign that included 66 different chemical modifications per siRNA sequence. The resulting sequences and modification pattern are shown in Table 4. All experiments were done as described in Examples 2 and 3 above.

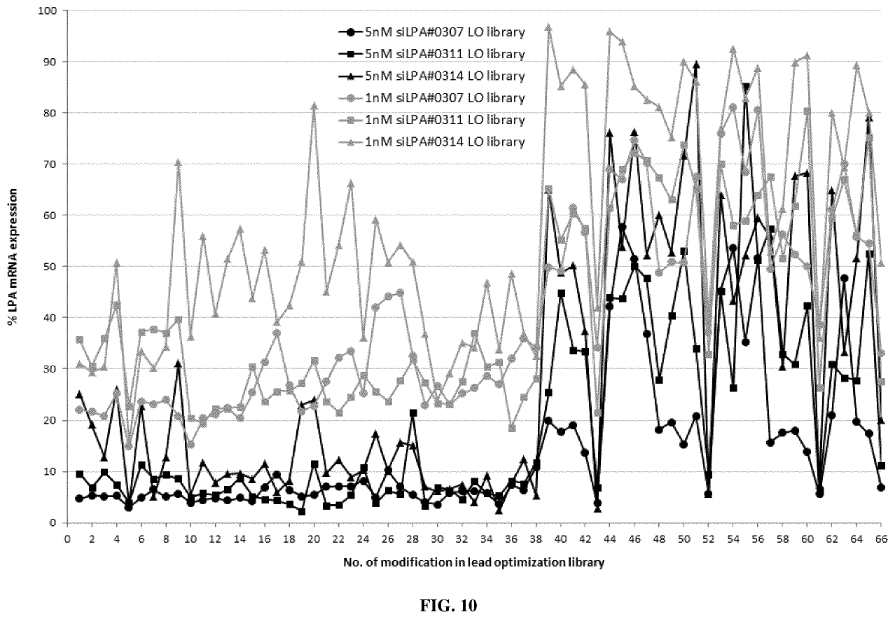

The in vitro activity of these optimization libraries was tested in freshly isolated primary hepatocytes from female apo(a) transgenic mice under free uptake conditions using 0.2 nM, 1 nM, and 5 nM concentrations of LPA GalNAc-siRNAs. As depicted in FIG. 10, the optimization libraries based on selected sequences siLPA #0307 and siLPA #0311 were identified to exhibit a higher overall in vitro activity as compared to lead sequence siLPA #0314.

In order to evaluate improved stability features of the optimized LPA GalNAc-siRNAs, the optimization libraries were assayed for their in vitro half-lives in 50% mouse serum. As demonstrated in Table 10, a large number of modifications were identified with improved nuclease stability as compared to the respective parent molecules.

TABLE 10

| siLPA ID | t_{1/2} | siLPA ID | t_{1/2} | siLPA ID | t_{1/2} | |
|---|---|---|---|---|---|---|
| siLPA#0307 | >48 h | siLPA#0311 | >72 h | siLPA#0314 | 72 | h |
| (parent) | | (parent) | | (parent) | | |
| siLPA#0317 | >72 h | siLPA#0383 | >72 h | siLPA#0449 | 168 | h |
| siLPA#0318 | >72 h | siLPA#0384 | >72 h | siLPA#0450 | 168 | h |
| siLPA#0319 | >72 h | siLPA#0385 | >72 h | siLPA#0451 | >96 | h |
| siLPA#0320 | >72 h | siLPA#0386 | >72 h | siLPA#0452 | 168 | h |
| siLPA#0321 | >72 h | siLPA#0387 | >96 h | siLPA#0453 | >96 | h |
| siLPA#0322 | >72 h | siLPA#0388 | >96 h | siLPA#0454 | 168 | h |
| siLPA#0323 | >72 h | siLPA#0389 | >96 h | siLPA#0455 | >96 | h |
| siLPA#0324 | >72 h | siLPA#0390 | >96 h | siLPA#0456 | >96 | h |
| siLPA#0325 | >72 h | siLPA#0391 | >96 h | siLPA#0457 | >96 | h |
| siLPA#0326 | >72 h | siLPA#0392 | >96 h | siLPA#0458 | >96 | h |
| siLPA#0327 | >72 h | siLPA#0393 | >96 h | siLPA#0459 | 168 | h |
| siLPA#0328 | >72 h | siLPA#0394 | >96 h | siLPA#0460 | 168 | h |
| siLPA#0329 | >72 h | siLPA#0395 | >96 h | siLPA#0461 | >96 | h |
| siLPA#0330 | >72 h | siLPA#0396 | >96 h | siLPA#0462 | 168 | h |
| siLPA#0331 | >72 h | siLPA#0397 | >96 h | siLPA#0463 | 168 | h |
| siLPA#0332 | >72 h | siLPA#0398 | >96 h | siLPA#0464 | 168 | h |
| siLPA#0333 | >72 h | siLPA#0399 | >96 h | siLPA#0465 | 168 | h |
| siLPA#0334 | >72 h | siLPA#0400 | >96 h | siLPA#0466 | >96 | h |
| siLPA#0335 | >72 h | siLPA#0401 | >96 h | siLPA#0467 | >96 | h |
| siLPA#0336 | >72 h | siLPA#0402 | >72 h | siLPA#0468 | 168 | h |
| siLPA#0337 | >72 h | siLPA#0403 | >72 h | siLPA#0469 | 168 | h |
| siLPA#0338 | >72 h | siLPA#0404 | >72 h | siLPA#0470 | 168 | h |
| siLPA#0339 | >72 h | siLPA#0405 | >72 h | siLPA#0471 | 168 | h |
| siLPA#0340 | >72 h | siLPA#0406 | >72 h | siLPA#0472 | 168 | h |
| siLPA#0341 | >72 h | siLPA#0407 | >96 h | siLPA#0473 | 168 | h |
| siLPA#0342 | >72 h | siLPA#0408 | >96 h | siLPA#0474 | >96 | h |
| siLPA#0343 | >72 h | siLPA#0409 | >96 h | siLPA#0475 | 168 | h |
| siLPA#0344 | >72 h | siLPA#0410 | >96 h | siLPA#0476 | 168 | h |
| siLPA#0345 | >72 h | siLPA#0411 | >72 h | siLPA#0477 | >96 | h |
| siLPA#0346 | >72 h | siLPA#0412 | >72 h | siLPA#0478 | >96 | h |
| siLPA#0347 | >72 h | siLPA#0413 | >96 h | siLPA#0479 | >96 | h |
| siLPA#0348 | >48 h | siLPA#0414 | >96 h | siLPA#0480 | >96 | h |
| siLPA#0349 | >72 h | siLPA#0415 | >96 h | siLPA#0481 | 168 | h |
| siLPA#0350 | >72 h | siLPA#0416 | >96 h | siLPA#0482 | 168 | h |
| siLPA#0351 | >72 h | siLPA#0417 | >96 h | siLPA#0483 | 168 | h |
| siLPA#0352 | >72 h | siLPA#0418 | >96 h | siLPA#0484 | 168 | h |
| siLPA#0353 | >72 h | siLPA#0419 | >96 h | siLPA#0485 | 168 | h |
| siLPA#0354 | >72 h | siLPA#0420 | >96 h | siLPA#0486 | 168 | h |
| siLPA#0355 | >72 h | siLPA#0421 | >96 h | siLPA#0487 | >72 | h |
| siLPA#0356 | >72 h | siLPA#0422 | >96 h | siLPA#0488 | >72 | h |
| siLPA#0357 | >72 h | siLPA#0423 | >96 h | siLPA#0489 | >72 | h |
| siLPA#0358 | >72 h | siLPA#0424 | >96 h | siLPA#0490 | >72 | h |
| siLPA#0359 | >72 h | siLPA#0425 | >96 h | siLPA#0491 | >72 | h |
| siLPA#0360 | >72 h | siLPA#0426 | >72 h | siLPA#0492 | >72 | h |
| siLPA#0361 | >72 h | siLPA#0427 | >72 h | siLPA#0493 | >72 | h |
| siLPA#0362 | >72 h | siLPA#0428 | >72 h | siLPA#0494 | >72 | h |
| siLPA#0363 | >72 h | siLPA#0429 | >72 h | siLPA#0495 | >72 | h |
| siLPA#0364 | >72 h | siLPA#0430 | >72 h | siLPA#0496 | 168 | h |
| siLPA#0365 | >72 h | siLPA#0431 | >72 h | siLPA#0497 | 168 | h |
| siLPA#0366 | >72 h | siLPA#0432 | >72 h | siLPA#0498 | 168 | h |
| siLPA#0367 | >72 h | siLPA#0433 | >72 h | siLPA#0499 | 168 | h |
| siLPA#0368 | >72 h | siLPA#0434 | >72 h | siLPA#0500 | 168 | h |
| siLPA#0369 | >72 h | siLPA#0435 | >72 h | siLPA#0501 | >72 | h |
| siLPA#0370 | >72 h | siLPA#0436 | >48 h | siLPA#0502 | >72 | h |
| siLPA#0371 | >72 h | siLPA#0437 | >48 h | siLPA#0503 | >72 | h |
| siLPA#0372 | >72 h | siLPA#0438 | >48 h | siLPA#0504 | >72 | h |
| siLPA#0373 | >72 h | siLPA#0439 | >72 h | siLPA#0505 | >96 | h |
| siLPA#0374 | >72 h | siLPA#0440 | >72 h | siLPA#0506 | >96 | h |
| siLPA#0375 | >72 h | siLPA#0441 | >72 h | siLPA#0507 | >96 | h |
| siLPA#0376 | >72 h | siLPA#0442 | >72 h | siLPA#0508 | >96 | h |
| siLPA#0377 | >72 h | siLPA#0443 | >96 h | siLPA#0509 | >96 | h |
| siLPA#0378 | >72 h | siLPA#0444 | >72 h | siLPA#0510 | 168 | h |
| siLPA#0379 | >72 h | siLPA#0445 | >72 h | siLPA#0511 | 168 | h |
| siLPA#0380 | >72 h | siLPA#0446 | >72 h | siLPA#0512 | 168 | h |
| siLPA#0381 | >72 h | siLPA#0447 | >72 h | siLPA#0513 | 168 | h |
| siLPA#0382 | >72 h | siLPA#0448 | >96 h | siLPA#0514 | 168 | h |

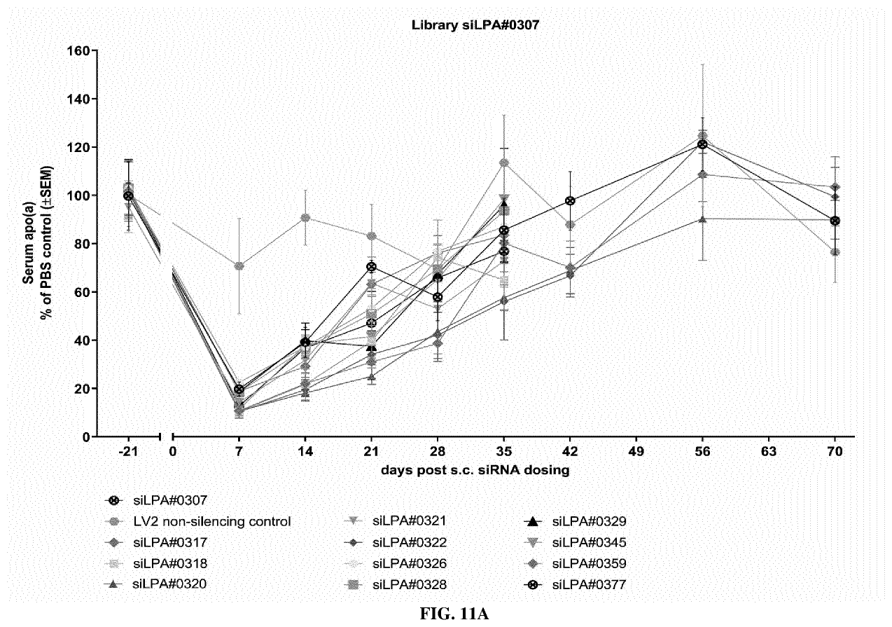

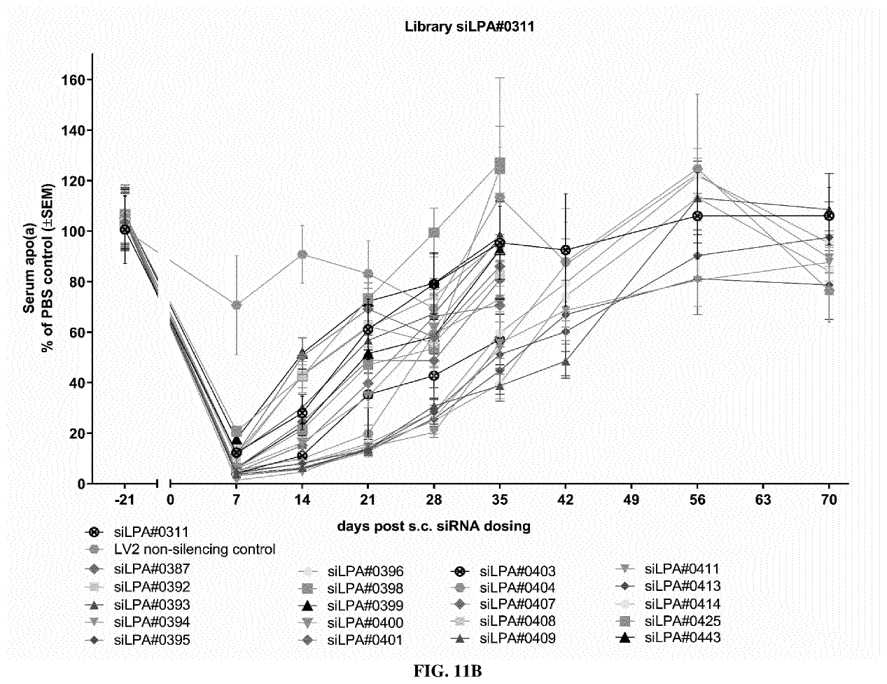

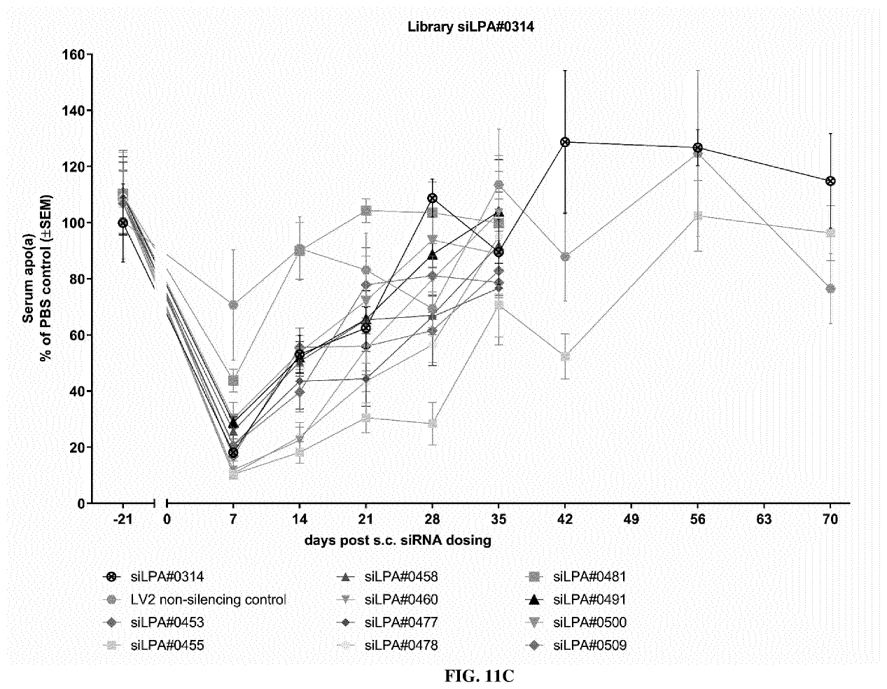

Next, in total 41 out of 198 optimized LPA GalNAc-siRNAs based on the three different parent sequences were selected for in vivo pharmacology testing in apo(a) transgenic mice and compared to the respective parent molecules siLPA #0307, siLPA #0311 and siLPA #0314 (FIGS. 11A-C). After subcutaneous administration of the selected compounds at a single 3 mg/kg dose, target protein levels were reduced between 56% and 99% (KDₘₐₓ) compared to animals treated with PBS vehicle control. Depending on the compound, the levels returned to 50% of the maximum knockdown (KD₅₀) between ˜day 8 and ˜day 42 post treatment. A large number of optimized molecules were identified with an improved in vivo pharmacology profile (KDₘₐₓ and KD₅₀) when compared to the respective parent sequences.

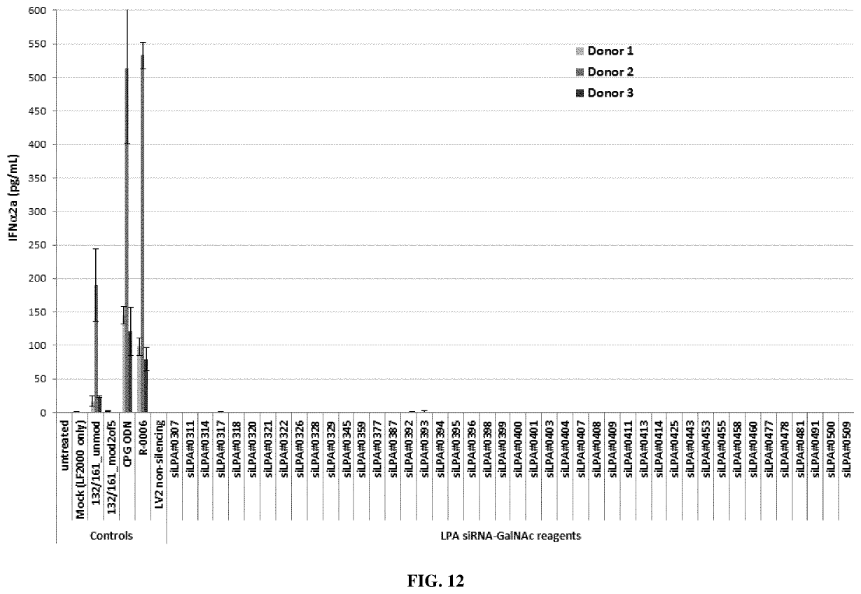

Towards the selection of advanced, optimized LPA GalNAc-siRNAs, further in vitro experiments were undertaken. The immune stimulatory potential was measured in the human PBMC assay using IFNα2a secretion to the supernatant as readout (FIG. 12). No signs of immune stimulation in human PBMCs were observed for any of the tested LPA GalNAc-siRNAs.

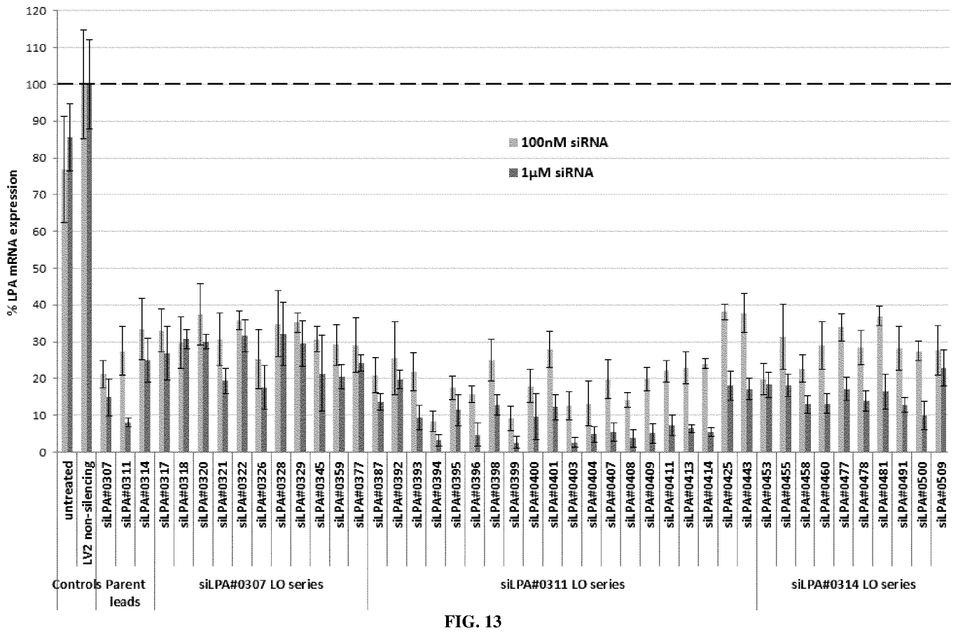

The cross-species activity of the 41 selected, optimized LPA GalNAc-siRNAs was evaluated in primary cynomolgus hepatocytes (FIG. 13). Interestingly, although all tested sequence modifications share one mismatch to macaque/the cynomolgus mRNA, the retained knockdown potencies differ largely among compounds.

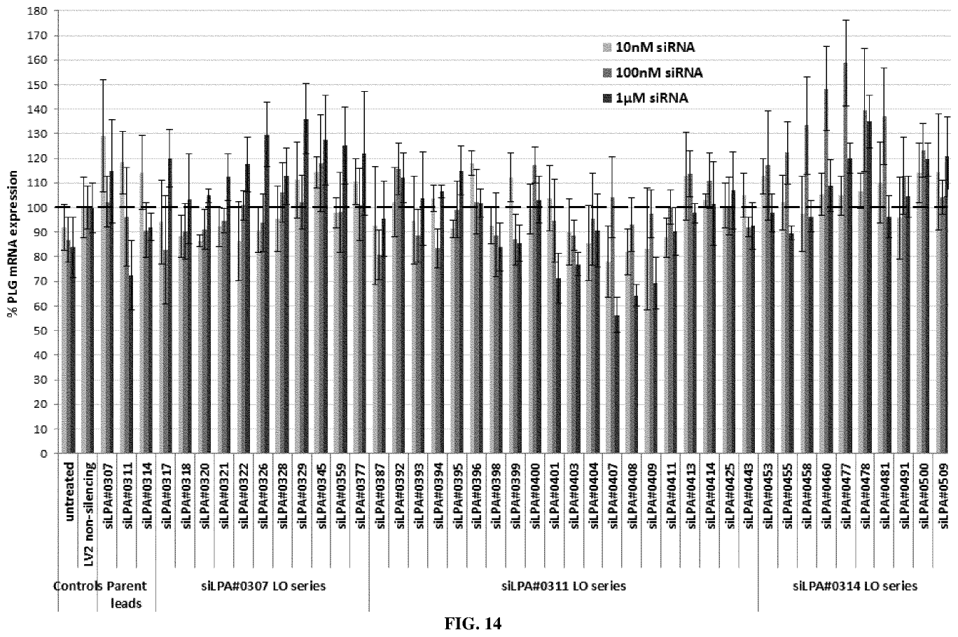

In order to test for relative specificity of the 41 selected, optimized LPA GalNAc-siRNAs, their effect on mRNA expression levels of human plasminogen using primary human hepatocytes under free uptake conditions was measured (FIG. 14). Only a few molecules were identified with a minor effect on PLG expression levels.

Finally, some advanced, optimized LPA GalNAc-siRNAs (siLPA #0317, siLPA #0393, siLPA #0394, siLPA #0411, siLPA #0414, and siLPA #0455) were assayed in IC₅₀ experiments under free uptake conditions using primary transgenic apo(a) mouse hepatocytes (Table 11).

TABLE 11
Activity of selected GalNAc-siRNAs in apo(a) mouse hepatocytes

| Compound | Iₘₐₓ % | IC₅₀ [nM] |
|---|---|---|
| siLPA#0317 | 94.7 | 0.0471 |
| siLPA#0393 | 93.9 | 0.0683 |
| siLPA#0394 | 96.2 | 0.0616 |
| siLPA#0411 | 86.7 | 0.128 |
| siLPA#0414 | 87.9 | 0.396 |
| siLPA#0455 | 85.5 | 0.367 |

Taken together, the inventors have presented data that demonstrate the successful identification of optimized LPA GalNAc-siRNAs that exhibit significantly improved in vitro and in vivo pharmacology profiles.

LPA Sequences

Human LPA mRNA sequence-NM_00577.2. (SEQ ID NO. 1632)

| | |
|---|---|
| 1 | aggtaccttt ggggctggct ttctcaagga agcccagctc cctgtgattg agaatgaagt |
| 61 | gtgcaatcgc tatgactggg attgggacac actttctggg cactgctggc cagtcccaaa |
| 121 | atggaacata aggaagtggt tcttctactt cttttatttc tgaaatcagc agcacctgag |
| 181 | caaagccatg tggtccagga ttgctaccat ggtgatggac agagttatcg aggcacgtac |
| 241 | tccaccactg tcacaggaag gacctgccaa gcttggtcat ctatgacacc acatcaacat |
| 301 | aataggacca cagaaaacta cccaaatgct ggcttgatca tgaactactg caggaatcca |
| 361 | gatgctgtgg cagctcctta ttgttatacg agggatcccg gtgtcaggtg ggagtactgc |
| 421 | aacctgacgc aatgctcaga cgcagaaggg actgccgtcg cgcctccgac tgttaccccg |
| 481 | gttccaagcc tagaggctcc ttccgaacaa gcaccgactg agcaaaggcc tggggtgcag |
| 541 | gagtgctacc atggtaatgg acagagttat cgaggcacat actccaccac tgtcacagga |
| 601 | agaacctgcc aagcttggtc atctatgaca ccacactcgc atagtcggac cccagaatac |
| 661 | tacccaaatg ctggcttgat catgaactac tgcaggaatc cagatgctgt ggcagctcct |
| 721 | tattgttata cgagggatcc cggtgtcagg tgggagtact gcaacctgac gcaatgctca |
| 781 | gacgcagaag ggactgccgt cgcgcctccg actgttaccc cggttccaag cctagaggct |
| 841 | ccttccgaac aagcaccgac tgagcaaagg cctggggtgc aggagtgcta ccatggtaat |
| 901 | ggacagagtt atcgaggcac atactccacc actgtcacag gaagaacctg ccaagcttgg |
| 961 | tcatctatga caccacactc gcatagtcgg accccagaat actacccaaa tgctggcttg |
| 1021 | atcatgaact actgcaggaa tccagatgct gtggcagctc cttattgtta tacgagggat |
| 1081 | cccggtgtca ggtgggagta ctgcaacctg acgcaatgct cagacgcaga agggactgcc |
| 1141 | gtcgcgcctc cgactgttac cccggttcca agcctagagg ctccttccga acaagcaccg |
| 1201 | actgagcaga ggcctggggt gcaggagtgc taccacggta atggacagag ttatcgaggc |
| 1261 | acatactcca ccactgtcac tggaagaacc tgccaagctt ggtcatctat gacaccacac |
| 1321 | tcgcatagtc ggaccccaga atactaccca aatgctggct tgatcatgaa ctactgcagg |
| 1381 | aatccagatg ctgtggcagc tccttattgt tatacgaggg atcccggtgt caggtgggag |
| 1441 | tactgcaacc tgacgcaatg ctcagacgca gaagggactg ccgtcgcgcc tccgactgtt |
| 1501 | accccggttc caagcctaga ggctccttcc gaacaagcac cgactgagca aaggcctggg |
| 1561 | gtgcaggagt gctaccatgg taatggacag agttatcgag gcacatactc caccactgtc |
| 1621 | acaggaagaa cctgccaagc ttggtcatct atgacaccac actcgcatag tcggacccca |
| 1681 | gaatactacc caaatgctgg cttgatcatg aactactgca ggaatccaga tgctgtggca |
| 1741 | gctccttatt gttatacgag ggatcccggt gtcaggtggg agtactgcaa cctgacgcaa |
| 1801 | tgctcagacg cagaagggac tgccgtcgcg cctccgactg ttaccccggt tccaagccta |
| 1861 | gaggctcctt ccgaacaagc accgactgag caaaggcctg gggtgcagga gtgctaccat |
| 1921 | ggtaatggac agagttatcg aggcacatac tccaccactg tcacaggaag aacctgccaa |
| 1981 | gcttggtcat ctatgacacc acactcgcat agtcggaccc cagaatacta cccaaatgct |
| 2041 | ggcttgatca tgaactactg caggaatcca gatgctgtgg cagctcctta ttgttatacg |
| 2101 | agggatcccg gtgtcaggtg ggagtactgc aacctgacgc aatgctcaga cgcagaaggg |
| 2161 | actgccgtcg cgcctccgac tgttaccccg gttccaagcc tagaggctcc ttccgaacaa |
| 2221 | gcaccgactg agcaaaggcc tggggtgcag gagtgctacc atggtaatgg acagagttat |
| 2281 | cgaggcacat actccaccac tgtcacagga agaacctgcc aagcttggtc atctatgaca |
| 2341 | ccacactcgc atagtcggac cccagaatac tacccaaatg ctggcttgat catgaactac |
| 2401 | tgcaggaatc cagatgctgt ggcagctcct tattgttata cgagggatcc cggtgtcagg |
| 2461 | tgggagtact gcaacctgac gcaatgctca gacgcagaag ggactgccgt cgcgcctccg |
| 2521 | actgttaccc cggttccaag cctagaggct ccttccgaac aagcaccgac tgagcagagg |
| 2581 | cctggggtgc aggagtgcta ccacggtaat ggacagagtt atcgaggcac atactccacc |
| 2641 | actgtcactg gaagaacctg ccaagcttgg tcatctatga caccacactc gcatagtcgg |
| 2701 | accccagaat actacccaaa tgctggcttg atcatgaact actgcaggaa tccagatcct |
| 2761 | gtggcagccc cttattgtta tacgagggat cccagtgtca ggtgggagta ctgcaacctg |
| 2821 | acacaatgct cagacgcaga agggactgcc gtcgcgcctc caactattac cccgattcca |
| 2881 | agcctagagg ctccttctga acaagcacca actgagcaaa ggcctggggt gcaggagtgc |
| 2941 | taccacggaa atggacagag ttatcaaggc acatacttca ttactgtcac aggaagaacc |
| 3001 | tgccaagctt ggtcatctat gacaccacac tcgcatagtc ggaccccagc atactaccca |
| 3061 | aatgctggct tgatcaagaa ctactgccga aatccagatc ctgtggcagc cccttggtgt |
| 3121 | tatacaacag atcccagtgt caggtgggag tactgcaacc tgacacgatg ctcagatgca |
| 3181 | gaatggactg ccttcgtccc tccgaatgtt attctggctc caagcctaga ggcttttttt |
| 3241 | gaacaagcac tgactgagga aacccccggg gtacaggact gctactacca ttatggacag |
| 3301 | agttaccgag gcacatactc caccactgtc acaggaagaa cttgccaagc ttggtcatct |
| 3361 | atgacaccac accagcatag tcggacccca gaaaactacc caaatgctgg cctgaccagg |
| 3421 | aactactgca ggaatccaga tgctgagatt cgcccttggt gttacaccat ggatcccagt |
| 3481 | gtcaggtggg agtactgcaa cctgacacaa tgcctggtga cagaatcaag tgtccttgca |
| 3541 | actctcacgg tggtcccaga tccaagcaca gaggcttctt ctgaagaagc accaacggag |
| 3601 | caaagccccg gggtccagga ttgctaccat ggtgatggac agagttatcg aggctcattc |
| 3661 | tctaccactg tcacaggaag gacatgtcag tcttggtcct ctatgacacc acactggcat |
| 3721 | cagaggacaa cagaatatta tccaaatggt ggcctgacca ggaactactg caggaatcca |
| 3781 | gatgctgaga ttagtccttg gtgttatacc atggatccca atgtcagatg ggagtactgc |
| 3841 | aacctgacac aatgtccagt gacagaatca agtgtccttg cgacgtccac ggctgtttct |
| 3901 | gaacaagcac caacggagca aagccccaca gtccaggact gctaccatgg tgatggacag |
| 3961 | agttatcgag gctcattctc caccactgtt acaggaagga catgtcagtc ttggtcctct |
| 4021 | atgacaccac actggcatca gagaaccaca gaatactacc caaatggtgg cctgaccagg |
| 4081 | aactactgca ggaatccaga tgctgagatt cgcccttggt gttataccat ggatcccagt |
| 4141 | gtcagatggg agtactgcaa cctgacgcaa tgtccagtga tggaatcaac tctcctcaca |
| 4201 | actcccacgg tggtcccagt tccaagcaca gagcttcctt ctgaagaagc accaactgaa |
| 4261 | aacagcactg gggtccagga ctgctaccga ggtgatggac agagttatcg aggcacactc |
| 4321 | tccaccacta tcacaggaag aacatgtcag tcttggtcgt ctatgacacc acattggcat |
| 4381 | cggaggatcc cattatacta tccaaatgct ggcctgacca ggaactactg caggaatcca |
| 4441 | gatgctgaga ttcgcccttg gtgttacacc atggatccca gtgtcaggtg ggagtactgc |
| 4501 | aacctgacac gatgtccagt gacagaatcg agtgtcctca caactcccac agtggccccg |
| 4561 | gttccaagca cagaggctcc ttctgaacaa gcaccacctg agaaaagccc tgtggtccag |
| 4621 | gattgctacc atggtgatgg acggagttat cgaggcatat cctccaccac tgtcacagga |
| 4681 | aggacctgtc aatcttggtc atctatgata ccacactggc atcagaggac cccagaaaac |
| 4741 | tacccaaatg ctggcctgac cgagaactac tgcaggaatc cagattctgg gaaacaaccc |
| 4801 | tggtgttaca caaccgatcc gtgtgtgagg tgggagtact gcaatctgac acaatgctca |
| 4861 | gaaacagaat caggtgtcct agagactccc actgttgttc cagttccaag catggaggct |
| 4921 | cattctgaag cagcaccaac tgagcaaacc cctgtggtcc ggcagtgcta ccatggtaat |
| 4981 | ggccagagtt atcgaggcac attctccacc actgtcacag gaaggacatg tcaatcttgg |
| 5041 | tcatccatga caccacaccg gcatcagagg accccagaaa actacccaaa tgatggcctg |
| 5101 | acaatgaact actgcaggaa tccagatgcc gatacaggcc cttggtgttt taccatggac |
| 5161 | cccagcatca ggtgggagta ctgcaacctg acgcgatgct cagacacaga agggactgtg |
| 5221 | gtcgctcctc cgactgtcat ccaggttcca agcctagggc ctccttctga acaagactgt |
| 5281 | atgtttggga atgggaaagg ataccggggc aagaaggcaa ccactgttac tgggacgcca |
| 5341 | tgccaggaat gggctgccca ggagccccat agacacagca cgttcattcc agggacaaat |
| 5401 | aaatgggcag gtctggaaaa aaattactgc cgtaaccctg atggtgacat caatggtccc |
| 5461 | tggtgctaca caatgaatcc aagaaaactt tttgactact gtgatatccc tctctgtgca |
| 5521 | tcctcttcat ttgattgtgg gaagcctcaa gtggagccga agaaatgtcc tggaagcatt |
| 5581 | gtaggggggt gtgtggccca cccacattcc tggccctggc aagtcagtct cagaacaagg |
| 5641 | tttggaaagc acttctgtgg aggcacctta atatccccag agtgggtgct gactgctgct |
| 5701 | cactgcttga agaagtcctc aaggccttca tcctacaagg tcatcctggg tgcacaccaa |
| 5761 | gaagtgaacc tcgaatctca tgttcaggaa atagaagtgt ctaggctgtt cttggagccc |
| 5821 | acacaagcag atattgcctt gctaaagcta agcaggcctg ccgtcatcac tgacaaagta |
| 5881 | atgccagctt gtctgccatc cccagactac atggtcaccg ccaggactga atgttacatc |
| 5941 | actggctggg gagaaaccca aggtaccttt gggactggcc ttctcaagga agcccagctc |
| 6001 | cttgttattg agaatgaagt gtgcaatcac tataagtata tttgtgctga gcatttggcc |
| 6061 | agaggcactg acagttgcca gggtgacagt ggagggcctc tggtttgctt cgagaaggac |
| 6121 | aaatacattt tacaaggagt cacttcttgg ggtcttggct gtgcacgccc caataagcct |
| 6181 | ggtgtctatg ctcgtgtttc aaggtttgtt acttggattg agggaatgat gagaaataat |
| 6241 | taattggacg ggagacagag tgaagcatca acctacttag aagctgaaac gtgggtaagg |
| 6301 | atttagcatg ctggaaataa tagacagcaa tcaaacgaag acactgttcc cagctaccag |
| 6361 | ctatgccaaa ccttggcatt tttggtattt ttgtgtataa gcttttaagg tctgactgac |
| 6421 | aaattctgta ttaaggtgtc atagctatga catttgttaa aaataaactc tgcacttatt |
| 6481 | ttgatttga |

human LPA mRNA sequence-NM_005577.3 (SEQ ID NO: 1627)

| | |
|---|---|
| 1 | ctgggattgg gacacacttt ctgggcactg ctggccagtc ccaaaatgga acataaggaa |
| 61 | gtggttcttc tacttctttt atttctgaaa tcagcagcac ctgagcaaag ccatgtggtc |
| 121 | caggattgct accatggtga tggacagagt tatcgaggca cgtactccac cactgtcaca |
| 181 | ggaaggacct gccaagcttg gtcatctatg acaccacatc aacataatag gaccacagaa |
| 241 | aactacccaa atgctggctt gatcatgaac tactgcagga atccagatgc tgtggcagct |
| 301 | ccttattgtt atacgaggga tcccggtgtc aggtgggagt actgcaacct gacgcaatgc |
| 361 | tcagacgcag aagggactgc cgtcgcgcct ccgactgtta ccccggttcc aagcctagag |
| 421 | gctccttccg aacaagcacc gactgagcaa aggcctgggg tgcaggagtg ctaccatggt |
| 481 | aatggacaga gttatcgagg cacatactcc accactgtca caggaagaac ctgccaagct |
| 541 | tggtcatcta tgacaccaca ctcgcatagt cggaccccag aatactaccc aaatgctggc |
| 601 | ttgatcatga actactgcag gaatccagat gctgtggcag ctccttattg ttatacgagg |
| 661 | gatcccggtg tcaggtggga gtactgcaac ctgacgcaat gctcagacgc agaagggact |
| 721 | gccgtcgcgc ctccgactgt taccccggtt ccaagcctag aggctccttc cgaacaagca |
| 781 | ccgactgagc aaaggcctgg ggtgcaggag tgctaccatg gtaatggaca gagttatcga |
| 841 | ggcacatact ccaccactgt cacaggaaga acctgccaag cttggtcatc tatgacacca |
| 901 | cactcgcata gtcggacccc agaatactac ccaaatgctg gcttgatcat gaactactgc |
| 961 | aggaatccag atgctgtggc agctccttat tgttatacga gggatcccgg tgtcaggtgg |
| 1021 | gagtactgca acctgacgca atgctcagac gcagaaggga ctgccgtcgc gcctccgact |
| 1081 | gttaccccgg ttccaagcct agaggctcct tccgaacaag caccgactga gcagaggcct |
| 1141 | ggggtgcagg agtgctacca cggtaatgga cagagttatc gaggcacata ctccaccact |
| 1201 | gtcactggaa gaacctgcca agcttggtca tctatgacac cacactcgca tagtcggacc |
| 1261 | ccagaatact acccaaatgc tggcttgatc atgaactact gcaggaatcc agatgctgtg |
| 1321 | gcagctcctt attgttatac gagggatccc ggtgtcaggt gggagtactg caacctgacg |
| 1381 | caatgctcag acgcagaagg gactgccgtc gcgcctccga ctgttacccc ggttccaagc |
| 1441 | ctagaggctc cttccgaaca agcaccgact gagcaaaggc ctggggtgca ggagtgctac |
| 1501 | catggtaatg gacagagtta tcgaggcaca tactccacca ctgtcacagg aagaacctgc |
| 1561 | caagcttggt catctatgac accacactcg catagtcgga ccccagaata ctacccaaat |
| 1621 | gctggcttga tcatgaacta ctgcaggaat ccagatgctg tggcagctcc ttattgttat |
| 1681 | acgagggatc ccggtgtcag gtgggagtac tgcaacctga cgcaatgctc agacgcagaa |
| 1741 | gggactgccg tcgcgcctcc gactgttacc ccggttccaa gcctagaggc tccttccgaa |
| 1801 | caagcaccga ctgagcaaag gcctggggtg caggagtgct accatggtaa tggacagagt |
| 1861 | tatcgaggca catactccac cactgtcaca ggaagaacct gccaagcttg gtcatctatg |
| 1921 | acaccacact cgcatagtcg gaccccagaa tactacccaa atgctggctt gatcatgaac |
| 1981 | tactgcagga atccagatgc tgtggcagct ccttattgtt atacgaggga tcccggtgtc |
| 2041 | aggtgggagt actgcaacct gacgcaatgc tcagacgcag aagggactgc cgtcgcgcct |
| 2101 | ccgactgtta ccccggttcc aagcctagag gctccttccg aacaagcacc gactgagcaa |
| 2161 | aggcctgggg tgcaggagtg ctaccatggt aatggacaga gttatcgagg cacatactcc |
| 2221 | accactgtca caggaagaac ctgccaagct tggtcatcta tgacaccaca ctcgcatagt |
| 2281 | cggaccccag aatactaccc aaatgctggc ttgatcatga actactgcag gaatccagat |
| 2341 | gctgtggcag ctccttattg ttatacgagg gatcccggtg tcaggtggga gtactgcaac |
| 2401 | ctgacgcaat gctcagacgc agaagggact gccgtcgcgc ctccgactgt taccccggtt |
| 2461 | ccaagcctag aggctccttc cgaacaagca ccgactgagc agaggcctgg ggtgcaggag |
| 2521 | tgctaccacg gtaatggaca gagttatcga ggcacatact ccaccactgt cactggaaga |
| 2581 | acctgccaag cttggtcatc tatgacacca cactcgcata gtcggacccc agaatactac |
| 2641 | ccaaatgctg gcttgatcat gaactactgc aggaatccag atcctgtggc agccccttat |
| 2701 | tgttatacga gggatcccag tgtcaggtgg gagtactgca acctgacaca atgctcagac |
| 2761 | gcagaaggga ctgccgtcgc gcctccaact attaccccga ttccaagcct agaggctcct |
| 2821 | tctgaacaag caccaactga gcaaaggcct ggggtgcagg agtgctacca cggaaatgga |
| 2881 | cagagttatc aaggcacata cttcattact gtcacaggaa gaacctgcca agcttggtca |
| 2941 | tctatgacac cacactcgca tagtcggacc ccagcatact acccaaatgc tggcttgatc |
| 3001 | aagaactact gccgaaatcc agatcctgtg gcagcccctt ggtgttatac aacagatccc |
| 3061 | agtgtcaggt gggagtactg caacctgaca cgatgctcag atgcagaatg gactgccttc |
| 3121 | gtccctccga atgttattct ggctccaagc ctagaggctt tttttgaaca agcactgact |
| 3181 | gaggaaaccc ccggggtaca ggactgctac taccattatg gacagagtta ccgaggcaca |
| 3241 | tactccacca ctgtcacagg aagaacttgc caagcttggt catctatgac accacaccag |
| 3301 | catagtcgga ccccagaaaa ctacccaaat gctggcctga ccaggaacta ctgcaggaat |
| 3361 | ccagatgctg agattcgccc ttggtgttac accatggatc ccagtgtcag gtgggagtac |
| 3421 | tgcaacctga cacaatgcct ggtgacagaa tcaagtgtcc ttgcaactct cacggtggtc |
| 3481 | ccagatccaa gcacagaggc ttcttctgaa gaagcaccaa cggagcaaag ccccggggtc |
| 3541 | caggattgct accatggtga tggacagagt tatcgaggct cattctctac cactgtcaca |
| 3601 | ggaaggacat gtcagtcttg gtcctctatg acaccacact ggcatcagag gacaacagaa |
| 3661 | tattatccaa atggtggcct gaccaggaac tactgcagga atccagatgc tgagattagt |
| 3721 | ccttggtgtt ataccatgga tcccaatgtc agatgggagt actgcaacct gacacaatgt |
| 3781 | ccagtgacag aatcaagtgt ccttgcgacg tccacggctg tttctgaaca agcaccaacg |
| 3841 | gagcaaagcc ccacagtcca ggactgctac catggtgatg gacagagtta tcgaggctca |
| 3901 | ttctccacca ctgttacagg aaggacatgt cagtcttggt cctctatgac accacactgg |
| 3961 | catcagagaa ccacagaata ctacccaaat ggtggcctga ccaggaacta ctgcaggaat |
| 4021 | ccagatgctg agattcgccc ttggtgttat accatggatc ccagtgtcag atgggagtac |
| 4081 | tgcaacctga cgcaatgtcc agtgatggaa tcaactctcc tcacaactcc cacggtggtc |
| 4141 | ccagttccaa gcacagagct tccttctgaa gaagcaccaa ctgaaaacag cactggggtc |
| 4201 | caggactgct accgaggtga tggacagagt tatcgaggca cactctccac cactatcaca |
| 4261 | ggaagaacat gtcagtcttg gtcgtctatg acaccacatt ggcatcggag gatcccatta |
| 4321 | tactatccaa atgctggcct gaccaggaac tactgcagga atccagatgc tgagattcgc |
| 4381 | ccttggtgtt acaccatgga tcccagtgtc aggtgggagt actgcaacct gacacgatgt |
| 4441 | ccagtgacag aatcgagtgt cctcacaact cccacagtgg ccccggttcc aagcacagag |
| 4501 | gctccttctg aacaagcacc acctgagaaa agccctgtgg tccaggattg ctaccatggt |
| 4561 | gatggacgga gttatcgagg catatcctcc accactgtca caggaaggac ctgtcaatct |
| 4621 | tggtcatcta tgataccaca ctggcatcag aggaccccag aaaactaccc aaatgctggc |
| 4681 | ctgaccgaga actactgcag gaatccagat tctgggaaac aaccctggtg ttacacaacc |
| 4741 | gatccgtgtg tgaggtggga gtactgcaat ctgacacaat gctcagaaac agaatcaggt |
| 4801 | gtcctagaga ctcccactgt tgttccagtt ccaagcatgg aggctcattc tgaagcagca |
| 4861 | ccaactgagc aaacccctgt ggtccggcag tgctaccatg gtaatggcca gagttatcga |
| 4921 | ggcacattct ccaccactgt cacaggaagg acatgtcaat cttggtcatc catgacacca |
| 4981 | caccggcatc agaggacccc agaaaactac ccaaatgatg gcctgacaat gaactactgc |
| 5041 | aggaatccag atgccgatac aggcccttgg tgttttacca tggaccccag catcaggtgg |
| 5101 | gagtactgca acctgacgcg atgctcagac acagaaggga ctgtggtcgc tcctccgact |
| 5161 | gtcatccagg ttccaagcct agggcctcct tctgaacaag actgtatgtt tgggaatggg |
| 5221 | aaaggatacc ggggcaagaa ggcaaccact gttactggga cgccatgcca ggaatgggct |
| 5281 | gcccaggagc cccatagaca cagcacgttc attccaggga caaataaatg ggcaggtctg |
| 5341 | gaaaaaaatt actgccgtaa ccctgatggt gacatcaatg gtccctggtg ctacacaatg |
| 5401 | aatccaagaa aactttttga ctactgtgat atccctctct gtgcatcctc ttcatttgat |
| 5461 | tgtgggaagc ctcaagtgga gccgaagaaa tgtcctggaa gcattgtagg ggggtgtgtg |
| 5521 | gcccacccac attcctggcc ctggcaagtc agtctcagaa caaggtttgg aaagcacttc |
| 5581 | tgtggaggca ccttaatatc cccagagtgg gtgctgactg ctgctcactg cttgaagaag |
| 5641 | tcctcaaggc cttcatccta caaggtcatc ctgggtgcac accaagaagt gaacctcgaa |
| 5701 | tctcatgttc aggaaataga agtgtctagg ctgttcttgg agcccacaca agcagatatt |
| 5761 | gccttgctaa agctaagcag gcctgccgtc atcactgaca aagtaatgcc agcttgtctg |
| 5821 | ccatccccag actacatggt caccgccagg actgaatgtt acatcactgg ctggggagaa |
| 5881 | acccaaggta cctttgggac tggccttctc aaggaagccc agctccttgt tattgagaat |
| 5941 | gaagtgtgca atcactataa gtatatttgt gctgagcatt tggccagagg cactgacagt |
| 6001 | tgccagggtg acagtggagg gcctctggtt tgcttcgaga aggacaaata cattttacaa |
| 6061 | ggagtcactt cttggggtct tggctgtgca cgccccaata agcctggtgt ctatgctcgt |
| 6121 | gtttcaaggt ttgttacttg gattgaggga atgatgagaa ataattaatt ggacgggaga |
| 6181 | cagagtgaag catcaaccta cttagaagct gaaacgtggg taaggattta gcatgctgga |
| 6241 | aataatagac agcaatcaaa cgaagacact gttcccagct accagctatg ccaaaccttg |
| 6301 | gcatttttgg tatttttgtg tataagcttt taaggtctga ctgacaaatt ctgtattaag |
| 6361 | gtgtcatagc tatgacattt gttaaaaata aactctgcac ttattttgat ttga |

human LPA polypeptide sequence (SEQ ID NO: 1628)
MEHKEVVLLLLLFLKSAAPEQSHVVQDCYHGDGQSYRGTYSTTVTGRTCQAWSSMTPHQHNRTTENYPNAGLIMNYC
RNPDAVAAPYCYTRDPGVRWEYCNLTQCSDAEGTAVAPPTVTPVPSLEAPSEQAPTEQRPGVQECYHGNGQSYRGTY
STTVTGRTCQAWSSMTPHSHSRTPEYYPNAGLIMNYCRNPDAVAAPYCYTRDPGVRWEYCNLTQCSDAEGTAVAPPT
VTPVPSLEAPSEQAPTEQRPGVQECYHGNGQSYRGTYSTTVTGRTCQAWSSMTPHSHSRTPEYYPNAGLIMNYCRNP
DAVAAPYCYTRDPGVRWEYCNLTQCSDAEGTAVAPPTVTPVPSLEAPSEQAPTEQRPGVQECYHGNGQSYRGTYSTT
VTGRTCQAWSSMTPHSHSRTPEYYPNAGLIMNYCRNPDAVAAPYCYTRDPGVRWEYCNLTQCSDAEGTAVAPPTVTP
VPSLEAPSEQAPTEQRPGVQECYHGNGQSYRGTYSTTVTGRTCQAWSSMTPHSHSRTPEYYPNAGLIMNYCRNPDAV
AAPYCYTRDPGVRWEYCNLTQCSDAEGTAVAPPTVTPVPSLEAPSEQAPTEQRPGVQECYHGNGQSYRGTYSTTVTG
RTCQAWSSMTPHSHSRTPEYYPNAGLIMNYCRNPDAVAAPYCYTRDPGVRWEYCNLTQCSDAEGTAVAPPTVTPVPS
LEAPSEQAPTEQRPGVQECYHGNGQSYRGTYSTTVTGRTCQAWSSMTPHSHSRTPEYYPNAGLIMNYCRNPDAVAAP
YCYTRDPGVRWEYCNLTQCSDAEGTAVAPPTVTPVPSLEAPSEQAPTEQRPGVQECYHGNGQSYRGTYSTTVTGRTC
QAWSSMTPHSHSRTPEYYPNAGLIMNYCRNPDPVAAPYCYTRDPSVRWEYCNLTQCSDAEGTAVAPPTITPIPSLEA
PSEQAPTEQRPGVQECYHGNGQSYQGTYFITVTGRTCQAWSSMTPHSHSRTPAYYPNAGLIKNYCRNPDPVAAPWCY
TTDPSVRWEYCNLTRCSDAEWTAFVPPNVILAPSLEAFFEQALTEETPGVQDCYYHYGQSYRGTYSTTVTGRTCQAW
SSMTPHQHSRTPENYPNAGLTRNYCRNPDAEIRPWCYTMDPSVRWEYCNLTQCLVTESSVLATLTVVPDPSTEASSE
EAPTEQSPGVQDCYHGDGQSYRGSFSTTVTGRTCQSWSSMTPHWHQRTTEYYPNGGLTRNYCRNPDAEISPWCYTMD
PNVRWEYCNLTQCPVTESSVLATSTAVSEQAPTEQSPTVQDCYHGDGQSYRGSFSTTVTGRTCQSWSSMTPHWHQRT
TEYYPNGGLTRNYCRNPDAEIRPWCYTMDPSVRWEYCNLTQCPVMESTLLTTPTVVPVPSTELPSEEAPTENSTGVQ
DCYRGDGQSYRGTLSTTITGRTCQSWSSMTPHWHRRIPLYYPNAGLTRNYCRNPDAEIRPWCYTMDPSVRWEYCNLT
RCPVTESSVLTTPTVAPVPSTEAPSEQAPPEKSPVVQDCYHGDGRSYRGISSTTVTGRTCQSWSSMIPHWHQRTPEN
YPNAGLTENYCRNPDSGKQPWCYTTDPCVRWEYCNLTQCSETESGVLETPTVVPVPSMEAHSEAAPTEQTPVVRQCY
HGNGQSYRGTFSTTVTGRTCQSWSSMTPHRHQRTPENYPNDGLIMNYCRNPDADTGPWCFTMDPSIRWEYCNLTRCS
DTEGTVVAPPTVIQVPSLGPPSEQDCMFGNGKGYRGKKATTVTGTPCQEWAAQEPHRHSTFIPGINKWAGLEKNYCR
NPDGDINGPWCYTMNPRKLFDYCDIPLCASSSFDCGKPQVEPKKCPGSIVGGCVAHPHSWPWQVSLRTRFGKHFCGG
TLISPEWVLTAAHCLKKSSRPSSYKVILGAHQEVNLESHVQEIEVSRLFLEPTQADIALLKLSRPAVITDKVMPACL
PSPDYMVTARTECYITGWGETQGTFGTGLLKEAQLLVIENEVCNHYKYICAEHLARGTDSCQGDSGGPLVCFEKDKY
ILQGVTSWGLGCARPNKPGVYARVSRFVTWIEGMMRNN
cynomolgus LPA mRNA sequence (SEQ ID NO: 1629)

| | |
|---|---|
| 1 | gatgctgcat acttaatgtc gaaaggttgc ttcatccaag agcctggagt tttcagagac |
| 61 | actgtcctga aactatgtcc tgaaactatg tcattgaaac tgaaacattg tcctgaagct |
| 121 | ggtattgggc aataccagcg cctgcaggca acagctcgga tgcacttaag atttaaatat |
| 181 | tacccacaga agttctggct tgtctgggaa aaccttttgc taaacagaag agcaacattt |
| 241 | tttttttttt cttttctgga atttgtaaac agcatttatt ctcagcctta ccttccaaac |
| 301 | gttgcacttg gaacattgct gggccccgtg gaaacagaag cgaacgtcag ccaggccggc |
| 361 | agggggcggc agaccccaca cttcgccggg cgccctcacc tccctgggag ggagtgtgca |
| 421 | gctgccaaaa tcttcggcgg ggttcagtcc aagcgacttc agccagcaga tggtcattct |
| 481 | cctgtgaccg tgtgtactac agactgtttc aaaaccgggc aggcaattaa caatgggaat |
| 541 | tctgccatca tcgctgacaa agtcatccca gtttgtctgc catccccaaa ttatgtggtc |
| 601 | gccaaccaga ctgaatgtta tgtcactggc tggggagaaa cccaagcact acctgagcaa |
| 661 | agccatgtgg tccaggattg ctaccatggt gatggacaga gttatcaagg cacatcctcc |
| 721 | accactgtca caggaaggac ctgccaagct tggtcatcta tggaaccaca tcagcataat |
| 781 | agaaccacag aaaactaccc aaatgctggc ttgatcagga actactgcag gaatccagat |
| 841 | cctgtggcag ccccttattg ttatacgatg gatcccaatg tcaggtggga gtactgcaac |
| 901 | ctgacacaat gctcagacgc agaagggact gccgtcgcac ctccgaatgt caccccggtt |
| 961 | ccaagcctag aggctccttc cgaacaagca ccgactgagc aaaggcctgg ggtgcaggag |
| 1021 | tgctaccacg gtaatggaca gagttatcga ggcacatact tcaccactgt gacaggaaga |
| 1081 | acctgccaag cttggtcatc tatgacaccg cactctcata gtcggacccc ggaaaactac |
| 1141 | ccaaatggtg gcttgatcag gaactactgc aggaatccag atcctgtggc agccccttat |
| 1201 | tgttatacca tggatcccaa tgtcaggtgg gagtactgca acctgacaca atgctcagac |
| 1261 | gcagaaggga ttgccgtcac acctctgact gttaccccgg ttccaagcct agaggctcct |
| 1321 | tccaagcaag caccaactga gcaaaggcct ggtgtccagg agtgctacca cggtaatgga |
| 1381 | cagagttatc gaggcacata cttcaccact gtgacaggaa gaacctgcca agcttggtca |
| 1441 | tctatgacac cacattctca tagtcgtacc ccagaaaact acccaaatgg tggcttgatc |
| 1501 | aggaactact gcaggaatcc agatcctgtg gcagcccctt attgttatac catggatccc |
| 1561 | aatgtcaggt gggagtactg caacctgaca caatgctcag acgcagaagg gactgccgtc |
| 1621 | gcacctccga ctgtcacccc ggttccaagc ctagaggctc cttccgaaca agcaccgact |
| 1681 | gagcaaaggc ctggggtgca ggagtgctac cacggtaatg gacagagtta tcgaggcaca |
| 1741 | tacttcacca ctgtgacagg aagaacctgc caagcttggt catctatgac accgcactct |
| 1801 | catagtcgga ccccggaaaa ctacccaaat ggtggcttga tcaggaacta ctgcaggaat |
| 1861 | ccagatcctg tggcagcccc ttattgttat accatggatc ccaatgtcag gtgggagtac |
| 1921 | tgcaacctga cacaatgctc agacgcagaa gggactgccg tcgcacctcc gaatgtcacc |
| 1981 | ccggttccaa gcctagaggc tccttctgag caagcaccaa ctgagcaaag gcttggggtg |
| 2041 | caggagtgct accacggtaa tggacagagt tatcgaggca catacttcac cactgtgaca |
| 2101 | ggaagaacct gccaagcttg gtcatctatg acaccacact ctcatagtcg gaccccagaa |
| 2161 | aactacccaa atgctggctt ggtcaagaac tactgccgaa atccagatcc tgtggcagcc |
| 2221 | ccttggtgtt atacaacgga tcccagtgtc aggtgggagt actgcaacct gacacgatgc |
| 2281 | tcagatgcag aagggactgc tgttgtgcct ccaaatatta ttccggttcc aagcctagag |
| 2341 | gcttttcttg aacaagaacc gactgaggaa acccccgggg tacaggagtg ctactaccat |
| 2401 | tatggacaga gttatagagg cacatactcc accactgtta caggaagaac ttgccaagct |
| 2461 | tggtcatcta tgacaccaca ccagcatagt cggaccccaa aaaactatcc aaatgctggc |
| 2521 | ctgaccagga actactgcag gaatccagat gctgagattc gcccttggtg ttataccatg |
| 2581 | gatcccagtg tcaggtggga gtactgcaac ctgacacaat gtctggtgac agaatcaagt |
| 2641 | gtccttgaaa ctctcacagt ggtcccagat ccaagcacac aggcttcttc tgaagaagca |
| 2701 | ccaacggagc aaagtcccga ggtccaggac tgctaccatg gtgatggaca gagttatcga |
| 2761 | ggctcattct ccaccactgt cacaggaagg acatgtcagt cttggtcctc tatgacacca |
| 2821 | cactggcatc agaggacaac agaatattat ccagatggtg gcctgaccag gaactactgc |
| 2881 | aggaatccag atgctgagat tcgcccttgg tgttatacca tggatcccag tgtcaggtgg |
| 2941 | gagtactgca acctgacaca atgtccagtg acagaatcaa gtgtcctcgc aacgtccatg |
| 3001 . | gctgtttctg aacaagcacc aatggagcaa agccccgggg tccaggactg ctaccatggt |
| 3061 | gatggacaga gttatcgagg ttcattctcc accactgtca caggaaggac atgtcagtct |
| 3121 | tggtcctcta tgacaccaca ctggcatcag aggaccatag aatactaccc aaatggtggc |
| 3181 | ctgaccaaga actactgcag gaatccagat gctgagattc gcccttggtg ttataccatg |
| 3241 | gatcccagag tcagatggga gtactgcaac ctgacacaat gtgtggtgat ggaatcaagt |
| 3301 | gtccttgcaa ctcccatggt ggtcccagtt ccaagcagag aggttccttc tgaagaagca |
| 3361 | ccaactgaaa acagccctgg ggtccaggac tgctaccaag gtgatggaca gagttatcga |
| 3421 | ggcacattct ccaccactat cacaggaaga acatgtcagt cttggttgtc tatgacacca |
| 3481 | catcggcatc ggaggatccc attacgctat ccaaatgctg gcctgaccag gaactattgc |
| 3541 | agaaatccag atgctgagat tcgcccttgg tgttacacca tggatcccag tgtcaggtgg |
| 3601 | gagtactgca acctgacaca atgtccagtg acagaatcaa gtgtcctcac aactcccacg |
| 3661 | gtggtcccgg ttccaagcac agaggctcct tctgaacaag caccacctga gaaaagccct |
| 3721 | gtggtccagg attgctacca tggtgatgga cagagttatc gaggcacatc ctccaccact |
| 3781 | gtcacaggaa ggaactgtca gtcttggtca tctatgatac cacactggca tcagaggacc |
| 3841 | ccagaaaact acccaaatgc tggcctgacc aggaactact gcaggaatcc agattctggg |
| 3901 | aaacaaccct ggtgttacac gactgatcca tgtgtgaggt gggagtactg caacctgaca |
| 3961 | caatgctcag aaacagaatc aggtgtccta gagactccca ctgttgttcc ggttccaagc |
| 4021 | atggaagctc attctgaagc agcaccaact gagcaaactc ctgtggtcca gcagtgctac |
| 4081 | catggtaatg gacagagtta tcgaggcaca ttctccacca ctgtcacagg aaggacatgt |
| 4141 | caatcttggt catccatgac accacaccag cataagagga ccccggaaaa ccacccaaat |
| 4201 | gatgacttga caatgaacta ctgcaggaat ccagatgctg acacaggccc ttggtgtttt |
| 4261 | accatggacc ccagcgtcag gcgggagtac tgcaacctga cgcgatgctc agacacagaa |
| 4321 | gggactgtgg tcacacctcc gactgttatc ccggttccaa gcctagaggc tccttctgaa |
| 4381 | caagcatcct cttcatttga ttgtgggaag cctcaagtgg agccaaagaa atgtcctgga |
| 4441 | agcattgtag gtgggtgtgt ggcccaccca cattcctggc cctggcaagt cagtcttaga |
| 4501 | acaaggtttg gaaagcactt ctgtggaggc accttaatat ccccagagtg ggtgctgact |
| 4561 | gctgcttgct gcttggagac gttctcaagg ccttccttct acaaggtcat cctgggtgca |
| 4621 | caccaagaag tgaatctcga atctcacgtt caagaaatag aagtgtctag gttgttcttg |
| 4681 | gagcccatag gagcagatat tgccttgcta aagctaagca ggcctgccat catcactgac |
| 4741 | aaagtaatcc cagcctgtct gccgtctcca aattacgtga tcaccgtctg gactgaatgt |
| 4801 | tacatcactg gctggggaga aacccaaggt acctttgggg ctggccttct caaggaagcc |
| 4861 | cagcttcatg tgattgagaa tacagtgtgc aatcactacg agtttctgaa tggaagagtc |
| 4921 | aaatccaccg agctctgtgc tgggcatttg gccggaggca ctgacagatg ccagggtgac |
| 4981 | agtggagggc ctgtggtttg cttcgacaag gacaaataca ttttacgagg aataacttct |
| 5041 | tggggtcctg gctgtgcatg ccccaataag cctggtgtct atgttcgtgt ttcaagcttt |
| 5101 | gtcacttgga ttgagggagt gatgagaaat aattaattga acaagagaca gagtgaagca |
| 5161 | ttgactcacc tagaggctag aatgggggta gggatttagc acgctggaaa taacggacag |
| 5221 | taatcaaacg aagacactgt ccccagctac caactatgcc aaacctcagc atttttggta |
| 5281 | ttattgtgta taagcttttc ccgtctgact gctgggttct ccaataaggt gacatagcta |
| 5341 | tgccatttgt taaaaataaa ctctgtactt attttgattt gagtaaa |

cynomolgus LPA polypeptide sequence (SEQ ID NO: 1630)
MSKGCFIQEPGVFRDTVLKLCPETMSLKLKHCPEAGIGQYQRLQATARMHLRFKYYPQKFWLVWENLLLN
RRATFFFFSFLEFVNSIYSQPYLPNVALGTLLGPVETEANVSQAGRGRQTPHFAGRPHLPGRECAAAKIF
GGVQSKRLQPADGHSPVTVCTTDCFKTGQAINNGNSAIIADKVIPVCLPSPNYVVANQTECYVTGWGETQ
ALPEQSHVVQDCYHGDGQSYQGTSSTTVTGRTCQAWSSMEPHQHNRTTENYPNAGLIRNYCRNPDPVAAP
YCYTMDPNVRWEYCNLTQCSDAEGTAVAPPNVTPVPSLEAPSEQAPTEQRPGVQECYHGNGQSYRGTYFT
TVTGRTCQAWSSMTPHSHSRTPENYPNGGLIRNYCRNPDPVAAPYCYTMDPNVRWEYCNLTQCSDAEGIA
VTPLTVTPVPSLEAPSKQAPTEQRPGVQECYHGNGQSYRGTYFTTVTGRTCQAWSSMTPHSHSRTPENYP
NGGLIRNYCRNPDPVAAPYCYTMDPNVRWEYCNLTQCSDAEGTAVAPPTVTPVPSLEAPSEQAPTEQRPG
VQECYHGNGQSYRGTYFTTVIGRTCQAWSSMTPHSHSRTPENYPNGGLIRNYCRNPDPVAAPYCYTMDPN
VRWEYCNLTQCSDAEGTAVAPPNVTPVPSLEAPSEQAPTEQRLGVQECYHGNGQSYRGTYFTTVIGRICQ
AWSSMTPHSHSRTPENYPNAGLVKNYCRNPDPVAAPWCYTTDPSVRWEYCNLTRCSDAEGTAVVPPNIIP
VPSLEAFLEQEPTEETPGVQECYYHYGQSYRGTYSTTVTGRTCQAWSSMTPHQHSRTPKNYPNAGLTRNY
CRNPDAEIRPWCYTMDPSVRWEYCNLTQCLVTESSVLETLTVVPDPSTQASSEEAPTEQSPEVQDCYHGD
GQSYRGSFSTTVTGRTCQSWSSMTPHWHQRTTEYYPDGGLTRNYCRNPDAEIRPWCYTMDPSVRWEYCNL
TQCPVTESSVLATSMAVSEQAPMEQSPGVQDCYHGDGQSYRGSFSTTVIGRICQSWSSMTPHWHQRTIEY
YPNGGLIKNYCRNPDAEIRPWCYTMDPRVRWEYCNLTQCVVMESSVLATPMVVPVPSREVPSEEAPTENS
PGVQDCYQGDGQSYRGIFSTTITGRTCQSWLSMTPHRHRRIPLRYPNAGLTRNYCRNPDAEIRPWCYTMD
PSVRWEYCNLTQCPVTESSVLTTPTVVPVPSTEAPSEQAPPEKSPVVQDCYHGDGQSYRGTSSTTVTGRN
CQSWSSMIPHWHQRTPENYPNAGLTRNYCRNPDSGKQPWCYTTDPCVRWEYCNLTQCSETESGVLETPTV
VPVPSMEAHSEAAPTEQTPVVQQCYHGNGQSYRGTFSTTVTGRTCQSWSSMTPHQHKRTPENHPNDDLTM
NYCRNPDADTGPWCFTMDPSVRREYCNLTRCSDTEGTVVTPPTVIPVPSLEAPSEQASSSFDCGKPQVEP
KKCPGSIVGGCVAHPHSWPWQVSLRTRFGKHFCGGTLISPEWVLTAACCLETFSRPSFYKVILGAHQEVN
LESHVQEIEVSRLFLEPIGADIALLKLSRPAIITDKVIPACLPSPNYVITVWTECYITGWGETQGTFGAG
LLKEAQLHVIENTVCNHYEFLNGRVKSTELCAGHLAGGTDRCQGDSGGPVVCFDKDKYILRGITSWGPGC
ACPNKPGVYVRVSSFVTWIEGVMRNN

1. A double-stranded ribonucleic acid (dsRNA) that inhibits expression of a human LPA gene by targeting a target sequence on an RNA transcript of the LPA gene, wherein the dsRNA comprises a sense strand comprising a sense sequence, and an antisense strand comprising an antisense sequence, and wherein the target sequence is nucleotides 2958-2976, 4639-4657, 4892-5000, 220-238, 223-241, 302-320, 1236-1254, 2946-2964, 2953-2971, 2954-2972, 2959-2977, 4635-4653, 4636-4654, 4842-4860, 4980-4998, 6385-6403, or 6470-6488 of SEQ ID NO: 1632, and wherein the sense sequence is at least 90% identical to the target sequence.

2. The dsRNA of claim 1, wherein the sense strand and antisense strand are complementary to each other over a region of 15-25 contiguous nucleotides.

3. The dsRNA of any one of claim 1 or 2, wherein the sense strand and the antisense strand are no more than 30 nucleotides in length.

4. The dsRNA of any one of claims 1 to 3, wherein the target sequence is nucleotides 2958-2976, 4639-4657, or 4982-5000 of SEQ ID NO: 1632.

5. The dsRNA of any one of claims 1 to 4, wherein the dsRNA comprises an antisense sequence that is at least 90% identical to a nucleotide sequence selected from the group consisting of SEQ ID NOs: 303, 306, 318, 389, 403, 406, 407, 409, 410, 467, 468, 471, 499, 520, 522, 578, and 597.

6. The dsRNA of claim 1, wherein the sense sequence and the antisense sequence are complementary, wherein:
a) the sense sequence comprises a nucleotide sequence selected from the group consisting of SEQ ID NOs: 4, 7, 19, 90, 104, 107, 108, 110, 111, 168, 169, 172, 200, 221, 223, 279, and 298; or
b) the antisense sequence comprises a nucleotide sequence selected from the group consisting of SEQ ID NOs: 303, 306, 318, 389, 403, 406, 407, 409, 410, 467, 468, 471, 499, 520, 522, 578, and 597.

7. The dsRNA of claim 6, wherein the sense strand and antisense strand of the dsRNA respectively comprise the nucleotide sequences of:
a) SEQ ID NOs: 4 and 303;
b) SEQ ID NOs: 7 and 306;
c) SEQ ID NOs: 19 and 318;
d) SEQ ID NOs: 90 and 389;
e) SEQ ID NOs: 104 and 403;
f) SEQ ID NOs: 107 and 406;
g) SEQ ID NOs: 108 and 407;
h) SEQ ID NOs: 110 and 409;
i) SEQ ID NOs: 111 and 410;
j) SEQ ID NOs: 168 and 467;
k) SEQ ID NOs: 169 and 468;
l) SEQ ID NOs: 172 and 471;
m) SEQ ID NOs: 200 and 499;
n) SEQ ID NOs: 221 and 520;
o) SEQ ID NOs: 223 and 522;
p) SEQ ID NOs: 279 and 578; or
q) SEQ ID NOs: 298 and 597.

8. The dsRNA of claim 7, wherein the sense strand and antisense strand of the dsRNA respectively comprise the nucleotide sequences of:
a) SEQ ID NOs: 110 and 409;
b) SEQ ID NOs: 172 and 471; or
c) SEQ ID NOs: 223 and 522.

9. The dsRNA of any one of claims 1 to 8, wherein the dsRNA comprises one or more modified nucleotides, wherein at least one of the one or more modified nucleotides is 2′-deoxy-2′-fluoro-ribonucleotide, 2′-deoxyribonucleotide, or 2′-O-methyl-ribonucleotide.

10. The dsRNA of any one of claims 1 to 9, wherein the dsRNA comprises an inverted 2′-deoxyribonucleotide at the 3′-end of its sense or antisense strand.

11. The dsRNA of any one of claims 1 to 10, wherein one or both of the sense strand and the antisense strand further comprise:
a) a 5′ overhang comprising one or more nucleotides; and/or
b) a 3′ overhang comprising one or more nucleotides.

12. The dsRNA of claim 11, wherein an overhang in the dsRNA comprises two or three nucleotides.

13. The dsRNA of claim 11 or 12, wherein an overhang in the dsRNA comprises one or more thymines.

14. The dsRNA of any one of claim 1 to −13, wherein the sense sequence and the antisense sequence comprise alternating 2′-O-methyl ribonucleotides and 2′-deoxy-2′-fluoro ribonucleotides.

15. The dsRNA of claim 1, wherein:
a) the sense strand comprises a nucleotide sequence selected from the group consisting of SEQ ID NOs: 602, 605, 617, 688, 702, 705, 706, 708, 709, 766, 767, 770, 798, 819, 821, 877, and 896; or
b) the antisense strand comprises a nucleotide sequence selected from the group consisting of SEQ ID NOs: 901, 904, 916, 987, 1001, 1004, 1005, 1007, 1008, 1065, 1066, 1069, 1097, 1118, 1120, 1176, and 1195.

16. The dsRNA of claim 15, wherein:
a) the sense strand comprises a nucleotide sequence selected from the group consisting of SEQ ID NOs:708, 770, and 821; or
b) the antisense strand comprises a nucleotide sequence selected from the group consisting of SEQ ID NOs: 1007, 1069, and 1120.

17. The dsRNA of claim 16, wherein the sense strand and antisense strand of the dsRNA respectively comprise the nucleotide sequences of:
a) SEQ ID NOs: 602 and 901;
b) SEQ ID NOs: 605 and 904;
c) SEQ ID NOs: 617 and 916;
d) SEQ ID NOs: 688 and 987;
e) SEQ ID NOs: 702 and 1001;
f) SEQ ID NOs: 705 and 1004;
g) SEQ ID NOs: 706 and 1005;
h) SEQ ID NOs: 708 and 1007;
i) SEQ ID NOs: 709 and 1008;
j) SEQ ID NOs: 766 and 1065;
k) SEQ ID NOs: 767 and 1066;
l) SEQ ID NOs: 770 and 1069;
m) SEQ ID NOs: 798 and 1097;
n) SEQ ID NOs: 819 and 1118;
o) SEQ ID NOs: 821 and 1120;
p) SEQ ID NOs: 877 and 1176; or
q) SEQ ID NOs: 896 and 1195.

18. The dsRNA of claim 17, wherein the sense strand and antisense strand of the dsRNA respectively comprise the nucleotide sequences of:
a) SEQ ID NOs: 708 and 1007;
b) SEQ ID NOs: 770 and 1069; or
c) SEQ ID NOs: 821 and 1120.

19. The dsRNA of any one of claims 1 to 18, wherein the dsRNA is conjugated to one or more ligands with or without a linker.

20. The dsRNA of claim 19, wherein the ligand is N-acetylgalactosamine (GalNAc) and the dsRNA is conjugated to one or more GalNAc.

21. The dsRNA of any one of claims 1 to 20, wherein the dsRNA is a small interfering RNA (siRNA).

22. The dsRNA of any one of claims 1 to 21, wherein one or both strands of the dsRNA comprise one or more compounds having the structure of 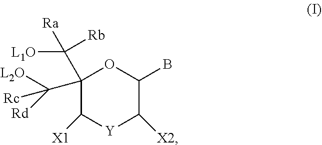
wherein:
B is a heterocyclic nucleobase,
one of L1 and L2 is an internucleoside linking group linking the compound of formula (I) to said strand(s) and the other of L1 and L2 is H, a protecting group, a phosphorus moiety or an internucleoside linking group linking the compound of formula (I) to said strand(s),
Y is O, NH, NR1 or N—C(═O)—R1, wherein R1 is:
a (C1-C20) alkyl group, optionally substituted by one or more groups selected from an halogen atom, a (C1-C6) alkyl group, a (C3-C8) cycloalkyl group, a (C3-C14) heterocycle, a (C6-C14) aryl group, a (C5-C14) heteroaryl group, —O—Z1, —N(Z1)(Z2), —S—Z1, —CN, —C(=J)-O—Z1, —O—C(=J)-Z1, —C(=J)-N(Z1)(Z2), and —N(Z1)-C(=J)-Z2, wherein
J is O or S,
each of Z1 and Z2 is, independently, H, a (C1-C6) alkyl group, optionally substituted by one or more groups selected from a halogen atom and a (C1-C6) alkyl group,
a (C3-C8) cycloalkyl group, optionally substituted by one or more groups selected from a halogen atom and a (C1-C6) alkyl group,
a group —[C(═O)]m-R2-(O—CH₂—CH₂)p-R3, wherein
m is an integer meaning 0 or 1,
p is an integer ranging from 0 to 10,
R2 is a (C1-C20) alkylene group optionally substituted by a (C1-C6) alkyl group, —O—Z3, —N(Z3)(Z4), —S—Z3, —CN, —C(═K)—O—Z3, —O—C(═K)—Z3, —C(═K)—N(Z3)(Z4), or —N(Z3)-C(═K)—Z4, wherein
K is O or S,
each of Z3 and Z4 is, independently, H, a (C1-C6) alkyl group, optionally substituted by one or more groups selected from a halogen atom and a (C1-C6) alkyl group,
and
R3 is selected from the group consisting of a hydrogen atom, a (C1-C6) alkyl group, a (C1-C6) alkoxy group, a (C3-C8) cycloalkyl group, a (C3-C14) heterocycle, a (C6-C14) aryl group or a (C5-C14) heteroaryl group,
or R3 is a cell targeting moiety,
X1 and X2 are each, independently, a hydrogen atom, a (C1-C6) alkyl group, and
each of Ra, Rb, Rc and Rd is, independently, H or a (C1-C6) alkyl group, or a pharmaceutically acceptable salt thereof.

23. The dsRNA of claim 22, comprising one or more compounds of formula (I) wherein Y is
a) NR1, R1 is a non-substituted (C1-C20) alkyl group;
b) NR1, R1 is a non-substituted (C1-C16) alkyl group, which includes an alkyl group selected from a group comprising methyl, isopropyl, butyl, octyl, and hexadecyl;
c) NR1, R1 is a (C3-C8) cycloalkyl group, optionally substituted by one or more groups selected from a halogen atom and a (C1-C6) alkyl group;
d) NR1, R1 is a cyclohexyl group;
e) NR1, R1 is a (C1-C20) alkyl group substituted by a (C6-C14) aryl group;
f) NR1, R1 is a methyl group substituted by a phenyl group;
g) N—C(═O)—R1, R1 is an optionally substituted (C1-C20) alkyl group; or
h) N—C(═O)—R1, R1 is methyl or pentadecyl.

24. The dsRNA of claim 22 or 23, comprising one or more compounds of formula (I) wherein B is selected from a group consisting of a pyrimidine, a substituted pyrimidine, a purine and a substituted purine, or a pharmaceutically acceptable salt thereof.

25. The dsRNA of any one of claims 22 to 24, wherein R3 is of the formula (II): 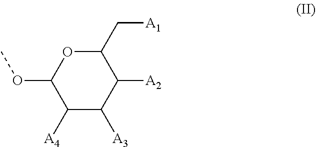
wherein A1, A2 and A3 are OH,
A4 is OH or NHC(═O)—R5, wherein R5 is a (C1-C6) alkyl group, optionally substituted by an halogen atom, or a pharmaceutically acceptable salt thereof.

26. The dsRNA of any one of claims 22 to 25, wherein R3 is N-acetyl-galactosamine, or a pharmaceutically acceptable salt thereof.

27. The dsRNA of any one of claims 22 to 26, comprising one or more nucleotides from Table A.

28. The dsRNA of claims 22 to 27, comprising from 2 to 10 compounds of formula (I), or a pharmaceutically acceptable salt thereof.

29. The dsRNA of claim 28, wherein the 2 to 10 compounds of formula (I) are on the sense strand.

30. The dsRNA of any one of claims 22 to 29, wherein the sense strand comprises two to five compounds of formula (I) at the 5′ end, and/or comprises one to three compounds of formula (I) at the 3′ end.

31. The dsRNA of claim 30, wherein
a) the two to five compounds of formula (I) at the 5′ end of the sense strand comprise lgT3, optionally comprising three consecutive lgT3 nucleotides; and/or
b) the one to three compounds of formula (I) at the 3′ end of the sense strand comprise 1T4; optionally comprising two consecutive 1T4.

32. The dsRNA of any one of claims 1 to 31, comprising one or more internucleoside linking groups independently selected from the group consisting of phosphodiester, phosphotriester, phosphorothioate, phosphorodithioate, alkyl-phosphonate and phosphoramidate backbone linking groups, or a pharmaceutically acceptable salt thereof.

33. The dsRNA of any one of claims 1 to 32, selected from the dsRNAs in Tables 1-4.

34. The dsRNA of any one of claims 1 to 33, wherein the sense strand and antisense strand of the dsRNA respectively comprise the nucleotide sequences of:
a) SEQ ID NOs: 1231 and 1429;
b) SEQ ID NOs: 1307 and 1505;
c) SEQ ID NOs: 1308 and 1506;
d) SEQ ID NOs: 1325 and 1523;
e) SEQ ID NOs: 1328 and 1526; or
f) SEQ ID NOs: 1369 and 1567.

35. A pharmaceutical composition comprising the dsRNA of any one of claims 1 to 34 and a pharmaceutically acceptable excipient.

36. The dsRNA of any one of claims 1 to 34 or the composition of claim 35 for their use in inhibiting LPA expression, reducing Lp(a) levels, or treating an Lp(a)-associated condition in a human in need thereof.

37. The dsRNA or composition for their use according to claim 36, wherein the human has or is at risk of having a lipid metabolism disorder or a cardiovascular disease (CVD).

38. The dsRNA or composition for their use according to claim 36, wherein the human has or is at risk of having hypercholesterolemia, dyslipidemia, myocardial infarction, atherosclerotic cardiovascular disease, atherosclerosis, peripheral artery disease, calcific aortic valve disease, thrombosis, or stroke.

39. A method of treating and/or preventing one or more Lp(a)-associated conditions comprising administering one or more dsRNAs as defined in any one of claims 1 to 34, and/or one or more pharmaceutical compositions as defined in claim 35.
